(19) United States
(12) Patent Application Publication
(10) Pub. No.: US 2025/0152658 A1
(43) Pub. Date: May 15, 2025

(54) CYCLIC PEPTIDE FOR TRAPPING INTERLEUKIN-1 BETA

(71) Applicant: Merck Sharp & Dohme LLC, Rahway, NJ (US)

(72) Inventors: Faben A. Cruz, Burlingame, CA (US); Danqing Feng, Green Brook, NJ (US); Yan Guo, Westfield, NJ (US); Jennifer Hanisak, Stewartsville, NJ (US); Jennifer L. Hickey, Fords, NJ (US); Ahmet Kekec, Hoboken, NJ (US); Bing Li, Towaco, NJ (US); Michael Man-Chu Lo, Bedminster, NJ (US); Dmitri A. Pissarnitski, Scotch Plains, NJ (US); Christopher W. Plummer, Westfield, NJ (US); Stefania Colarusso, Rome (IT); Dipannita Kalyani, Union, NJ (US); Quang T. Truong, Morganville, NJ (US)

(73) Assignee: Merck Sharp & Dohme LLC, Rahway, NJ (US)

(21) Appl. No.: 18/944,230

(22) Filed: Nov. 12, 2024

Related U.S. Application Data
(60) Provisional application No. 63/598,567, filed on Nov. 14, 2023.
(60) Provisional application No. 63/717,339, filed on Nov. 7, 2024.

(51) Int. Cl. A61K 38/08; C07K 7/64

(52) U.S. Cl. CPC A61K 38/08; C07K 7/64

(57) ABSTRACT

Provided are compounds of the Formula (I), or their pharmaceutically acceptable salts, wherein X¹, X², X³, R¹-R⁹, R^{A}, R^{B}, R^{D}, A¹, A², and subscripts t and w are as herein described.

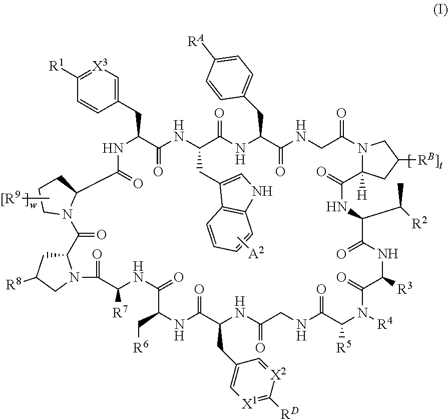

The compounds and their pharmaceutically acceptable salts can trap IL-1β and are expected to have utility as therapeutic agents, for example, for treating cardiovascular disease and inflammatory disorders. The disclosure also provides pharmaceutical compositions which comprise the compounds disclosed herein or pharmaceutically acceptable salts thereof. The disclosure also relates to methods for use of the compounds or their pharmaceutically acceptable salts in the therapy and prophylaxis of cardiovascular disease and inflammatory disorders and for preparing pharmaceuticals for this purpose.

### CROSS-REFERENCE TO RELATED APPLICATIONS

This application claims the benefit of U.S. Provisional Patent Application Nos. 63/598,567 filed Nov. 14, 2023, and 63/717,339 filed Nov. 7, 2024, the entire contents of which are incorporated by reference herein.

### REFERENCE TO SEQUENCE LISTING SUBMITTED ELECTRONICALLY

The contents of the electronic sequence listing (25836-WO-PCT_SL.xml; Size: 2,104,813 bytes; and Date of Creation: Oct. 31, 2024) are herein incorporated by reference in their entirety.

### FIELD

The present disclosure relates to certain cyclic peptides that trap interleukin-1β (IL-1β), pharmaceutical compositions comprising such peptides, and methods for using the compounds for treating, inhibiting, or ameliorating one or more cardiovascular disease states that could benefit from trapping IL-1β, including atherosclerosis.

### BACKGROUND

Atherosclerosis is a disease of the arteries characterized by the accumulation of cholesterol plaques on the interior wall of the artery. Progression of atherosclerosis can result in hardening or narrowing of the arteries and increases the risk of plaque ruptures. These ruptures release cholesterol globules and other material into the bloodstream which may result in blockage of blood flow to the brain, heart, or other organ. Medically, these are known as Major Adverse Cardiac Events (MACE).

Risk factors for the development and progression of Atherosclerotic Cardiovascular Disease (ASCVD) include high cholesterol, high blood pressure, diet high in saturated fat, smoking, obesity, diabetes, lack of exercise, and elevated levels of C-reactive protein (CRP), a marker of inflammation.

The first line of treatment to prevent the progression of ASCVD is a healthy diet and exercise, however, compliance is generally poor. Pharmacological treatments for ASCVD have largely focused on cholesterol-lowering medications such as statins, cholesterol absorption inhibitors, and low-density lipoprotein (LDL) receptor inhibitors. These medications are highly effective at reducing the buildup of fatty acid deposits and improving arterial health. Other medications that are prescribed for ASCVD which do not ameliorate the disease state include blood thinners, such as aspirin, to prevent clumping of platelets in narrow arteries, and blood pressure medications to reduce the risk and severity of heart attacks. Surgical options for more aggressive intervention in advanced cases of ASCVD include angioplasty, stent placement, endarterectomy (surgical removal of plaques), and bypass surgery.

While cholesterol-lowering medications have served as an important standard of care for slowing the progression of atherosclerosis, clinical data support an additional critical role for inflammation in the progression of ASCVD that has remained untreated. Biomarkers of inflammation such as CRP are associated with increased risk of cardiovascular events, independent of cholesterol levels. The Canakinumab Anti-inflammatory Thrombosis Outcomes Study (CANTOS) was the first clinical trial to show that reducing vascular inflammation in the absence of concomitant lipid lowering reduces the rates of cardiovascular events. N Engl J Med 2017; 377:1119-1131. Canakinumab is an anti-interleukin-1 beta (IL-1β) human monoclonal antibody approved for clinical use in rheumatologic disorders. IL-1β is a proinflammatory cytokine that induces IL-6 and thereby elevates the downstream inflammatory biomarker high sensitivity CRP (hsCRP). Therefore, CANTOS provides proof of concept that therapies targeting IL-1β could reduce rates of MACE in certain patients in a manner that is complimentary and potentially additive to the LDL-lowering standard of care.

There is a need for additional, non-surgical therapeutic approaches beyond the standard of care cholesterol-lowering medications for slowing the progression of atherosclerosis and decreasing the risk of MACE. In addition, patients suffering from inflammatory disorders would benefit from orally administered agents which block the same cytokine, IL-1β, as canakinumab.

### SUMMARY

The present disclosure provides certain cyclic peptides that reduce inflammation by binding to the IL-1β cytokine and prevent engagement with the TL-1 receptor, resulting in inhibition of downstream pro-inflammatory signaling. These cyclic peptides can be valuable pharmaceutically active compounds for the treatment of cardiovascular diseases and inflammatory disorders. In one aspect, the present disclosure provides compounds of Formula (I)

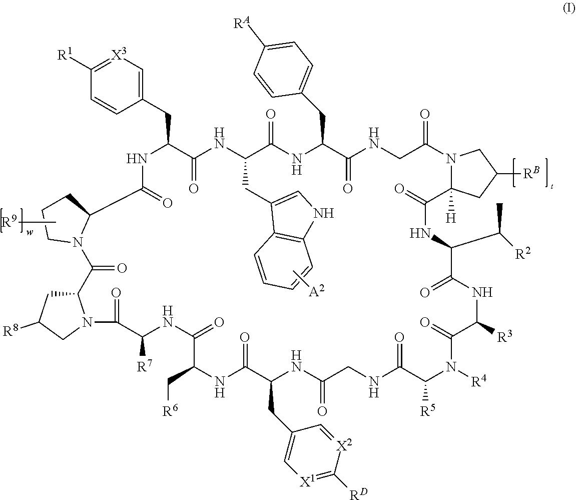

and their pharmaceutically acceptable salts.

The compounds can trap IL-1β and thereby affect the downstream pro-inflammatory signaling pathway which may be associated with cardiovascular disorders. Accordingly, in another aspect, the present disclosure provides a method for treating a cardiovascular disorder (e.g., atherosclerosis, vascular inflammation) comprising administering a therapeutically effective amount of the compound of the disclosure to a subject in need thereof. In some embodiments, the administration comprises an oral administration of the compound.

The disclosure furthermore provides processes for preparing compounds of the disclosure and pharmaceutical compositions which comprise compounds of the disclosure and a pharmaceutically acceptable carrier.

### BRIEF DESCRIPTION OF THE DRAWINGS

FIG. 1 describes the amino acid sequences of SEQ ID NOS: 1-465 and their associated properties.

### DETAILED DESCRIPTION

### Compounds of the Disclosure

In one embodiment, the present disclosure provides a compound having structural Formula (I) as shown above wherein:
R¹ is
   (i) R^{1a}—C(O)N(H)—CH₂CH₂—O—, wherein
      R^{1a} is
         (a) C₁-C₃ alkyl or
         (b) (CH₃)₃N—CH₂CH₂-M-, wherein:
         M is —CH₂—, —CH₂CH₂—, —CH₂CH₂CH₂—, —O—CH₂CH₂—, or —O—CH₂CH₂—O—CH₂CH₂—;
   (ii) (CH₃)₃N—CH₂CH₂—O—;
   (iii) C¹, wherein C¹ is:
      (a) phenyl or a 5- to 6-membered monocyclic heteroaryl, wherein said 5- to 6-membered monocyclic heteroaryl contains 1 to 3 heteroatoms independently selected from the group consisting of N, O, and S;
      (b) a 5- to 6-membered heterocycloalkyl, wherein said 5- to 6-membered heterocycloalkyl is saturated and contains 1 or 2 heteroatoms selected from the group consisting of N, O, and S;
         wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, C₂-C₃ acyl, and C₁-C₃ alkoxymethyl;
   (iii) (CH₃)₃N—CH₂CH₂—O—; or
   (iv) a group

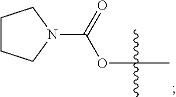 [SMILES: *OC(=O)N1CCCC1]

R^{A} is:
   (i) —CO₂H;
   (ii) —CO₂NH₂;
   (iii) —S(O)₂NH₂; or
   (iv) a 5-membered heteroaryl containing 2 or 3 heteroatoms independently selected from the group consisting of N, O, and S;
      wherein the 5-membered heteroaryl is unsubstituted or substituted by 1 to 2 substituents independently selected from the group consisting of halo, C₁-C₃ alkyl, C₁-C₃ alkoxy, oxo, and C₁-C₃ alkoxymethyl;
R^{B} is amino, hydroxy, or fluoro;
R² is
   (i) naphthyl; or
   (ii) a 9- to 10-membered bicyclic heteroaryl, wherein said 9- to 10-membered bicyclic heteroaryl contains 1 to 2 heteroatoms independently selected from the group consisting of N, O, and S;
      wherein R² is unsubstituted or substituted by 1 to 2 R^{2a} substituents independently selected from the group consisting of halo, C₁-C₃ alkyl and C₁-C₃ alkoxy;
R³ is —(CH₂)ₘCO₂H, —(CH₂)ₘCO₂NH₂, —(CH₂)ₘCO₂N(CH₃)₂, —(CH₂)ₘCH₂N(H)C(O)NH₂, or —(CH₂)ₘ—C³;
   wherein C³ is a 5-membered heteroaryl containing 2 to 4 heteroatoms selected from the group consisting of N, O, and S;
      wherein C³ is unsubstituted or substituted by 1 to 2 substituent selected from the group consisting of halo and C₁-C₃ alkyl;
R⁴ is C₁-C₃ alkyl;
R⁵ is H, C₁-C₃ alkyl, or —CH₂CO₂H;
   or alternatively R⁴ and R⁵ together with the atoms to which they are attached form a pyrrolidinyl, piperidinyl or azepinyl ring;
X¹, X², and X³ are independently C(H) or N;
R^{D} is:
   (i) ring C^{D}, wherein ring C^{D} is:
      (a) phenyl; or
      (b) cyclohexyl;
      wherein ring C^{D} is substituted by one A¹; wherein A¹ is —CO₂H, —CONH₂, or —N(H)SO₂CH₃;
   (ii) hydroxy;
   (iii) —CO₂NH₂; or
   (iv) —OCH₂CH₂C(O)N(H)C(O)CH₃;
A² is H, halo or C₁-C₃ alkyl;
R⁶ is —H, —NH₂, —C(O)N(CH₃)₂; —(CH₂)ₙNH₂; —(CH₂)ₙN(H)CH₃; —(CH₂)ₙN(CH₃)₃; —OH; —(CH₂)ₙOH; —OCH₃; —(CH₂)ₙOCH₃; —(CH₂)ₙCH₃; —(CH₂)ₙN(H)C(O)NH₂; —(CH₂)ₙCH₂C(O)OH; —(CH₂)ₙN(H)C(O)CH₂CH₂O(CH₂CH₂—O)q-CH₂CH₂N(CH₃)₃; or —C⁶;
   C⁶ is
      (i) a 5- to 6-membered monocyclic aryl or heteroaryl, wherein said 5- to 6-membered monocyclic heteroaryl contains 1 to 3 heteroatoms independently selected from the group consisting of N, O, and S;
      (ii) a 9- to 10-membered bicyclic aryl or heteroaryl, wherein said 9- to 10-membered bicyclic heteroaryl contains 1 to 4 heteroatoms independently selected from the group consisting of N, O, and S; and
      (iii) a 3- to 8-membered mono- or bicyclic cycloalkyl;
         wherein C⁶ is unsubstituted or substituted by 1 to 3 R^{C6} substituents independently selected from the group consisting of halo, amino, hydroxy, carboxy, C₁-C₃ alkyl, C₁-C₃ fluoroalkyl, C₁-C₃ alkoxy, and C₁-C₃ alkoxymethyl;
R⁷ is H, C₁-C₄ alkyl, —(CH₂)ᵤOH, —(CH₂)ᵤC(O)N(CH₃)₂, —(CH₂)ᵤN(CH₃)₃, —(CH₂)ᵤOCH₃, —(CH₂)ᵤC(O)NC(O)NH₂, —(CH₂)ᵤN(H)C(O)CH₂CH₂O(CH₂CH₂—O)ᵥ—CH₂CH₂N(CH₃)₃, or —CH₂—C⁷;
   C⁷ is:
      (i) a 5-membered heteroaryl containing 2 to 4 heteroatoms selected from the group consisting of N, O, and S; or
      (ii) a 5- to 6-membered heterocycloalkyl, wherein said 5- to 6-membered heterocycloalkyl is saturated and contains 1 O atom;
      wherein C⁷ is unsubstituted or substituted by 1 to 2 substituents selected from the group consisting of halo, C₁-C₃ alkyl, C₁-C₃ alkoxy, and C₁-C₃ alkoxymethyl;
R⁸ is —H, —F, —OH, —NH₂, —N(CH₃)₃, (CH₃)₃N—(CH₂)ᵣ—C(O)—, or (CH₃)₃N—CH₂CH₂—O—(CH₂CH₂—O)ₛ—CH₂CH₂—C(O)—
R⁹ is —C⁹ or —CH₂—C⁹;
   C⁹ is phenyl or a 5- to 6-membered monocyclic heteroaryl, wherein said 5- to 6-membered monocyclic heteroaryl contains 1 to 2 heteroatoms independently selected from the group consisting of N, O, and S;
      wherein C⁹ is unsubstituted or substituted by 1 to 3 R^{C9} substituents independently selected from the group consisting of halo, amino, hydroxy, C₁-C₃ alkyl, C₁-C₃ fluoroalkyl, and C₁-C₃ alkoxy;
      wherein C⁹ is optionally substituted by 1 ring R^{C9C}, the ring R^{C9C} is:
         (a) phenyl; or
         (b) a 5- to 6-membered monocyclic heteroaryl containing 1 to 2 heteroatoms independently selected from the group consisting of N, O, and S;
subscript m is 1 or 2;
subscript n is 1, 2, or 3; and
subscript q is 1, 2, or 3;
subscript r is 3, 4, 5, or 6;
subscript s is 1 or 2;
subscript t is 0 or 1;
subscript u is 0, 1, 2, 3, or 4;
subscript v is 1, 2, or 3; and
subscript w is 0 or 1; or
   a pharmaceutically acceptable salt thereof.

In another embodiment, the present disclosure provides a compound Formula (I), wherein:
C¹ is phenyl;
R² is indolyl or naphthyl;
C⁶ is phenyl, indolyl, pyridyl, pyridazinyl, or bicyclo[1.1.1]pentanyl; and
C⁹ is phenyl or pyrimidinyl.

In another embodiment, the present disclosure provides a compound of Formula (I), wherein R¹ is CH₃—C(O)N(H)—CH₂CH₂—O— or 4-fluorophenyl.

In another embodiment, the present disclosure provides a compound of Formula (I), wherein R¹ is (CH₃)₃N—CH₂CH₂-M-, wherein M is CH₂—, —CH₂CH₂CH₂—, —O—CH₂CH₂—, or —O—CH₂CH₂—O—CH₂CH₂—.

In another embodiment, the present disclosure provides a compound of Formula (I), wherein R¹ is C¹.

In another embodiment, the present disclosure provides a compound of Formula (I), wherein A² is H, fluoro, chloro, or methyl.

In another embodiment, the present disclosure provides a compound of Formula (I), wherein R² is unsubstituted or substituted naphthyl or indolyl.

In another embodiment, the present disclosure provides a compound of Formula (I), wherein R³ is —(CH₂)ₘCO₂H.

In another embodiment, the present disclosure provides a compound of Formula (I), wherein subscript m is 1.

In another embodiment, the present disclosure provides a compound of Formula (I), wherein R⁴ and R⁵ are methyl.

In another embodiment, the present disclosure provides a compound of Formula (I), wherein:
X¹ and X² are C(H) and
A¹ is carboxy.

In another embodiment, the present disclosure provides a compound of Formula (I), wherein:
(i) a substituted or unsubstituted 5- to 6-membered monocyclic aryl or heteroaryl, wherein said 5- to 6-membered monocyclic heteroaryl contains 1 to 2 N atoms; or
(ii) a substituted or unsubstituted 9- to 10-membered bicyclic heteroaryl, wherein said bicyclic heteroaryl contains 1 to 2 N atoms.

In another embodiment, the present disclosure provides a compound of Formula (I), wherein R⁶ is indolyl.

In another embodiment, the present disclosure provides a compound of Formula (I), wherein R⁷ is H.

In another embodiment, the present disclosure provides a compound of Formula (I), wherein R⁸ is —H, —OH, —NH₂, (CH₃)₃N—(CH₂)₅—C(O)—, or (CH₃)₃N—CH₂CH₂—O—CH₂CH₂—O—CH₂CH₂—C(O)—.

In another embodiment, the present disclosure provides a compound of Formula (I), wherein R⁸ is H or —NH₂.

In another embodiment, the present disclosure provides a compound of Formula (I), wherein R⁹ is substituted on position 4 of the illustrated pyrrolidinyl ring.

In another embodiment, the present disclosure provides a compound of Formula (I), wherein C⁹ is substituted or unsubstituted phenyl or pyrimidinyl.

In another embodiment, the present disclosure provides a compound of Formula (I), wherein R⁹ is —CH₂-(4-fluorophenyl) or pyrimidin-5-yl.

In another embodiment, the present disclosure provides a compound of Formula (I), wherein:
R¹ is CH₃—C(O)N(H)—CH₂CH₂—O— or 4-fluorophenyl;
R² is unsubstituted or substituted naphthyl or indolyl;
R³ is —(CH₂)ₘCO₂H;
R⁴ and R⁵ are methyl;
X¹ and X² are C(H);
A¹ is carboxy;
A² is H;
R⁶ is
   (i) a substituted or unsubstituted 5- to 6-membered monocyclic aryl or heteroaryl, wherein said 5- to 6-membered monocyclic heteroaryl contains 1 to 2 N atoms; or
   (ii) a substituted or unsubstituted 9- to 10-membered bicyclic heteroaryl, wherein said 9- to 10-membered bicyclic heteroaryl contains 1 to 2 N atoms;
R⁷ is H;
R⁸ is H or —NH₂; and
R⁹ is substituted on position 4 of the illustrated pyrrolidinyl ring.

In one aspect of this embodiment, the present disclosure provides a compound of Formula (I), wherein:
R⁶ is indolyl;
R⁹ is —CH₂-(4-fluorophenyl) or pyrimidin-5-yl, substituted on the 4-position of the illustrated pyrrolidinyl ring; and
subscript m is 1.

In some embodiments, the compound of Formula (I) has Formula (IA)

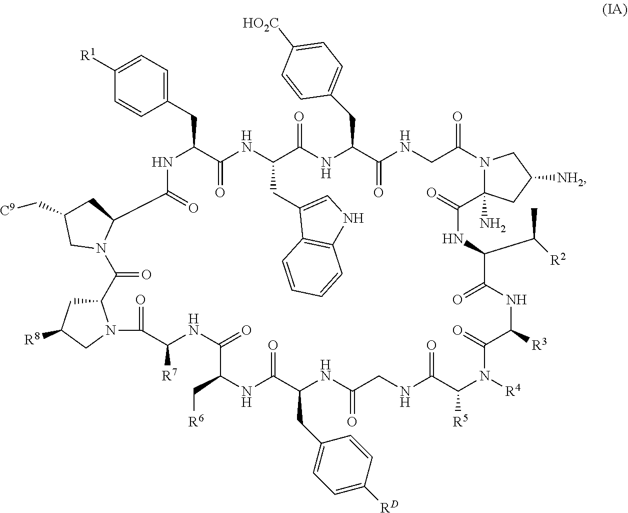

wherein:
R¹ is:
   (i) R^{1a}—C(O)N(H)—CH₂CH₂—O—, wherein R^{1a} is:
      (a) methyl; or
      (b) (CH₃)₃N—CH₂CH₂—O—CH₂CH₂—;
   (ii) (CH₃)₃N—CH₂CH₂—O—; or
   (iii) 4-carboxyphenyl;
R² is a group

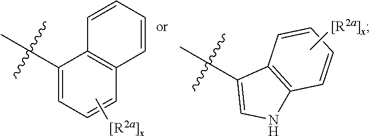

R^{2a} is halo or methyl;
subscript x is 0 or 1;
R³ is —CH₂CO₂H or —CH₂-tetrazolyl;
R⁴ and R⁵ are methyl;
   or alternatively R⁴ and R⁵ together with the atoms to which they are attached form a piperidinyl ring;
R^{D} is 4-carboxyphenyl or hydroxy;
R⁶ is a group selected from:

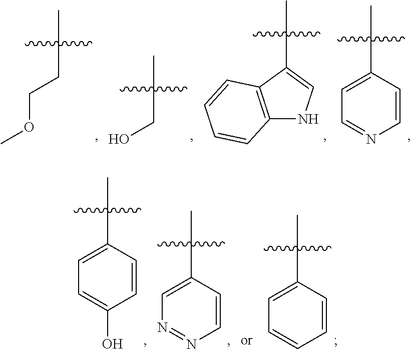

R⁷ is H or methyl;
R⁸ is —H, —NH₂, or —OH;
C⁹ is a group selected from

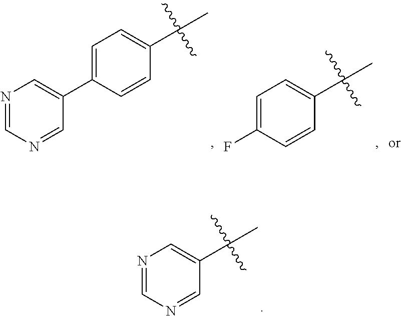 [SMILES: *c1ccc(-c2cncnc2)cc1 ; *c1ccc(F)cc1 ; *c1cncnc1]

In other embodiments, the compound of Formula (I) has Formula (IB):

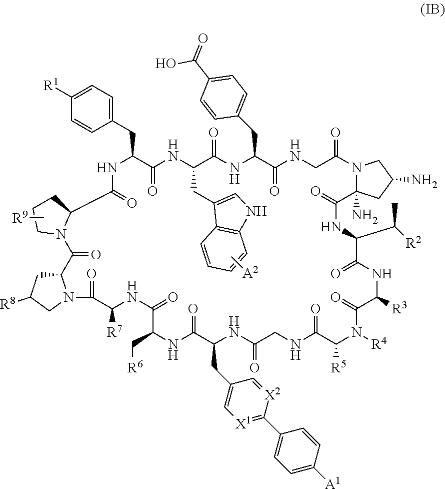

wherein:
R¹ is
   (i) R^{1a}—C(O)N(H)—CH₂CH₂—O—, wherein
      R^{1a} is
         (a) C₁-C₃ alkyl or
         (b) (CH₃)₃N—CH₂CH₂-M-, wherein
         M is —CH₂—, —CH₂CH₂—, —CH₂CH₂CH₂—, —O—CH₂CH₂—, or —O—CH₂CH₂—O—CH₂CH₂—; or
   (ii) C¹, wherein C¹ is phenyl or a 5- to 6-membered monocyclic aryl or heteroaryl, wherein said 5- to 6-membered monocyclic heteroaryl contains 1 to 2 heteroatoms independently selected from the group consisting of N, O, and S;
      wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, and C₂-C₃ acyl;
R² is
   (i) naphthyl; or
   (ii) a 9- to 10-membered bicyclic heteroaryl, wherein said 9- to 10-membered bicyclic heteroaryl contains 1 to 2 heteroatoms independently selected from the group consisting of N, O, and S;
      wherein R² is unsubstituted or substituted by 1 to 2 R^{2a} substituents independently selected from the group consisting of halo;
R³ is —(CH₂)ₘCO₂H, —(CH₂)ₘCO₂NH₂, —(CH₂)ₘCO₂N(CH₃)₂, or —(CH₂)ₘ-tetrazolyl;
R⁴ is C₁-C₃ alkyl;
R⁵ is H or C₁-C₃ alkyl;
   or alternatively R⁴ and R⁵ together with the atoms to which they are attached form a pyrrolidinyl, piperidinyl or azepinyl ring;
X¹ and X² are independently C(H) or N;
A¹ is —CO₂H or —N(H)SO₂CH₃;
A² is H, halo or C₁-C₃ alkyl;
R⁶ is —(CH₂)ₙNH₂; —(CH₂)ₙN(H)CH₃; —(CH₂)ₙOH; —(CH₂)ₙOCH₃; —(CH₂)ₙN(H)CH₂CH₂O(CH₂CH₂—O)q-CH₂CH₂N(CH₃)₃; or —C⁶;
   C⁶ is
      (i) a 5- to 6-membered monocyclic aryl or heteroaryl, wherein said 5- to 6-membered monocyclic heteroaryl contains 1 to 2 heteroatoms independently selected from the group consisting of N, O, and S;
      (ii) a 9- to 10-membered bicyclic aryl or heteroaryl, wherein said 9- to 10-membered bicyclic heteroaryl contains 1 to 4 heteroatoms independently selected from the group consisting of N, O, and S; and
      (iii) a 3- to 8-membered mono- or bicyclic cycloalkyl;
         wherein C⁶ is unsubstituted or substituted by 1 to 3 R^{C6} substituents independently selected from the group consisting of halo, amino, hydroxy, carboxy, C₁-C₃ alkyl, C₁-C₃ fluoroalkyl, and C₁-C₃ alkoxy;
R⁷ is H or C₁-C₄ alkyl;
R⁸ is —H, —OH, —NH₂, (CH₃)₃N—(CH₂)ᵣ—C(O)—, or (CH₃)₃N—CH₂CH₂—O—(CH₂CH₂—O)ₛ—CH₂CH₂—C(O)—
R⁹ is —C⁹ or —CH₂—C⁹;
   C⁹ is phenyl or a 5- to 6-membered monocyclic heteroaryl, wherein said 5- to 6-membered monocyclic heteroaryl contains 1 to 2 heteroatoms independently selected from the group consisting of N, O, and S;
      wherein C⁹ is unsubstituted or substituted by 1 to 3 R^{C9} substituents independently selected from the group consisting of halo, amino, hydroxy, C₁-C₃ alkyl, C₁-C₃ fluoroalkyl, C₁-C₃ alkoxy, and phenyl;
subscript m is 1 or 2;
subscript n is 1, 2, or 3; and
subscript q is 1 or 2;
subscript r is 3, 4, 5, or 6;
subscript s is 1 or 2; or
   a pharmaceutically acceptable salt thereof.

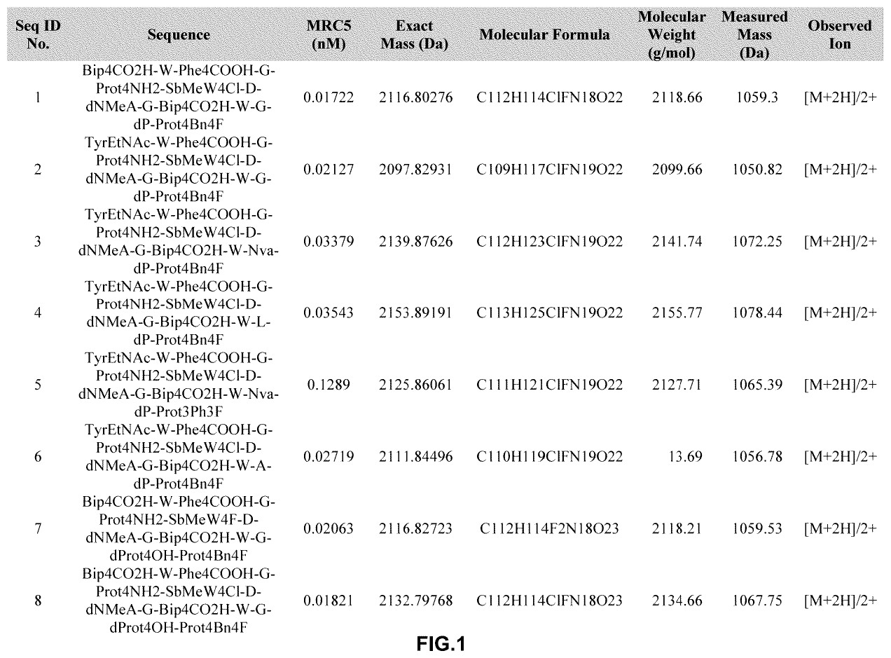

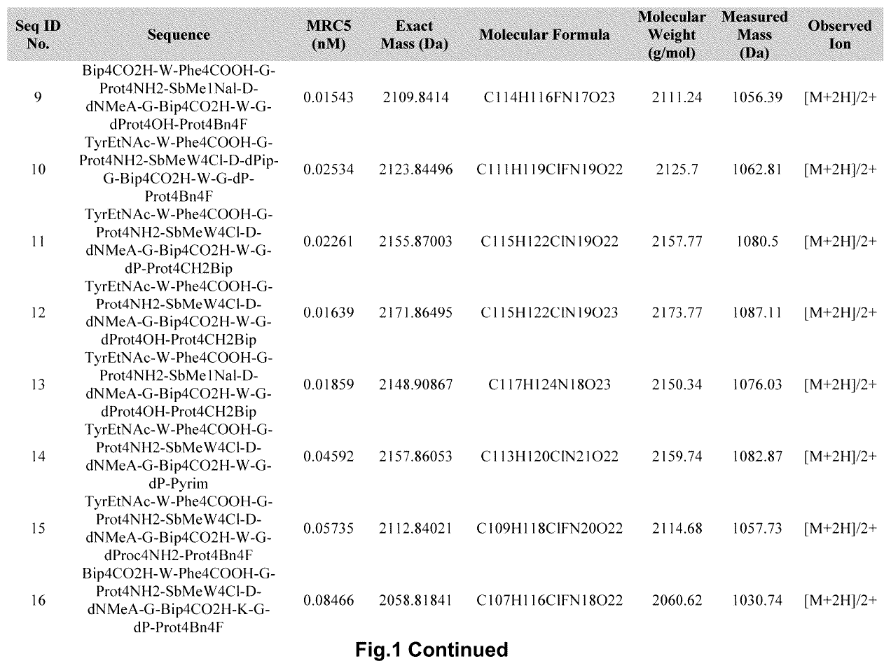

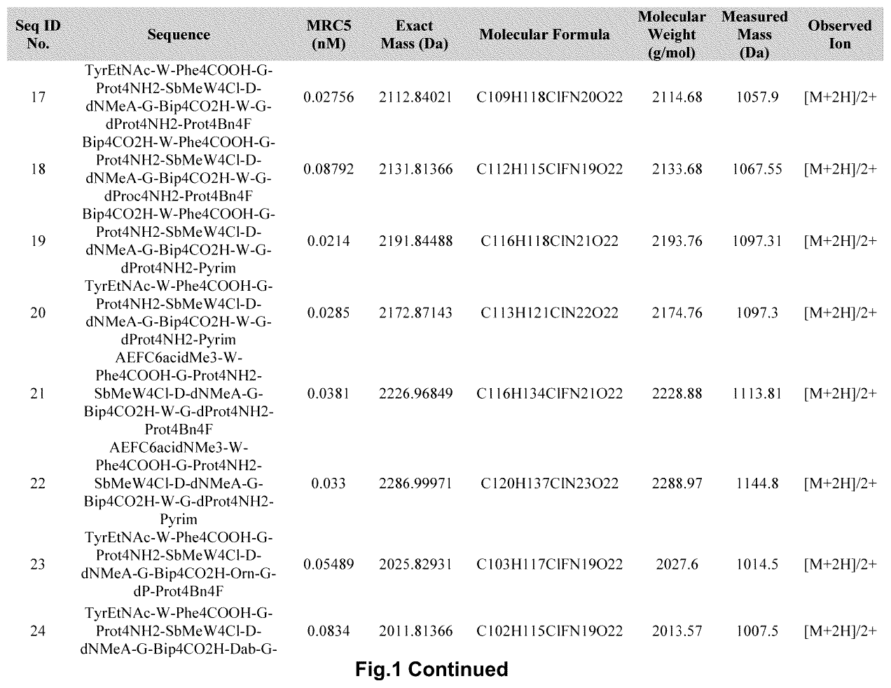

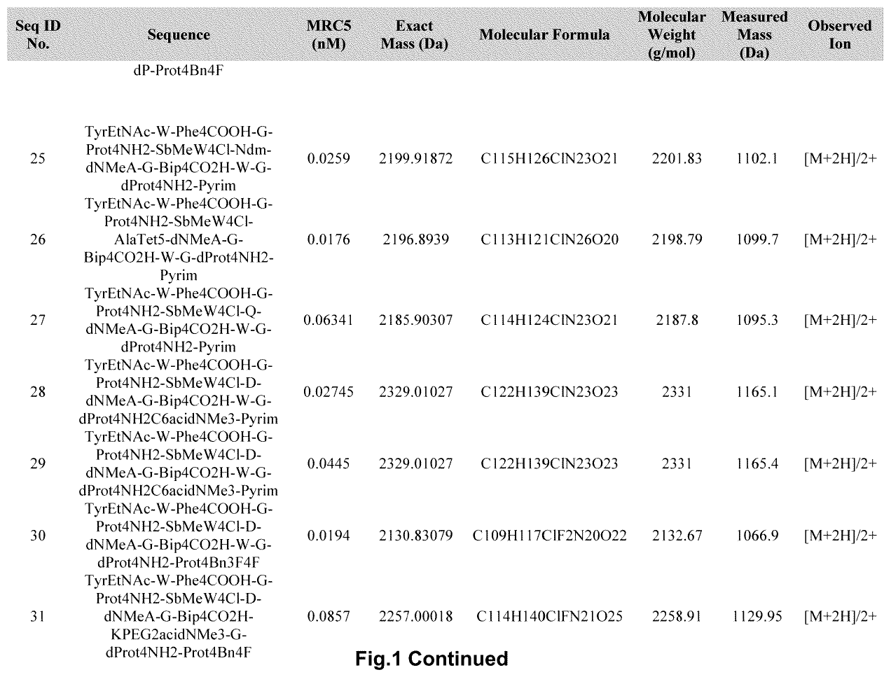

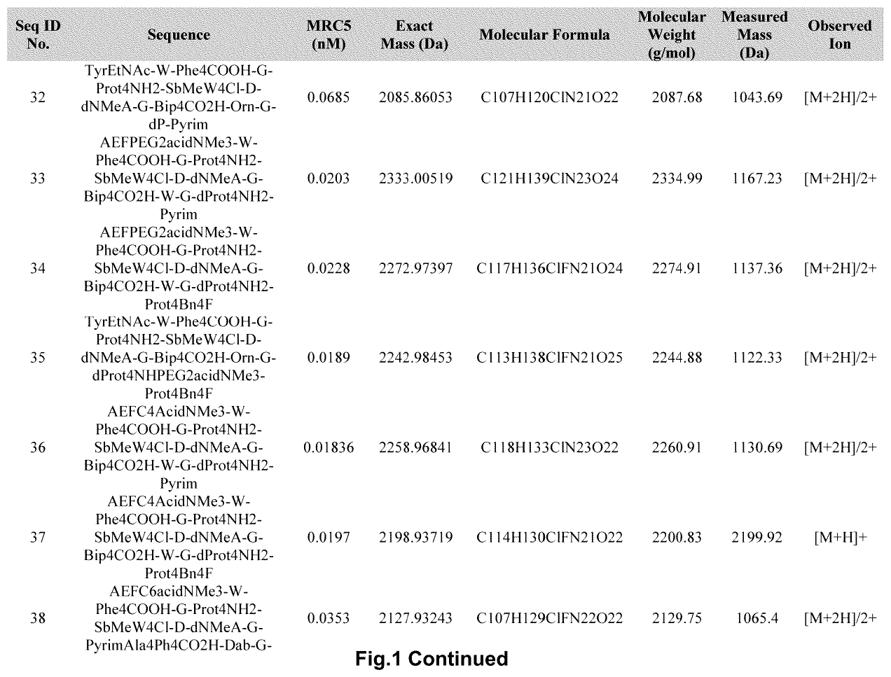

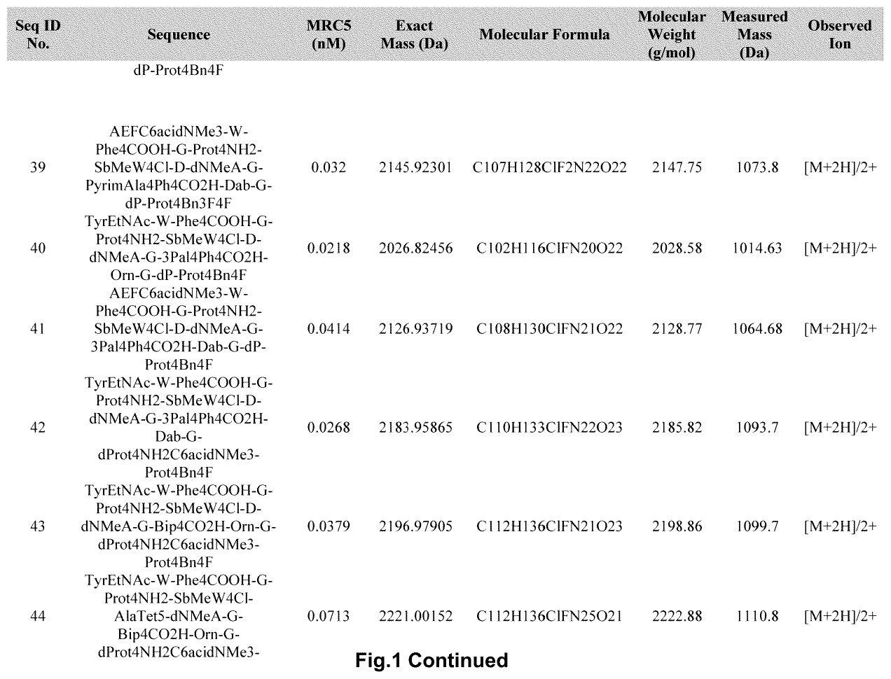

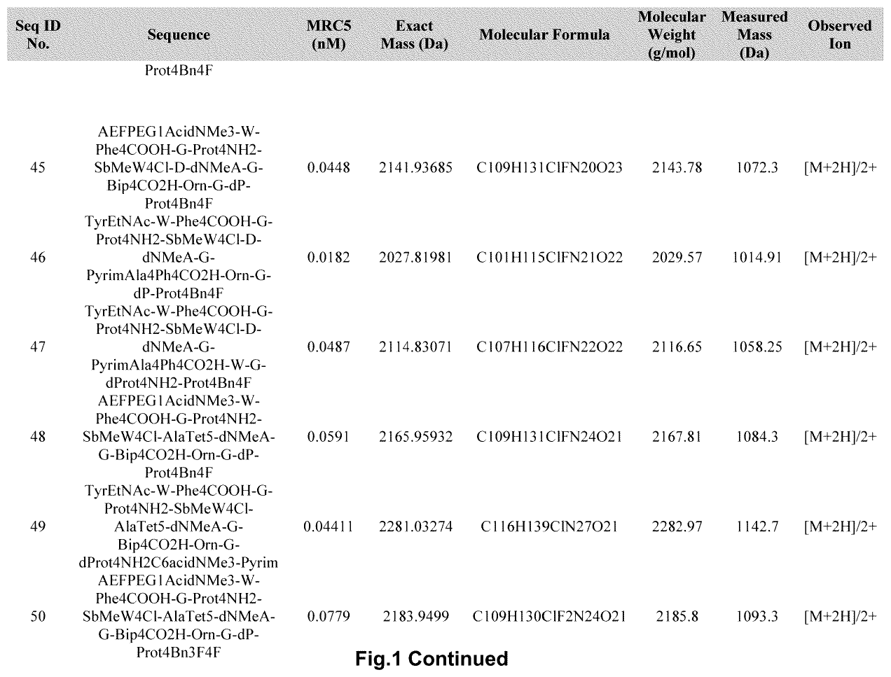

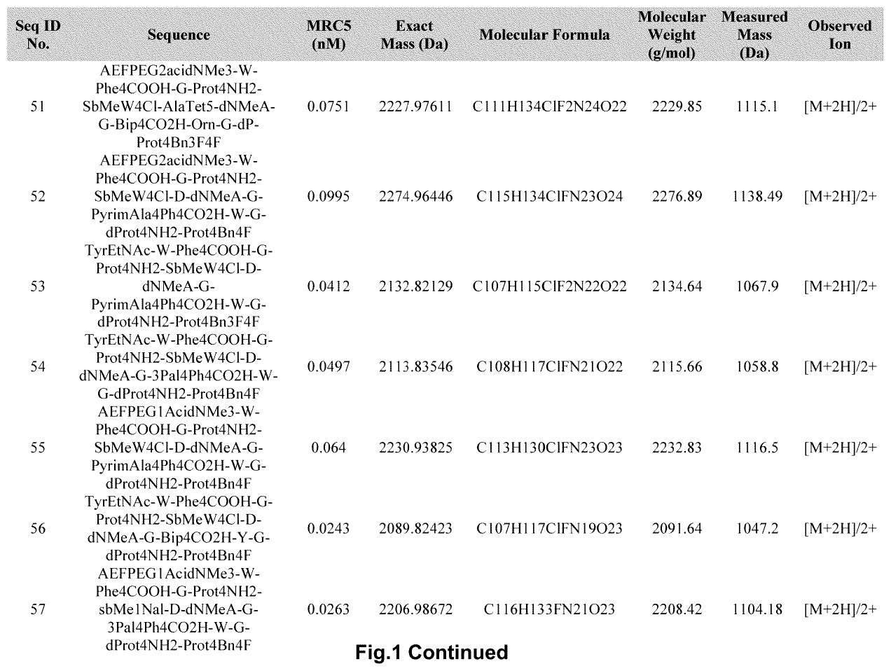

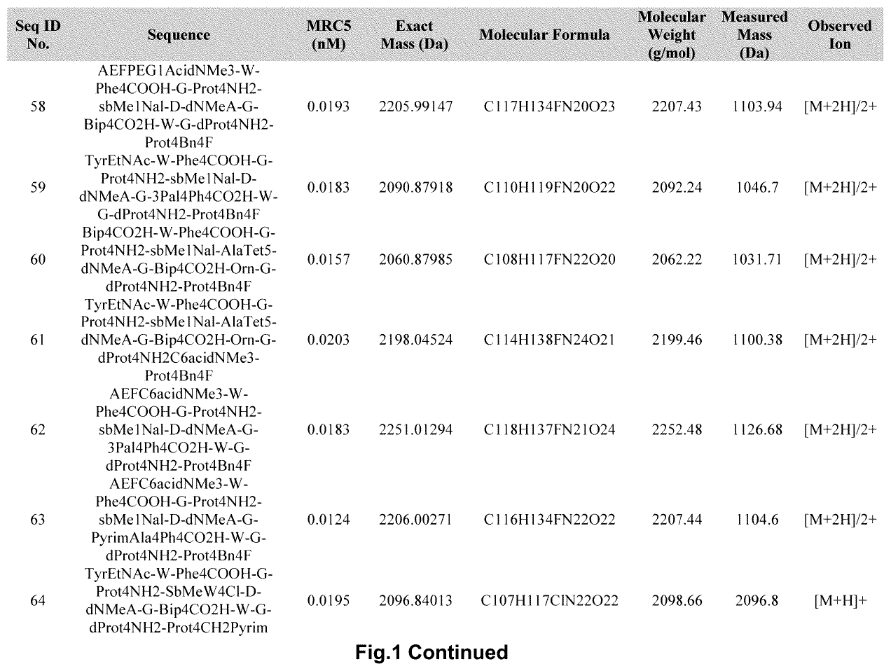

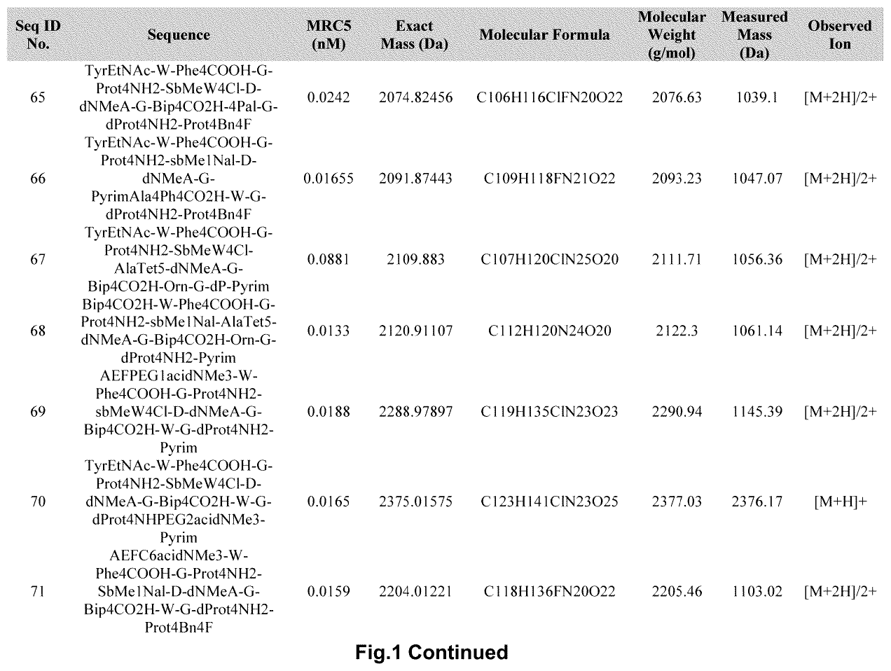

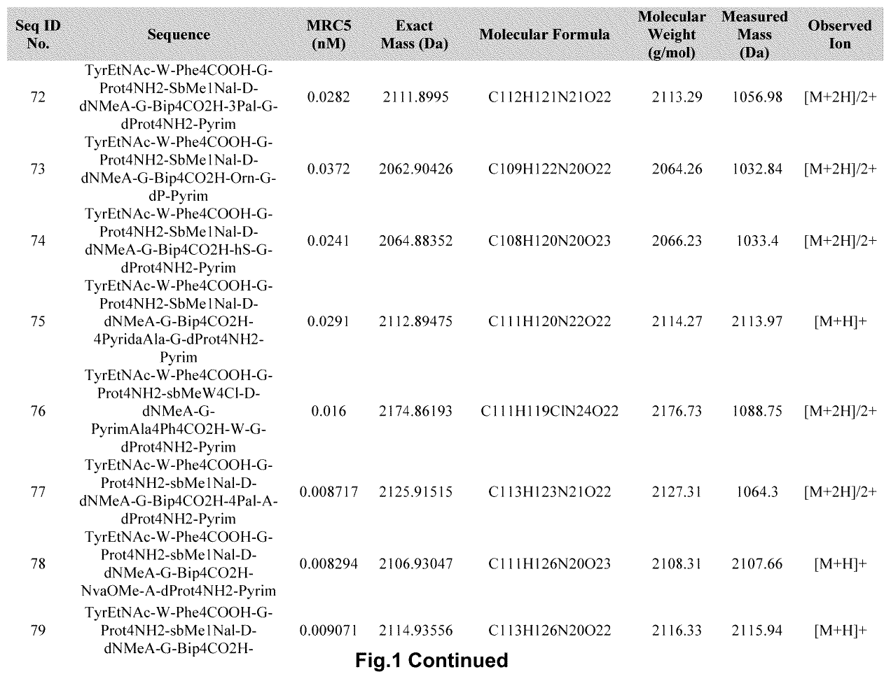

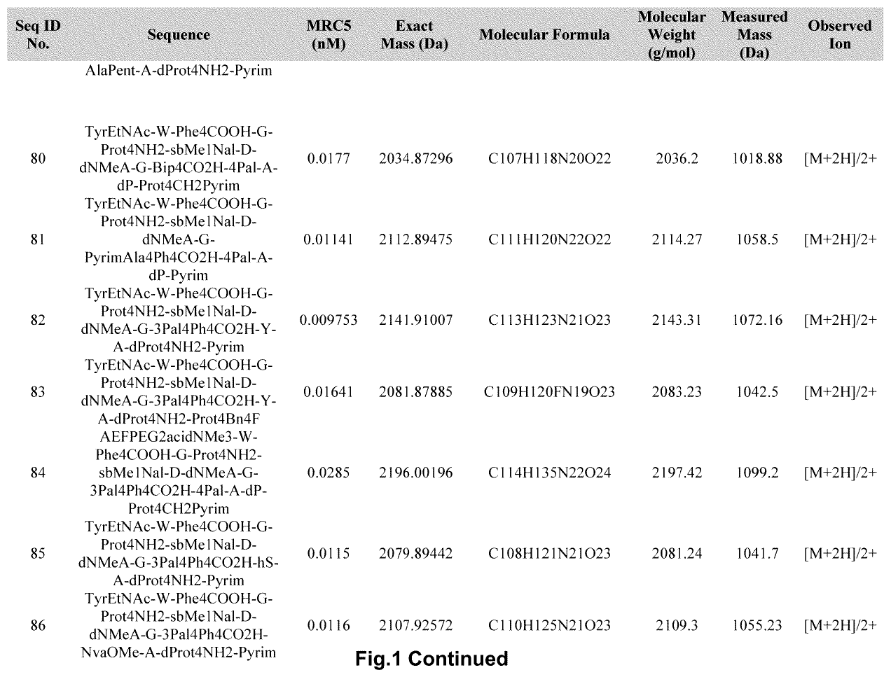

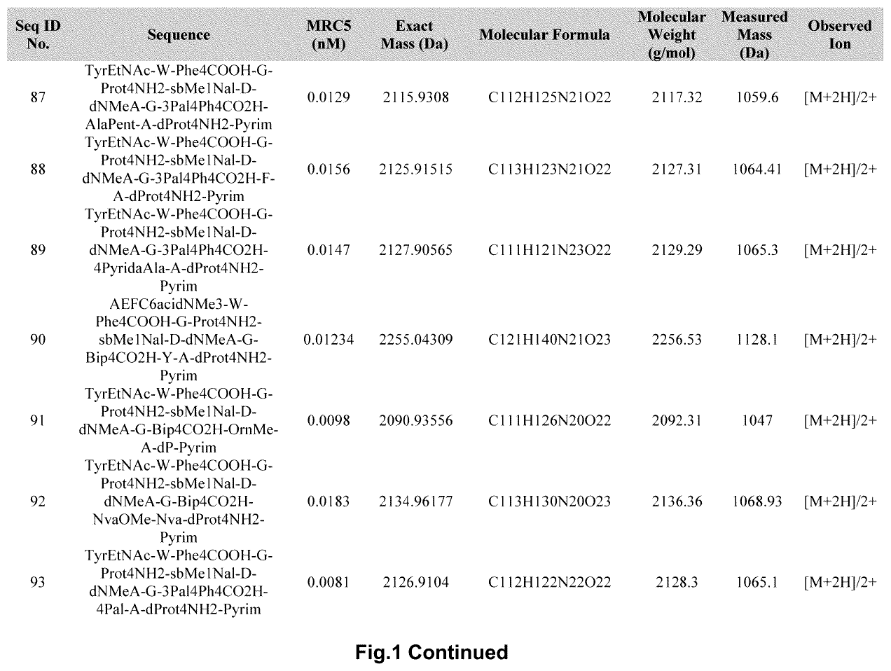

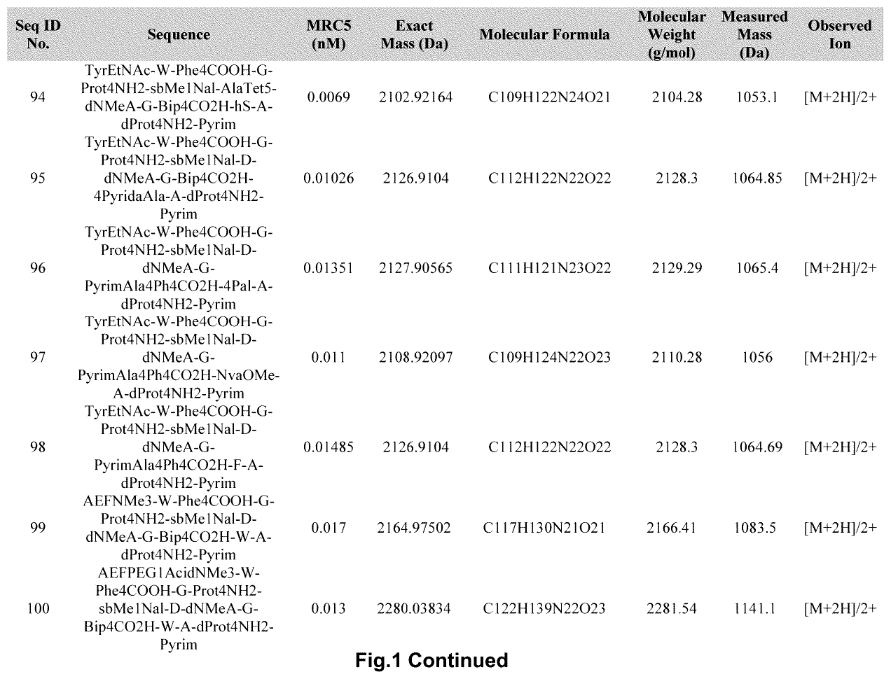

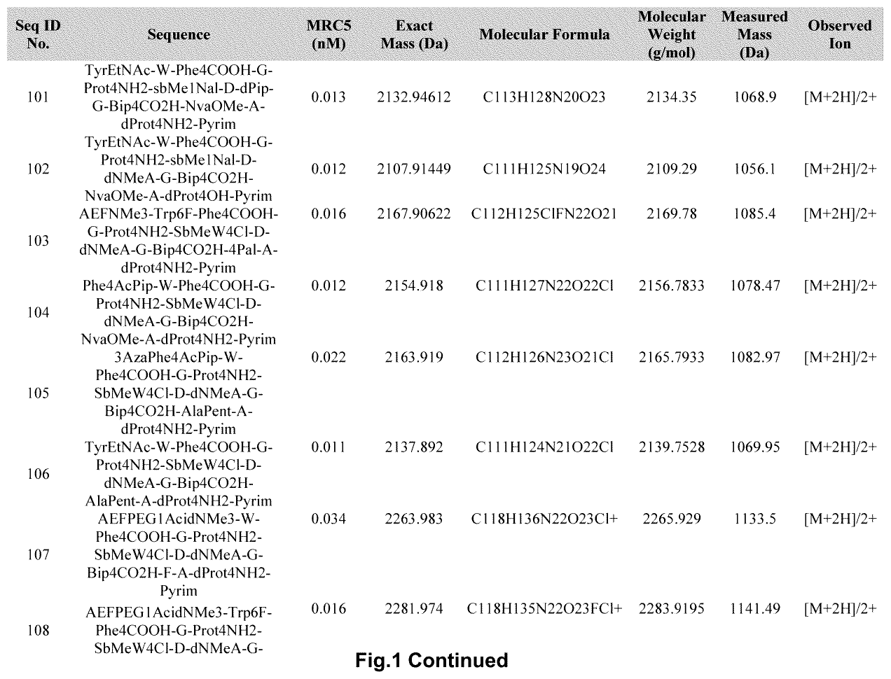

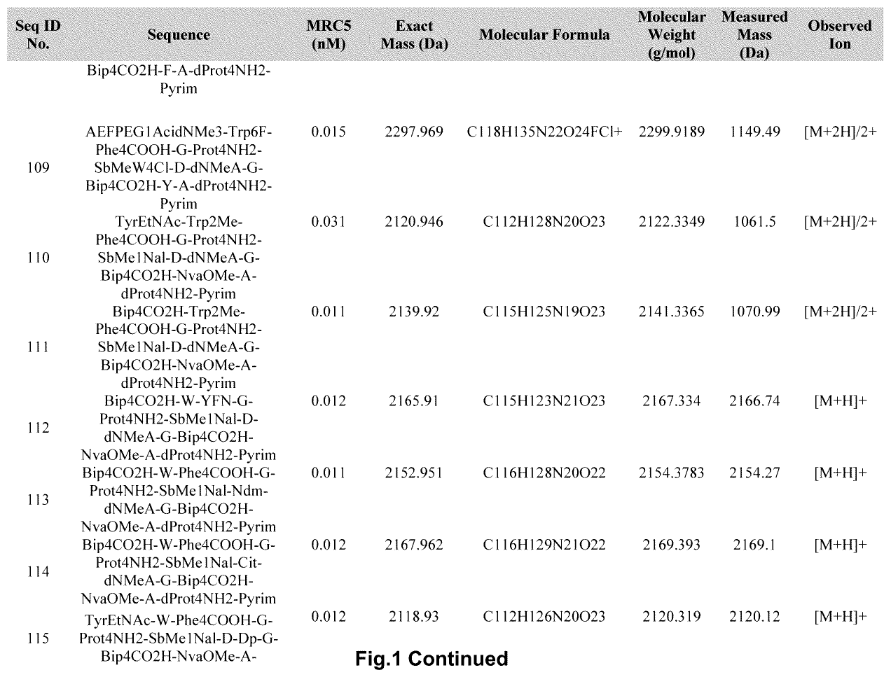

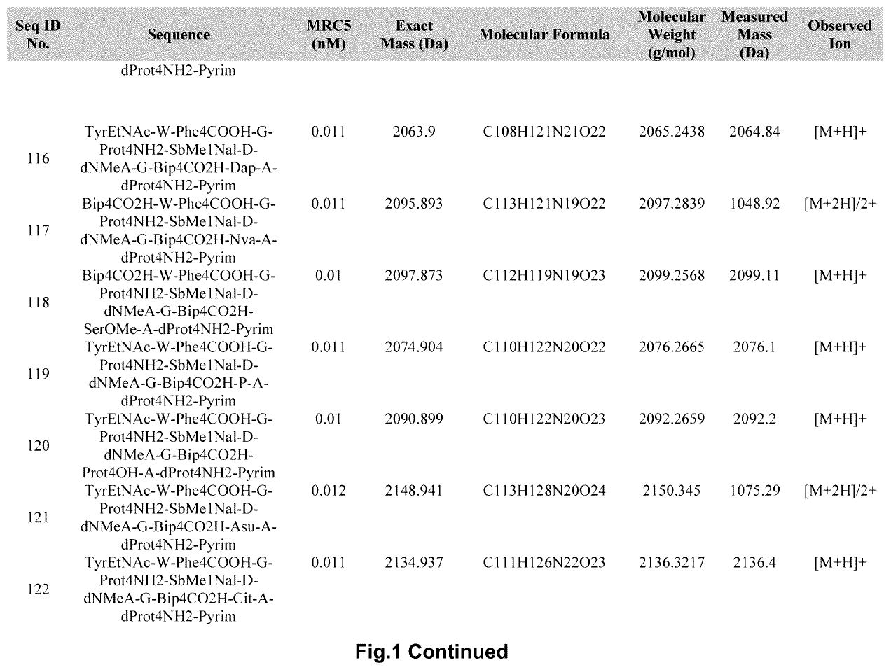

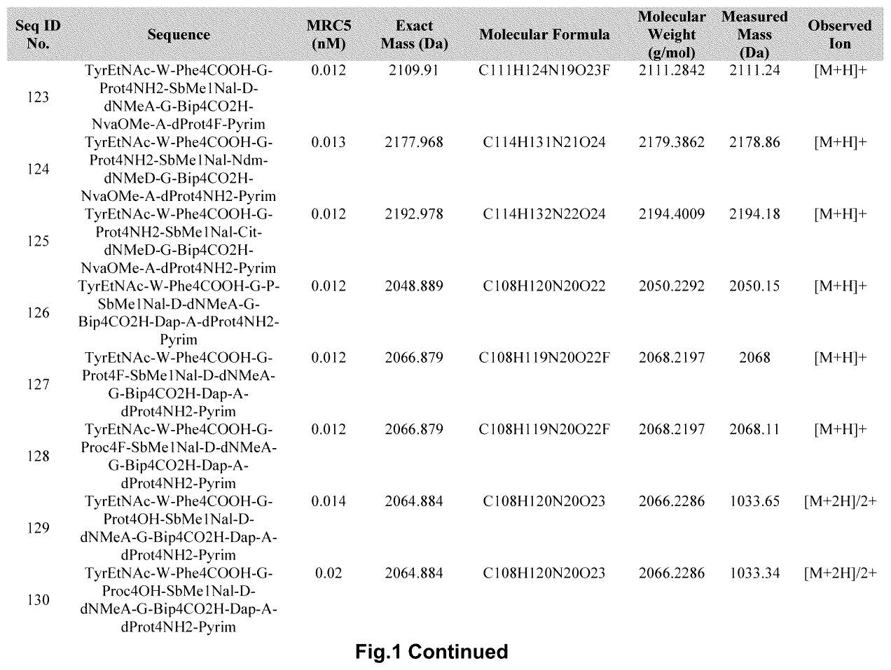

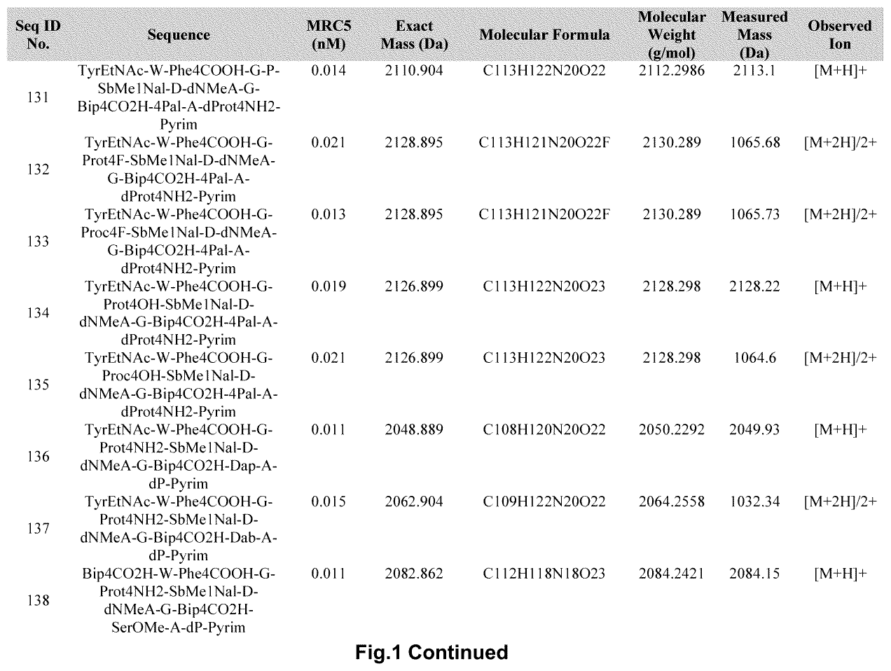

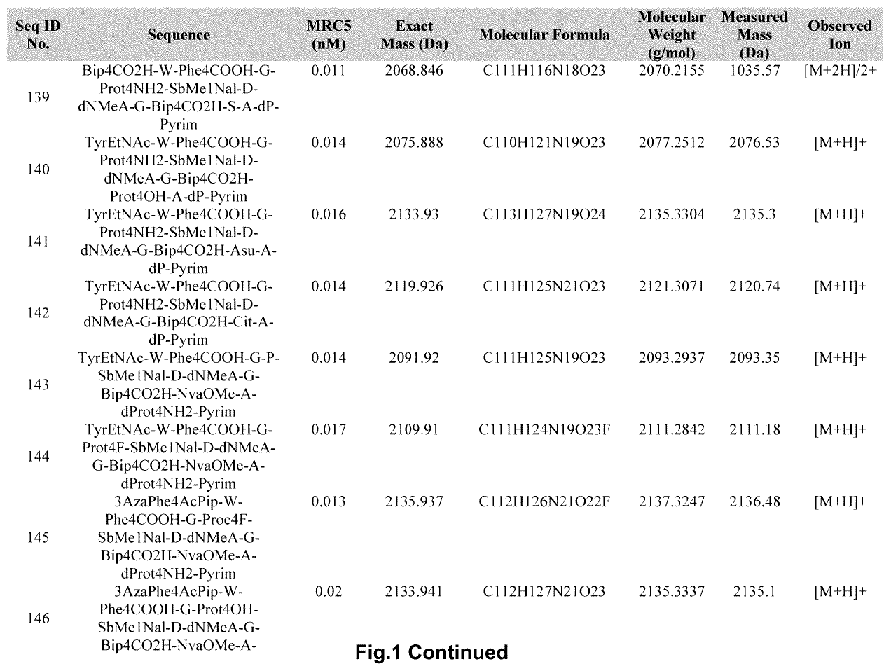

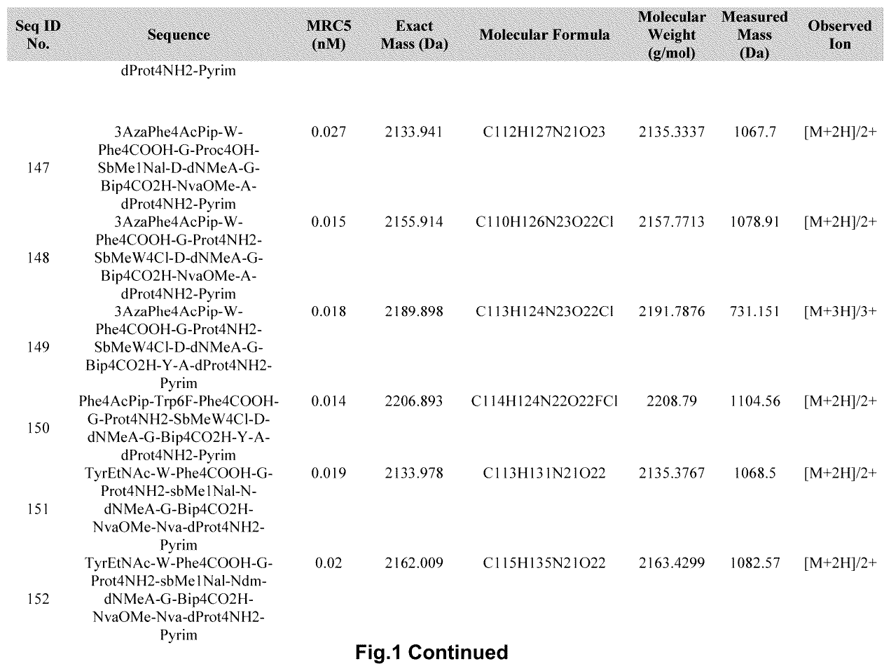

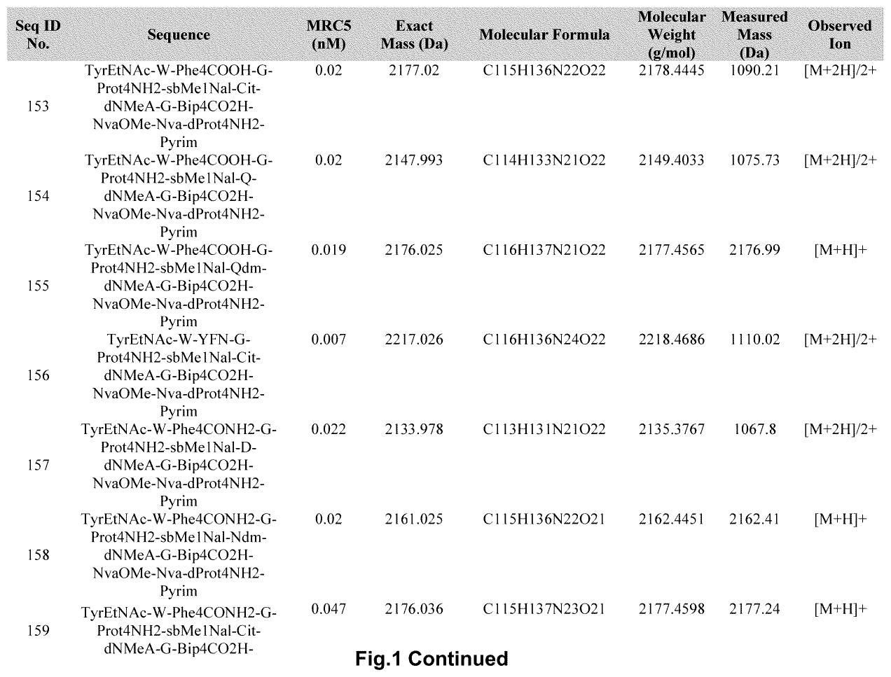

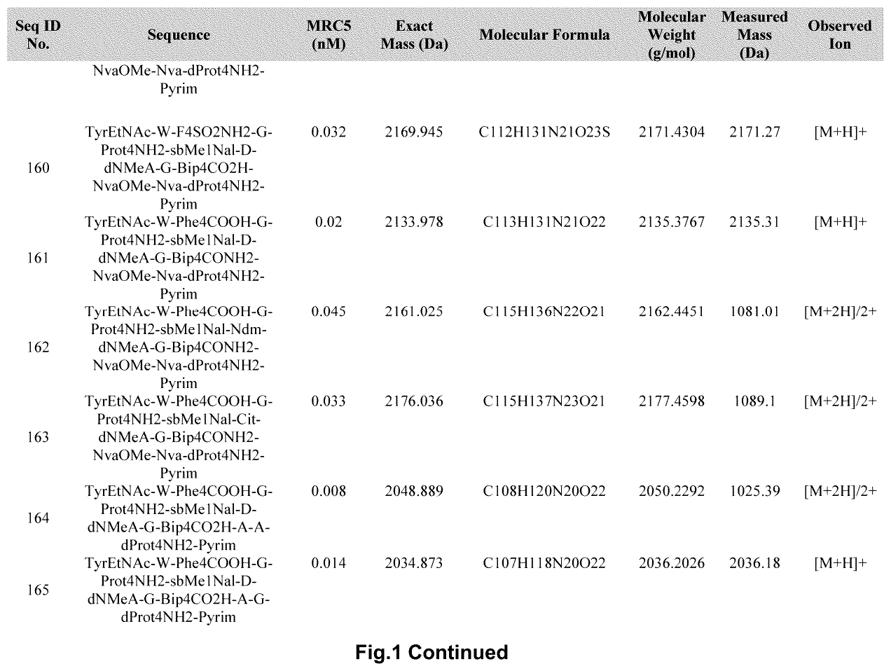

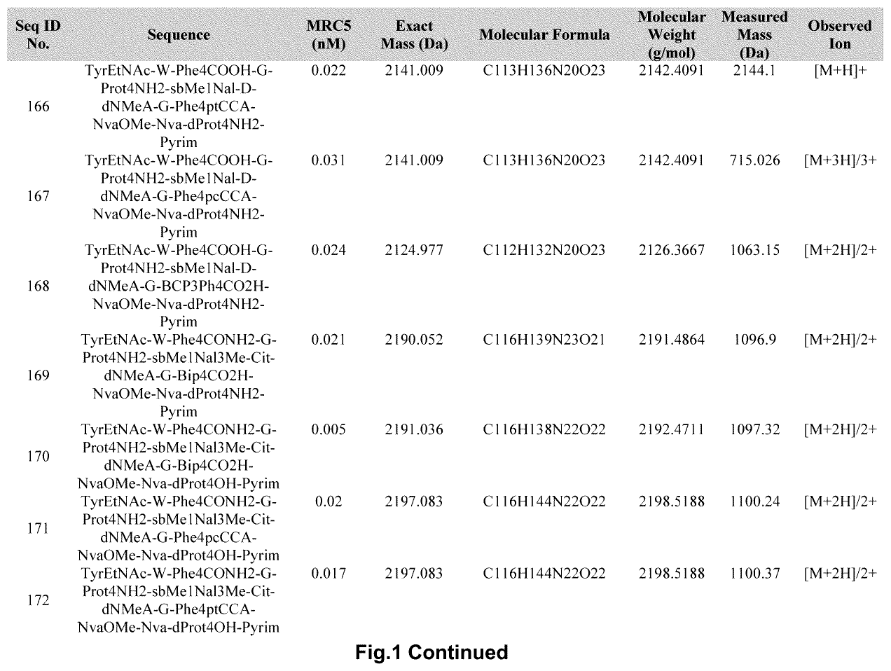

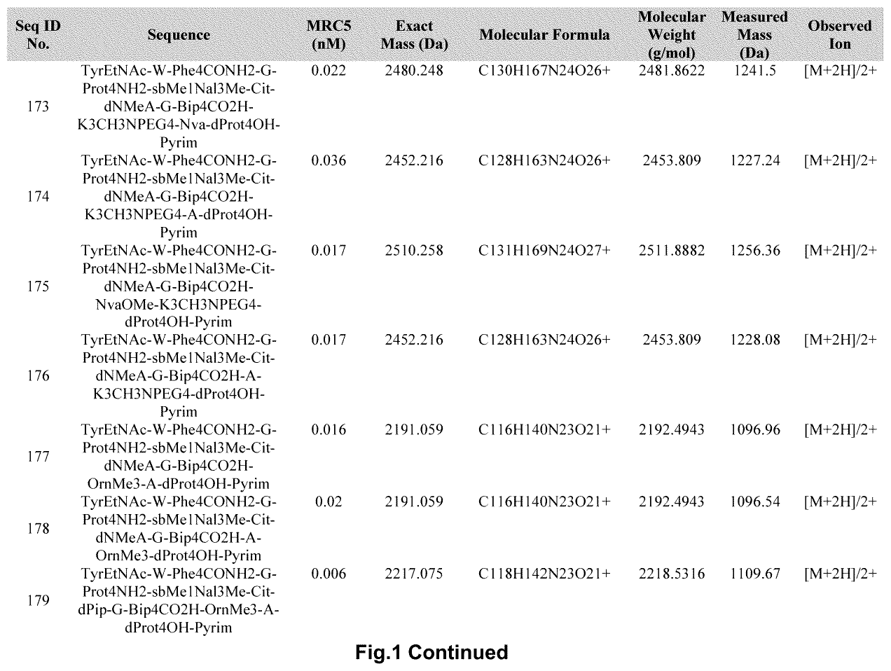

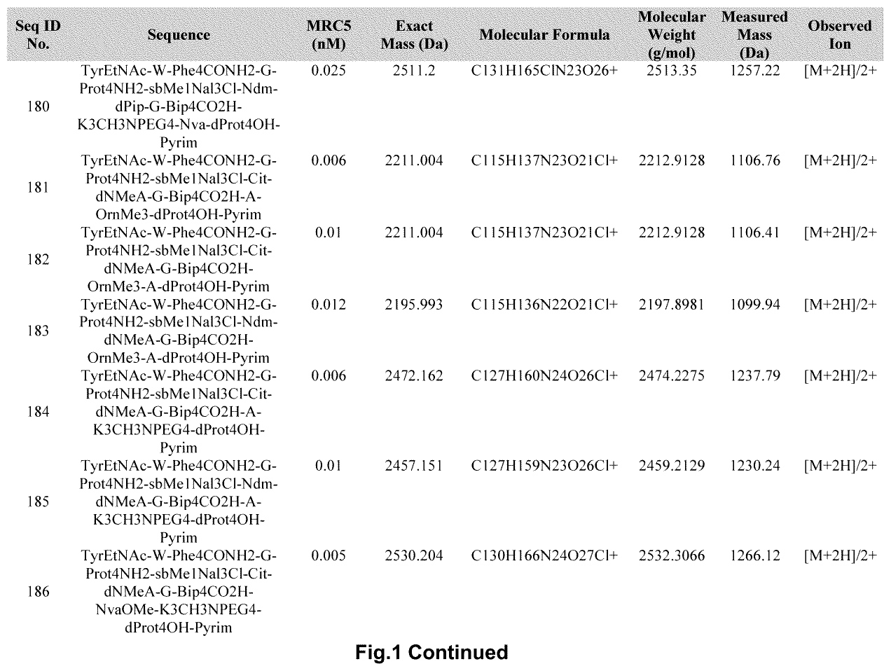

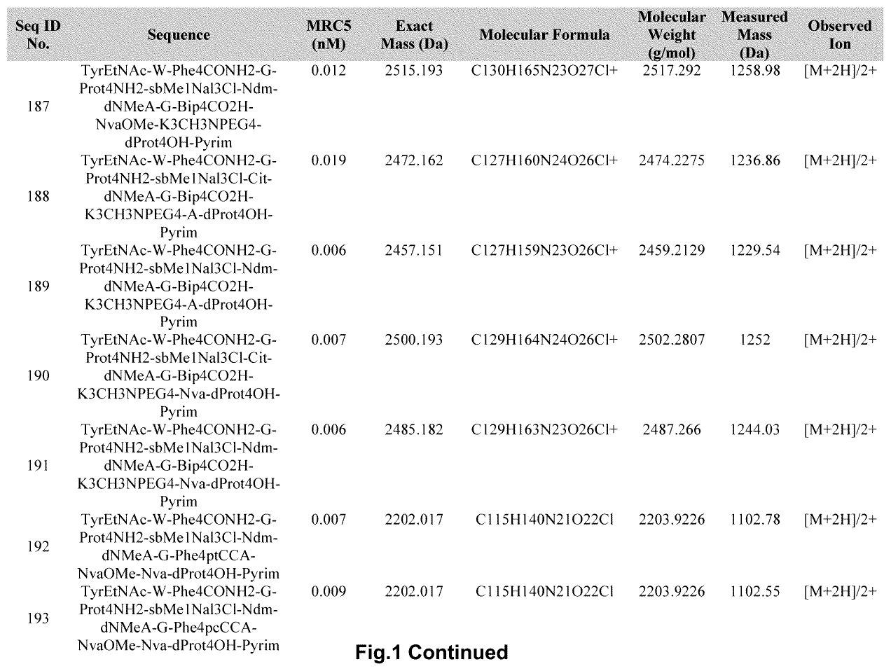

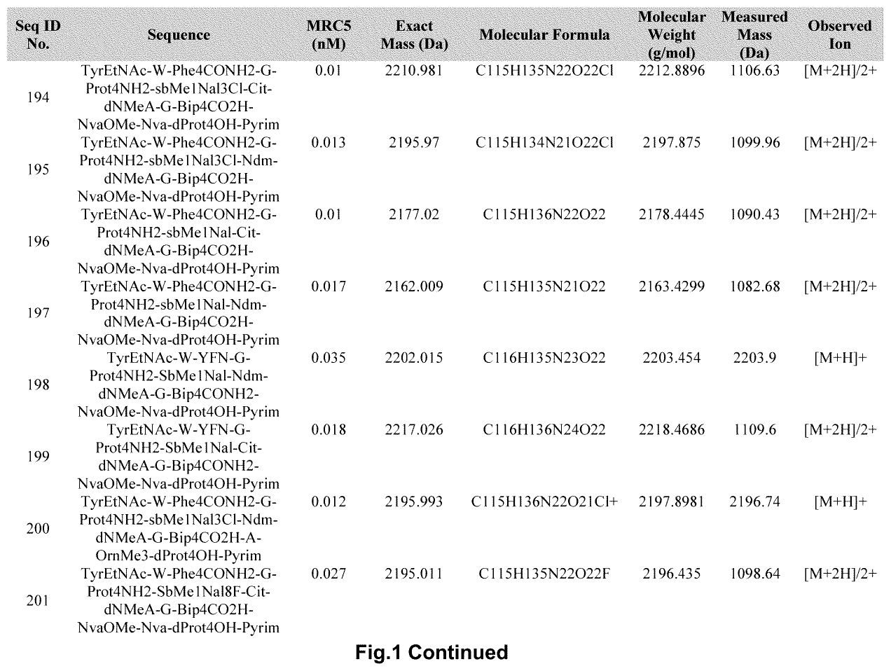

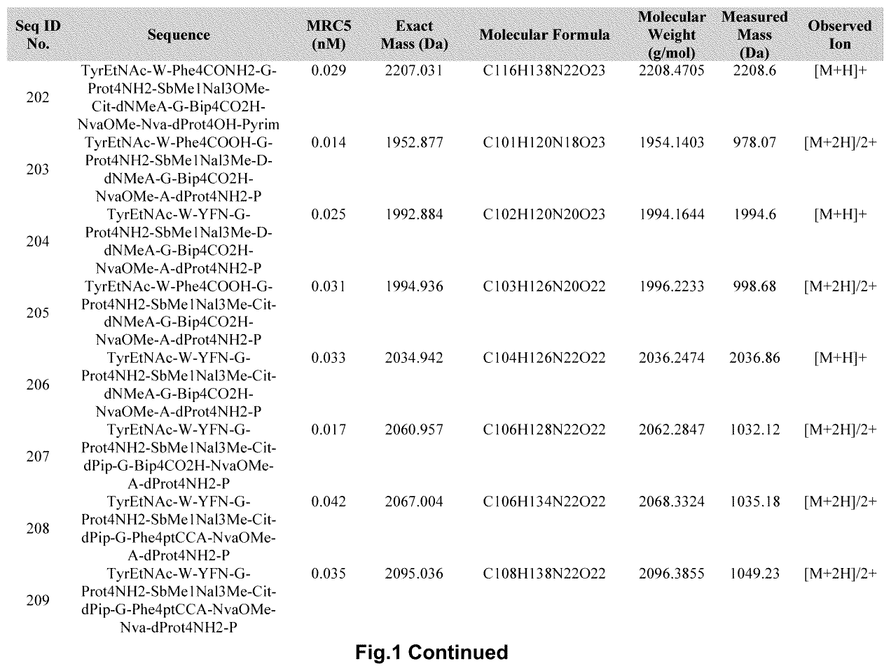

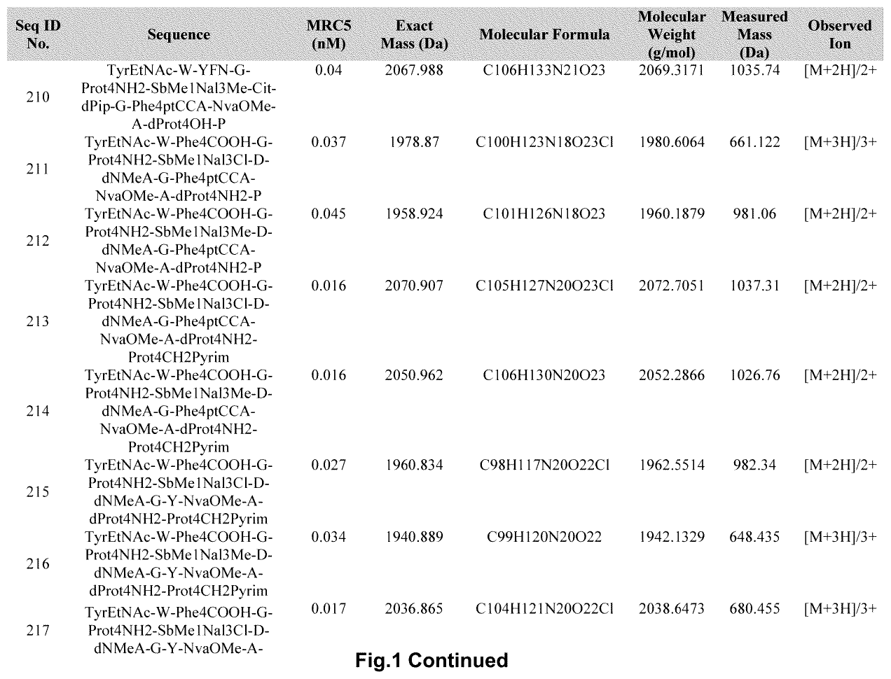

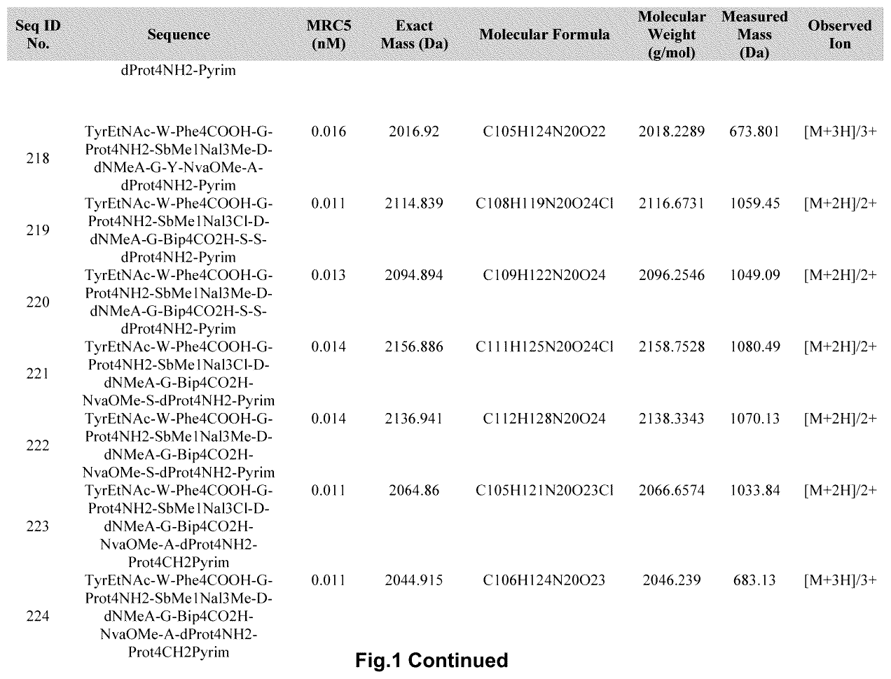

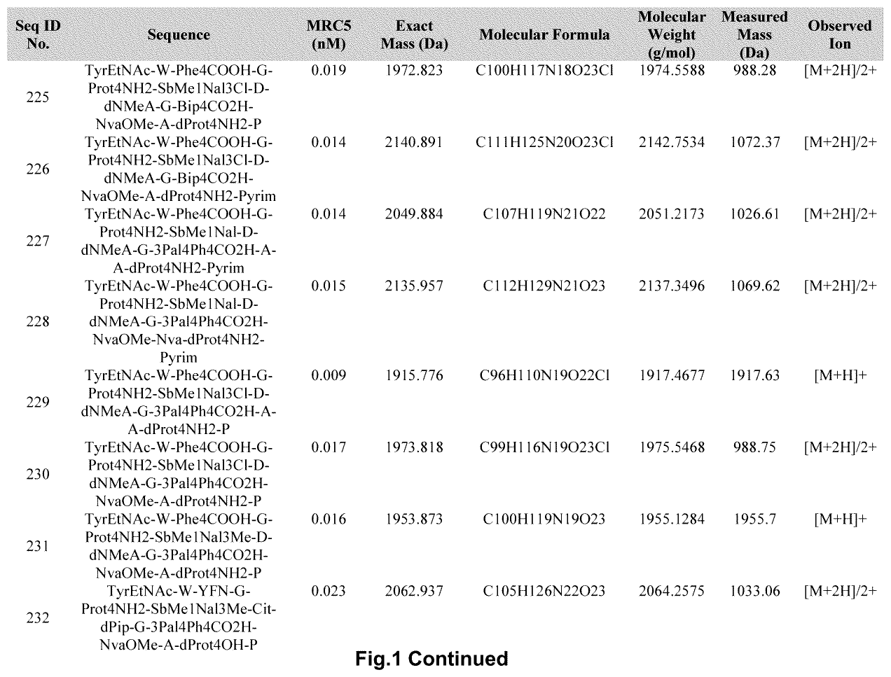

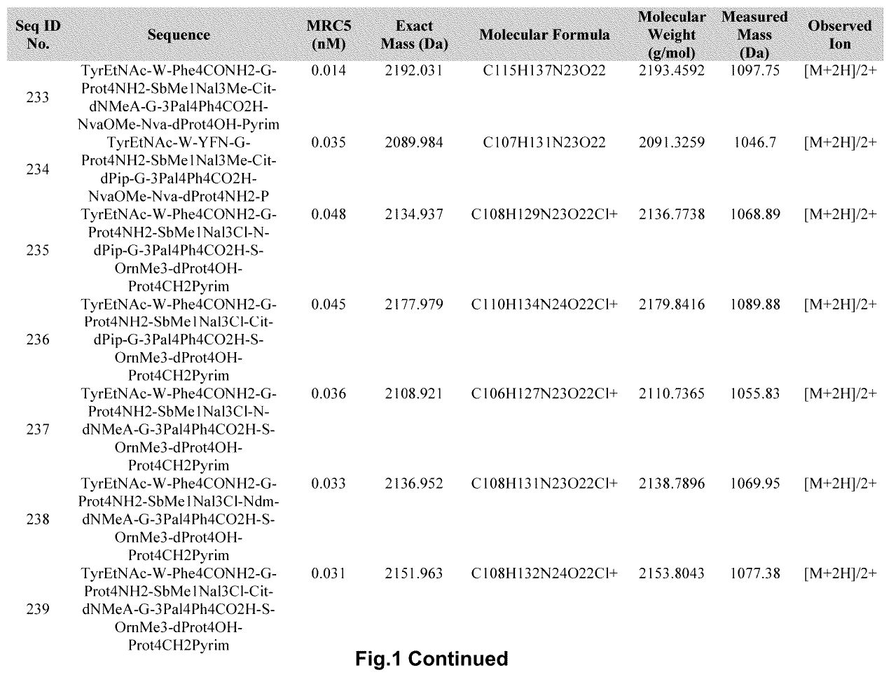

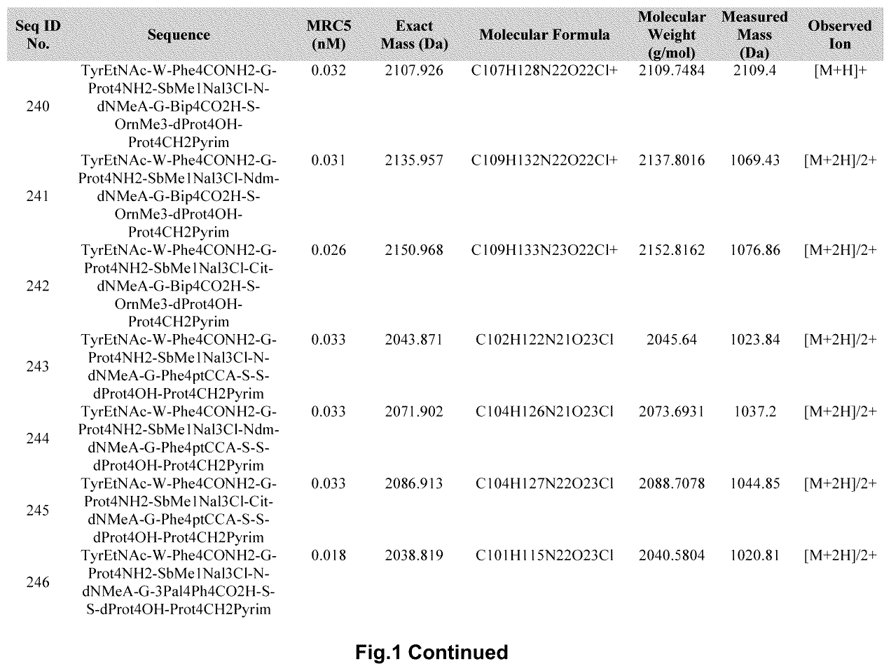

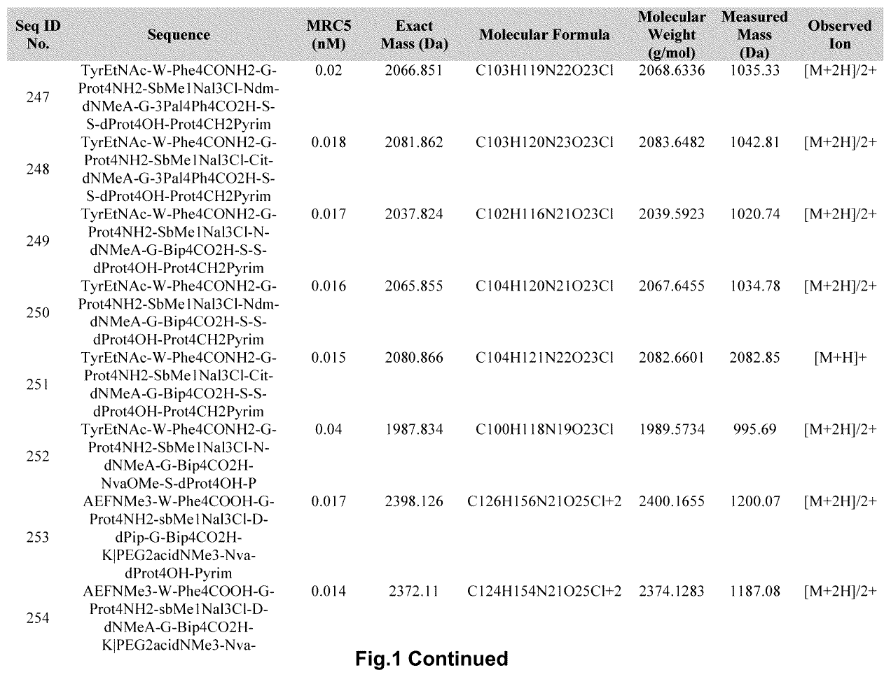

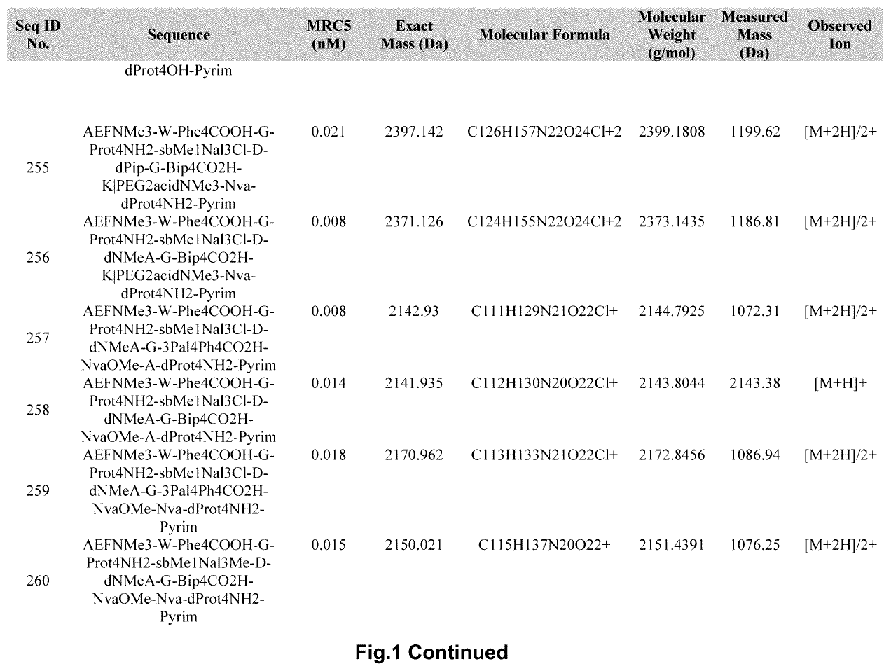

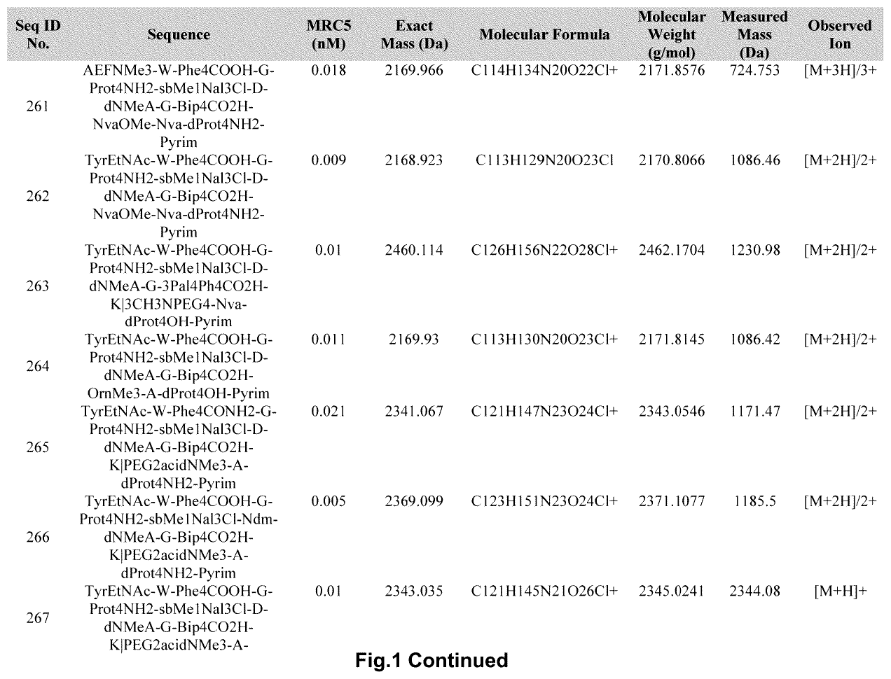

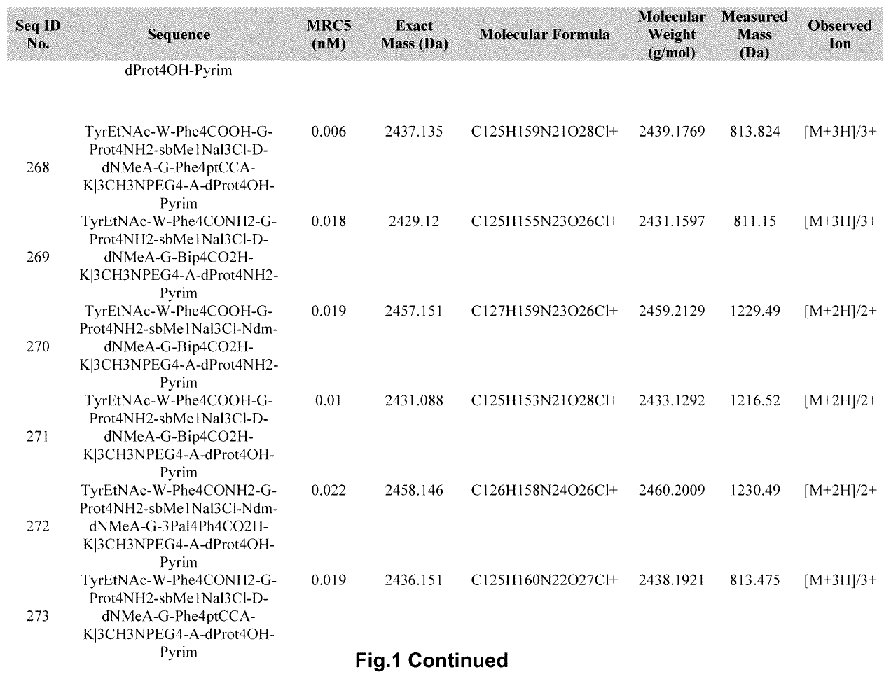

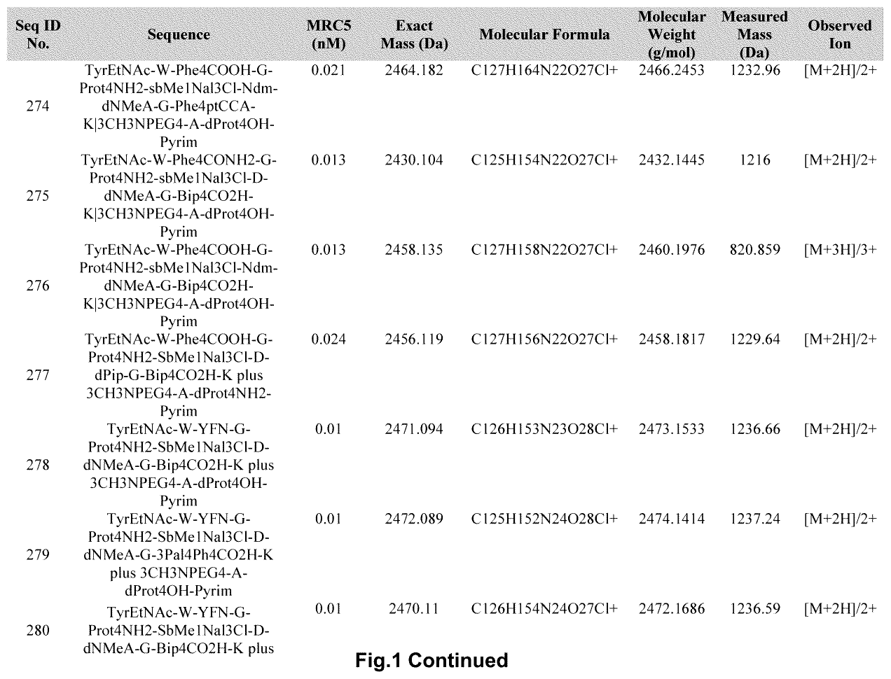

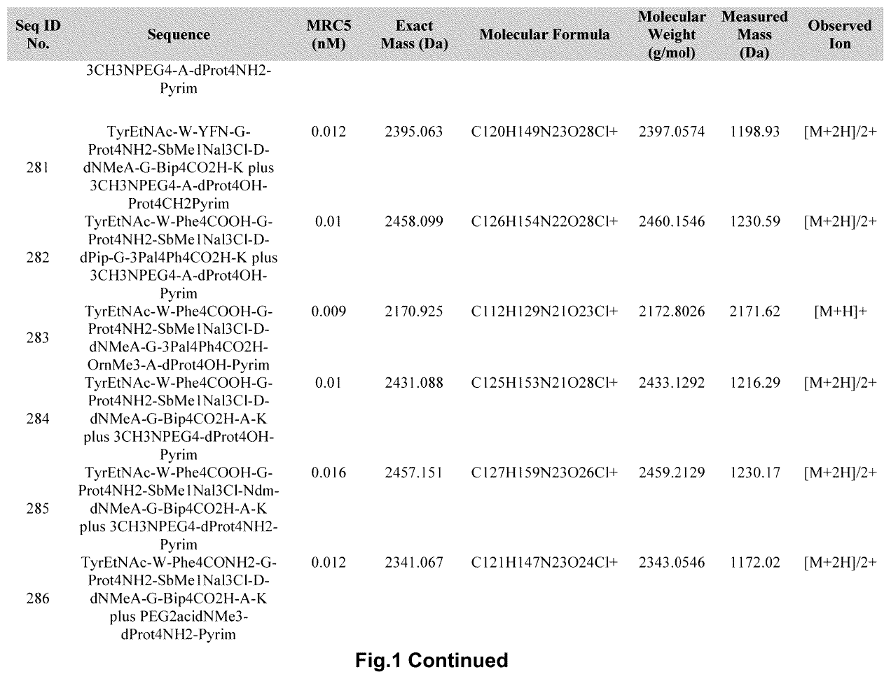

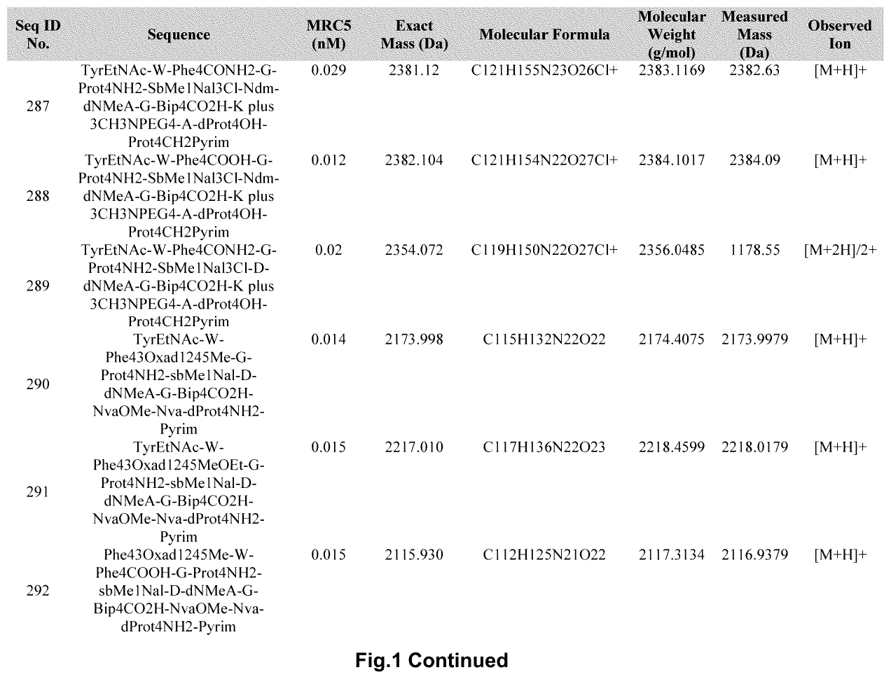

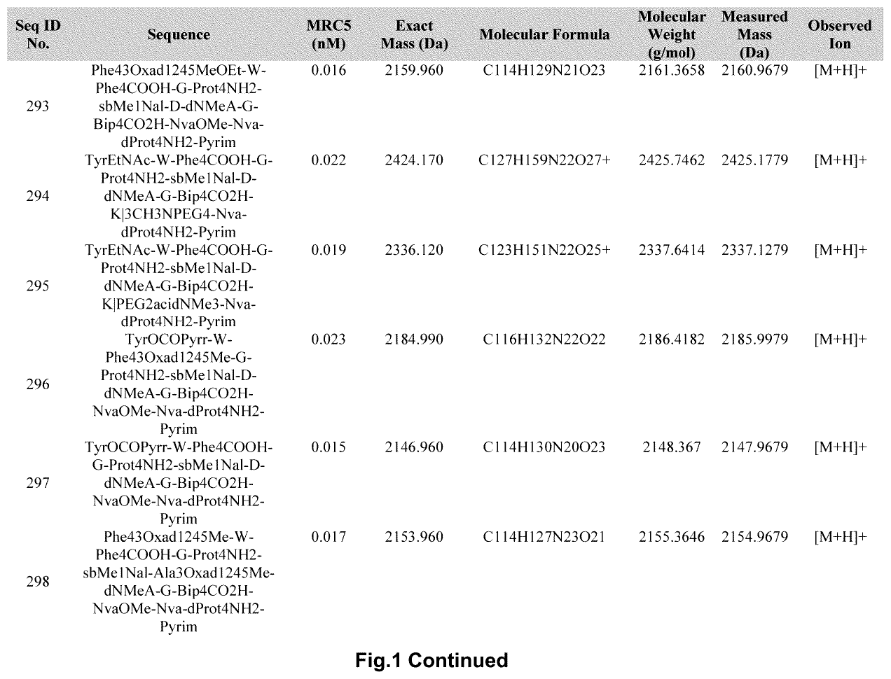

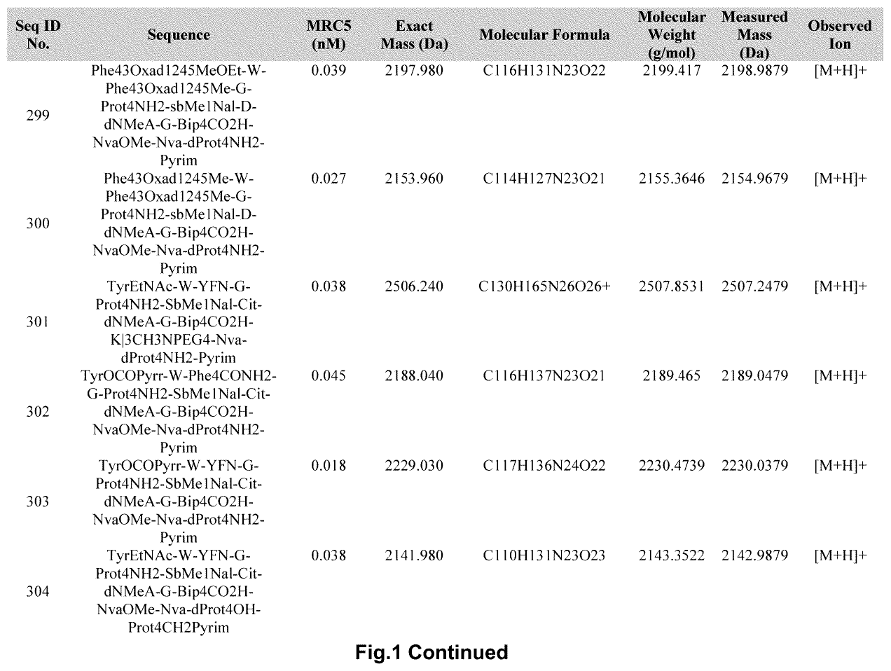

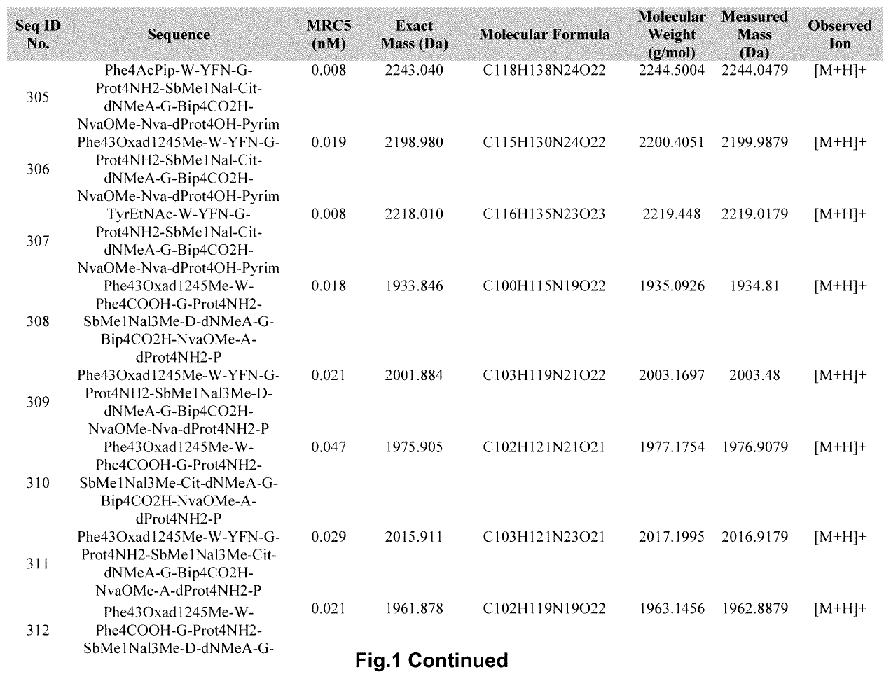

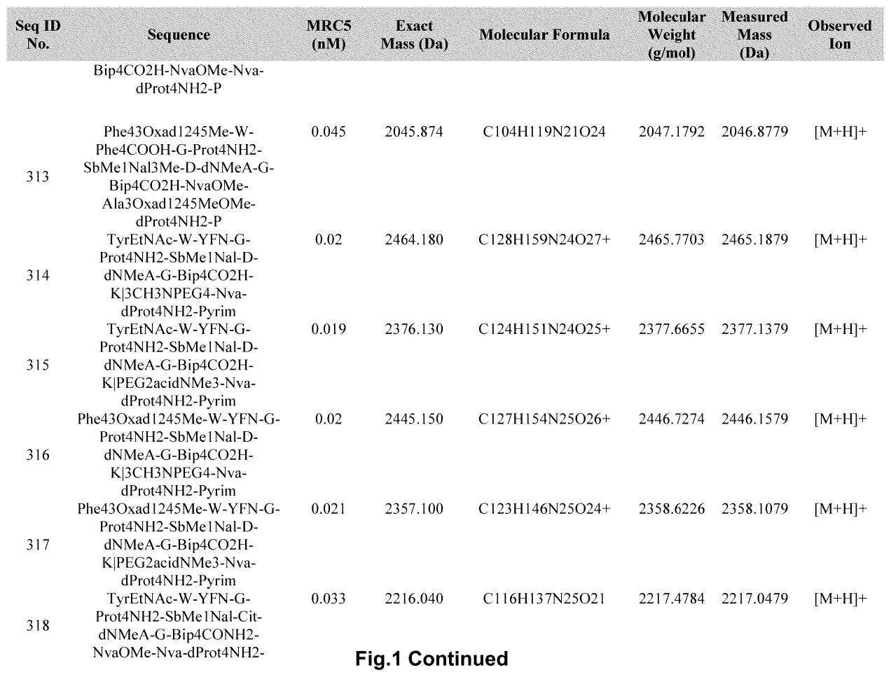

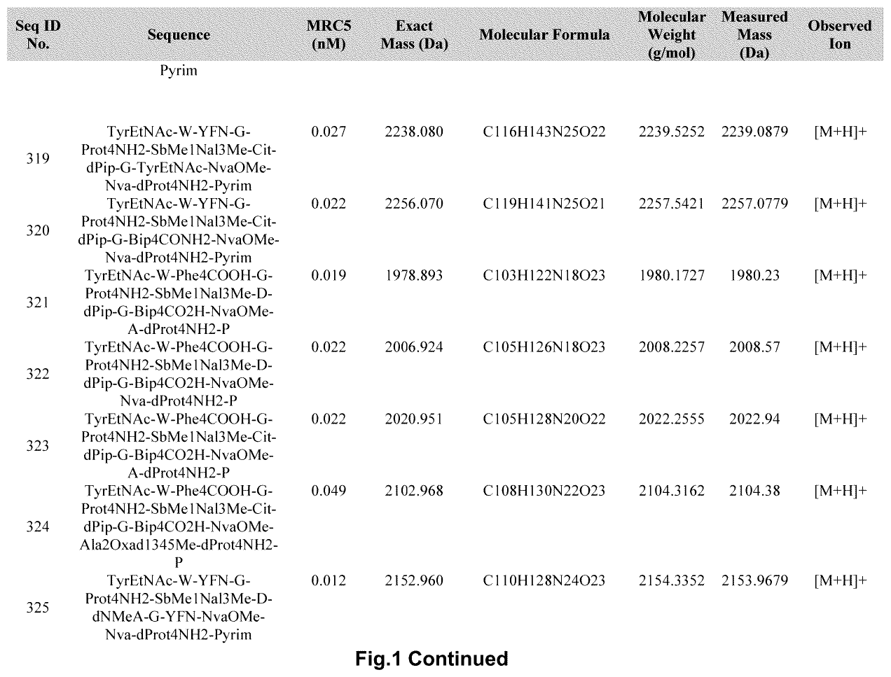

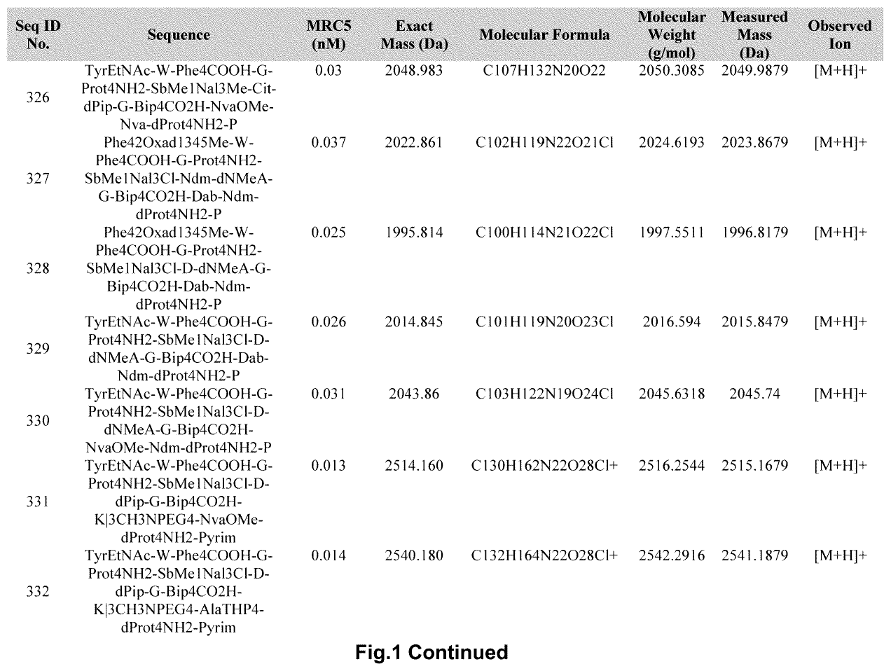

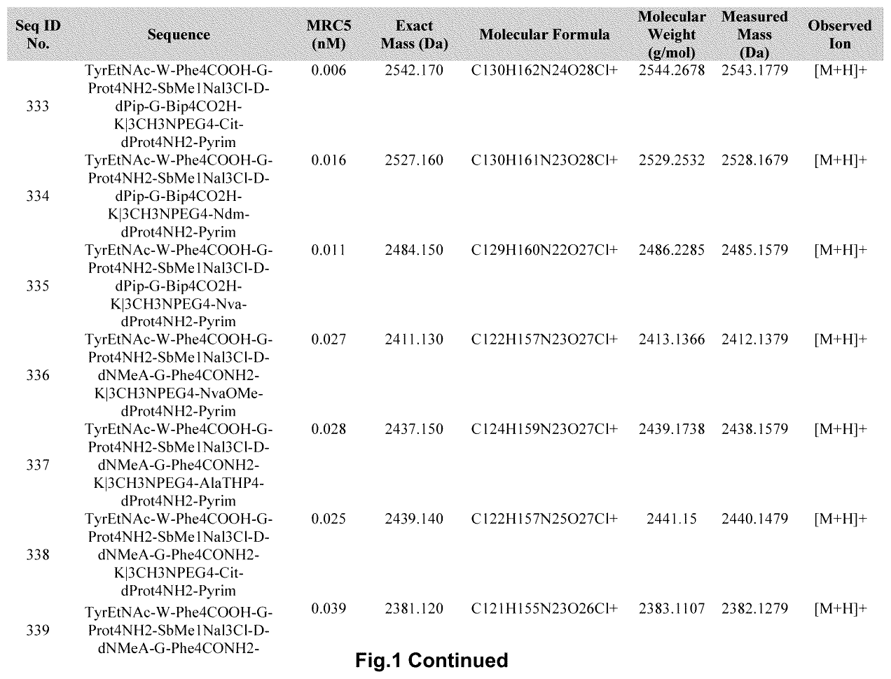

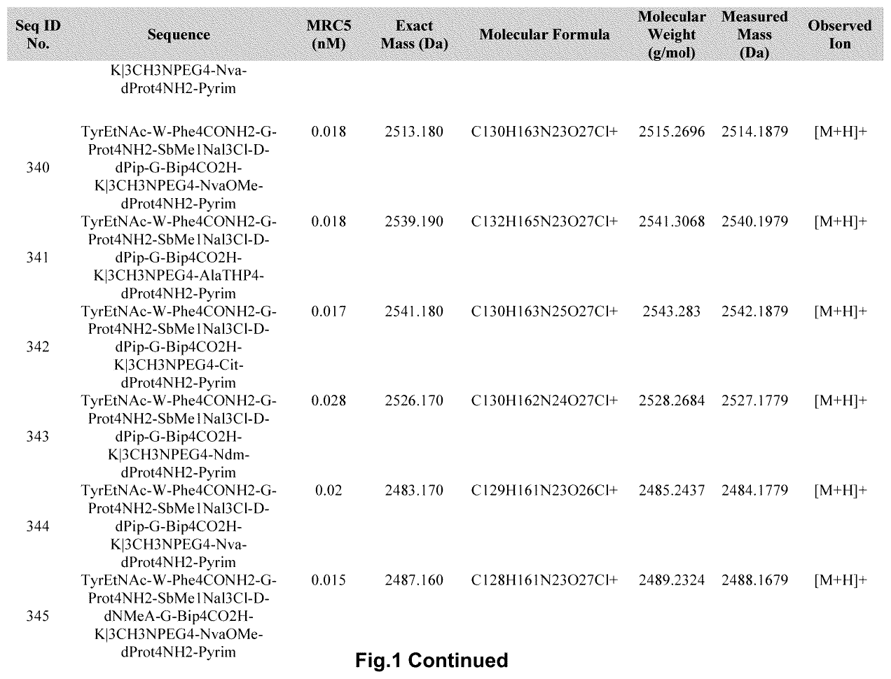

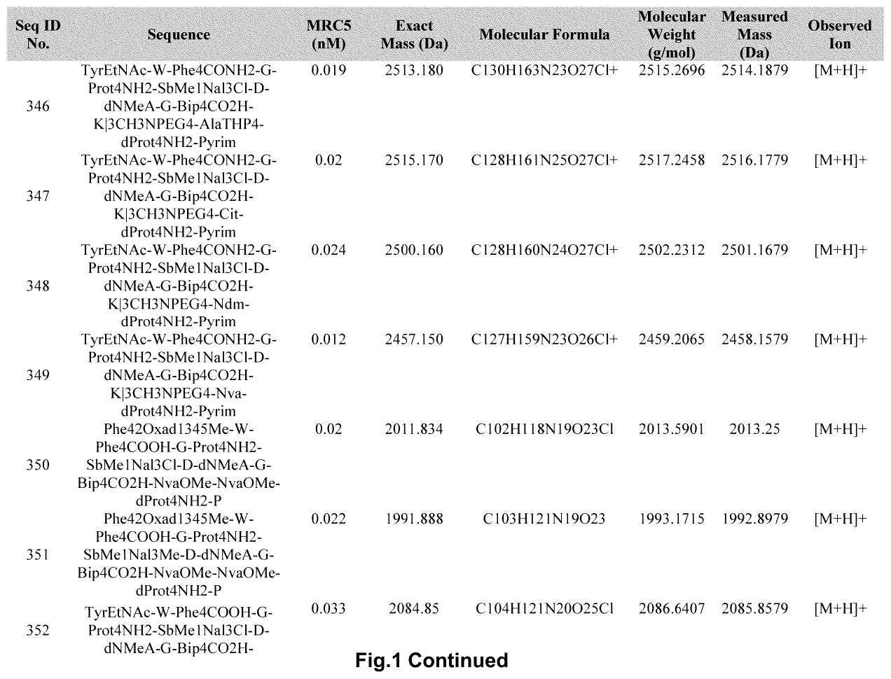

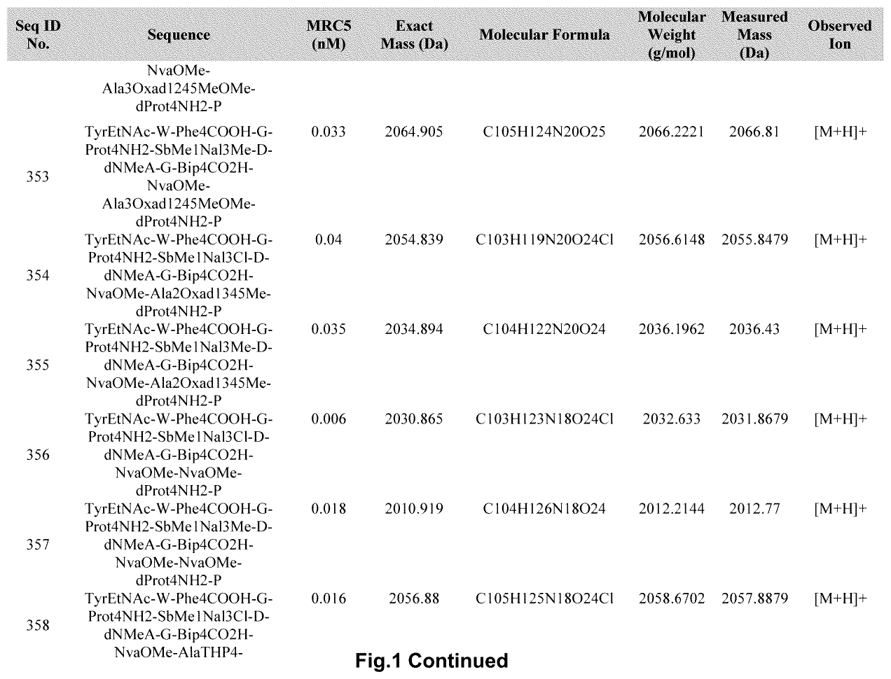

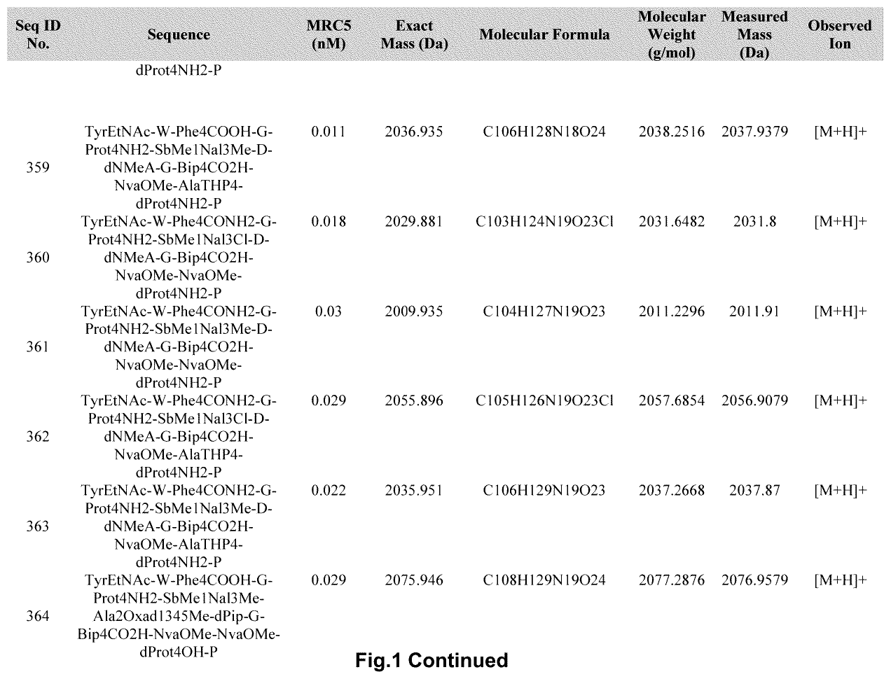

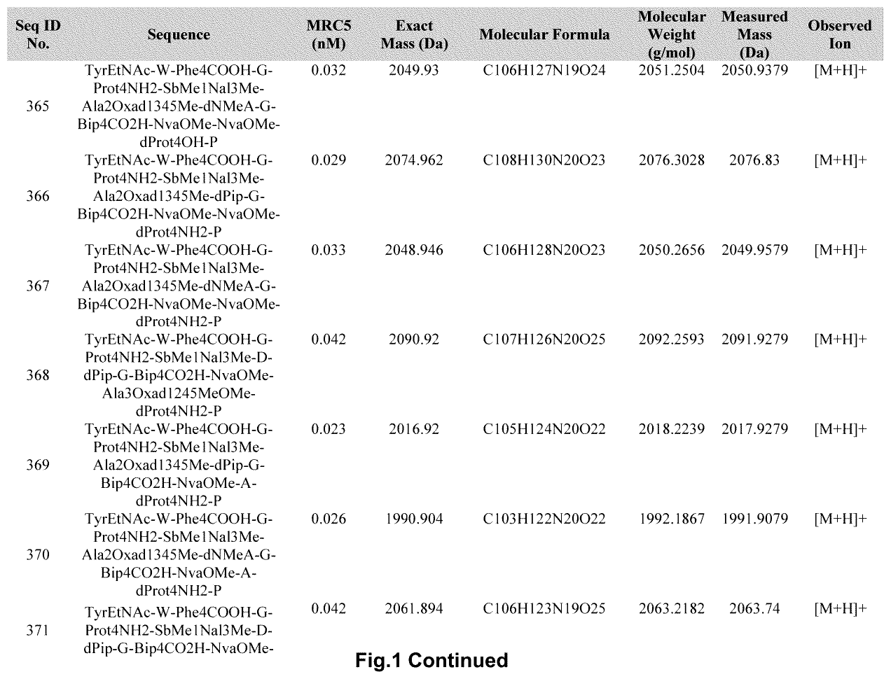

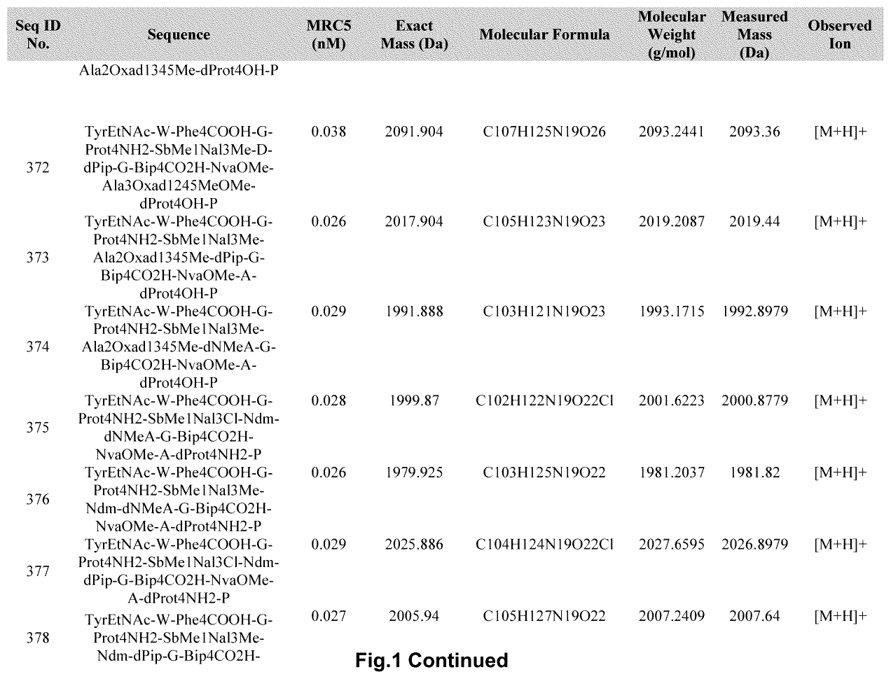

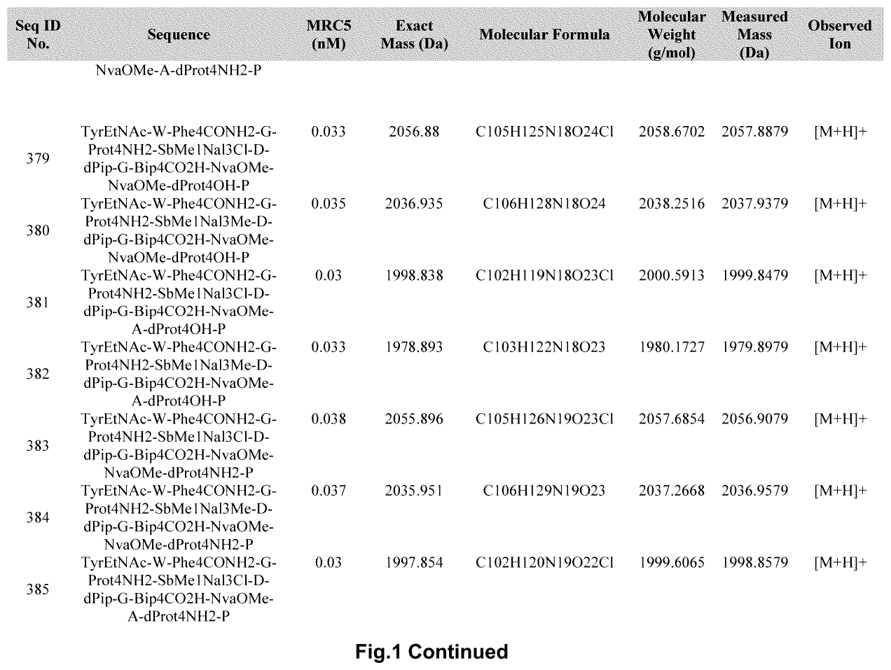

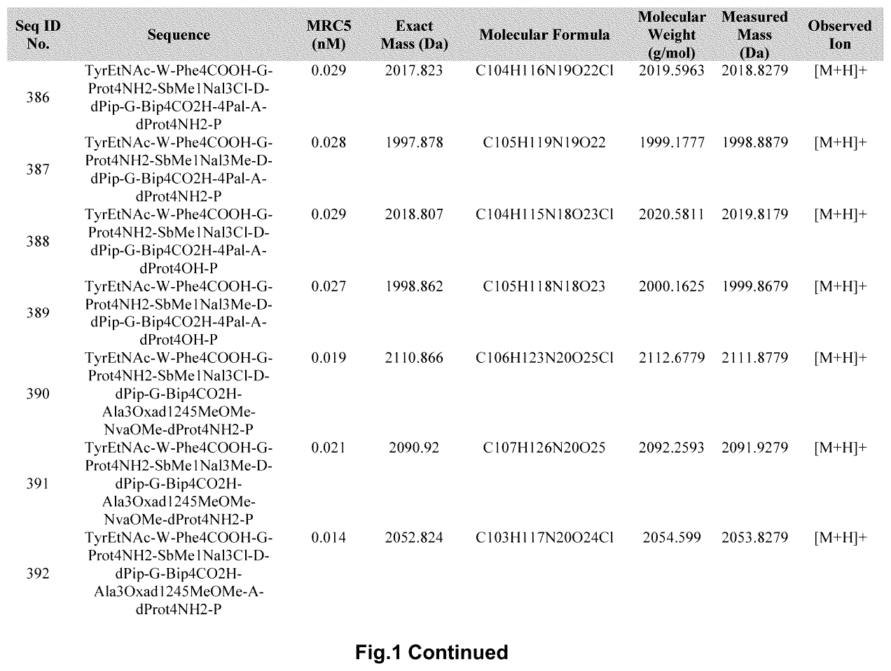

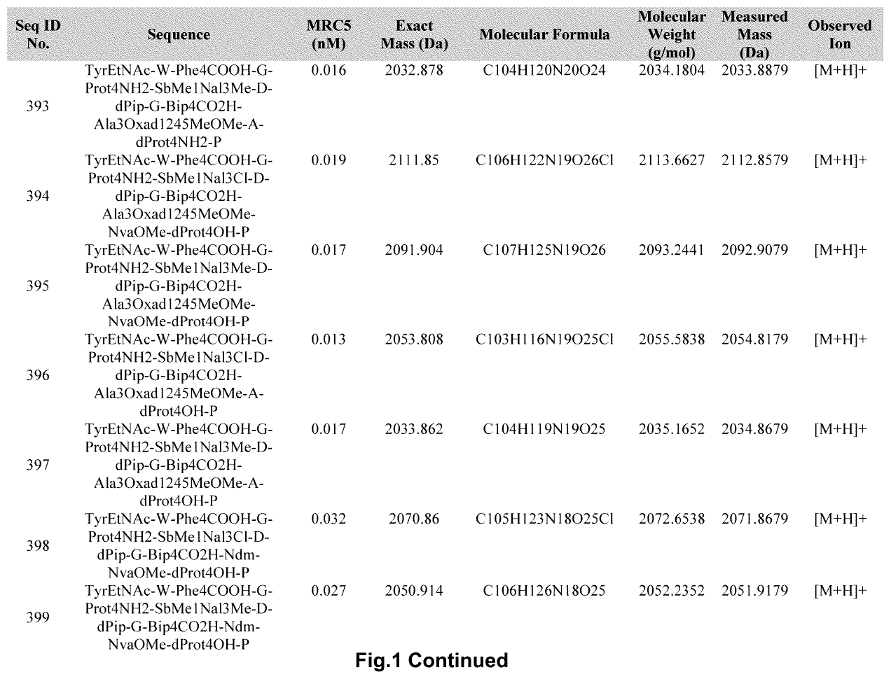

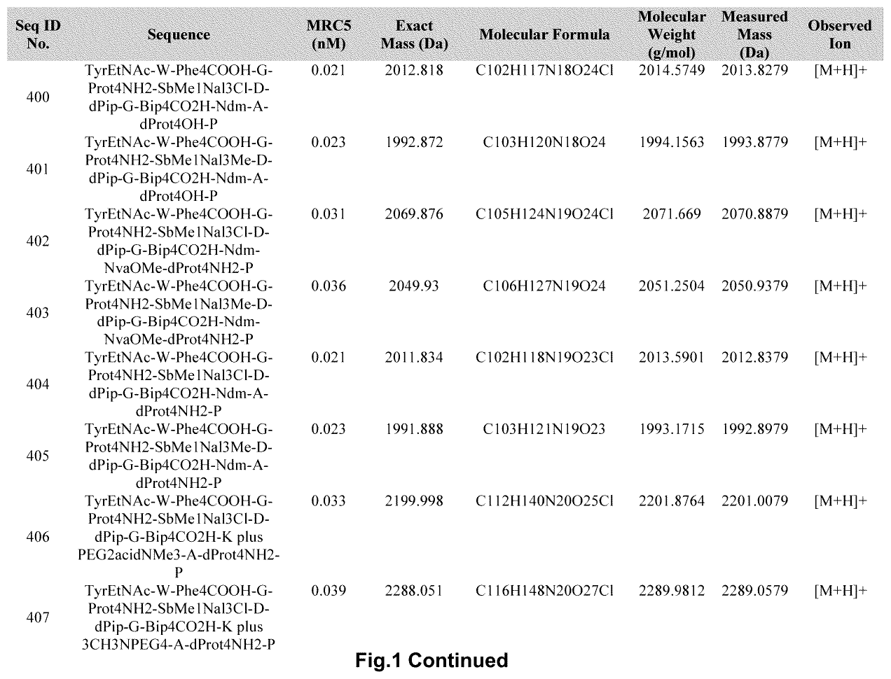

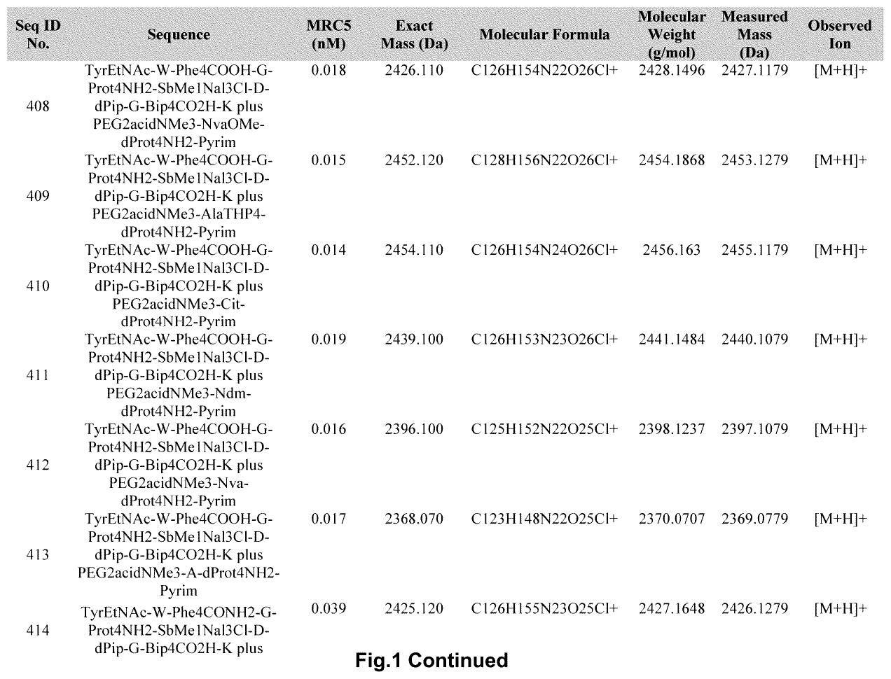

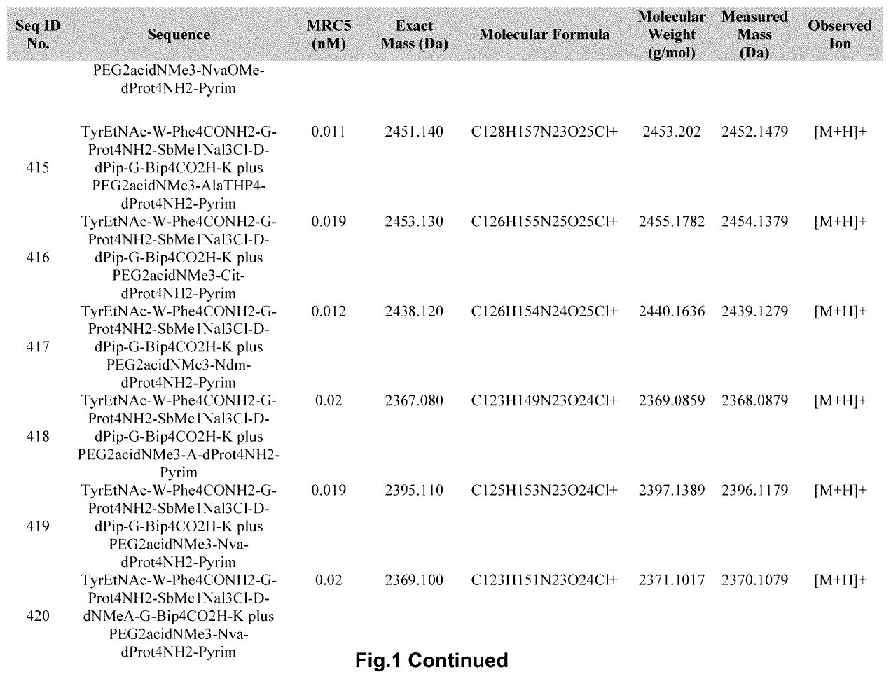

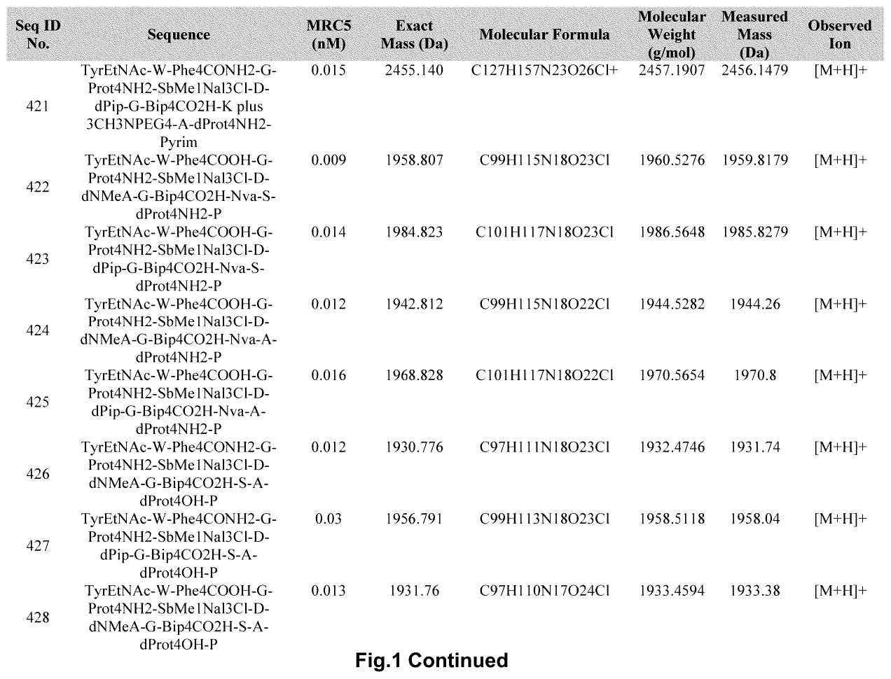

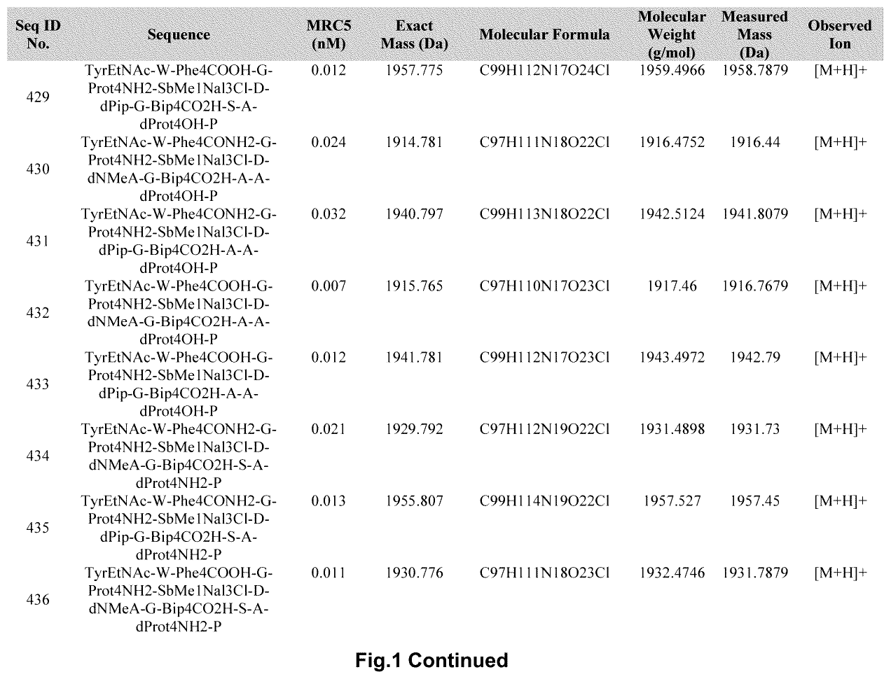

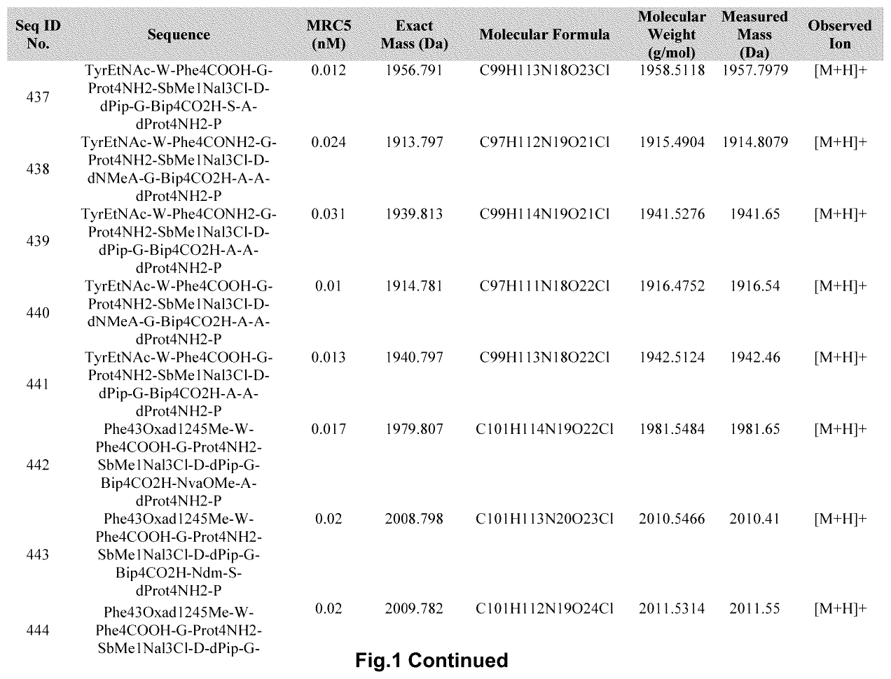

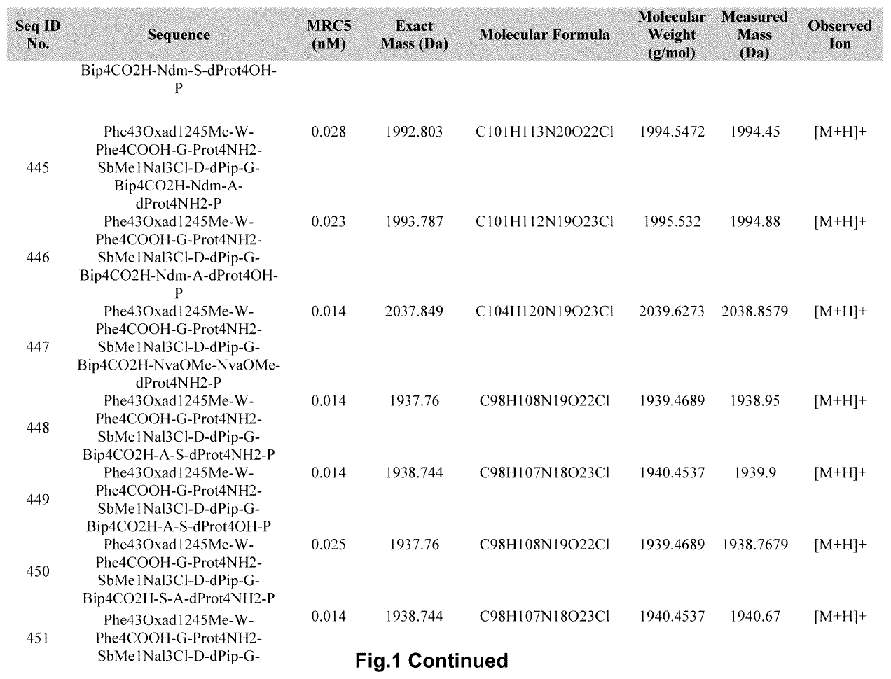

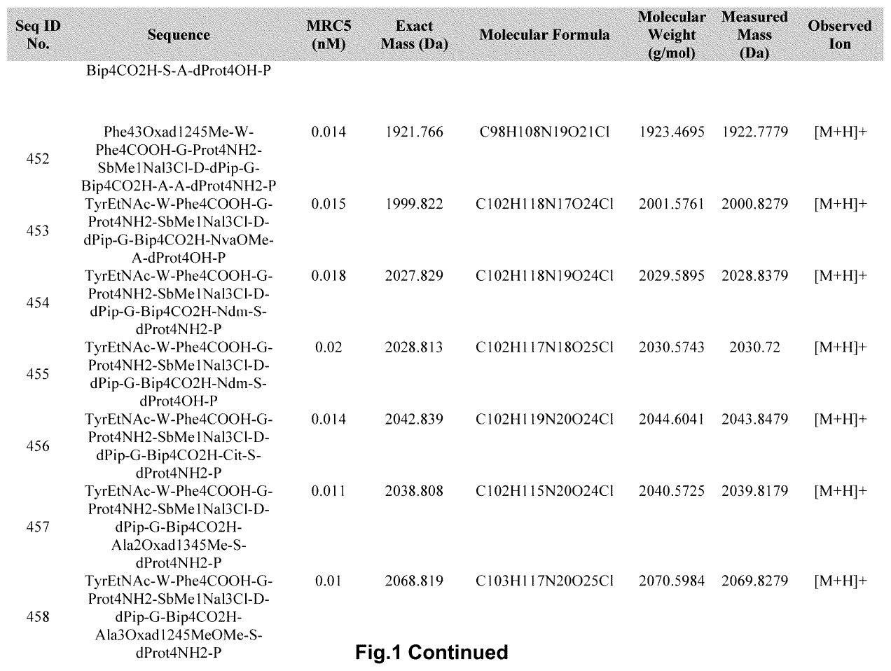

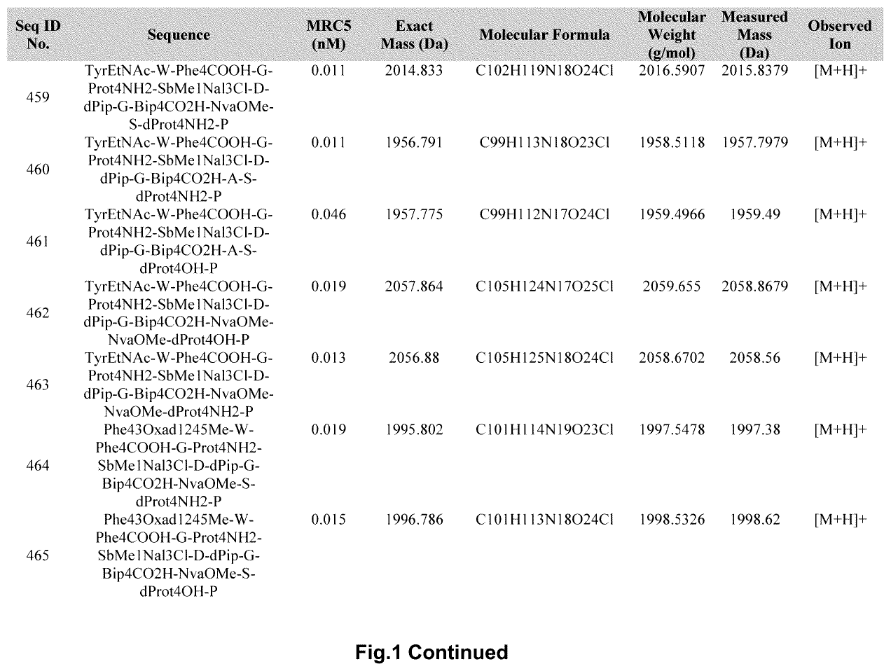

In certain embodiments, the present disclosure provides a compound of Formula (I), wherein the compound is selected from the group consisting of SEQ ID NOS: 1-465 as set forth in FIG. 1. In specific embodiments, the compound is selected from the group consisting of SEQ ID NOS: 1-103 as set forth in FIG. 1.

In specific embodiments, the present disclosure provides a compound of Formula (I), wherein the compound selected from the group consisting of (SEQ ID NOs: 1, 2, 78, 80, 82, 92, 94, 95, 98, 99, 100, 101, 102, 215, 216, 217, and 218, respectively):

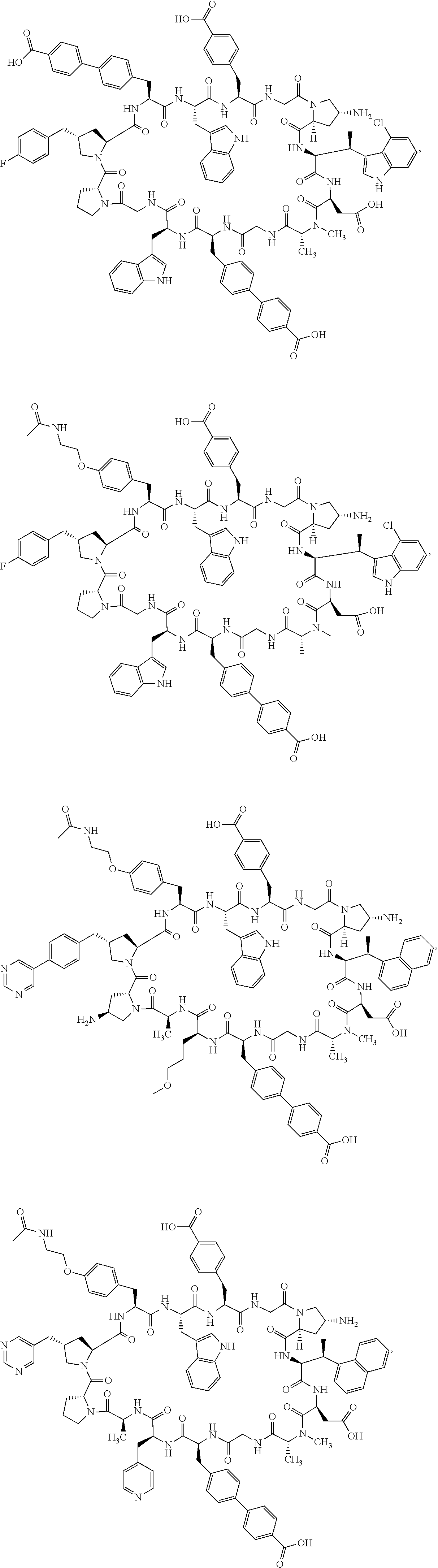

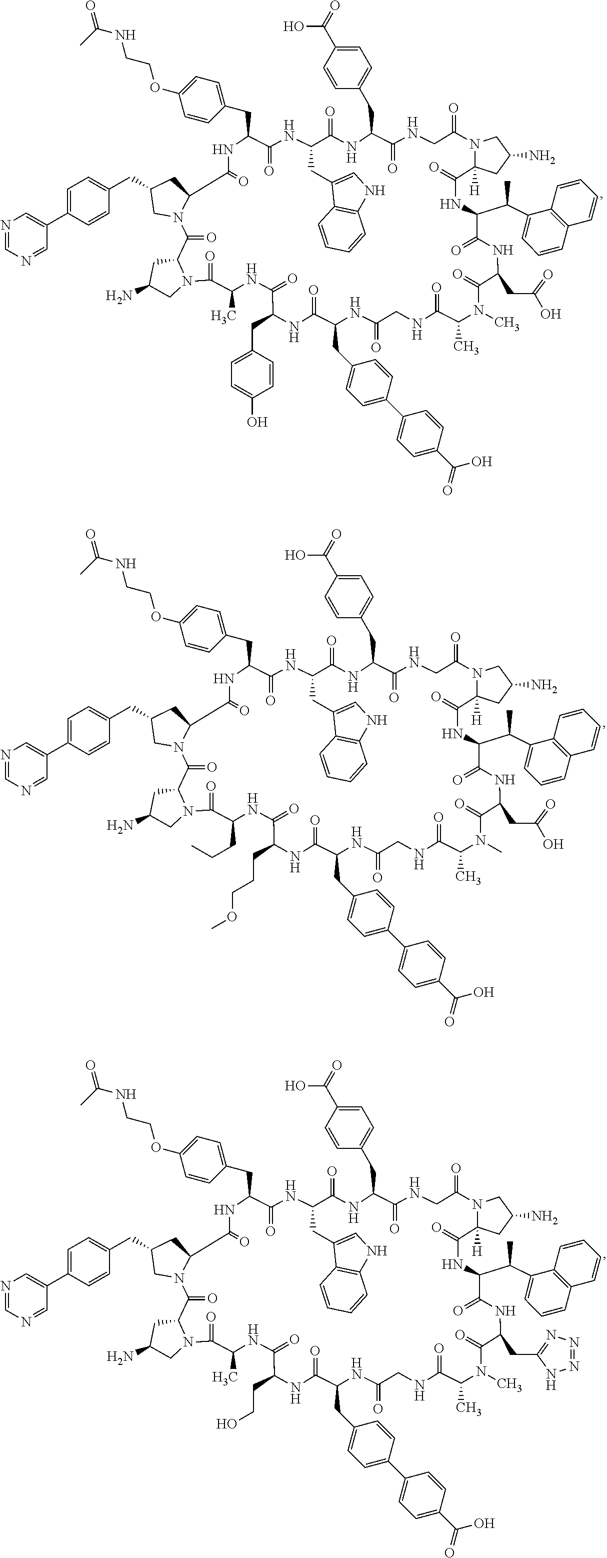

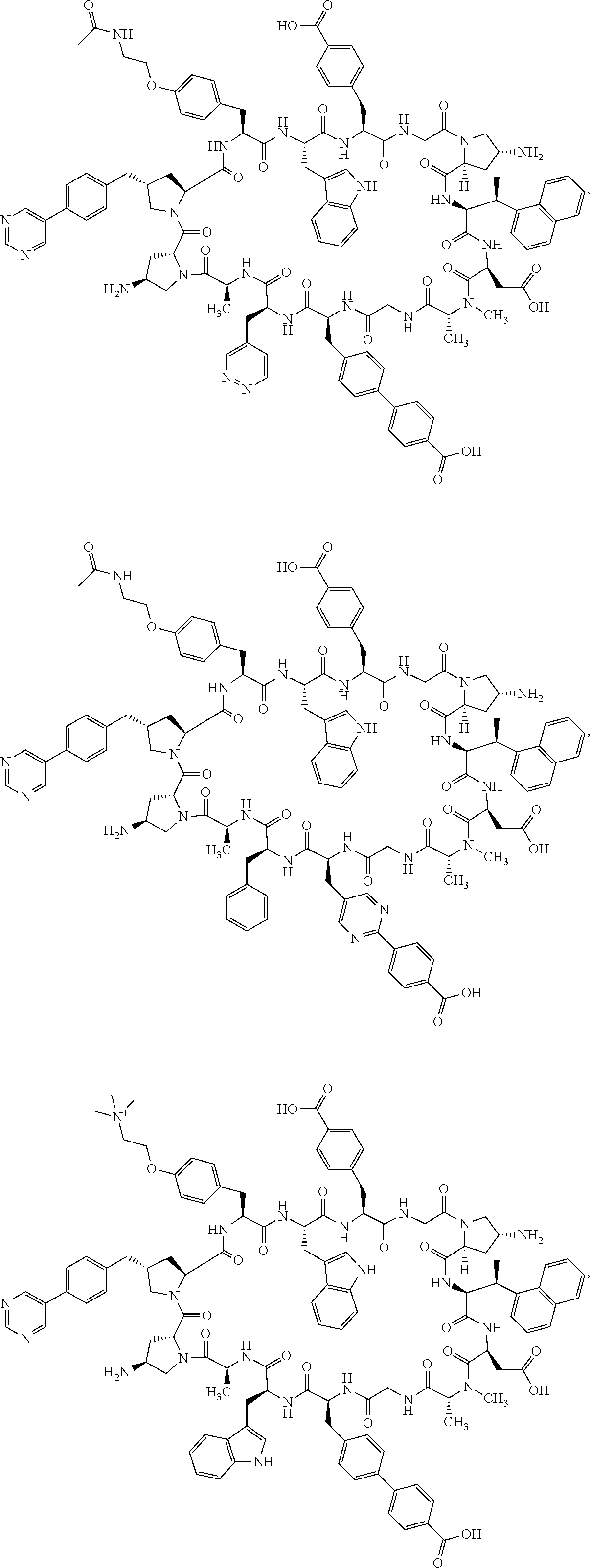

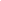

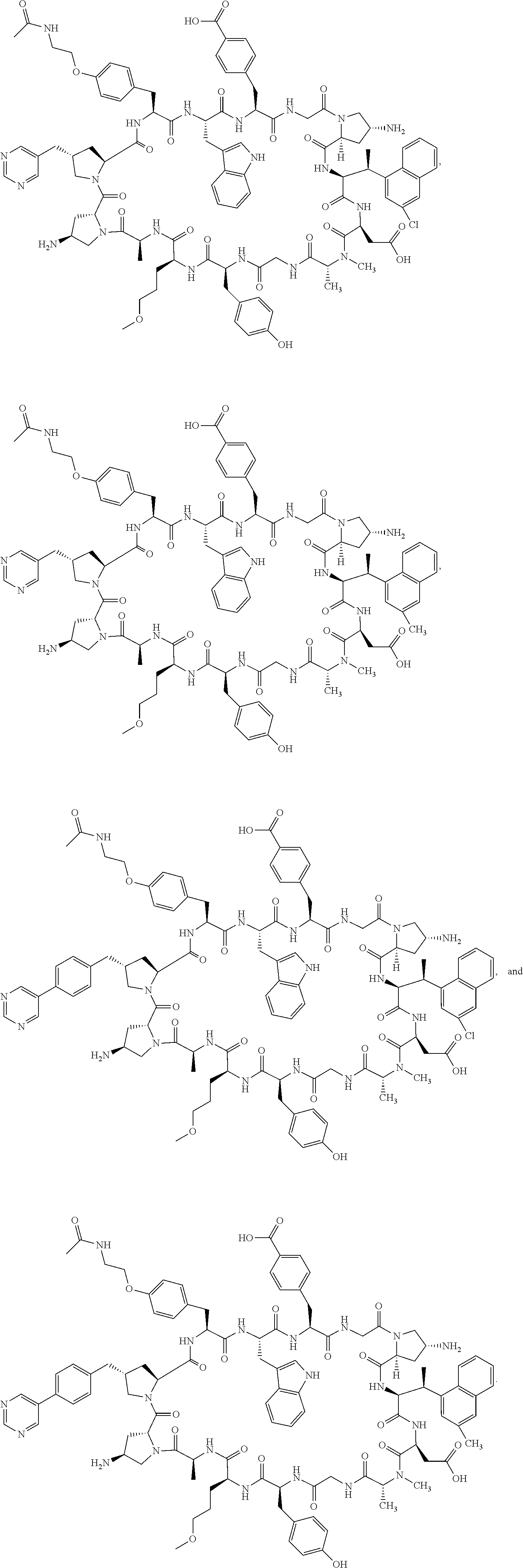

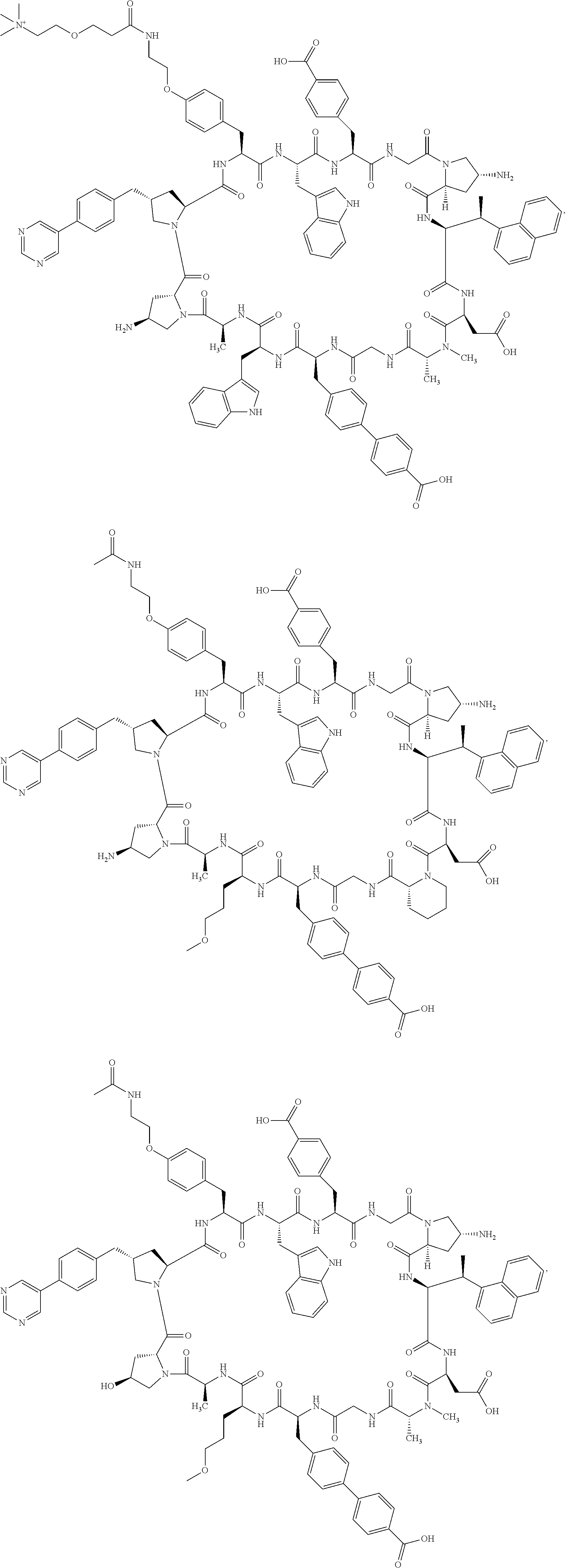

While not being bound by any specific theory, the Applicants believe that the compounds of the disclosure trap interleukin-1β, prevent signaling through the IL-1 receptor and hence reduce the downstream markers IL-6 and CRP. Hence the compounds can be useful to treat the inflammatory components of cardiovascular diseases such as ASCVD and heart failure with preserved ejection fraction (HFpEF). The compounds can also be useful to treat inflammatory disorders such as hidradenitis suppurativa (acne inversa), inflammatory bowel disease, and osteoarthritis.

### Definitions

Unless defined otherwise, all technical and scientific terms used herein have the same meaning as is commonly understood by one of skill in the art to which this invention belongs.

As used throughout this disclosure, “a compound of the disclosure”, “a compound of the present disclosure” and “a compound disclosed herein” are used interchangeably are to be understood to include the disclosed cyclic peptides and compounds of Formula (I). Reference to the compounds of Formula (I) herein encompass the compounds of each of Formulas (IA) and (IB), and all embodiments and classes thereof. The compounds of Formula (I) can form salts which are also within the scope of the present disclosure. Reference to a compound of the disclosure (or compound of Formula (I)) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to an amino group, pyrrolidine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

“Acyl” means an alkyl-C(O)— group, wherein alkyl is as defined below. The bond to the parent group is through the carbon atom of the carbonyl group.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Amino” means a H₂N— group. The bond to the parent group is through the nitrogen atom.

“Amino acid” refers to naturally-occurring α-amino acids and their stereoisomers, as well as unnatural amino acids (such as β-amino acids and substituted amino acids) and their stereoisomers. In the sequences given for the peptides (compounds) according to the present disclosure, the amino acid residues have their conventional meaning. Thus, “G” is glycine, “W” is tryptophan, “A” is alanine, “S” is serine, and so on. It is to be understood that “D” isomers are designated by a “d” before the one letter code or amino acid name, such that for example dA is the D isomer of L-alanine. Amino acid residues not encompassed by the foregoing have the definitions provided in the Table in the Examples section below.

“Aryl”, as used herein, represents a monocyclic 6-membered or bicyclic 10-membered ring system, wherein at least one ring is aromatic, and all the ring atoms are carbon.

“Bicyclic ring system” refers to two joined rings. The rings may be fused, i.e., share two adjacent atoms, or “spirocyclic”, i.e., share only a single atom.

“Carboxy” means a HO₂C— group. The bond to the parent group is through the carbon atom of the carbonyl component.

“Cycloalkyl” means a saturated cyclic hydrocarbon radical. In particular embodiments, the cycloalkyl group has 3-12 carbon atoms, forming 1-3 carbocyclic rings that. The rings may be fused, or “spirocyclic”, i.e., share only a single atom, or “bridged”, i.e., share three or more atoms with two bridgehead atoms being connected by a bridge containing at least one atom. Examples of cycloalkyl include cyclopropyl, cyclobutyl, cyclopentyl, cyclohexyl, cycloheptyl, adamantyl, bicyclo[1.1.1]pentanyl, and the like.

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heterocycloalkyl” or “heterocyclyl” means a non-aromatic monocyclic, bicyclic or tricyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. The rings of bi- and tricyclic ring may be fused, or “spirocyclic”, i.e., share only a single atom, or “bridged”, i.e., share three or more atoms with two bridgehead atoms being connected by a bridge containing at least one atom. There are no adjacent oxygen and/or sulfur atoms present in the ring system. In some embodiments, heterocycloalkyls contain about 5 to about 6 ring atoms. The prefix aza, oxa or thia before the heterocycloalkyl root name means that at least a nitrogen, oxygen or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, and the like.

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaromatic groups include pyridinyl, pyrimidinyl, pyrrolyl, pyridazinyl, isoxazolyl, thiazolyl, oxazolyl, indolyl, benzoxazolyl, benzothiazolyl, and imidazolyl.

When any variable (e.g., R^{C1}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{C9}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present disclosure.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of the disclosure and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds of the disclosure, can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When the compound of the present disclosure is acidic (or has a functional group which may be anionic), its corresponding salt can be conveniently prepared from pharmaceutically acceptable non-toxic bases, including inorganic bases and organic bases. Examples of suitable inorganic cations include, but are not limited to, alkali metal ions such as Li⁺, Na⁺, and K⁺, alkaline earth metal cations such as Ca2⁺, and Mg2⁺, and other cations such as Al³⁺ and Zn⁺. Examples of suitable organic cations include, but are not limited to, ammonium ion (i.e., NH₄⁺) and substituted ammonium ions. Examples of suitable substituted ammonium ions are those derived from methylamine, ethylamine, diethylamine, triethylamine and ethylenediamine. When a compound of the present disclosure is basic, its corresponding salt can be conveniently prepared from pharmaceutically acceptable non-toxic acids, including inorganic acids and organic acids. Example of such acid addition salts include salts formed from hydrohalic acids (e.g., hydrochloric, hydrobromic, hydroiodic), formic acid, acetic acid, capric acid, and citric acids. Salts containing acetate, formate, caprate, chloride, or sodium salts are typical for use with the compounds of the present disclosure. In some embodiments, salts of compounds of the present disclosure can be formed by exchange well-known to those of ordinary skill in the art, such as by anion exchange, e.g., replacement of trifluoroacetate ions with chloride ions.

Furthermore, compounds of the present disclosure may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of the instant disclosure may form solvates with water (i.e., a hydrate) or common organic solvents such as, but not limited to, acetic acid or acetonitrile. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of this disclosure which results in conversion in vivo to a compound within the scope of this disclosure is also within the scope of this disclosure.

The present disclosure also relates to processes for the preparation of the compounds of Formula (I) which are described in the following Examples and by which the compounds of the disclosure are obtainable.

“Treatment” and “treating” refer to all processes in which there may be a slowing, interrupting, arresting, controlling, or stopping of the progression of a disease or disorder described herein. The terms do not necessarily indicate a total elimination of all disease or disorder symptoms.

“Preventing,” or “prophylaxis,” as used herein, refers to reducing the likelihood of contracting disease or disorder described herein, or reducing the severity of a disease or disorder described herein.

The terms “therapeutically effective (or efficacious) amount” and similar descriptions such as “an amount efficacious for treatment” or “an effective dose” are intended to mean that amount of a compound of the disclosure that will elicit the biological or medical response of a tissue, a system, animal or human that is being sought by a researcher, veterinarian, medical doctor or other clinician. In a preferred embodiment, the term “therapeutically effective amount” means an amount of a compound of the disclosure that alleviates at least one clinical symptom in a human patient. The terms “prophylactically effective (or efficacious) amount” and similar descriptions such as “an amount efficacious for prevention” are intended to mean that amount of a compound of the disclosure that will prevent or reduce the risk of occurrence of the biological or medical event that is sought to be prevented in a tissue, a system, animal or human by a researcher, veterinarian, medical doctor or other clinician.

### Dosages of the Compounds of the Present Disclosure

The dosage regimen utilizing a compound of the present disclosure is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compound of the present disclosure is within the skill of the art. For administration to a human in the curative or prophylactic treatment of the conditions and disorders identified herein, for example, typical dosages of the compounds of the present disclosure can be about 0.05 mg/kg/day to about 50 mg/kg/day. In some embodiments, a patient is administered from about 5 mg/day to about 120 mg/day, such as from 10 mg/day, 20 mg/day, 30 mg/day, 40 mg/day, 50 mg/day, 60 mg/day, 70 mg/day, 80 mg/day, mg/day, 90 mg/day, or 100 mg/day of a compound of the present disclosure. In certain embodiments, a patient is administered from about 0.2 mg/kg to about 5 mg/kg, such as from 0.5 mg/kg, 0.75 mg/kg, 1.0 mg/kg, 1.25 mg/kg, or 1.5 mg/kg of a compound of the present disclosure. Such doses may be administered in a single dose or may be divided into multiple doses.

### Pharmaceutical Compositions

The compounds of the disclosure and their pharmaceutically acceptable salts can be administered to animals, preferably to mammals, and particularly to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of the disclosure and their pharmaceutically acceptable salts for use as pharmaceuticals, their use for modulating the activity of the cytokine IL-1β, and in particular, their use in the therapy and prophylaxis of the below-mentioned diseases or disorders as well as their use for preparing medicaments for these purposes. In certain embodiments, the compounds of the disclosure and their pharmaceutically acceptable salts trap IL-1β.

Furthermore, the present disclosure provides pharmaceutical compositions which comprise as active component an effective dose of at least one compound of the disclosure and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of the compound of the disclosure and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., atherosclerosis, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example, subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The present disclosure also provides pharmaceutical compositions comprising a compound of Formula (I). The compound of Formula (I) can be used in combination with any suitable pharmaceutical carrier or excipient. Such pharmaceutical compositions comprise a therapeutically effective amount of one or more compounds of Formula (I), and pharmaceutically acceptable excipient(s) and/or carrier(s). The specific pharmaceutic composition will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, absorption enhancers, administration aids, and combinations thereof.

Carriers are compounds and substances that improve and/or prolong the delivery of an active ingredient to a subject in the context of a pharmaceutical composition. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide, liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the compounds of Formula (I). In addition to their use in the pharmaceutical compositions of the present disclosure, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils, e.g., vegetable oils, may be used to provide oil-in-water or water in oil suspensions. Excipients which promote absorption from the gastrointestinal tract, e.g., permeation enhancers, such as sodium caprate can be included. In certain situations, delayed release preparations may be advantageous and compositions which can deliver the compounds of the present disclosure in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents or antioxidants. They may also contain therapeutically-active agents in addition to the compounds of the present disclosure.

### Methods of Using the Compounds of the Disclosure

The present application provides a method of IL-1 mediated cell signaling comprising contacting a cell with a compound of the disclosure or a pharmaceutically acceptable salt thereof. Inhibition of IL-1 mediated cell signaling can be assessed by detecting decreases in the levels of downstream biomarker IL-6 and CRP (e.g., hsCRP).

The present application also provides methods of using the compounds of the disclosure (or their pharmaceutically acceptable salts) or pharmaceutical compositions containing such compounds to treat disease conditions, including but not limited to, conditions implicated by IL-1β.

In some embodiments, the present disclosure provides a method of treating cardiovascular disease, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the cardiovascular disease is vascular inflammation. In some embodiments, the cardiovascular disease is atherosclerosis. In some embodiments, the cardiovascular disease is heart failure with preserved ejection fraction (HFpEF). In other embodiments the cardiovascular disease is heart failure with reduced ejection fraction (HFrEF).

In some embodiments, the present disclosure provides a method of treating a chronic kidney disease, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating inflammatory disorders, the method comprising administering a therapeutically effective amount of a compound of the disclosure (or a pharmaceutically acceptable salt thereof) or any of the foregoing pharmaceutical compositions comprising such a compound to a subject in need of such treatment. In certain embodiments, the inflammatory disorder is selected from the group consisting of hidradenitis suppurativa (acne inversa), inflammatory bowel disease, arthritis, and nonalcoholic steatohepatitis (NASH).

In some embodiments, the inflammatory disorder is hidradenitis suppurativa (acne inversa).

In certain embodiments, the inflammatory disorder is inflammatory bowel disease, such as Crohn's disease or ulcerative colitis.

In some embodiments, the inflammatory disorder is arthritis, e.g., osteoarthritis, rheumatoid arthritis, psoriatic arthritis, or gouty arthritis.

In other embodiments, the inflammatory disorder is nonalcoholic steatohepatitis (NASH).

### Combination Therapies

One or more additional pharmacologically active agents may be administered in combination with a compound of the disclosure. An additional active agent (or agents) is intended to mean a pharmaceutically active agent (or agents) that is active in the body, including pro-drugs that convert to pharmaceutically active form after administration, which are different from the compound of Formula I, and also includes free-acid, free-base and pharmaceutically acceptable salts of said additional active agents. Generally, any suitable additional active agent or agents, including but not limited to anti-hypertensive agents, anti-atherosclerotic agents such as a lipid modifying compound, anti-diabetic agents and/or anti-obesity agents, anti-inflammatory agents, may be used in any combination with the compound of the disclosure in a single dosage formulation (a fixed dose drug combination), or may be administered to the subject in one or more separate dosage formulations which allows for concurrent or sequential administration of the active agents (co-administration of the separate active agents).

Examples of additional active agents which may be employed in treating cardiovascular disorders include but are not limited to angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, i.e., COZAAR®, valsartan (including combinations with sacubitril), candesartan, olmesartan, telmesartan and any of these drugs used in combination with hydrochlorothiazide such as HYZAAR®); sGC activators (e.g., riociguat and vericiguat), PCSK9 inhibitors (e.g., evolocumab, alirocumab, MK-0616 and those disclosed in WO2019/246349), neutral endopeptidase inhibitors (e.g., thiorphan and phosphoramidon), aldosterone antagonists, aldosterone synthase inhibitors, renin inhibitors, endothelin receptor antagonists, phosphodiesterase-5 inhibitors (e.g., sildenafil, tadalafil and vardenafil), vasodilators, calcium channel blockers (e.g., amlodipine, nifedipine, verapamil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa) central alpha adrenergic agonists, peripheral vasodilators (e.g., hydralazine); lipid lowering agents e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), fluvastatin (particularly the sodium salt sold in LESCOL®), crivastatin, and pitavastatin; a cholesterol absorption inhibitor such as ezetimibe (ZETIA®) in combination with any other lipid lowering agents such as the HMG-CoA reductase inhibitors noted above and particularly with simvastatin (VYTORIN®) or with atorvastatin calcium; niacin in immediate-release or controlled release forms and/or with an HMG-CoA reductase inhibitor; niacin receptor agonists such as acipimox and acifran, as well as niacin receptor partial agonists; metabolic altering agents including insulin and insulin mimetics (e.g., insulin degludec, insulin glargine, insulin lispro), dipeptidyl peptidase-IV (DPP-4) inhibitors (e.g., sitagliptin, alogliptin, omarigliptin, linagliptin, vildagliptin); insulin sensitizers, including (i) PPARy agonists, such as the glitazones (e.g., pioglitazone, mitoglitazone, lobeglitazone, rosiglitazone, and balaglitazone), and other PPAR ligands, including (1) PPARα/γ dual agonists (e.g., chiglitazar, muraglitazar, aleglitazar, sodelglitazar, and naveglitazar); (2) PPARα agonists such as fenofibric acid derivatives (e.g., gemfibrozil, clofibrate, ciprofibrate, fenofibrate, bezafibrate), (3) selective PPAR γ modulators (SPPAR γ M's), (e.g., such as those disclosed in WO 02/060388, WO 02/08188, WO 2004/019869, WO 2004/020409, WO 2004/020408, and WO 2004/066963); and (4) PPAR γ partial agonists; (ii) biguanides, such as metformin and its pharmaceutically acceptable salts, in particular, metformin hydrochloride, and extended-release formulations thereof, such as Glumetza™, Fortamet™, and GlucophageXR™; and (iii) protein tyrosine phosphatase-1 B (PTP-1 B) inhibitors; insulin or insulin analogs (e.g., insulin detemir, insulin glulisine, insulin degludec, insulin glargine, insulin lispro and inhalable formulations of each); leptin and leptin derivatives and agonists; amylin and amylin analogs (e.g., pramlintide); sulfonylurea and non-sulfonylurea insulin secretagogues (e.g., tolbutamide, glyburide, glipizide, glimepiride, mitiglinide, meglitinides, nateglinide and repaglinide); α-glucosidase inhibitors (e.g., acarbose, voglibose and miglitol); glucagon receptor antagonists; incretin mimetics, such as GLP-1, GLP-1 analogs, derivatives, and mimetics; and GLP-1 receptor agonists (e.g., dulaglutide, semaglutide, albiglutide, exenatide, liraglutide, lixisenatide, taspoglutide, including intranasal, transdermal, and once-weekly formulations thereof); bile acid sequestering agents (e.g., colestilan, colestimide, colesevalam hydrochloride, colestipol, cholestyramine, and dialkylaminoalkyl derivatives of a cross-linked dextran), acyl CoA:cholesterol acyltransferase inhibitors, (e.g., avasimibe); antiobesity compounds; agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs); inhibitors of 11β-hydroxysteroid dehydrogenase type 1, (e.g., such as those disclosed in U.S. Pat. No. 6,730,690); inhibitors of fructose 1,6-bisphosphatase, (e.g., such as those disclosed in U.S. Pat. Nos. 6,054,587; 6,110,903; 6,284,748; 6,399,782; and 6,489,476); inhibitors of acetyl CoA carboxylase-1 or 2 (ACC1 or ACC2); AMP-activated Protein Kinase (AMPK) activators; other agonists of the G-protein-coupled receptors: (i) GPR-109, (ii) GPR-119, and (iii) GPR-40; SSTR3 antagonists (e.g., such as those disclosed in WO 2009/001836); neuromedin U receptor agonists (e.g., such as those disclosed in WO 2009/042053, including, but not limited to, neuromedin S (NMS)); SCD modulators; GPR-105 antagonists (e.g., such as those disclosed in WO 2009/000087); SGLT inhibitors (e.g., empagliflozin, dapagliflozin, canagliflozin, ertugliflozin, remogloflozin, tofogliflozin, and ipragliflozin); inhibitors of acyl coenzyme A:diacylglycerol acyltransferase 1 and 2 (DGAT-1 and DGAT-2); inhibitors of fatty acid synthase; inhibitors of acyl coenzyme A:monoacylglycerol acyltransferase 1 and 2 (MGAT-1 and MGAT-2); agonists of the TGR5 receptor (also known as GPBAR1, BG37, GPCR19, GPR131, and M-BAR); ileal bile acid transporter inhibitors; PACAP, PACAP mimetics, and PACAP receptor 3 agonists; PPAR agonists; protein tyrosine phosphatase-1 B (PTP-1 B) inhibitors; IL-1β antibodies, (e.g., gevokizumab and canakinumab); and bromocriptine mesylate and rapid-release formulations thereof; or with other drugs beneficial for the treatment of the above-mentioned conditions or disorders including the free-acid, free-base, and pharmaceutically acceptable salt forms of the above active agents where chemically possible.

Examples of additional active agents which may be employed in treating inflammatory disorders include but are not limited to steroidal and non-steroidal anti-inflammatory agents, glucocorticoids, and therapeutic hormones. In particular embodiments, in treating hidradenitis suppurativa (acne inversa), the additional active agent can be an antibiotic, an injectable steroid, a therapeutic hormone, a TNF inhibitor (e.g., infliximab, adalimumab, etanercept, golimumab, certolizumab), a pain medication (e.g., codeine, hydrocodone, morphine, pregabalin, gabapentin, Intralesional triamcinolone, a corticosteroid, naproxen, ketoprofen, diclofenac, ibuprofen, acetaminophen). In other embodiments, in treating an inflammatory bowel disease, the additional active agent can be methotrexate, a TNF inhibitor, an oral sphingosine 1-phosphate receptor modulator (e.g., fingolimod, siponimod, ozanimod, ponesimod) or a selective JAK inhibitor (e.g., tofacitinib, baricitinib, upadacitinib). In some embodiments, in treating osteoarthritis, the additional active agent can be a pain medication (examples listed above). In other embodiments, in treating gouty arthritis, the additional active agent can be colcichine, a non-steroidal anti-inflammatory agent, or a glucocorticoid.

### Examples

### Methods of Preparing the Compounds of the Disclosure

The compounds described herein can be prepared according to the procedures of the following schemes and examples, using appropriate materials and are further exemplified by the following specific examples. The examples also include methods for testing such compounds in cellular assays. The compounds illustrated in the examples are not, however, to be construed as forming the only genus that is considered as the disclosure.

The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. Starting materials and intermediates for the final compounds are purchased, made from known procedures, or as otherwise illustrated. The examples are provided for the purpose of further illustration only and are not intended to be limitations on the disclosure.

NMR data were obtained on a 300 MHz or 400 MHz instrument in CDC₃, DMSO-d₆, or Methanol-d₄ with the chemical shifts reported relative to tetramethylsilane standard. Resonance signals are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

Throughout the synthetic schemes and examples, abbreviations and acronyms may be used with the following meanings unless otherwise indicated:

Abbreviations

| Abbreviation | Definition |
|---|---|
| 3AzaPhe4AcPip | (S)-3-(6-(4-Acetylpiperazin-1-yl)pyridin-3-yl)-2- |
| | aminopropanoic acid |
| 3Pal | (2S)-2-amino-3-(3-pyridyl)propanoic acid |
| 3Pal4Ph4CO2H | 4-[5-[(2S)-2-amino-2-carboxy-ethyl]-2-pyridyl]benzoic acid |
| 3Pal4Ph4CO2H | (S)-4-(5-(2-amino-2-carboxyethyl)pyridin-2-yl)benzoic acid |
| 4CzIPN | 1,2,3,5-Tetrakis(carbazol-9-yl)-4,6-dicyanobenzene |
| 4Pal | (2S)-2-amino-3-(4-pyridyl)propanoic acid |
| 4PyridaAla | (2S)-2-amino-3-pyridazin-4-yl-propanoic acid |
| A | 1-Alanine |
| AA | Amino acid |
| Ac | Acetyl |
| ACN or MeCN | Acetonitrile |
| AcOH | Acetic acid |
| AEF | (S)-2-amino-3-(4-(2-aminoethoxy)phenyl)propanoic acid |
| AEFC4AcidNMe3 | (S)-4-((2-(4-(2-amino-2-carboxyethyl)phenoxy)ethyl)amino)- |
| | N,N,N-trimethyl-4-oxobutan-1-aminium |
| AEFC6AcidNMe3 | (S)-6-((2-(4-(2-amino-2-carboxyethyl)phenoxy)ethyl)amino)- |
| | N,N,N-trimethyl-6-oxohexan-1-aminium |
| AEFNMe3 | (S)-2-(4-(2-amino-2-carboxyethyl)phenoxy)-N,N,N- |
| | trimethylethan-1-aminium |
| AEFPEG1AcidNMe3 | (S)-2-(3-((2-(4-(2-amino-2- |
| | carboxyethyl)phenoxy)ethyl)amino)-3-oxopropoxy)-N,N,N- |
| | trimethylethan-1-aminium |
| AEFPEG2acidNMe3 | (S)-2-(2-(3-((2-(4-(2-amino-2- |
| | carboxyethyl)phenoxy)ethyl)amino)-3-oxopropoxy)ethoxy)- |
| | N,N,N-trimethylethan-1-aminium |
| Ala2Oxad1345Me | (S)-2-Amino-3-(5-methyl-1,2,4-oxadiazol-3-yl)propanoic |
| | acid |
| Ala3Oxad1245Me | (S)-2-Amino-3-(5-(methoxymethyl)-1,2,4-oxadiazol-3- |
| | yl)propanoic acid |
| Ala3Oxad1245MeOMe | (S)-2-Amino-3-(5-(methoxymethyl)-1,2,4-oxadiazol-3- |
| | yl)propanoic acid |
| AlaPent | (2S)-2-amino-3-(1-bicyclo[1.1.1]pentanyl)propanoic acid |
| AlaTet5 | (S)-2-amino-3-(2H-tetrazol-5-yl)propanoic acid |
| AlaTHP4 | (S)-2-Amino-3-(tetrahydro-2H-pyran-4-yl)propanoic acid |
| ASCVD | Atherosclerotic cardiovascular disease |
| Asu | (S)-2-Aminooctanedioic acid |
| BCP3Ph4CO2H | (S)-4-(3-(2-Amino-2-carboxyethyl)bicyclo[1.1.1]pentan-1- |
| | yl)benzoic acid |
| Bip4CO2H | 4-[4-[(2S)-2-amino-2-carboxy-ethyl]phenyl]benzoic acid |
| Bip4CONH2 | (S)-2-Amino-3-(4′-carbamoyl-[1,1′-biphenyl]-4-yl)propanoic |
| | acid |
| Boc | tert-Butoxy-carbonyl |
| Boc₂O | Di-tert-butyl dicarbonate |
| CAN | Ammonium Cerium(IV) Nitrate |
| CANTOS | Canakinumab Anti-inflammatory Thrombosis Outcomes |
| | Study |
| Cbz | Benzyl carbamate |
| CDCl₃ | Deuterated chloroform |
| Cha | (S)-2-((((9H-Fluoren-9-yl)methoxy)carbonyl)amino)-3- |
| | cyclohexylpropanoic acid |
| Cit | (2S)-2-amino-5-ureido-pentanoic acid |
| COD | 1,5-Cyclooctadiene |
| CRP | C-reactive protein |
| Cy | Cyclohexane |
| D | 1-Aspartic acid |
| Dab | (2S)-2,4-diaminobutanoic acid |
| Dap | (S)-2-Amino-3-(carboxyamino)propanoic acid |
| dba | Dibenzylideneacetone |
| DCC | N,N′-dicyclohexylcarbodiimide |
| DCC | N,N′-Dicyclohexylcarbodiimide |
| DCE | Dichloroethane |
| DCM | Dichloromethane |
| DIC | N,N′-Diisopropylcarbodiimide |
| DIEA or DIPEA | N,N-Diisopropylethylamine or Hunig's base |
| DMA | N,N-dimethylacetamide |
| DMAP | 4-dimethylaminopyridine |
| DMF | N,N-dimethylformamide |
| DMSO | Dimethylsulfoxide |
| dNMeA | D-N-methyl-Alanine |
| dNMeD | N-Methyl-D-aspartic acid |
| dP | D-proline |
| dPip | (R)-piperidine-2-carboxylic acid |
| dProc4NH2 | (2R,4R)-4-aminopyrrolidine-2-carboxylic acid |
| dProt4F | (2R,4S)-4-Fluoropyrrolidine-2-carboxylic acid |
| dProt4NH2 | (2R,4S)-4-aminopyrrolidine-2-carboxylic acid |
| dProt4NH2C6acidNMe3 | 6-(((3S,5R)-5-carboxypyrrolidin-3-yl)amino)-N,N,N- |
| | trimethyl-6-oxohexan-1-aminium |
| dProt4NHPEG2acidNMe3 | 2-(2-(3-(((3S,5R)-5-carboxypyrrolidin-3-yl)amino)-3- |
| | oxopropoxy)ethoxy)-N,N,N-trimethylethan-1-aminium |
| dProt4NMe3 | (3S,5R)-5-Carboxy-N,N,N-trimethylpyrrolidin-3-aminium |
| dProt4OH | (2R,4S)-4-hydroxypyrrolidine-2-carboxylic acid |
| dtbbpy | 4,4′-di-tert-butyl-2,2′-dipyridyl |
| dtbpf | (di-tert-butylphosphino)ferrocene |
| E | 1-Glutamic acid |
| EA | ethyl acetate |
| EDCI | N-[3-(Methylamino)propyl]-N′-ethylcarbodiimide |
| | hydrochloride |
| ESI | electrospray ionization |
| EtOAc | Ethyl acetate |
| F | 1-Phenylalanine |
| F4SO2NH2 | (S)-2-Amino-3-(4-sulfamoylphenyl)propanoic acid |
| Fmoc | 9H-fluoren-9-ylmethoxycarbonyl |
| Fmoc-OSU | (9H-Fluoren-9-yl)methyl (2,5-dioxopyrrolidin-1-yl) carbonate |
| G | Glycine |
| h | hour |
| HATU | 1-[Bis(dimethylamino)methylene]-1H-1,2,3-triazolo[4,5- |
| | b]pyridinium 3-oxid hexafluorophosphate or N- |
| | [(Dimethylamino)-1H-1,2,3-triazolo-[4,5-b]pyridin-1- |
| | ylmethylene]-N-methylmethanaminium hexafluorophosphate |
| | N-oxide |
| hCha | (S)-2-Amino-4-cyclohexylbutanoic acid |
| HFIP | Hexafluoroisopropanol |
| HFpEF | Heart failure with preserved ejection fraction |
| HPLC | High-performance liquid chromatography |
| hS | 1-homoserine |
| hsCRP | High sensitivity C-reactive protein |
| IPA | Isopropanol |
| K | 1-Lysine |
| K3CH3NPEG4 | (S)-21-Amino-21-carboxy-N,N,N-trimethyl-15-oxo-3,6,9,12- |
| | tetraoxa-16-azahenicosan-1-aminium |
| KPEG2acidNMe3 | (S)-2-(2-(3-((5-amino-5-carboxypentyl)amino)-3- |
| | oxopropoxy)ethoxy)-N,N,N-trimethylethan-1-aminium |
| LCMS | Liquid chromatography mass spectrometry |
| LDA | Lithium diisopropylamide |
| LDL | Low-density lipoprotein |
| LED | Light emitting diode |
| MACE | Major adverse cardiac events |
| MeOH | Methanol |
| min | Minute |
| mL | milliliter |
| MS | Mass spectrometry |
| N | L-Asparagine |
| NdM | N4,N4-dimethyl-L-asparagine |
| NIS | N-iodosuccinimide |
| NMO | N-Methylmorpholine N-oxide |
| NMP | N-Methyl-2-pyrrolidone |
| NMR | Nuclear magnetic resonance |
| NsCl | 4-Nitrobenzenesulfonyl chloride |
| Nva | (S)-2-Aminopentanoic acid |
| NvaOMe | (S)-2-amino-5-methoxypentanoic acid |
| o.n. | Over night |
| OMpe | β-methylpentyl ester |
| Orn | 1-Ornithine |
| OrnMe | (S)-2-amino-5-(methylamino)pentanoic acid |
| OrnMe3 | (S)-4-Amino-4-carboxy-N,N,N-trimethylbutan-1-aminium |
| OSu | O-succinimide |
| Oxyma | Ethyl cyano(hydroxyimino)acetate |
| P | L-Proline |
| Pbf | 2,2,4,6,7-pentamethyldihydrobenzofuran-5-sulfonyl |
| PDC | Pyridinium dichromate |
| PE | Petroleum ether |
| PfTrpB-7E6 | Engineered tryptophan synthase from Pyrococcus furiosus |
| Phe3CONH2 | (S)-2-Amino-3-(3-carbamoylphenyl)propanoic acid |
| Phe42Oxad1345Me | (S)-2-Amino-3-(4-(5-methyl-1,3,4-oxadiazol-2- |
| | yl)phenyl)propanoic acid |
| Phe43Oxad1245Me | (S)-2-Amino-3-(4-(5-methyl-1,2,4-oxadiazol-3- |
| | yl)phenyl)propanoic acid |
| Phe43Oxad1245MeOEt | (S)-2-Amino-3-(4-(5-(ethoxymethyl)-1,2,4-oxadiazol-3- |
| | yl)phenyl)propanoic acid |
| Phe4AcPip | (S)-3-(4-(4-Acetylpiperazin-1-yl)phenyl)-2-aminopropanoic |
| | acid |
| Phe4BR4 | (S)-2-amino-3-(4′-(methylsulfonamido)-[1,1′-biphenyl]-4- |
| | yl)propanoic acid |
| Phe4CONH2 | (S)-2-Amino-3-(4-carbamoylphenyl)propanoic acid |
| Phe4COOH | 4-[(2S)-2-amino-2-carboxy-ethyl]benzoic acid |
| Phe4pcCCA | (S)-2-Amino-3-(4-((1s,4R)-4-(tert- |
| | butoxycarbonyl)cyclohexyl)phenyl)propanoic acid |
| Phe4ptCCA | (S)-2-Amino-3-(4-((1r,4S)-4-(tert- |
| | butoxycarbonyl)cyclohexyl)phenyl)propanoic acid |
| PLP | 3-hydroxy-2-methyl-5-([phosphonooxy]methyl)-4- |
| | pyridinecarboxaldehyde or pyridoxal 5-phosphate |
| PPh₃ | Triphenylphosphine |
| Proc4F | (2S,4S)-4-Fluoropyrrolidine-2-carboxylic acid |
| Proc4OH | (2S,4S)-4-Hydroxypyrrolidine-2-carboxylic acid |
| Prot3Ph3F | (2S,3R)-3-(3-fluorophenyl)pyrrolidine-2-carboxylic acid |
| Prot4Bn3F4F | (2S,4R)-4-(3,4-difluorobenzyl)pyrrolidine-2-carboxylic acid |
| Prot4Bn4F | (2S,4R)-4-(4-fluorobenzyl)pyrrolidine-2-carboxylic acid |
| Prot4CH2Bip | (2S,4R)-4-([1,1′-biphenyl]-4-ylmethyl)pyrrolidine-2- |
| | carboxylic acid |
| Prot4CH2Pyrim | (2S,4R)-4-(pyrimidin-5-ylmethyl)pyrrolidine-2-carboxylic |
| | acid |
| Prot4F | (2S,4R)-4-Fluoropyrrolidine-2-carboxylic acid |
| Prot4NH2 | (2S,4R)-4-aminopyrrolidine-2-carboxylic acid |
| Prot4OH | (2S,4R)-4-Hydroxypyrrolidine-2-carboxylic acid |
| psi | Pound-force per square inch |
| Py | Pyridine |
| PyAOP | (7-Azabenzotriazol-1-yloxy)tripyrrolidinophosphonium |
| | hexafluorophosphate |
| Pyrim | (2S,4R)-4-(4-(pyrimidin-5-yl)benzyl) pyrrolidine-2- |
| | carboxylic acid |
| PyrimAla4Ph4CO2H | 4-[5-[(2S)-2-amino-2-carboxy-ethyl]pyrimidin-2-yl]benzoic |
| | acid |
| Q | 1-Glutamine |
| Qdm | N5,N5-Dimethyl-L-glutamine |
| R | 1-Arginine |
| RP | Reverse phase |
| rpm | Revolutions per minute |
| RT | Retention time |
| rt | Room temperature |
| S | 1-Serine |
| sbMe1Nal | (2S,3S)-2-amino-3-(1-naphthyl)butanoic acid |
| SbMe1Nal3Cl | (2S,3S)-2-Amino-3-(3-chloronaphthalen-1-yl)butanoic acid |
| SbMe1Nal3Me | (2S,3S)-2-Amino-3-(3-methylnaphthalen-1-yl)butanoic acid |
| SbMe1Nal3OMe | (2S,3S)-2-Amino-3-(3-methoxynaphthalen-1-yl)butanoic acid |
| SbMe1Nal8F | (2S,3S)-2-Amino-3-(3-methoxynaphthalen-1-yl)butanoic acid |
| SbMeW4Cl | (2S,3S)-2-amino-3-(4-chloro-1H-indol-3-yl)butanoic acid |
| SbMeW4F | (2S,3S)-2-amino-3-(4-fluoro-1H-indol-3-yl)butanoic acid |
| SerOMe | O-Methyl-L-serine |
| SFC | Supercritical fluid chromatography |
| SPPS | Solid-Phase Peptide Synthesis |
| Su | Succinimide |
| T | 1-threonine |
| TBAB | Tetrabutylammonium bromide |
| TBAF | Tetrabutylammonium fluoride |
| TBAI | Tetrabutylammonium iodide |
| tBu | tert-Butyl |
| t-BuOH | tert-Butanol |
| TEA | Triethylamine |
| TFA | Trifluoroacetic acid |
| THF | Tetrahydrofuran |
| TIS | Triisopropylsilane |
| TMSCl | Trimethylsilyl chloride |
| Trp2Me | (S)-2-Amino-3-(2-methyl-1H-indol-3-yl)propanoic acid |
| Trp6F | (S)-2-Amino-3-(6-fluoro-1H-indol-3-yl)propanoic acid |
| TyrEtNAc | (2S)-3-[4-(2-acetamidoethoxy)phenyl]-2-amino-propanoic |
| | acid |
| TyrOCOPyrr | (S)-2-Amino-3-(4-((pyrrolidine-1- |
| | carbonyl)oxy)phenyl)propanoic acid |
| UPLC | Ultra Performance Liquid Chromatography or Ultrahigh |
| | Pressure Liquid Chromatography |
| UV | ultraviolet |
| V | 1-valine |
| W | 1-Tryptophan |
| wt | weight |
| Y | 1-tyrosine |
| YFN | (S)-2-Amino-3-(4-(5-oxo-4,5-dihydro-1,2,4-oxadiazol-3- |
| | yl)phenyl)propanoic acid |

The following examples are meant to be illustrative and should not be construed as further limiting. The contents of the figures and all references, patents, and published patent applications cited throughout this application are expressly incorporated herein by reference.

### Intermediate Syntheses:

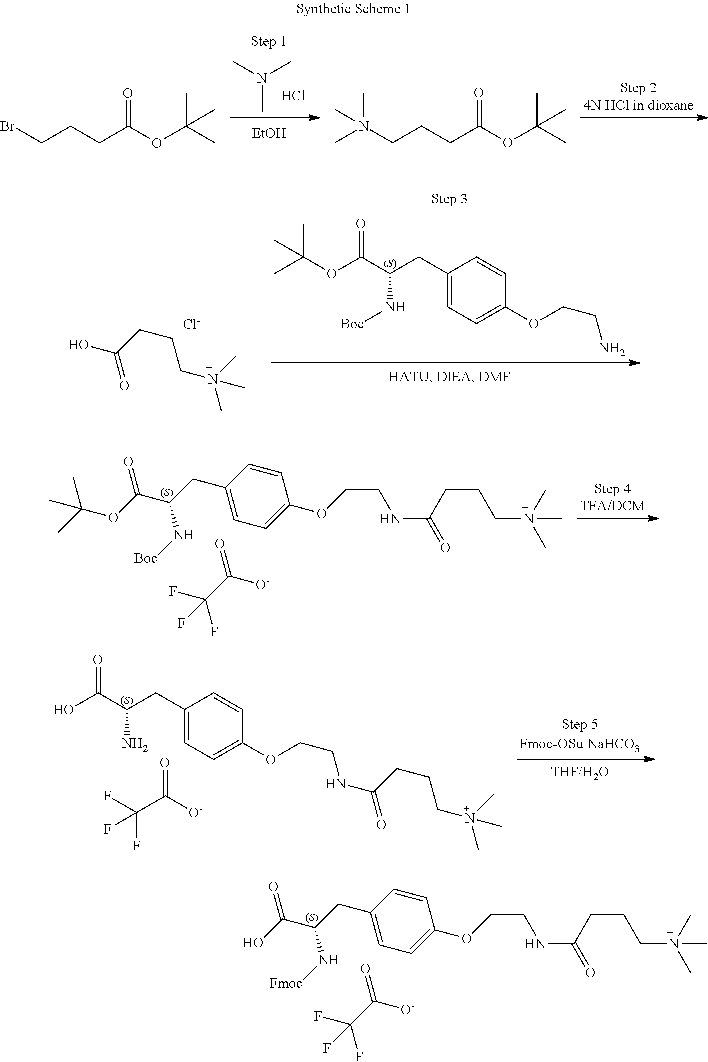

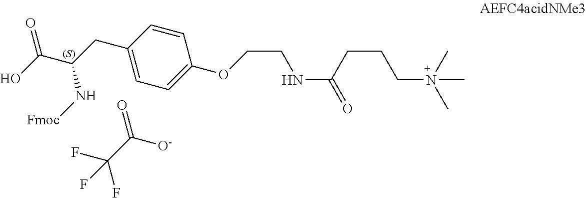

### (S)-4-((2-(4-(2-((((9H-fluoren-9-yl)methoxy)carbonyl)amino)-2-carboxyethyl)phenoxy)ethyl)amino)-N,N,N-trimethyl-4-oxobutan-1-aminium 2,2,2-trifluoroacetate

Step 1: To a stirred solution of tert-butyl 4-bromobutanoate (3 g, 13.45 mmol) and trimethylamine hydrochloride (10.28 g, 108 mmol) in EtOH (60 mL) was added NaHCO₃ (12.44 g, 148 mmol) at room temperature. The mixture was stirred at 50° C. for 16 h. The mixture was cooled to room temperature and the solid was filtered out. The filtrate was concentrated under reduced pressure and the residue was suspended in DCM (30 mL). The solid was filtered out and the filtrate was concentrated under reduced pressure to give 4-(tert-butoxy)-N,N,N-trimethyl-4-oxobutan-1-aminium (2 g, 7.91 mmol, 58.8% yield) as a colorless oil. MS ESI calculated for C₁₁H₂₄NO₂⁺ [M]⁺ 202.18, found 202.20.

Step 2: To a stirred solution of 4-(tert-butoxy)-N,N,N-trimethyl-4-oxobutan-1-aminium (2 g, 9.89 mmol) in 4 N HCl in dioxane (50 mL) was stirred at 25° C. for 2 h. The solvent was concentrated under reduced pressure to give 3-carboxy-N,N,N-trimethylpropan-1-aminium chloride (1.3 g, 8.89 mmol, 90% yield) as an off-white solid. MS ESI calculated for C₇H₁₆NO₂⁺ [M-Cl]⁺ 146.12, found 146.15.

Step 3: To a stirred solution of tert-butyl (S)-3-(4-(2-aminoethoxy)phenyl)-2-((tert-butoxycarbonyl)amino)propanoate (5 g, 13.14 mmol) in DMF were added 3-carboxy-N,N,N-trimethylpropan-1-aminium chloride (5.76 g, 39.4 mmol) and DIEA (8.49 g, 65.7 mmol) at room temperature. The solution was stirred at 0° C. for 10 min. HATU (6.00 g, 15.77 mmol) was added to the solution and the solution was stirred at 0° C. for 2 h. The solution was purified by RP-flash with the following conditions: C¹⁸ column, 330 g, 1%-1% in 5 min, 1%-40% in 25 min, MeCN in water (0.05% TFA) to give (S)-4-((2-(4-(3-(tert-butoxy)-2-((tert-butoxycarbonyl)amino)-3-oxopropyl)phenoxy)ethyl)amino)-N,N,N-trimethyl-4-oxobutan-1-aminium as an off-white solid. MS ESI calculated for C₂₇H₄₆N₃O₆ [M-CF₃COO]⁺ 508.34, found 508.25.

Step 4: To a stirred solution of (S)-4-((2-(4-(3-(tert-butoxy)-2-((tert-butoxycarbonyl)amino)-3-oxopropyl)phenoxy)ethyl)amino)-N,N,N-trimethyl-4-oxobutan-1-aminium 2,2,2-trifluoroacetate (6.4 g, 10.29 mmol) in DCM (80 mL) was added TFA (120 mL) at room temperature. The solution was stirred at room temperature for 3 h. The solvent was concentrated under reduced pressure to give (S)-4-((2-(4-(2-amino-2-carboxyethyl)phenoxy)ethyl)amino)-N,N,N-trimethyl-4-oxobutan-1-aminium 2,2,2-trifluoroacetate (4.7 g, 10.10 mmol, 98% yield) as a yellow oil. MS ESI calculated for C₁₈H₃₀N₃O₄ [M-CF₃COO]⁺ 352.22, found 352.10.

Step 5: To a mixture of (S)-4-((2-(4-(2-amino-2-carboxyethyl)phenoxy)ethyl)amino)-N,N,N-trimethyl-4-oxobutan-1-aminium 2,2,2-trifluoroacetate (6.4 g, 13.75 mmol) in THF (60 mL) and water (60 mL) were added NaHCO₃ (5.78 g, 68.7 mmol) and Fmoc-OSu (4.64 g, 13.75 mmol) at 25° C. The mixture was stirred at 25° C. for 2 h. The pH value of the reaction mixture was adjusted to 3 with 1 N HCl. The mixture was concentrated under reduced pressure. The residue was purified by RP-flash with the following conditions: C¹⁸ column, 330 g, 1%-1% in 5 min, 1%-50% in 30 min, MeCN in water (0.05% TFA) to give (S)-4-((2-(4-(2-((((9H-fluoren-9-yl)methoxy)carbonyl)amino)-2-carboxyethyl)phenoxy)ethyl)amino)-N,N,N-trimethyl-4-oxobutan-1-aminium 2,2,2-trifluoroacetate (4.7434 g, 6.55 mmol, 47.7% yield) as a light yellow semi-solid. MS ESI calculated for C₃₃H₄₀N₃O₆ [M-CF₃COO]⁺ 574.29, found 574.15. ¹H NMR (300 MHz, CD₃OD) δ 7.76-7.74 (m, 2H), 7.59-7.56 (m, 2H), 7.40-7.35 (m, 2H), 7.31-7.26 (m, 2H), 7.16-7.14 (m, 2H), 6.84-6.81 (m, 2H), 4.38-4.12 (m, 4H), 3.98-3.95 (m, 2H), 3.52 (t, J=5.4 Hz, 2H), 3.32-3.24 (m, 2H), 3.13-3.03 (m, 10H), 2.91-2.85 (m, 1H), 2.30 (t, J=6.9 Hz, 2H), 2.05-1.99 (m, 2H).

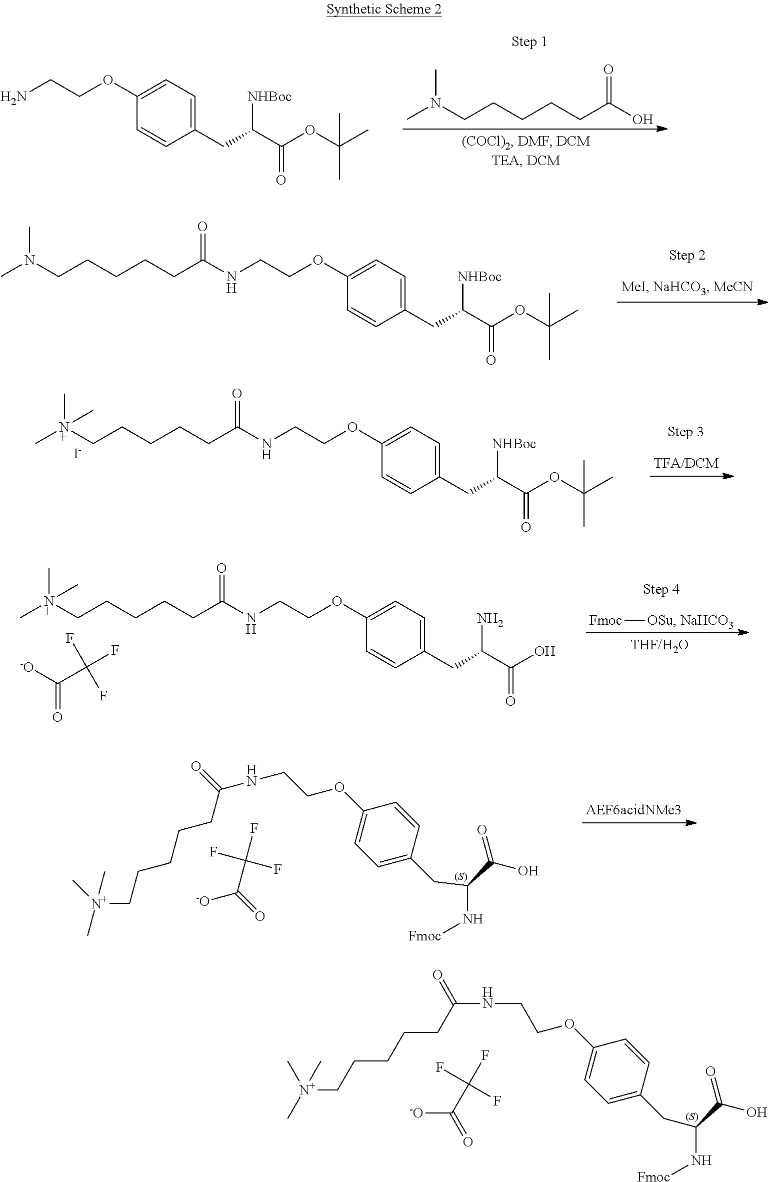

### (S)-6-((2-(4-(2-((((9H-fluoren-9-yl)methoxy)carbonyl)amino)-2-carboxyethyl)phenoxy)ethyl)amino)-N,N,N-trimethyl-6-oxohexan-1-aminium trifluoroacetate

Step 1: To a stirred mixture of 6-(dimethylamino)hexanoic acid (2 g, 12.56 mmol) in DCM (20 mL) was added N,N-dimethylformamide (0.092 g, 1.256 mmol) at 25° C., then the solution of oxalyl dichloride (4.78 g, 37.7 mmol) in DCM (10 mL) was added at 0° C. The resulting mixture was stirred for 1 h at 30° C. The reaction mixture was concentrated under reduced pressure to give crude 6-(dimethylamino)hexanoyl chloride (2.232 g, 12.56 mmol, 100% yield) as a yellow oil, which was dissolved in DCM (7 mL). TEA (5.78 g, 57.1 mmol) and the solution of 6-(dimethylamino)hexanoyl chloride (2.232 g, 12.56 mmol) in DCM (7 mL) were added to the stirred mixture of tert-butyl (S)-3-(4-(2-aminoethoxy)phenyl)-2-((tert-butoxycarbonyl)amino)propanoate (4.34 g, 11.42 mmol) in DCM (13 mL) at 0° C. After the resulting mixture was stirred for 2 h at 25° C., it was concentrated under reduced pressure. The residue was purified by silica gel column chromatography, eluted with 0-30% MeOH (1% NH₄OH) in DCM and the product-containing fractions were collected and evaporated in vacuo to afford tert-butyl (S)-2-((tert-butoxycarbonyl)amino)-3-(4-(2-(6-(dimethylamino)hexanamido)ethoxy)phenyl)propanoate (1.7 g, 3.26 mmol, 29% yield) as a light white solid. MS ESI calculated for C₂₈H₄₈N₃O₆ [M+H]⁺ 522.35, found 522.30.

Step 2: To a stirred mixture of tert-butyl (S)-2-((tert-butoxycarbonyl)amino)-3-(4-(2-(6-(dimethylamino)hexanamido)ethoxy)phenyl)propanoate (1.7 g, 3.26 mmol) in MeCN (17 mL) were added NaHCO₃ (2.74 g, 32.6 mmol) and Mel (1.019 mL, 16.29 mmol) at 25° C. The resulting mixture was stirred for 16 h at 25° C. The solid was filtered out and the filtrate was concentrated under reduced pressure to give crude (S)-6-((2-(4-(3-(tert-butoxy)-2-((tert-butoxycarbonyl)amino)-3-oxopropyl)phenoxy)ethyl)amino)-N,N,N-trimethyl-6-oxohexan-1-aminium iodide (2.163 g, 3.26 mmol, 100% yield) as a yellow oil. MS ESI calculated for C₂₉H₅₀IN₃O₆[M-I]⁺ 536.37, found 536.35.

Step 3: To a stirred mixture of (S)-6-((2-(4-(3-(tert-butoxy)-2-((tert-butoxycarbonyl)amino)-3-oxopropyl)phenoxy)ethyl)amino)-N,N,N-trimethyl-6-oxohexan-1-aminium iodide (2.163 g, 3.26 mmol) in DCM (10 mL) was added TFA (10 mL, 130 mmol) at 25° C. After the resulting mixture was stirred for 2 h at 25° C., it was concentrated under reduced pressure to give crude (S)-6-((2-(4-(2-amino-2-carboxyethyl)phenoxy)ethyl)amino)-N,N,N-trimethyl-6-oxohexan-1-aminium 2,2,2-trifluoroacetate (1.609 g, 3.26 mmol, 100% yield) as a yellow oil. MS ESI calculated for C₂₂H₃₄F₃N₃O₆[M-CF₃COO]⁺ 380.25, found 380.15.

Step 4: To a solution of (S)-6-((2-(4-(2-amino-2-carboxyethyl)phenoxy)ethyl)amino)-N,N,N-trimethyl-6-oxohexan-1-aminium 2,2,2-trifluoroacetate (1.609 g, 3.26 mmol) in THF (8 mL) and water (8 mL) were added NaHCO₃ (1.369 g, 16.30 mmol) and Fmoc-Osu (1.002 g, 2.93 mmol) at 25° C. The resulting mixture was stirred at room temperature for 16 h, then the pH was adjusted to 3 with 1 M HCl. The resulting mixture was purified by RP-flash with the following conditions: C¹⁸ column (120 g); Mobile Phase A: water (0.1% TFA), Mobile Phase B: ACN; (Gradient: 5% B hold 5 min, up to 38% B within 30 min, 38% B hold 2.6 min; up to 95% B within 2 min, 95% B hold 5 min); Flow rate: 60 mL/min; Detector: UV 210 nm; RT=31 min. The product-containing fractions were collected and evaporated in vacuo to give (S)-6-((2-(4-(2-((((9H-fluoren-9-yl)methoxy)carbonyl)amino)-2-carboxyethyl)phenoxy)ethyl)amino)-N,N,N-trimethyl-6-oxohexan-1-aminium trifluoroacetate (1.12 g, 1.591 mmol, 49% yield) as a white solid. MS ESI calculated for C₃₇H₄₄F₃N₃O₈ [M-CF₃COO]⁺ 602.32, found 602.25. ¹H NMR (400 MHz, CD₃OD) δ 7.78 (d, J=7.6 Hz, 2H), 7.61-7.57 (m, 2H), 7.41-7.39 (m, 2H), 7.37-7.28 (m, 2H), 7.17-7.14 (m, 2H), 6.84-6.82 (m, 2H), 4.35-4.14 (m, 4H), 3.97-3.95 (m, 2H), 3.53-3.50 (m, 2H), 3.31-3.04 (m, 12H), 2.88-2.86 (m, 1H), 2.24-2.20 (m, 2H), 1.75-1.63 (m, 4H), 1.34-1.32 (m, 2H). ¹⁹F NMR (376 MHz, CD₃OD) δ −77.401.

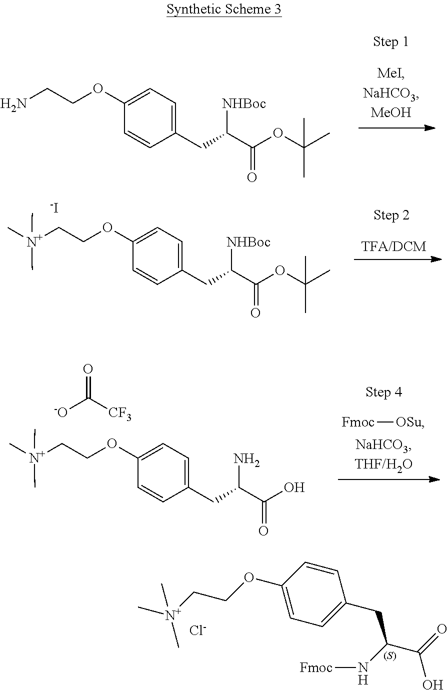

### (S)-2-(4-(2-((((9H-fluoren-9-yl)methoxy)carbonyl)amino)-2-carboxyethyl)phenoxy)-N,N,N-trimethylethan-1-aminium chloride

Step 1: To a stirred solution of tert-butyl (S)-3-(4-(2-aminoethoxy)phenyl)-2-((tert-butoxycarbonyl)amino)propanoate (7.5 g, 17.74 mmol) in MeOH (100 mL) were added Mel (12.59 g, 89 mmol) and NaHCO₃ (7.45 g, 89 mmol) at room temperature. The mixture was stirred at 40° C. for 16 h then cooled to room temperature. The solid was filtered out and the filtrate was concentrated under reduced pressure to give (S)-2-(4-(3-(tert-butoxy)-2-((tert-butoxycarbonyl)amino)-3-oxopropyl)phenoxy)-N,N,N-trimethylethan-1-aminium iodide (10 g, 16.35 mmol, 92% yield) as a light yellow solid. MS ESI calculated for C₂₃H₃₉N₂O₅ [M-I]⁺ 423.29, found 423.15.

Step 2: To a stirred solution of (S)-2-(4-(3-(tert-butoxy)-2-((tert-butoxycarbonyl)amino)-3-oxopropyl)phenoxy)-N,N,N-trimethylethan-1-aminium iodide (10 g, 18.17 mmol) in DCM (20 mL) was added TFA (40 mL) at room temperature. The resulting solution was stirred at 25° C. for 2 h. The solvent was concentrated under reduced pressure to give (S)-2-(4-(2-amino-2-carboxyethyl)phenoxy)-N,N,N-trimethylethan-1-aminium 2,2,2-trifluoroacetate (7 g, 14.72 mmol, 81% yield) as a yellow solid. MS ESI calculated for C₁₄H₂₃N₂O₃ [M-CF₃COO]⁺ 267.17, found 267.05.

Step 3: To a stirred solution of (S)-2-(4-(2-amino-2-carboxyethyl)phenoxy)-N,N,N-trimethylethan-1-aminium 2,2,2-trifluoroacetate (8 g, 21.03 mmol) in THF (80 mL) and water (80 mL) were added Fmoc-OSu (7.09 g, 21.03 mmol) and NaHCO₃ (5.30 g, 63.1 mmol) at room temperature. The resulting solution was stirred at 25° C. for 16 h. The pH of the solution was adjusted to 3 with 6 N HCl. The solvent was concentrated under reduced pressure and the residue was purified by RP flash with the following conditions: 330 g C¹⁸ column, 2%-2% in 5 min, 2%-40% in 30 min, 98%-98% in 5 min, MeCN in water (2 mmol HCl), RT=35 min to give (S)-2-(4-(2-((((9H-fluoren-9-yl)methoxy)carbonyl)amino)-2-carboxyethyl)phenoxy)-N,N,N-trimethylethan-1-aminium chloride (5.3489 g, 9.98 mmol, 47.5% yield) as an off-white solid. MS ESI calculated for C₂₉H₃₃N₂O₅ [M-Cl]⁺ 489.24, found 489.25. ¹H NMR (300 MHz, DMSO-d₆) δ 12.85 (br, 1H), 7.89 (d, J=7.5 Hz, 2H), 7.74-7.71 (m, 3H), 7.67-7.65 (m, 2H), 7.44-7.24 (m, 4H), 6.91 (d, J=7.5 Hz, 2H), 4.41-4.40 (m, 2H), 4.24-4.10 (m, 4H), 3.79-3.76 (m, 2H), 3.17 (s, 9H), 3.08-3.06 (m, 1H), 2.86-2.78 (m, 1H).

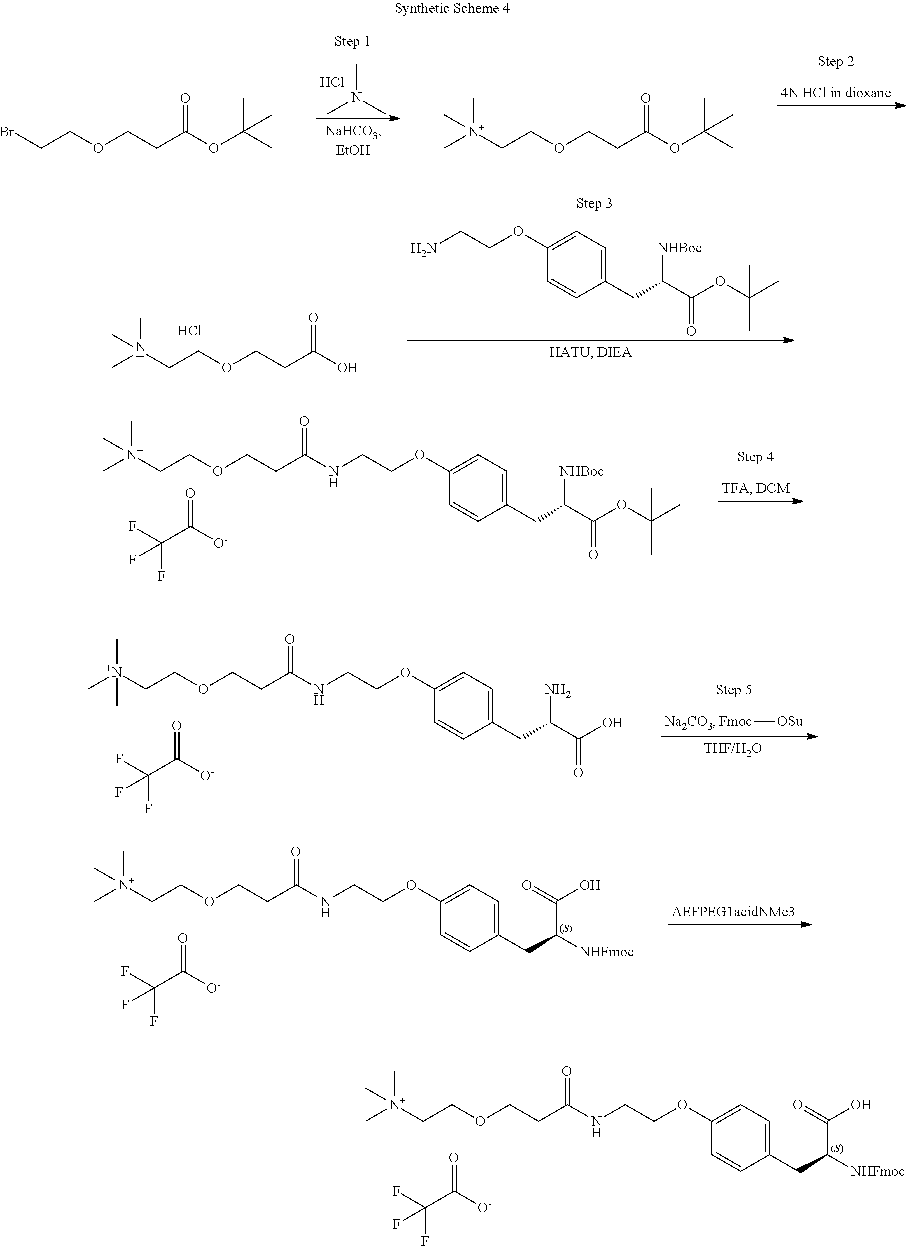

### (S)-2-(3-((2-(4-(2-((((9H-fluoren-9-yl)methoxy)carbonyl)amino)-2-carboxyethyl)phenoxy)ethyl)amino)-3-oxopropoxy)-N,N,N-trimethylethan-1-aminium 2,2,2-trifluoroacetate

Step 1: To a stirred solution of tert-butyl 3-(2-bromoethoxy)propanoate (2.5 g, 9.88 mmol) and trimethylamine hydrochloride (9.44 g, 99 mmol) in EtOH (30 mL) was added NaHCO₃ (12.44 g, 148 mmol) at room temperature. The mixture was stirred at 50° C. for 16 h then cooled to room temperature and the solid was filtered out. The filtrate was concentrated under reduced pressure and the residue was suspended in DCM (30 mL). The solid was filtered out and the filtrate was concentrated under reduced pressure to give 2-(3-(tert-butoxy)-3-oxopropoxy)-N,N,N-trimethylethan-1-aminium (4.2 g, 9.04 mmol, 92% yield) as a colorless oil. MS ESI calculated for C₁₂H₂₆NO₃ [M]⁺ 232.19, found 232.10.

Step 2: The solution of 2-(3-(tert-butoxy)-3-oxopropoxy)-N,N,N-trimethylethan-1-aminium (4.2 g, 9.04 mmol) in 4 N HC/dioxane (50 mL) was stirred at 25° C. for 2 h. The solvent was concentrated under reduced pressure to give 2-(2-carboxyethoxy)-N,N,N-trimethylethan-1-aminium chloride (3.8 g, 7.18 mmol, 79% yield) as an off-white solid. MS ESI calculated for C₈H₁₈NO₃ [M-Cl]⁺ 176.13, found 176.15.

Step 3: To a stirred solution of 2-(2-carboxyethoxy)-N,N,N-trimethylethan-1-aminium chloride (5.01 g, 9.46 mmol) and DIEA (6.89 mL, 39.4 mmol) in DMF (40 mL) was added HATU (3.60 g, 9.46 mmol) at room temperature. The solution was stirred at 25° C. for 10 min. Tert-butyl (S)-3-(4-(2-aminoethoxy)phenyl)-2-((tert-butoxycarbonyl)amino)propanoate (3 g, 7.88 mmol) was added and the resulting solution was stirred at room temperature for 2 h. After concentrated under reduced pressure, the residue was purified by RP-flash with the following conditions: C¹⁸ column, 330 g, 1%-1% in 5 min, 1%-37% in 25 min, MeCN in water (0.05%) to give (S)-2-(3-((2-(4-(3-(tert-butoxy)-2-((tert-butoxycarbonyl)amino)-3-oxopropyl)phenoxy)ethyl)amino)-3-oxopropoxy)-N,N,N-trimethylethan-1-aminium 2,2,2-trifluoroacetate (5 g, 7.29 mmol, 92% yield) as a light yellow solid. MS ESI calculated for C₂₈H₄₈N₃O₇ [M-CF₃COO]⁺ 538.35, found 538.25.

Step 4: To a stirred solution of (S)-2-(3-((2-(4-(3-(tert-butoxy)-2-((tert-butoxycarbonyl)amino)-3-oxopropyl)phenoxy)ethyl)amino)-3-oxopropoxy)-N,N,N-trimethylethan-1-aminium 2,2,2-trifluoroacetate (5 g, 7.29 mmol) in DCM (10 mL) was added TFA (40 mL) at room temperature. The resulting solution was stirred at room temperature for 3 h. The solvent was concentrated under reduced pressure to give (S)-2-(3-((2-(4-(2-amino-2-carboxyethyl)phenoxy)ethyl)amino)-3-oxopropoxy)-N,N,N-trimethylethan-1-aminium 2,2,2-trifluoroacetate (3.7 g, 6.72 mmol, 92% yield) as a yellow oil. MS ESI calculated for C₁₉H₃₂N₃O₅ [M-CF₃COO]⁺ 382.23, found 382.15.

Step 5: To a stirred solution of (S)-2-(3-((2-(4-(2-amino-2-carboxyethyl)phenoxy)ethyl)amino)-3-oxopropoxy)-N,N,N-trimethylethan-1-aminium 2,2,2-trifluoroacetate (3.7 g, 6.72 mmol) and Na₂CO₃ (2.137 g, 20.16 mmol) in THF (50 mL) and water (50 mL) was added Fmoc-OSu (2.267 g, 6.72 mmol) at room temperature. The resulting mixture was stirred at 25° C. for 16 h. The pH was adjusted to 3 with 1 N HCl and the solution was purified by RP-flash with the following conditions: C¹⁸ column, 330 g, 1%-1% in 5 min, 1%-37% in 30 min, MeCN in water (0.05% TFA) to give (S)-2-(3-((2-(4-(2-((((9H-fluoren-9-yl)methoxy)carbonyl)amino)-2-carboxyethyl)phenoxy)ethyl)amino)-3-oxopropoxy)-N,N,N-trimethylethan-1-aminium 2,2,2-trifluoroacetate (4.8452 g, 6.62 mmol, 98% yield) as an off-white solid. MS ESI calculated for C₃₄H₄₂N₃O₇ [M-CF₃COO]⁺ 604.30, found 604.25. ¹H NMR (400 MHz, DMSO-d₆) δ 8.19 (s, 1H), 7.90-7.88 (m, 2H), 7.74-7.72 (m, 3H), 7.44-7.40 (m, 2H), 7.34-7.32 (m, 2H), 7.30-7.28 (m, 2H), 7.20-7.18 (m, 2H), 4.23-4.12 (m, 4H), 3.94-3.91 (m, 2H), 3.78-3.77 (m, 2H), 3.68-3.65 (m, 2H), 3.49-3.47 (m, 2H), 3.41-3.39 (m, 2H), 3.04 (s, 9H), 3.00-2.99 (m, 1H), 2.83-2.81 (m, 1H), 2.38 (t, J=6.0 Hz, 2H). ¹⁹F NMR (376 MHz, DMSO-d₆) δ −74.41.

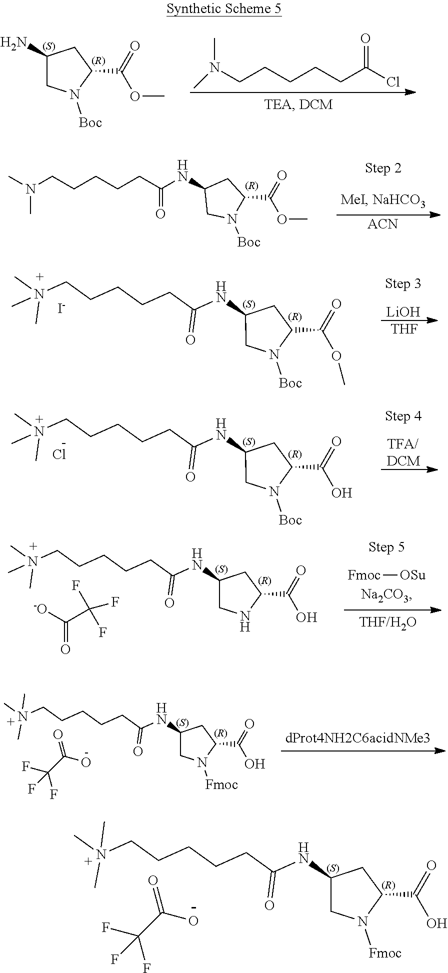

### 6-(((3S,5R)-1-(((9H-fluoren-9-yl)methoxy)carbonyl)-5-carboxypyrrolidin-3-yl)amino)-N,N,N-trimethyl-6-oxohexan-1-aminium 2,2,2-trifluoroacetate

Step 1: TEA (9.54 g, 94 mmol) and the solution of 6-(dimethylamino)hexanoyl chloride (3.35 g, 18.85 mmol) in DCM (7 mL) were added to the stirred mixture of 1-(tert-butyl) 2-methyl (2R,4S)-4-aminopyrrolidine-1,2-dicarboxylate (4.61 g, 18.85 mmol) in DCM (13 mL) at 0° C. After stirring for 2 h at 25° C., the reaction mixture was concentrated under reduced pressure. The residue was purified by silica gel column chromatography, eluted with 0-30% MeOH (1% NH₄OH) in DCM. The product-containing fractions were collected and evaporated in vacuum to afford 1-(tert-butyl) 2-methyl (2R,4S)-4-(6-(dimethylamino)hexanamido)pyrrolidine-1,2-dicarboxylate (2.4 g, 4.98 mmol, 26% yield) as a light white solid. MS ESI calculated for C₁₉H₃₆N₃O₅ [M+H]⁺ 386.26, found 386.35.

Step 2: To a stirred mixture of 1-(tert-butyl) 2-methyl (2R,4S)-4-(6-(dimethylamino)hexanamido)pyrrolidine-1,2-dicarboxylate (2.4 g, 6.23 mmol) and NaHCO₃ (7.84 g, 93 mmol) in ACN (50 mL) was added Mel (8.84 g, 62.3 mmol) at room temperature. After stirring at 25° C. for 16 h, the solid was filtered out and the filtrate was concentrated under reduced pressure to give 6-(((3S,5R)-1-(tert-butoxycarbonyl)-5-(methoxycarbonyl)pyrrolidin-3-yl)amino)-N,N,N-trimethyl-6-oxohexan-1-aminium iodide (3.28 g, 5.60 mmol, 90% yield) as a light yellow solid. MS ESI calculated for C₂₀H₃₈IN₃O₅[M-I]⁺ 400.28, found 400.35.

Step 3: To a stirred solution of 6-(((3S,5R)-1-(tert-butoxycarbonyl)-5-(methoxycarbonyl)pyrrolidin-3-yl)amino)-N,N,N-trimethyl-6-oxohexan-1-aminium iodide (3.28 g, 5.60 mmol) in THF (50 mL) was added LiOH (16.79 mL, 16.79 mmol, 1 N in water) at room temperature. After stirring at 25° C. for 1 h, the pH was adjusted to 6 with 1 N HCl. The solvent was concentrated under reduced pressure to give 6-(((3S,5R)-1-(tert-butoxycarbonyl)-5-carboxypyrrolidin-3-yl)amino)-N,N,N-trimethyl-6-oxohexan-1-aminium chloride (2.36 g, 5.03 mmol, 90% yield) as a yellow solid. MS ESI calculated for C₁₉H₃₆ClN₃O₅[M-Cl]⁺ 386.26, found 386.35.

Step 4: To a stirred solution of 6-(((3S,5R)-1-(tert-butoxycarbonyl)-5-carboxypyrrolidin-3-yl)amino)-N,N,N-trimethyl-6-oxohexan-1-aminium chloride (2.36 g, 5.03 mmol) in DCM (50 mL) was added TFA (50 mL) at room temperature. The resulting mixture was stirred at 25° C. for 1 h, then concentrated under reduced pressure to give 6-(((3S,5R)-5-carboxypyrrolidin-3-yl)amino)-N,N,N-trimethyl-6-oxohexan-1-aminium 2,2,2-trifluoroacetate (2.01 g, 4.53 mmol, 90% yield) as a yellow solid. MS ESI calculated for C₁₆H₂₈F₃N₃O₅[M—CF₃COO]⁺ 286.21, found 286.15.

Step 5: To a stirred solution of 6-(((3S,5R)-5-carboxypyrrolidin-3-yl)amino)-N,N,N-trimethyl-6-oxohexan-1-aminium 2,2,2-trifluoroacetate (2.01 g, 5.03 mmol) and NaHCO₃ (1.268 g, 15.10 mmol) in THF (50 mL) and water (50 mL) was added Fmoc-OSu (1.698 g, 5.03 mmol) at room temperature. After stirring at 25° C. for 16 h, the pH was adjusted to 3 with 1 N HCl and the solvent was evaporated under reduced pressure. The residue was dissolved in THF/MeOH (1:1, 50 mL), the solid was filtered out and the filtrated was purified by RP-flash with the following conditions: C¹⁸ column, 330 g, 2%-2% in 8 min, 2%-35% in 30 min, 35%-95% in 10 min, 95%-95% in 8 min, MeCN in water (0.05% TFA) to give 6-(((3S,5R)-1-(((9H-fluoren-9-yl)methoxy)carbonyl)-5-carboxypyrrolidin-3-yl)amino)-N,N,N-trimethyl-6-oxohexan-1-aminium 2,2,2-trifluoroacetate (2.85 g, 4.49 mmol, 89% yield) as a light yellow solid. MS ESI calculated for C₃₁H₃₈F₃N₃O₇[M-CF₃COO]⁺ 508.28, found 508.25. ¹H NMR (300 MHz, DMSO-d₆) δ 8.20 (d, J=6.9 Hz, 1H), 7.93-7.90 (m, 2H), 7.69-7.63 (m, 2H), 7.47-7.41 (m, 2H), 7.36-7.31 (m, 2H), 4.32-4.15 (m, 5H), 3.68-3.55 (m, 1H), 3.29-3.22 (m, 3H), 3.03 (s, 9H), 2.19-2.08 (m, 4H), 1.69-1.53 (m, 4H), 1.27-1.22 (m, 2H). ¹⁹F-NMR (282 MHz, DMSO-d₆) δ 74.261.

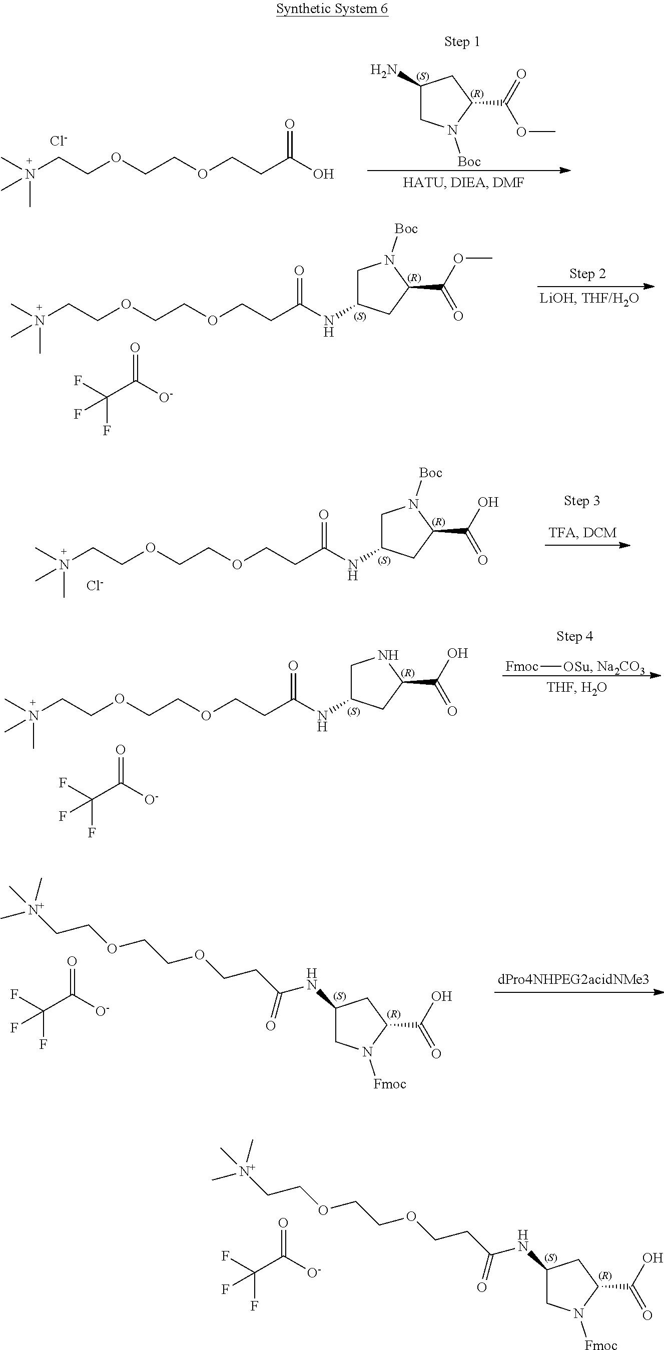

### 2-(2-(3-(((3S,5R)-1-(((9H-fluoren-9-yl)methoxy)carbonyl)-5-carboxypyrrolidin-3-yl)amino)-3-oxopropoxy)ethoxy)-N,N,N-trimethylethan-1-aminium 2,2,2-trifluoroacetate

Step 1: To a stirred solution of 2-(2-(2-carboxyethoxy)ethoxy)-N,N,N-trimethylethan-1-aminium chloride (2.64 g, 10.32 mmol) in DMF (40 mL) were added DIEA (1.501 mL, 8.60 mmol) and HATU (3.27 g, 8.60 mmol) at 0° C. under nitrogen atmosphere. The resulting solution was stirred at 0° C. for 10 min. then 1-(tert-butyl) 2-methyl (2R,4S)-4-aminopyrrolidine-1,2-dicarboxylate (2.1 g, 8.60 mmol) was added and stirred at 25° C. for 1 h. The solution was purified by RP-flash with the following conditions: C¹⁸ column, 330 g, 2%-2% in 5 min, 2%-30% in 20 min, 98%-98% in 5 min, MeCN in water (0.05% TFA), RT=35 min to give 2,2,2-trifluoroacetic acid, 2-(2-(3-(((3S,5R)-1-(tert-butoxycarbonyl)-5-(methoxycarbonyl)pyrrolidin-3-yl)amino)-3-oxopropoxy)ethoxy)-N,N,N-trimethylethan-1-aminium salt (4.4 g, 7.46 mmol, 87% yield) as a light-yellow oil. MS ESI calculated for C₂₁H₄₀N₃O₇ [M-CF₃COO]⁺ 446.29, found 446.35.

Step 2: To a stirred solution of 2,2,2-trifluoroacetic acid, 2-(2-(3-(((3S,5R)-1-(tert-butoxycarbonyl)-5-(methoxycarbonyl)pyrrolidin-3-yl)amino)-3-oxopropoxy)ethoxy)-N,N,N-trimethylethan-1-aminium salt (4.4 g, 7.85 mmol) in THF (50 mL) was added LiOH (23.55 mL, 23.55 mmol, 1 N in water) at room temperature. The resulting solution was stirred at 25° C. for 1 h. The pH was adjusted to 3 with 1 N HCl and the solvent was concentrated under reduced pressure to give 2-(2-(3-(((3S,5R)-1-(tert-butoxycarbonyl)-5-carboxypyrrolidin-3-yl)amino)-3-oxopropoxy)ethoxy)-N,N,N-trimethylethan-1-aminium chloride (3.7 g, 7.12 mmol, 91% yield) as a yellow oil. MS ESI calculated for C₂₀H₃₈N₃O₇ [M-Cl]⁺ 432.27, found 432.35.

Step 3: To a stirred solution of 2-(2-(3-(((3S,5R)-1-(tert-butoxycarbonyl)-5-carboxypyrrolidin-3-yl)amino)-3-oxopropoxy)ethoxy)-N,N,N-trimethylethan-1-aminium chloride (3.7 g, 7.12 mmol) in DCM (60 mL) was added TFA (20 mL) at room temperature. The solution was stirred at 25° C. for 1 h. The solvent was concentrated under reduced pressure to give 2-(2-(3-(((3S,5R)-5-carboxypyrrolidin-3-yl)amino)-3-oxopropoxy)ethoxy)-N,N,N-trimethylethan-1-aminium 2,2,2-trifluoroacetate (3.2 g, 6.47 mmol, 91% yield) as a yellow oil. MS ESI calculated for C₁₅H₃₀N₃O₅ [M-CF₃COO]⁺ 332.22, found 332.25.

Step 4: To a stirred solution of 2-(2-(3-(((3S,5R)-5-carboxypyrrolidin-3-yl)amino)-3-oxopropoxy)ethoxy)-N,N,N-trimethylethan-1-aminium 2,2,2-trifluoroacetate (3.8 g, 7.68 mmol) in THF (30 mL) and water (30 mL) were added Na₂CO₃ (2.441 g, 23.03 mmol) and Fmoc-OSu (2.59 g, 7.68 mmol) at room temperature. The resulting mixture was stirred at 25° C. for 16 h. The pH was adjusted to 3 with 6 N HCl and the solution was purified by RP-flash with the following conditions: 330 g C¹⁸ column, 2%-2% in 5 min, 2%-30% in 20 min, 98%-98% in 5 min, MeCN in water (0.05% TFA), RT=25 min to give 2-(2-(3-(((3S,5R)-1-(((9H-fluoren-9-yl)methoxy)carbonyl)-5-carboxypyrrolidin-3-yl)amino)-3-oxopropoxy)ethoxy)-N,N,N-trimethylethan-1-aminium 2,2,2-trifluoroacetate (4.1824 g, 6.14 mmol, 80% yield) as a colorless semi-solid. MS ESI calculated for C₃₀H₄₀N₃O₇ [M-CF₃COO]⁺ 554.29, found 554.20. ¹H NMR (400 MHz, DMSO-d₆) δ 8.24-8.23 (m, 1H), 7.92-7.89 (m, 2H), 7.68-7.64 (m, 2H), 7.45-7.32 (m, 4H), 4.48-4.23 (m, 4H), 4.16-4.15 (m, 1H), 3.83-3.82 (m, 2H), 3.67-3.51 (m, 9H), 3.27-3.25 (m, 1H), 3.09 (s, 9H), 2.36-2.32 (m, 2H), 2.20-2.11 (m, 2H).

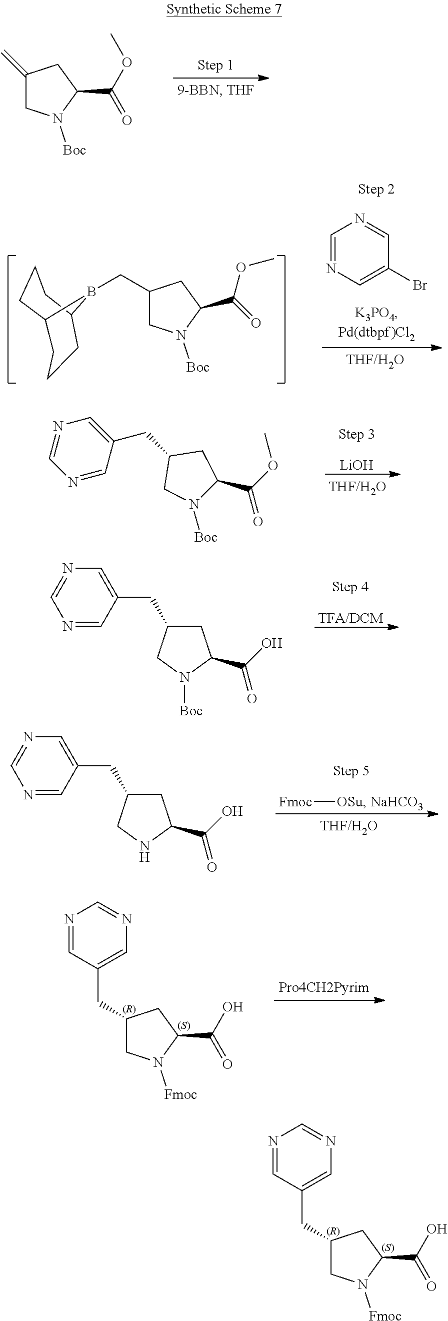

### (2S,4R)-1-(((9H-fluoren-9-yl)methoxy)carbonyl)-4-(pyrimidin-5-ylmethyl)pyrrolidine-2-carboxylic acid

Step 1: The solution of 1-(tert-butyl) 2-methyl (S)-4-methylenepyrrolidine-1,2-dicarboxylate (2.41 g, 9.99 mmol) and 9-BBN (80 mL, 40.0 mmol, 0.5 N in THF) was stirred at 40° C. for 1 h. The solution was cooled to room temperature and used to the next step directly. MS ESI calculated for C₁₂H₂₁BNO₆ [M-C₈H₁₄+H₂O₂—H]⁻ 286.15, found 286.25.

Step 2: To the solution of 1-(tert-butyl) 2-methyl (2S)-4-(((1R,5R)-9-borabicyclo[3.3.1]nonan-9-yl)methyl)pyrrolidine-1,2-dicarboxylate (14.5 g, 31.9 mmol) in THF (320 mL) was added K₃PO₄ (96 mL, 96 mmol, 1 N in water) at room temperature. The solution was stirred at room temperature for 20 min. 5-Bromopyrimidine (5.08 g, 31.9 mmol) and PdCl₂(dtbpf) (2.081 g, 3.19 mmol) were added and the resulting mixture was stirred at 60° C. for 1.5 h. The resulting solution was cooled to room temperature, diluted with brine (50 mL), extracted with EA (3×50 mL). The combined organic layer was washed with brine (2×25 mL), dried over anhydrous Na₂SO₄. After filtration, the filtrate was concentrated under reduced pressure and the residue was purified by a silica gel column chromatography, eluted with 0-90% EA in PE to afford a crude product. The crude product was separated by SFC with the following conditions: Column: Chiralpak IG, 3*25 cm, 5 m; Mobile Phase A: CO₂, Mobile Phase B: IPA:MeCN=1:1 (0.1% 2 M NH₃-MeOH); Flow rate: 100 mL/min; Gradient: isocratic 40% B; Column Temperature (° C.): 35; Back Pressure (bar): 100; Wavelength: 220 nm; RT1 (min): 8.93; RT2 (min): 11.32; Sample Solvent: MeOH (0.1% 2 M NH₃-MeOH); Injection Volume: 3.8 mL; Number of Runs: 5. The fractions at 8.93 min were collected and concentrated under reduced pressure to give 1-(tert-butyl) 2-methyl (2S,4R)-4-(pyrimidin-5-ylmethyl)pyrrolidine-1,2-dicarboxylate (4.3 g, 12.71 mmol, 40% yield) as a yellow oil. MS ESI calculated for C₁₆H₂₃N₃O₄ [M-Boc]⁺ 222.17, found 222.15. ¹H NMR (400 MHz, CDCl₃) δ 9.14 (d, J=4.7 Hz, 1H), 8.60 (s, 2H), 4.39-4.12 (m, 1H), 3.83-3.63 (m, 4H), 3.17-3.06 (m, 1H), 2.83-2.53 (m, 3H), 2.14-1.88 (m, 2H), 1.44-1.42 (m, 9H).

Step 3: To a stirred solution of 1-(tert-butyl) 2-methyl (2S,4R)-4-(pyrimidin-5-ylmethyl)pyrrolidine-1,2-dicarboxylate (4.3 g, 13.38 mmol) in THF (50 mL) was added LiOH (40.1 mL, 40.1 mmol, 1 N in water) at room temperature. The solution was stirred at 25° C. for 2 h. The pH of the solution was adjusted to 3 with 1 N HCl and the solvent was concentrated under reduced pressure to give (2S,4R)-1-(tert-butoxycarbonyl)-4-(pyrimidin-5-ylmethyl)pyrrolidine-2-carboxylic acid (4.1 g, 12.67 mmol, 95% yield) as a yellow solid. MS ESI calculated for C₁₅H₂₀N₃O₄ [M−H]⁻ 306.15, found 306.15.

Step 4: To a stirred solution of (2S,4R)-1-(tert-butoxycarbonyl)-4-(pyrimidin-5-ylmethyl)pyrrolidine-2-carboxylic acid (4.1 g, 12.67 mmol) in DCM (90 mL) was added TFA (30 mL) at room temperature. The solution was stirred at 25° C. for 1 h. The solvent was concentrated under reduced pressure to give (2S,4R)-4-(pyrimidin-5-ylmethyl)pyrrolidine-2-carboxylic acid (2.6 g, 11.29 mmol, 89% yield) as a yellow solid. MS ESI calculated for C₁₀H₁₄N₃O₂ [M+H]⁺ 208.10, found 208.15.

Step 5: To a stirred solution of (2S,4R)-4-(pyrimidin-5-ylmethyl)pyrrolidine-2-carboxylic acid (2.6 g, 12.55 mmol) in THF (50 mL) and water (50 mL) were added Fmoc-OSu (3.81 g, 11.29 mmol) and NaHCO₃ (5.27 g, 62.7 mmol) at room temperature. The resulting mixture was stirred at 25° C. for 16 h. The pH of the mixture was adjusted to 3 with 1 N HCl and the solution was purified by RP-flash with the following conditions: 330 g C¹⁸ column, 5%-5% in 5 min, 5%-40% in 25 min, 98%-98% in 5 min, MeCN in water (0.05% TFA), RT=30 min to give (2S,4R)-1-(((9H-fluoren-9-yl)methoxy)carbonyl)-4-(pyrimidin-5-ylmethyl)pyrrolidine-2-carboxylic acid (5.0 g, 11.53 mmol, 92% yield) as an off-white solid. MS ESI calculated for C₂₅H₂₄N₃O₄ [M+H]⁺ 430.17, found 430.10; ¹H NMR (300 MHz, CD₃OD) δ 9.18 (d, J=13.2 Hz, 1H), 8.68 (d, J=6.9 Hz, 2H), 7.80-7.75 (m, 2H), 7.61-7.58 (m, 2H), 7.41-7.27 (m, 4H), 4.42-4.31 (m, 3H), 4.23-4.17 (m, 1H), 3.62-3.50 (m, 1H), 3.18-3.00 (m, 1H), 2.76-2.70 (m, 2H), 2.68-2.66 (m, 1H), 2.12-2.02 (m, 2H).

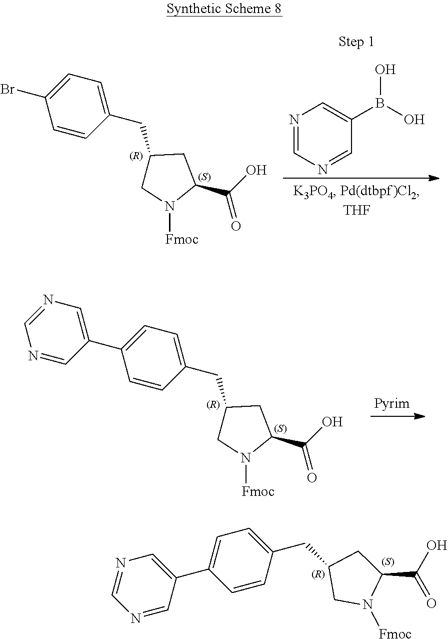

### (2S,4R)-1-(((9H-fluoren-9-yl)methoxy)carbonyl)-4-(4-(pyrimidin-5-yl)benzyl)pyrrolidine-2-carboxylic acid

Step 1: To a mixture of (2S,4R)-1-(((9H-fluoren-9-yl)methoxy)carbonyl)-4-(4-bromobenzyl)pyrrolidine-2-carboxylic acid (1.2 g, 2.370 mmol) in water (10 mL) and 1,4-dioxane (10 mL) were added pyrimidin-5-ylboronic acid (0.294 g, 2.370 mmol), K₃PO₄ (1.509 g, 7.11 mmol) and Pd(dtbpf)Cl₂ (0.232 g, 0.355 mmol) under argon at 25° C. The resulting mixture was stirred at 80° C. for 2 h, then the reaction was cooled to room temperature and directly purified by Rp-Flash with the following conditions: Column: C¹⁸ gel column (330 g); Mobile Phase A: water (0.01% TFA); Mobile Phase B: MeCN; (Gradient: 0% B hold 5 min, up to 52% B within 35 min, 52% B hold 3 min; up to 95% B within 2 min, 95% B hold 10 min); Flow rate: 60 mL/min; Detector: UV 254 & 210 nm; RT: 35 min. The product-containing fractions were collected and roto-evaporated in vacuo to give (2S,4R)-1-(((9H-fluoren-9-yl)methoxy)carbonyl)-4-(4-(pyrimidin-5-yl)benzyl)pyrrolidine-2-carboxylic acid (629 mg, 1.244 mmol, 52% yield) as a yellow solid. MS ESI calculated for C₃₁H₂₈N₃O₄ [M+H]⁺ 506.20, found 506.15. ¹H NMR (300 MHz, CD₃OD) δ 9.11-9.04 (m, 3H), 7.78-7.76 (m, 2H), 7.69-7.58 (m, 4H), 7.41-7.25 (m, 6H), 4.38-4.19 (m, 4H), 3.69-3.54 (m, 1H), 3.29-3.08 (m, 1H), 2.79-2.66 (m, 3H), 2.19-1.95 (m, 2H).

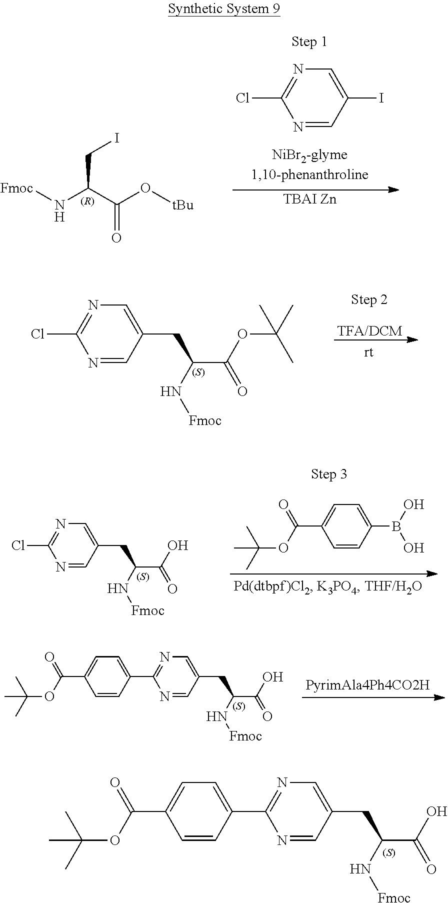

### (S)-2-((((9H-Fluoren-9-yl)methoxy)carbonyl)amino)-3-(2-(4-(tert-butoxycarbonyl)phenyl)pyrimidin-5-yl)propanoic acid

Step 1: To a stirred solution of NiBr₂-glyme (0.951 g, 2.432 mmol) in DMA (100 mL) was added 1,10-phenanthroline (0.527 g, 2.432 mmol) at 25° C. under nitrogen atmosphere. The resulted solution was stirred at 50° C. for 1 h then cooled to room temperature, to which 2-Chloro-5-iodopyrimidine (5.85 g, 24.32 mmol), tert-butyl (R)-2-((((9H-fluoren-9-yl)methoxy)carbonyl)amino)-3-iodopropanoate (6 g, 12.16 mmol), TBAI (4.66 g, 12.16 mmol) and zinc (1.590 g, 24.32 mmol) were added at room temperature. The resulting mixture was stirred at 25° C. for 2 h, then quenched with H₂O (200 mL), extracted with EA (2×500 mL). The combined organic layer was washed with brine (3×200 mL), dried over anhydrous Na₂SO₄ and filtered. The filtrate was concentrated under reduced pressure. The residue was purified by silica gel column chromatography, eluted with 0-20% EA in PE to give tert-butyl (S)-2-((((9H-fluoren-9-yl)methoxy)carbonyl)amino)-3-(2-chloropyrimidin-5-yl)propanoate (4 g, 8.35 mmol, 70% yield) as an off-white solid. MS ESI calculated for C₂₆H₂₇ClN₃O₄[M+H]⁺ 480.16, found 480.25.

Step 2: To a stirred solution of tert-butyl (S)-2-((((9H-fluoren-9-yl)methoxy)carbonyl)amino)-3-(2-chloropyrimidin-5-yl)propanoate (6 g, 12.50 mmol) in DCM (15 mL) was added TFA (30 mL) at room temperature. The solution was stirred at 25° C. for 3 h then concentrated under reduced pressure. The residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min, 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(2-chloropyrimidin-5-yl)propanoic acid (2.56 g, 6.04 mmol, 51% yield) as a yellow oil. MS ESI calculated for C₂₂H₁₉ClN₃O₄[M+H]⁺ 424.10, found 424.15.

Step 3: To a stirred mixture of (S)-2-((((9H-fluoren-9-yl)methoxy)carbonyl)amino)-3-(2-chloropyrimidin-5-yl)propanoic acid (2.56 g, 6.04 mmol), (4-(tert-butoxycarbonyl)phenyl)boronic acid (1.609 g, 7.25 mmol) and K₃PO₄ (6.41 g, 30.2 mmol) in water (20 mL) and dioxane (20 mL) was added Pd(dtbpf)Cl₂(0.590 g, 0.906 mmol) at room temperature. The resulting mixture was stirred at 80° C. for 2 h, then cooled to room temperature and concentrated under reduced pressure. The residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min, 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(2-(4-(tert-butoxycarbonyl)phenyl)pyrimidin-5-yl)propanoic acid (2.5629 g, 4.53 mmol, 75% yield) as a yellow oil. MS ESI calculated for C₃₃H₃₂N₃O₆ [M+H]⁺ 566.22, found 566.40. ¹H NMR (300 MHz, CD₃OD) δ 8.75 (s, 2H), 8.42 (d, J=8.3 Hz, 2H), 8.02 (d, J=8.2 Hz, 2H), 7.75 (d, J=7.5 Hz, 2H), 7.66-7.45 (m, 2H), 7.42-7.15 (m, 4H), 4.62-4.44 (m, 1H), 4.38-4.20 (m, 2H), 4.12 (t, J=7.0 Hz, 1H), 3.35-3.33 (m, 1H), 3.11-2.97 (m, 1H), 1.62 (s, 9H).

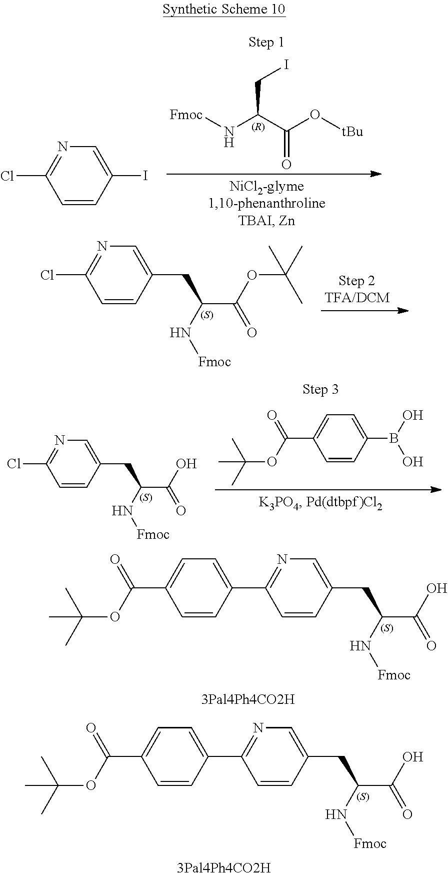

### (S)-2-((((9H-fluoren-9-yl)methoxy)carbonyl)amino)-3-(6-(4-(tert-butoxycarbonyl)phenyl)pyridin-3-yl)propanoic acid

Step 1: To a stirred solution of NiCl₂-glyme (0.918 g, 4.18 mmol) in DMA (100 mL) was added 1,10-phenanthroline (0.905 g, 4.18 mmol) at rt under nitrogen atmosphere. The resulted solution was stirred at 50° C. for 1 h. 2-Chloro-5-iodopyridine (5 g, 20.88 mmol), tert-butyl (R)-2-((((9H-fluoren-9-yl)methoxy)carbonyl)amino)-3-iodopropanoate (12.36 g, 25.06 mmol), TBAI (8.01 g, 20.88 mmol) and Zn (2.73 g, 41.8 mmol) were added to the mixture above at rt and the resulted mixture was stirred at 25° C. for 2 h. The reaction was quenched with H₂O (200 mL), extracted with EtOAc (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-30% EtOAc in PE to give tert-butyl (S)-2-((((9H-fluoren-9-yl)methoxy)carbonyl)amino)-3-(6-chloropyridin-3-yl)propanoate. MS ESI calculated for C₂₇H₂₈ClN₂O₄[M+H]⁺ 479.17, found 479.20.

Step 2: To a stirred solution of tert-butyl (S)-2-((((9H-fluoren-9-yl)methoxy)carbonyl)amino)-3-(6-chloropyridin-3-yl)propanoate (5 g, 10.48 mmol) in DCM (5 mL) was added TFA (10 mL) at rt. The solution was stirred at 25° C. for 1 h. The solvent was concentrated under reduced pressure and the residue was purified by RP-flash with the following conditions: Column: Flash C¹⁸ (330 g); Mobile Phase A: water (0.1% TFA), Mobile Phase B: ACN; (Gradient: 5% B hold 5 min, up to 30% B within 15 min, 30% B hold 5 min; up to 95% B within 20 min, 95% B hold 10 min); Flow rate: 90 mL/min; Detector: UV 210 nm; RT=40 min. The product-containing fractions were collected and evaporated in vacuo to give (S)-2-((((9H-fluoren-9-yl)methoxy)carbonyl)amino)-3-(6-chloropyridin-3-yl)propanoic acid. MS ESI calculated for C₂₃H₂₀ClN₂O₄[M+H]⁺ 423.10, found 423.10.

Step 3: To a stirred solution of (S)-2-((((9H-fluoren-9-yl)methoxy)carbonyl)amino)-3-(6-chloropyridin-3-yl)propanoic acid (3 g, 7.09 mmol) in THF (25 mL) and water (5 mL) were added (4-(tert-butoxycarbonyl)phenyl)boronic acid (1.890 g, 8.51 mmol) and K₃PO₄ (7.53 g, 35.5 mmol) at 25° C. under nitrogen. The resulting solution was stirred at 25° C. for 10 min. Pd(dtbpf)Cl₂ (0.694 g, 1.064 mmol) was added to the solution and the mixture was then stirred at 60° C. for 16 h. The reaction was cooled to rt and quenched with H₂O (200 mL) and extracted with EtOAc (2×500 mL). The combined organic layer was washed with brine (3×200 mL), dried over anhydrous Na₂SO₄, and filtered. The filtrate was concentrated under reduced pressure and the residue was purified by RP-flash with the following conditions: Column: Flash C¹⁸ (330 g); Mobile Phase A: water (0.1% TFA), Mobile Phase B: ACN; (Gradient: 5% B hold 5 min, up to 60% B within 15 min, 60% B hold 15 min; up to 95% B within 10 min, 95% B hold 10 min); Flow rate: 90 mL/min; Detector: UV 210 nm; RT=55 min. The product-containing fractions were collected and evaporated in vacuo to give (S)-2-((((9H-fluoren-9-yl)methoxy)carbonyl)amino)-3-(6-(4-(tert-butoxycarbonyl)phenyl)pyridin-3-yl)propanoic acid. MS ESI calculated for C₃₄H₃₃N₂O₆ [M+H]⁺ 565.23, found 565.15; ¹H NMR (400 MHz, Methanol-d₄) δ 8.66 (d, J=1.9 Hz, 1H), 8.12-8.07 (m, 3H), 7.99-7.92 (m, 3H), 7.77 (d, J=7.5 Hz, 2H), 7.59-7.56 (m, 2H), 7.37-7.33 (m, 2H), 7.30-7.22 (m, 2H), 4.60-4.56 (m, 1H), 4.29-4.27 (m, 2H), 4.14-4.10 (m, 1H), 3.45-3.41 (m, 1H), 3.14-3.10 (m, 1H), 1.62 (s, 9H).

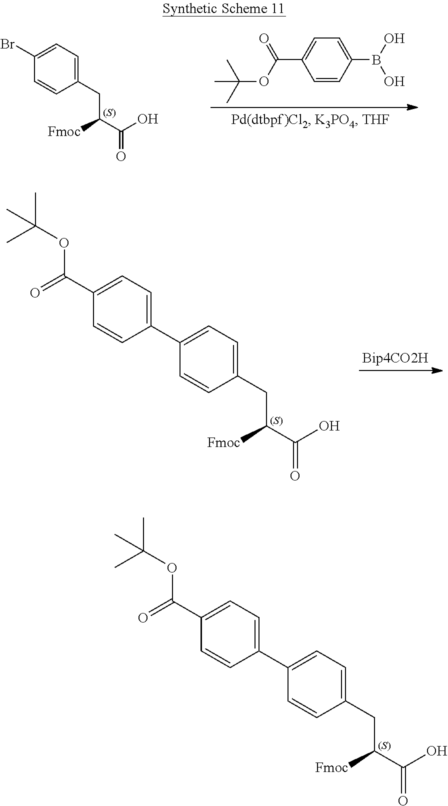

### (S)-2-((((9H-fluoren-9-yl)methoxy)carbonyl)amino)-3-(4′-(tert-butoxycarbonyl)-[1,1′-biphenyl]-4-yl)propanoic acid

Argon gas was bubbled through a mixture of (S)-2-((((9H-fluoren-9-yl)methoxy)carbonyl)amino)-3-(4-bromophenyl)propanoic acid (6 g, 12.87 mmol), (4-(tert-butoxycarbonyl)phenyl)boronic acid (4.29 g, 19.30 mmol) and K₃PO₄ (8.19 g, 38.6 mmol) in THF (40 mL) for 10 min, then [1,1′-bis(di-tert-butylphosphino)ferrocene]dichloropalladium (II) (0.839 g, 1.287 mmol) was added. After the resulting mixture was stirred at 50° C. for 16 h, it was diluted with EtOAc (300 mL) and washed with aqueous saturated NaHCO₃ (3×80 mL), brine (2×40 mL), dried over Na₂SO₄ and filtered. The filtrate was concentrated under reduced pressure. The residue was purified by silica gel column chromatography, eluted with gradient 0%-50% EtOAc in PE. The product-containing fractions were collected and roto-evaporated in vacuo. The residue was re-purified by combi-Flash with the following conditions: Column: Column: C18 gel column (330 g), 20-35 m; Mobile Phase A: 0.5% aq. TFA; Mobile Phase B: MeCN; (Gradient: 0% B hold 10 min, up to 62.3% B within 25 min, 62.3% B hold 6.2 min; up to 95% B within 2 min, 95% B hold 10 min); Flow rate: 90 mL/min; Detector: UV 254 & 210 nm; RT: 32.32 min. The product-containing fractions were collected and concentrated under reduced pressure to afford (S)-2-((((9H-fluoren-9-yl)methoxy)carbonyl)amino)-3-(4′-(tert-butoxycarbonyl)-[1,1-biphenyl]-4-yl)propanoic acid. MS ESI calculated for C₃₅H₃₄NO₆ [M+1]^{+ 564.23}, found 564.15. ¹H NMR (300 MHz, Methanol-d₄) δ 7.97-7.95 (m, 2H), 7.78-7.76 (m, 2H), 7.61-7.53 (m, 6H), 7.38-7.21 (m, 2H), 4.51-4.11 (m, 4H), 3.32-3.25 (m, 1H), 3.03-2.95 (m, 1H), 1.61 (s, 9H).

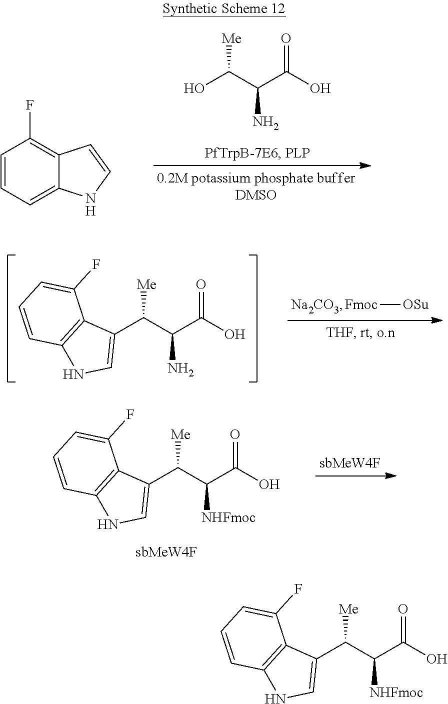

### (2S,3S)-2-amino-3-(4-fluoro-1H-indol-3-yl)butanoic acid

Into a 1-L 3-necked round-bottom flask purged and maintained with an inert atmosphere of nitrogen, was placed 4-fluoro-1H-indole (10 g, 1.00 eq), L-threonine (10.6 g, 1.20 eq), DMSO (100 mL), potassium phosphate buffer (0.2 M, 300 mL, pH=7.4). The reaction mixture was heated to 65° C., then PfTrpB-7E6 (2.5 g, 25 wt %) and 3-hydroxy-2-methyl-5-([phosphonooxy]methyl)-4-pyridinecarboxaldehyde (0.078 g, 0.004 eq) were added. The resulting solution was stirred overnight at 65° C. The mixture was then cooled to rt and used directly in the next step.

Into the above reaction mixture, THF (100 mL), sodium carbonate (23.56 g, 3.0 eq.) and 2,5-dioxopyrrolidin-1-yl 9H-fluoren-9-ylmethyl carbonate (29.96 g, 1.20 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. The mixture was applied onto a silica gel column with MeOH:DCM=1:5. HPLC-MS: (ES, m/z): [M+1]: 459. ¹H NMR (300 MHz, DMSO-d₆) δ 12.60 (s, 1H), 11.15 (s, 1H), 7.87 (d, J=7.6 Hz, 2H), 7.76-7.49 (m, 3H), 7.47-7.34 (m, 2H), 7.34-7.16 (m, 4H), 7.03 (td, J=7.9, 5.0 Hz, 1H), 6.73 (dd, J=11.8, 7.7 Hz, 1H), 4.36 (t, J=8.5 Hz, 1H), 4.31-4.02 (m, 3H), 3.51 (q, J=7.4 Hz, 1H), 1.31 (d, J=7.0 Hz, 4H), 0.78 (s, 1H).

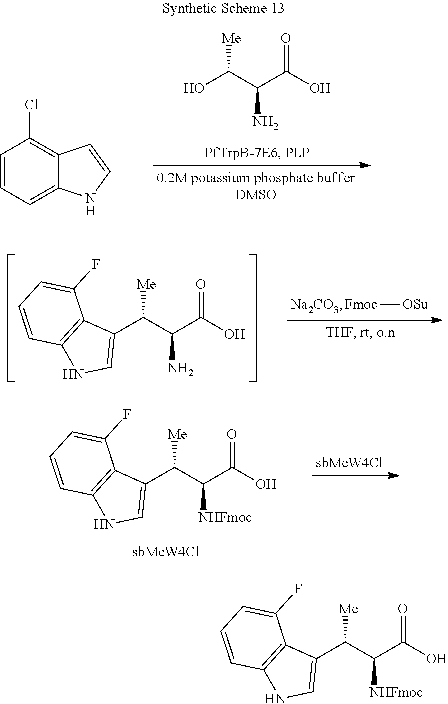

### (2S,3S)-2-amino-3-(4-chloro-1H-indol-3-yl)butanoic acid

Into a 1-L 3-necked round-bottom flask purged and maintained with an inert atmosphere of nitrogen, was placed 4-chloro-1H-indole (10 g, 1.00 eq.), L-threonine (14.09 g, 1.8 eq.), DMSO (100 mL), potassium phosphate buffer (0.2 M, 300 mL, PH=7.4), the reaction mixture was heated to 65° C., then PfTrpB-7E6 (7.5 g, 25 wt %) and 3-hydroxy-2-methyl-5-([phosphonooxy]methyl)-4-pyridinecarboxaldehyde (174 mg, 0.01 eq.) were added. The resulting solution was stirred for 36 h at 65° C. The mixture was then cooled to rt and used directly in the next step.

Into the above reaction mixture, THF (100 mL), sodium carbonate (20.9 g, 3.0 eq.) and 2,5-dioxopyrrolidin-1-yl 9H-fluoren-9-ylmethyl carbonate (31.0 g, 1.40 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. HPLC-MS: (ES, m/z): [M+1]: 475.

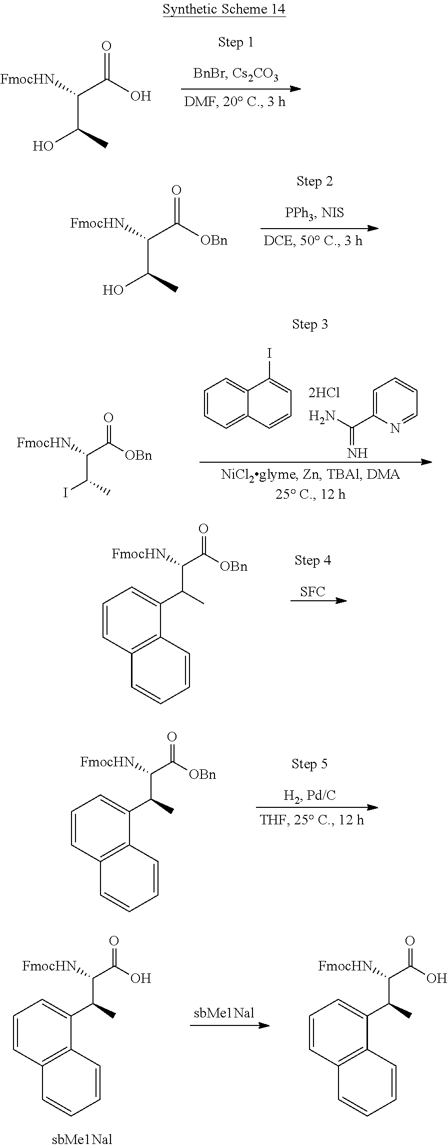

### (2S,3S)-2-amino-3-(naphthalen-1-yl)butanoic acid

Step 1: To a solution of (2S,3R)-2-((((9H-fluoren-9-yl)methoxy)carbonyl)amino)-3-hydroxybutanoic acid (250 g, 1.00 eq) in DMF (1.5 L) was added benzyl bromide (250 g, 2.00 eq) dropwise at 20° C. Then, cesium carbonate (477 g, 2.00 eq) was added and the solution was stirred at 20° C. for 3 h. The reaction was poured into ice H₂O (3 L) and extracted with EtOAc (500 mL×3). The organic phase was washed with 3% LiCl solution (500 mL×2) and brine (500 mL), dried over sodium sulfate and concentrated under vacuum at 40° C. The crude product was triturated with methyl tert-butyl ether:PE=6:1. ¹H NMR (400 MHz, CDCl₃): δ 7.77 (d, J=7.6 Hz, 1H), 7.40 (d, J=8.0 Hz, 1H), 7.31-7.36 (m, 10H), 5.65-5.71 m, 1H), 5.13-5.31 (m, 3H), 4.39-4.43 (m, 3H), 4.22-4.25 (m, 1H), 1.25 (d, J=6.4 Hz, 3H)

Step 2: To a 3-neck round-bottom flask was added (2S,3R)-benzyl 2-((((9H-fluoren-9-yl)methoxy)carbonyl)amino)-3-hydroxybutanoate (125 g, 1.00 eq) and DCE (750 mL) with an inert atmosphere of nitrogen. The reaction was cooled to 0° C. followed by the addition of NIS (195 g, 3.00 eq) and PPh₃ (228 g, 3.00 eq). The temperature was raised to 50° C. and the reaction mixture was stirred for 3 h. The reaction was poured into ice H₂O (500 mL) and extracted with DCM (500 mL×2). The organic phase was dried over sodium sulfate and concentrated under vacuum at 40° C. The residue was purified by silica gel column chromatography (PE/EtOAc=1/0 to 0/1). ¹H NMR (400 MHz, CDCl₃): δ 7.78 (d, J=7.6 Hz, 2H), 7.68 (d, J=7.2 Hz, 2H), 7.33-7.43 (m, 9H), 5.27-5.68 (m, 1H), 5.21-5.23 (m, 2H), 4.39-4.52 (m, 3H), 4.25-4.38 (m, 1H), 1.91-1.95 (m, 3H).

Step 3: To a 3-neck round-bottom flask was placed 2-((((9H-fluoren-9-yl) methoxy) carbonyl)amino)-3-iodobutanoate, 1-iodonaphthalene (42.2 g, 1.20 eq), TBAI (76.7 g, 1.50 eq), Zn (19.0 g, 2.10 eq) and DMA (750 mL). To a second 3-neck round-bottom flask was placed picolinimidamide·2HCl (42.2 g, 1.20 eq), NiCl₂·glyme (7.61 g, 0.25 eq) and DMA (750 mL) at 25° C. Under argon, the contents of the second flask were added to the first flask. The resulting mixture was then stirred for 12 h at 25° C. The reaction was poured into ice H₂O (3 L) and extracted with EtOAc (1 L×2). The organic phase was dried over sodium sulfate and concentrated under vacuum at 40° C. The crude product was purified by reversed-phase HPLC (MeCN:H₂O). HPLC-MS: [M+23]: 564. ¹H NMR (400 MHz, CDCl₃) δ: 8.17-8.24 (m, 1H), 8.15-8.17 (m, 1H), 7.77-7.87 (m, 2H), 7.76-7.77 (m, 4H), 7.30-7.41 (m, 10H), 5.30-5.38 (m, 1H), 4.96-5.04 (m, 3H), 4.85-4.87 (m, 1H), 4.30-4.34 (m, 1H), 4.18-4.26 (m, 4H), 1.43-1.45 (m, 3H).

Step 4: 143 g of (2S)-benzyl 2-((((9H-fluoren-9-yl) methoxy) carbonyl) amino)-3-(naphthalen-1-yl)butanoate was separated by SFC. The organic phase was concentrated under vacuum at 35° C.

Peak 1: (2S,3R)-benzyl 2-((((9H-fluoren-9-yl)methoxy)carbonyl) amino)-3-(naphthalene-1-yl)butanoate. ¹H NMR (400 MHz, DMSO-d₆): δ 8.11-8.12 (m, 2H), 8.10-8.11 (m, 1H), 7.88-7.90 (m, 2H), 7.54-7.88 (m, 1H), 7.44-7.53 (m, 2H), 7.42-7.44 (m, 4H), 7.33-7.42 (m, 3H), 7.27-7.33 (m, 6H), 7.08-7.09 (m, 2H), 4.91-4.94 (m, 1H), 4.79-4.82 (m, 1H), 4.58 (t, J=8.0 Hz), 4.17-4.25 (m, 4H), 1.39 (d, J=6.8 Hz, 3H). Peak 2: (2S,3S)-benzyl 2-((((9H-fluoren-9-yl)methoxy) carbonyl) amino)-3-(naphthalen-1-yl)butanoate. ¹H NMR (400 MHz, DMSO-d₆): δ 7.92-8.15 (m, 1H), 7.86-7.92 (m, 1H), 7.84-7.86 (m, 1H), 7.57-7.84 (m, 2H), 7.56-7.57 (m, 1H), 7.41-7.54 (m, 4H), 7.30-7.38 (m, 4H), 7.27-7.30 (m, 7H), 5.08-5.14 (m, 2H), 4.65 (t, J=8.0 Hz), 4.23-4.26 (m, 1H), 4.05-4.18 (m, 3H), 1.30 (d, J=6.8 Hz, 3H).

Step 5: To a 3-neck round-bottom flask was added (2S,3S)-benzyl 2-((((9H-fluoren-9-yl) methoxy) carbonyl)amino)-3-(naphthalen-1-yl)butanoate (40.0 g, 1.00 eq) and THF (200 mL). 10% wet Pd/C (7.00 g) was added and the reaction was purged 3 times with H₂ and stirred at 25° C. for 12 h under H₂(15 psi). The reaction was filtered through a CELITE pad and concentrated under vacuum at 35° C. The crude product was triturated with PE at 25° C. for 1 h. After filtration, the filter cake was dissolved in MeCN (100 mL) and concentrated under vacuum at 35° C. to remove residual solvent. HPLC-MS: [M+23]: 474. ¹H NMR (400 MHz, DMSO-d₆) δ 12.78 (s, 1H), 8.23 (d, J=7.6 Hz, 1H), 7.86-7.88 (m, 1H), 7.80-7.86 (m, 2H), 7.61-7.80 (m, 2H), 7.55-7.59 (m, 4H), 7.481-7.55 (m, 1H), 7.40-7.48 (m, 3H), 7.27-7.29 (m, 2H), 4.28-4.60 (m, 1H), 4.24-4.28 (m, 1H), 4.17-4.24 (m, 2H), 4.04-4.15 (m, 1H), 1.36 (d, J=6.8 Hz, 3H).

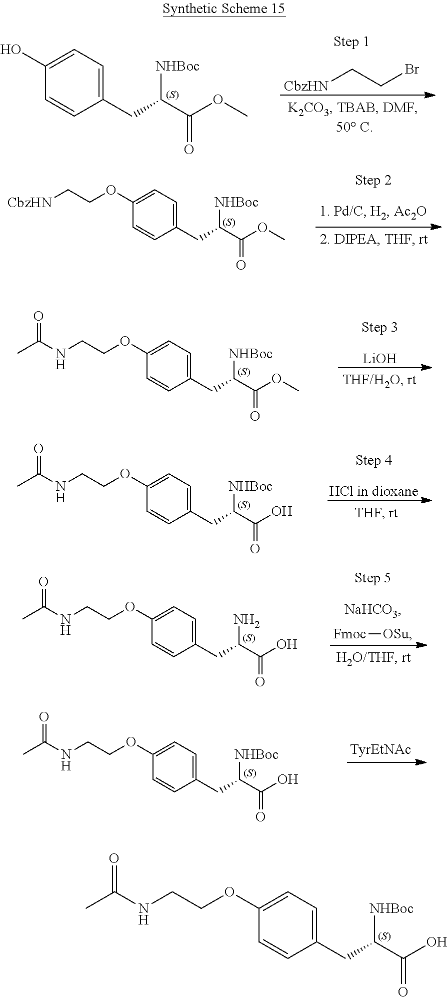

### L-Tyrosine O-ethyl acetamide or (S)-3-(4-(2-acetamidoethoxy)phenyl)-2-aminopropanoic acid

Step 1: To a stirred solution of methyl (tert-butoxycarbonyl)-L-tyrosinate (10.0 g, 33.9 mmol), benzyl (2-bromoethyl)carbamate (26.2 g, 102 mmol) and TBAB (5.46 g, 16.93 mmol) in DMF (150 mL) was added potassium carbonate (14.04 g, 102 mmol) at rt. The mixture was then stirred at 50° C. for 24 h. The mixture was cooled to rt, quenched with water (250 mL) and extracted with EtOAc (2×500 mL). The combined organic layers were washed with brine (3×150 mL), dried over anhydrous sodium sulfate and filtered. The filtrate was concentrated under reduced pressure, and the residue was purified by silica gel column chromatography, eluted with 0-30% EtOAc in PE. MS ESI calculated for C₂₅H₃₂N₂O₇ [M+Na]⁺ 495.22, found 495.10; ¹H NMR (300 MHz, CDCl₃) δ 7.38-7.32 (m, 5H), 7.04 (d, J=8.4 Hz, 2H), 6.81 (d, J=8.4 Hz, 2H), 5.31 (br, 1H), 5.21 (s, 2H), 4.97 (br, 1H), 4.56-4.53 (m, 1H), 4.03 (t, J=5.0 Hz, 2H), 3.72 (s, 3H), 3.64-3.58 (m, 2H), 3.06-3.01 (m, 2H), 1.43 (s, 9H).

Step 2: To a stirred solution of methyl (S)-3-(4-(2-(((benzyloxy)carbonyl)amino)ethoxy)phenyl)-2-((tert-butoxycarbonyl)amino)propanoate (16.0 g, 33.9 mmol) and acetic anhydride (6.39 mL, 67.7 mmol) in THF (200 mL) was added Pd/C (3.60 g, 33.9 mmol, dry, 10% wt) at rt under nitrogen atmosphere. The mixture was degassed with hydrogen 3 times and stirred at 20° C. for 4 h. DIPEA (17.74 mL, 102 mmol) was added to the mixture and stirred at 20° C. for 1 h. The mixture was filtered, and the filtrate was concentrated under reduced pressure. The residue was purified by silica gel column chromatography, eluted with 0-3% MeOH in DCM. MS ESI calculated for C₁₉H₂₈N₂O₆ [M+Na]⁺ 403.19, found 403.10; ¹H NMR (300 MHz, CDCl₃) δ 7.05 (d, J=8.4 Hz, 2H), 6.85-6.80 (m, 2H), 5.99 (br, 1H), 5.32 (br, 1H), 4.99-4.97 (m, 1H), 4.02 (t, J=5.0 Hz, 2H), 3.72 (s, 3H), 3.69-3.63 (m, 2H), 3.10-2.89 (m, 2H), 2.02 (s, 3H), 1.42 (s, 9H).

Step 3: To a stirred solution of methyl (S)-3-(4-(2-acetamidoethoxy)phenyl)-2-((tert-butoxycarbonyl)amino)propanoate (12.5 g, 32.9 mmol) THF (100 mL) was added lithium hydroxide (65.7 mL, 65.7 mmol, 1 N in water) at rt. The solution was stirred at 20° C. for 2 h. The pH of the solution was adjusted to 3 with 1 N HCl. The aqueous layer was extracted with EtOAc (2×250 mL). The combined organic layers were washed with brine (150 mL), dried over anhydrous sodium sulfate, filtered, and concentrated under reduced pressure. MS ESI [M+H]⁺: 367.10.

Step 4: To a stirred solution of (S)-3-(4-(2-acetamidoethoxy)phenyl)-2-((tert-butoxycarbonyl)amino)propanoic acid (12.5 g, 30.7 mmol) in THF (20 mL) was added 4 N HCl in dioxane (200 mL) at rt. The solution was stirred at 20° C. for 1 h. The solvent was concentrated under reduced pressure. MS ESI [M+H]⁺: 267.05.

Step 5: To a stirred mixture of (S)-3-(4-(2-acetamidoethoxy)phenyl)-2-aminopropanoic acid hydrochloride (9.50 g, 25.1 mmol) and NaHCO₃ (10.54 g, 126 mmol) in THF (100 mL) and water (100 mL) was added Fmoc-OSu (7.62 g, 22.59 mmol) at rt. The mixture was stirred at 20° C. for 1 h. The pH value of the solution was adjusted to 3 with 1 N HCl. The aqueous phase was extracted with EtOAc (2×500 mL). The combined organic layers were washed with brine (150 mL), dried over anhydrous sodium bicarbonate and filtered. The filtrate was concentrated under reduced pressure, and the residue was recrystallized from EtOAc (200 mL). The solid was collected by filtration and dried under vacuum. MS ESI [M+H]⁺: 489.05; ¹H NMR (300 MHz, Methanol-d₄) δ 7.79 (d, J=7.6 Hz, 2H), 7.62-7.57 (m, 2H), 7.42-7.26 (m, 4H), 7.17-7.14 (m, 2H), 6.83 (d, J=8.4 Hz, 2H), 4.41-4.31 (m, 2H), 4.29-4.10 (m, 2H), 3.96 (t, J=4.8 Hz, 2H), 3.51 (t, J=5.4 Hz, 2H), 3.19-3.13 (m, 1H), 2.92-2.84 (m, 1H), 1.94 (s, 3H).

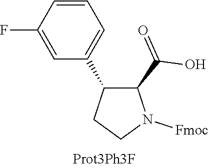

### (2S,3R)-1-(((9H-fluoren-9-yl)methoxy)carbonyl)-3-(3-fluorophenyl)pyrrolidine-2-carboxylic acid

To a solution of commercially available ((2S,3R)-3-(3-fluorophenyl)pyrrolidine-2-carboxylic acid (495 mg, 2.366 mmol) in acetone (16 mL) were added water (8 mL) and sodium bicarbonate (2S,3R)-3-(3-fluorophenyl)pyrrolidine-2-carboxylic acid (495 mg, 2.366 mmol), the mixture was stirred for 10 min (pH 9.5 targeted), Fmoc-OSu (878 mg, 2.60 mmol) was added and the resulting mixture was stirred overnight at room temperature. The reaction was acidified with sodium bisulfate (1M, 75 mL). The aqueous was washed with DCM, the organic phase was dried with anhydrous sodium sulfate, filtered. The filtrate was concentrated under reduced pressure. The crude material was purified by column chromatography using a RediSep Rf silica 80 g prepackaged column, eluting with a 0-10% gradient of MeOH in DCM over the course of 33 minutes, after concentration under reduced pressure to provide the title compound. HPLC-MS: [M+H]⁺: 432.2; ¹H NMR (500 MHz, CDCl₃) δ 7.80 (d, J=7.5 Hz, 2H), 7.62 (t, J=7.1 Hz, 2H), 7.44 (t, J=7.4 Hz, 2H), 7.40-7.25 (m, 3H), 7.05-6.89 (m, 3H), 4.60-4.46 (m, 2H), 4.35-4.25 (m, 1H), 3.79-3.50 (m, 3H), 2.48-2.30 (m, 1H), 2.10-1.96 (m, 1H).

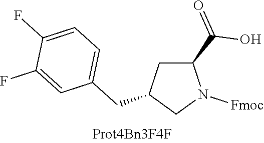

### (2S,4R)-1-(((9H-fluoren-9-yl)methoxy)carbonyl)-4-(3,4-difluorobenzyl)pyrrolidine-2-carboxylic acid

To a solution of commercially available (2S,4R)-4-(3,4-difluorobenzyl)pyrrolidine-2-carboxylic acid (1000 mg, 4.15 mmol) in acetone (16 mL) were added water (8 mL) and sodium bicarbonate (1741 mg, 20.73 mmol), the mixture was stirred for 10 min (pH 9.5 targeted), Fmoc-OSu (1538 mg, 4.56 mmol) was added and the resulting mixture was stirred overnight at room temperature. The reaction was acidified with sodium bisulfate (1M, 38 ml). The aqueous was washed with DCM, the organic phase was dried with anhydrous sodium sulfate, filtered. The filtrate was concentrated under reduced pressure. The crude material was purified by column chromatography using a RediSep Rf silica 40 g prepackaged column, eluting with a 0-10% gradient of MeOH in DCM over the course of 22 minutes, and concentrated under reduced pressure to provide the title compound. HPLC-MS: [M+H]⁺: 464.4; ¹H NMR (500 MHz, CDCl₃) δ 7.76 (d, J=7.1 Hz, 2H), 7.56 (d, J=6.8 Hz, 2H), 7.45-7.30 (m, 4H), 7.17-7.05 (m, 1H), 7.00-6.93 (m, 1H), 6.89-6.82 (m, 1H), 4.57-4.38 (m, 3H), 4.33-4.23 (m, 1H), 3.54-3.47 (m, 1H), 3.03 (t, J=9.3 Hz, 1H), 2.75-2.50 (m, 2H), 2.38-2.32 (m, 1H), 2.0-1.80 (m, 2H).

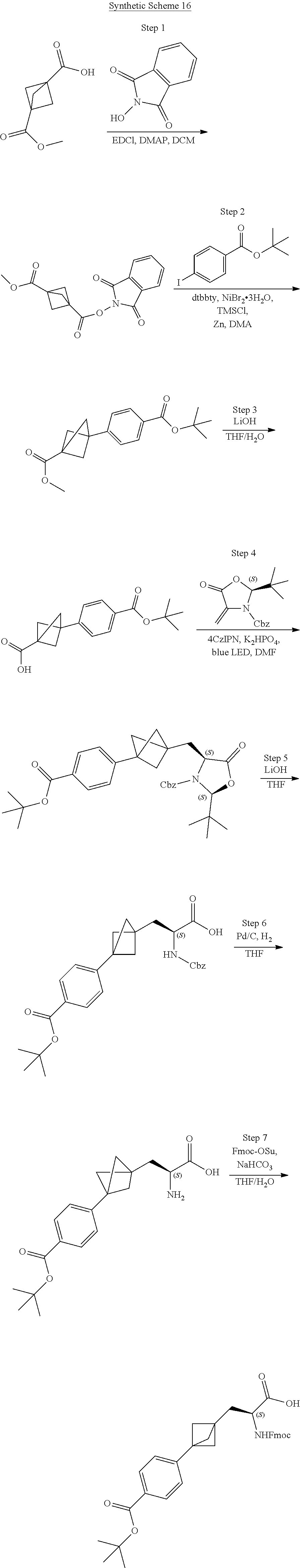

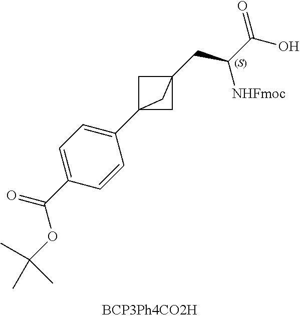

### (S)-2-((((9H-fluoren-9-yl)methoxy)carbonyl)amino)-3-(3-(4-(tert-butoxycarbonyl)phenyl)bicyclo[1.1.1]pentan-1-yl)propanoic acid

Step 1: To a stirred solution of 3-(methoxycarbonyl)bicyclo[1.1.1]pentane-1-carboxylic acid (5 g, 29.4 mmol), 2-hydroxyisoindoline-1,3-dione (5.27 g, 32.3 mmol) and DMAP (0.359 g, 2.94 mmol) in DCM (65 mL) was added EDCI (6.20 g, 32.3 mmol) at room temperature. The resulting mixture was stirred at 25° C. for 16 h. The solid was filtered out and the filtrate was concentrated under reduced pressure. The residue was purified by a silica gel column chromatography, eluted with 0-30% EA in PE to afford 1-(1,3-dioxoisoindolin-2-yl) 3-methyl bicyclo[1.1.1]pentane-1,3-dicarboxylate. ¹H NMR (400 MHz, CDCl₃) δ 7.96-7.83 (m, 2H), 7.86-7.75 (m, 2H), 3.73 (s, 3H), 2.56 (s, 6H).

Step 2: To a stirred solution of NiBr₂·3H₂O (1.549 g, 5.68 mmol) in DMA (180 mL) was added dtbbty (1.906 g, 7.10 mmol) at room temperature under nitrogen atmosphere. The resulting mixture was stirred at 50° C. for 30 min. The mixture was cooled to room temperature. Tert-butyl 4-iodobenzoate (7.2 g, 23.67 mmol), 1-(1,3-dioxoisoindolin-2-yl) 3-methyl bicyclo[1.1.1]pentane-1,3-dicarboxylate (7.46 g, 23.67 mmol), TMS-Cl (0.303 mL, 2.367 mmol) and zinc (7.74 g, 118 mmol) were added to the mixture at room temperature. The resulting mixture was stirred at 25° C. for 2 h. The reaction was quenched by brine (400 mL), extracted with EA (3×800 mL). The combined organic layer was washed with brine (3×300 mL), dried over anhydrous Na₂SO₄. After filtration, the filtrate was concentrated under reduced pressure and the residue was purified by a silica gel column chromatography, eluted with 0-40% EA in PE to afford methyl 3-(4-(tert-butoxycarbonyl)phenyl)bicyclo[1.1.1]pentane-1-carboxylate. ¹H NMR (300 MHz, CDCl₃) δ 7.98-7.88 (m, 2H), 7.29-7.19 (m, 2H), 3.72 (s, 3H), 2.34 (s, 6H), 1.59 (s, 9H).

Step 3: To a solution of methyl 3-(4-(tert-butoxycarbonyl)phenyl)bicyclo[1.1.1]pentane-1-carboxylate (2.7 g, 8.93 mmol) in THF (17.86 mL) was added LiOH (17.86 mL, 17.86 mmol, 1 N in water) at room temperature. The resulting mixture was stirred at room temperature for 2 h. The solution was acidified with 1 M HCl (15 mL). The solution was extracted with EA (3×150 mL). The combined organic layers were washed with brine (3×150 mL), dried over anhydrous Na₂SO₄ and filtered. The filtrate was concentrated under reduced pressure to afford 3-(4-(tert-butoxycarbonyl)phenyl)bicyclo[1.1.1]pentane-1-carboxylic acid (2.55 g, crude). MS ESI calculated for C₁₇H₁₉O₄[M−H]—287.14, found 287.05.

Step 4: To a stirred solution of benzyl (S)-2-(tert-butyl)-4-methylene-5-oxooxazolidine-3-carboxylate (602 mg, 2.081 mmol) in DMF (20 mL) were added 3-(4-(tert-butoxycarbonyl)phenyl)bicyclo[1.1.1]pentane-1-carboxylic acid (600 mg, 2.081 mmol), 4CzIPN (32.8 mg, 0.042 mmol) and K₂HPO₄ (906 mg, 5.20 mmol). The reaction solution was irradiated with a 34 W blue LED lamp and stirred at room temperature for 48 h under argon. The reaction was quenched with water (100 mL) and extracted with EA (3×150 mL). The combined organic layers were washed with brine (3×150 mL), dried over anhydrous Na₂SO₄ and filtered. The filtrate was concentrated under reduced pressure and the residue was purified by a silica gel column chromatography, eluted with gradient 0%-25% EA in PE to afford benzyl (2S,4S)-4-((3-(4-(tert-butoxycarbonyl)phenyl)bicyclo[1.1.1]pentan-1-yl)methyl)-2-(tert-butyl)-5-oxooxazolidine-3-carboxylate. MS ESI calculated for C₃₂H₄₀NO₆ [M-tBu+H]⁺ 478.28, found 478.15. ¹H NMR (400 MHz, CDCl₃) δ 7.94-7.84 (m, 2H), 7.41-7.35 (m, 5H), 7.18 (d, J=8.0 Hz, 2H), 5.56 (s, 1H), 5.24-5.12 (m, 2H), 4.34-4.31 (m, 1H), 2.23-2.17 (m, 1H), 2.09-2.01 (m, 1H), 1.99-1.83 (m, 6H), 1.59 (s, 9H), 0.97 (s, 9H).

Step 5: To a solution of benzyl (2S,4S)-4-((3-(4-(tert-butoxycarbonyl)phenyl)bicyclo[1.1.1]pentan-1-yl)methyl)-2-(tert-butyl)-5-oxooxazolidine-3-carboxylate (1.6 g, 3.00 mmol) in THF (16 mL) was added LiOH (7.50 mL, 7.50 mmol, 1 N in water) at 0° C. The solution was stirred at room temperature for 4 h. The solution was cooled to 0° C. and the pH value was adjusted to 3-4 with 1 M HCl (7.5 mL). The resulting solution was dilute with water (50 mL) and extracted with EA (3×100 mL). The organic layer was washed with brine (3×100 mL), dried over anhydrous Na₂SO₄ and filtered. The filtrate was concentrated under reduced pressure to afford (S)-2-(((benzyloxy)carbonyl)amino)-3-(3-(4-(tert-butoxycarbonyl)phenyl)bicyclo[1.1.1]pentan-1-yl)propanoic acid. MS ESI calculated for C₂₇H₃₁NO₆Na [M+Na]⁺ 488.20, found 488.10. ¹H NMR (400 MHz, CD₃OD) δ 7.88-7.83 (m, 2H), 7.41-7.35 (m, 2H), 7.35-7.25 (m, 3H), 7.24-7.19 (m, 2H), 5.19-5.04 (m, 2H), 4.24-4.20 (m, 1H), 2.13-2.01 (m, 1H), 2.03-1.87 (m, 7H), 1.58 (s, 9H).

Step 6: To a solution of (S)-2-(((benzyloxy)carbonyl)amino)-3-(3-(4-(tert-butoxycarbonyl)phenyl)bicyclo[1.1.1]pentan-1-yl)propanoic acid (1.65 g, 3.54 mmol) in THF (40 mL) were added Pd—C (0.377 g, 0.354 mmol, dry, 10% wt) at room temperature under nitrogen atmosphere. The resulting mixture was degassed with hydrogen three times and stirred at room temperature for 2 h. The resulting product ((S)-2-amino-3-(3-(4-(tert-butoxycarbonyl)phenyl)bicyclo[1.1.1]pentan-1-yl)propanoic acid (1.17 g) was used into the next step without further purification. MS ESI calculated for C₁₉H₂₆NO₄ [M+H]⁺ 332.17, found 332.15.

Step 7: A reaction mixture of (S)-2-amino-3-(3-(4-(tert-butoxycarbonyl)phenyl)bicyclo[1.1.1]pentan-1-yl)propanoic acid (1.17 g, 3.53 mmol) in THF (40 mL) and water (40 mL) were added NaHCO₃ (1.483 g, 17.65 mmol) and Fmoc-OSu (1.191 g, 3.53 mmol) at 0° C. The reaction mixture was stirred at room temperature for 16 h. The resulting mixture was filtered. Filter cake was washed with THF (2×100 mL). The solvent was concentrated under reduced pressure. The pH value of the solution was adjusted to 4 with 1 N HCl (17.6 mL) and extracted with EA (3×100 mL). The combined organic layers were washed with brine (3×100 mL), dried with anhydrous Na₂SO₄ and filtered. The resulting solution was purified by RP-Flash chromatography, eluted with 2-70% acetonitrile in water (0.05% TFA) to afford (S)-2-((((9H-fluoren-9-yl)methoxy)carbonyl)amino)-3-(3-(4-(tert-butoxycarbonyl)phenyl)bicyclo[1.1.1]pentan-1-yl)propanoic acid. MS ESI calculated for C₃₄H₃₄NO₆ [M−H]-552.25, found 552.40. ¹H NMR (300 MHz, CD₃OD) δ 7.90-7.76 (m, 2H), 7.73-7.65 (m, 4H), 7.40-7.24 (m, 6H), 4.52-4.37 (m, 2H), 4.25-4.18 (m, 2H), 2.14-2.03 (m, 1H), 1.99-1.86 (m, 7H), 1.60-1.54 (m, 9H).

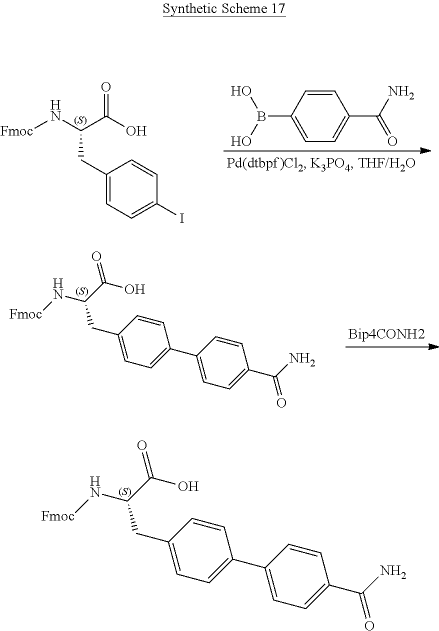

### (S)-2-((((9H-fluoren-9-yl)methoxy)carbonyl)amino)-3-(4′-carbamoyl-[1,1′-biphenyl]-4-yl)propanoic acid

To a stirred solution of (S)-2-((((9H-fluoren-9-yl)methoxy)carbonyl)amino)-3-(4-iodophenyl)propanoic acid (15 g, 29.2 mmol) in THF (246 mL) were added PdCl₂(dtbpf) (2.86 g, 4.38 mmol) and potassium phosphate tribasic (88 mL, 88 mmol, 1 N in water) at room temperature. The solution was stirred at 50° C. for 2 h. The resulting solution was cooled to room temperature. The pH was adjusted to 3 with 1H HCl and extracted with EA (3×250 mL). The organic layers were combined, washed with brine (4×200 mL), dried over anhydrous Na₂SO₄ and filtered. The filtrate was concentrated under reduced pressure and the residue was recrystallized from EtOH (100 mL) to give (S)-2-((((9H-fluoren-9-yl)methoxy)carbonyl)amino)-3-(4′-carbamoyl-[1,1′-biphenyl]-4-yl)propanoic acid. MS ESI calculated for C₃₁H₂₇N₂O₅ [M+H]⁺ 507.18, found 507.45. ¹H NMR (400 MHz, DMSO-d₆) δ 12.87 (s, 1H), 8.02 (s, 1H), 8.07-7.96 (m, 2H), 7.94-7.82 (m, 2H), 7.78-7.60 (m, 7H), 7.39-7.29 (m, 5H), 7.29-7.19 (m, 2H), 4.20-4.13 (m, 4H), 3.17-3.13 (m, 1H), 2.93-2.90 (m, 1H).

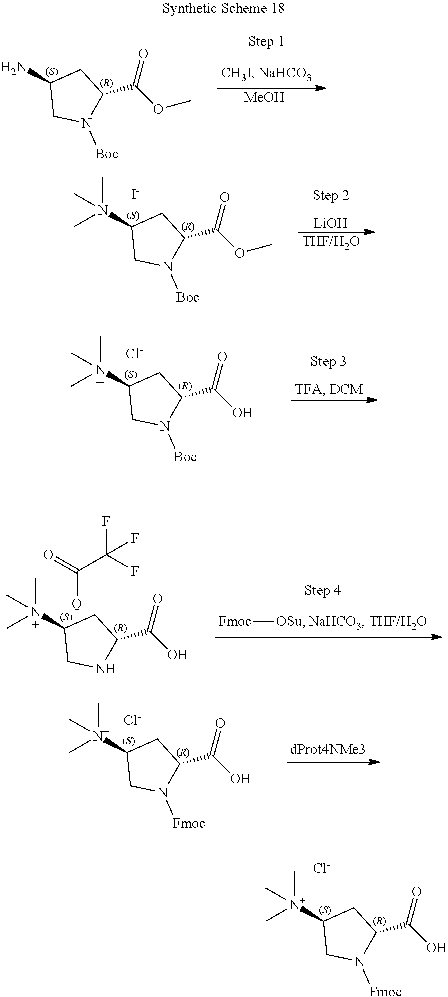

### (3S,5R)-1-(((9H-fluoren-9-yl)methoxy)carbonyl)-5-carboxy-N,N,N-trimethylpyrrolidin-3-aminium chloride

Step 1: To a mixture of 1-(tert-butyl) 2-methyl (2R,4S)-4-aminopyrrolidine-1,2-dicarboxylate (3 g, 12.28 mmol) in MeOH (30 mL) were added NaHCO₃ (8.25 g, 98 mmol) and CH₃I (4.61 mL, 73.7 mmol) at ambient temperature. The reaction was stirred at ambient temperature for 16 h. The resulting solution was filtered. The filtrate was concentrated in vacuo to afford crude product. The crude product was dissolved in DCM and filtered, the filter was concentrated in vacuo to afford (3S,5R)-1-(tert-butoxycarbonyl)-5-(methoxycarbonyl)-N,N,N-trimethylpyrrolidin-3-aminium iodide. MS ESI calculated for C₁₄H₂₇N₂O₄ [M-I]⁺ 287.20, found 287.15.

Step 2: To a mixture of (3S,5R)-1-(tert-butoxycarbonyl)-5-(methoxycarbonyl)-N,N,N-trimethylpyrrolidin-3-aminium iodide (6.5 g, 10.98 mmol) in THF (23 mL) was added LiOH (21.97 mL, 21.97 mmol, 1 M in water) at ambient temperature. The reaction was stirred at ambient temperature for 4 h. The resulting solution was acidified to pH 5 with 1 M HCl and concentrated in vacuo to afford (3S,5R)-1-(tert-butoxycarbonyl)-5-carboxy-N,N,N-trimethylpyrrolidin-3-aminium chloride. MS ESI calculated for C₁₃H₂₅N₂O₄ [M-Cl]⁺ 273.18, found 273.15.

Step 3: To a mixture of (3S,5R)-1-(tert-butoxycarbonyl)-5-carboxy-N,N,N-trimethylpyrrolidin-3-aminium chloride (6.6 g, 10.69 mmol) in DCM (20 mL) was added TFA (20 mL, 260 mmol) at ambient temperature. The reaction was stirred at ambient temperature for 2 h. The resulting solution was concentrated in vacuo to afford 2,2,2-trifluoroacetic acid, (3S,5R)-1-(tert-butoxycarbonyl)-5-carboxy-N,N,N-trimethylpyrrolidin-3-aminium salt. MS ESI calculated for C₈H₁₇N₂O₂ [M-CF₃COO]⁺ 173.13, found 173.25.

Step 4: To a mixture of 2,2,2-trifluoroacetic acid, (3S,5R)-5-carboxy-N,N,N-trimethylpyrrolidin-3-aminium salt (8.1 g, 14.10 mmol) in THF (40 mL) and water (40 mL) were added NaHCO₃ (5.92 g, 70.5 mmol) and Fmoc-OSu (4.28 g, 12.69 mmol) at room temperature. The reaction was stirred at room temperature for 4 h. The resulting solution was acidified to pH 5 with diluted HCl and concentrated in vacuo. The residue was suspended in DCM (with 10% MeOH) and filtered. The filtrate was concentrated in vacuo. The residue was purified by RP flash with the following conditions: Column: Flash C¹⁸ (330 g); Mobile Phase A: water (2 mmol HCl), Mobile Phase B: ACN; (Gradient: 2% B hold 5 min, up to 35% B within 15 min, 35% B hold 8 min; up to 98% B within 5 min, 99% B hold 5 min); Flow rate: 80 mL/min; Detector: UV 210 nm; RT=38 min. The product-containing fractions were collected and concentrated in vacuo to (3S,5R)-1-(((9H-fluoren-9-yl)methoxy)carbonyl)-5-carboxy-N,N,N-trimethylpyrrolidin-3-aminium chloride. MS ESI calculated for C₂₃H₂₇N₂O₄ [M-Cl]⁺ 395.20, found 395.20. ¹H NMR (300 MHz, DMSO-d₆) δ 13.30 (s, 1H), 7.94-7.89 (m, 2H), 7.72-7.64 (m, 2H), 7.46-7.33 (m, 4H), 4.57-4.14 (m, 5H), 3.86-3.78 (m, 2H), 3.12-3.10 (m, 9H), 2.83-2.73 (m, 1H), 2.50-2.35 (m, 1H).

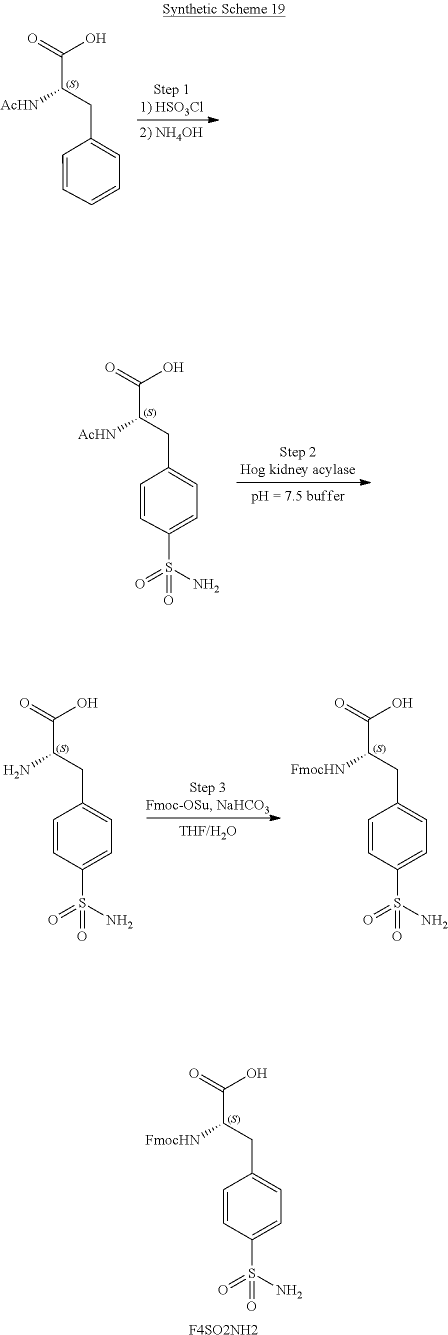

### (S)-2-((((9H-fluoren-9-yl)methoxy)carbonyl)amino)-3-(4-sulfamoylphenyl)propanoic acid

Step 1: A mixture of acetyl-L-phenylalanine (1 g, 4.83 mmol) in chlorosulfonic acid (5.34 g, 45.8 mmol) was stirred for 3 h at −10° C. Then the resulting solution was stirred for 2 h at 25° C. The reaction mixture was added to ice drop by drop, and the stirring continued. The resulting mixture was diluted with with EA (500 mL), washed with brine (3×250 mL), dried over anhydrous Na₂SO₄, the solvent was evaporated under reduced pressure to provide crude product. The crude product was dissolved in NH₄OH (15 mL, 28% wt) and stirred for 3 h at 100° C. The solvent was concentrated under reduced pressure and the pH was adjusted to 3 with 1 N HCl. The solid was collected by filtration and lyophilized to give (S)-2-acetamido-3-(4-sulfamoylphenyl) propanoic acid. MS ESI calculated for C₁₁H₁₅N₂O₅S [M+H]⁺ 287.06, found 287.05. ¹H NMR (300 MHz, DMSO-d₆) δ 8.32-8.30 (m, 1H), 7.75-7.70 (m, 2H), 7.43-7.41 (m, 2H), 7.28 (s, 2H), 4.49-4.31 (m, 1H), 3.13-3.11 (m, 1H), 2.92-2.91 (m, 1H), 1.78 (s, 3H).

Step 2: (S)-2-acetamido-3-(4-sulfamoylphenyl)propanoic acid (5.1 g, 17.81 mmol) was suspended in water (50 mL) and the pH value of the mixture was adjusted to 5 with LiOH (1 M) and adjusted to 7.5 with 0.25 M buffer. Hog kidney acylase (510 mg, 17.81 mmol) was added into the mixture at 25° C. Then the resulted mixture was stirred for 20 h at 25° C. The reaction was not further purified, the solution of (S)-2-amino-3-(4-sulfamoylphenyl)propanoic acid was directly used to the next reaction. MS ESI calculated for C₉H₁₃N₂O₄S [M+H]⁺ 245.05, found 245.10.

Step 3: To the solution of (S)-2-amino-3-(4-sulfamoylphenyl)propanoic acid (2.3 g, 9.42 mmol, dissolved in 50 mL of water) were added THF (50 mL), NaHCO₃ (3.95 g, 47.1 mmol) and Fmoc-OSu (3.18 g, 9.42 mmol) at room temperature. The resulting mixture was stirred at 25° C. for 16 h. The pH was adjusted to 3 with 1 N HCl. The reaction mixture was extracted with EA (3×120 mL). The organic layers were washed with brine (120 mL), dried over anhydrous Na₂SO₄. The solvent was concentrated under reduced pressure and the residue was purified by RP-flash with the following conditions: 330 g C¹⁸ column, 2%-2% in 5 min, 2%-30% in 30 min, ACN in water (0.05% NH₄HCO₃) to give (S)-2-((((9H-fluoren-9-yl)methoxy)carbonyl)amino)-3-(4-sulfamoylphenyl)propanoic acid. MS ESI calculated for C₂₄H₂₆N₃O₆S [M+NH₄]^{+ 484.12}, found 484.10. ¹H NMR (300 MHz, CD₃OD) δ 7.82-7.73 (m, 4H), 7.68-7.56 (m, 2H), 7.38-7.35 (m, 4H), 7.34-7.23 (m, 2H), 4.40-4.12 (m, 4H), 3.31-3.30 (m, 1H), 3.01-2.98 (m, 1H).

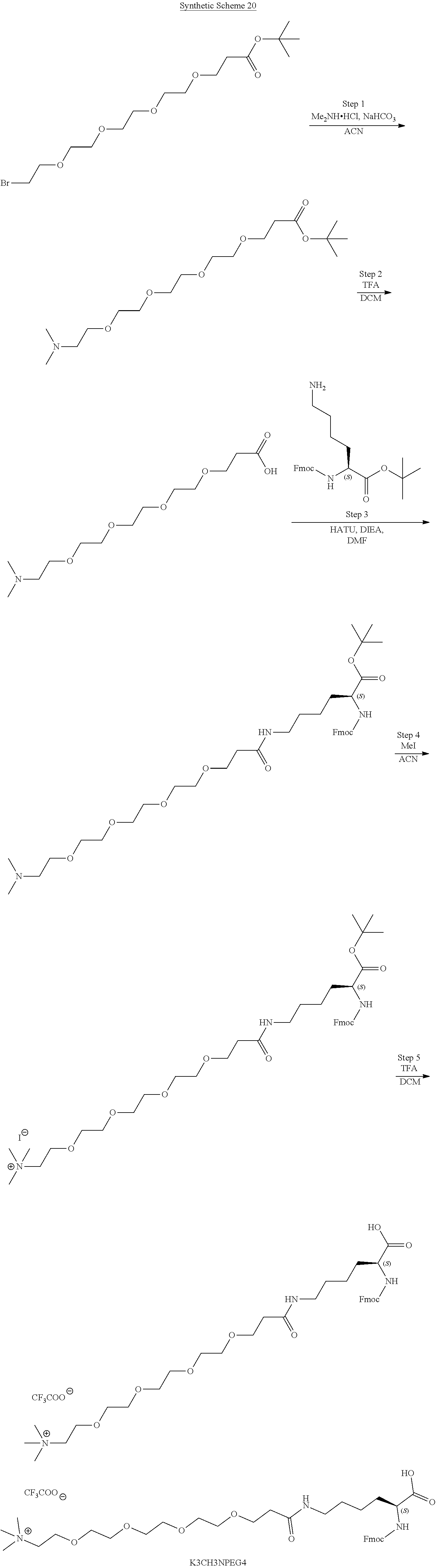

### (S)-5-carboxy-1-(9H-fluoren-9-yl)-N,N,N-trimethyl-3,11-dioxo-2,14,17,20,23-pentaoxa-4,10-diazapentacosan-25-aminium 2,2,2-trifluoroacetate

Step 1: To a stirred solution of tert-butyl 1-bromo-3,6,9,12-tetraoxapentadecan-15-oate (19 g, 49.3 mmol) in MeCN (100 mL) were added NaHCO₃ (12.43 g, 148 mmol) and dimethylamine hydrochloride (6.03 g, 74.0 mmol) at ambient temperature. Then the reaction mixture was stirred at 80° C. for 3 h. The reaction progress was monitored by LCMS and TLC. LCMS showed major was desired product and no SM remained. The reaction mixture was filtered and the filtrate was concentrate under reduced pressure. The residue was purified by silica gel column chromatography, eluting with gradient 0%-15% MeOH in DCM. The fractions containing desired product were combined and concentrated under reduced pressure to afford tert-butyl 2-methyl-5,8,11,14-tetraoxa-2-azaheptadecan-17-oate. MS ESI calculated for C₁₇H₃₆NO₆ [M+H]⁺ 350.25, found 350.35. ¹H-NMR (400 MHz, Chloroform-d) δ 3.91-3.88 (m, 2H), 3.73-3.69 (m, 2H), 3.67-3.66 (m, 4H), 3.65-3.64 (m, 4H), 3.63-3.61 (m, 4H), 3.11-3.08 (m, 2H), 2.74 (s, 6H), 2.52-2.49 (m, 2H), 1.45 (s, 9H).

Step 2: To a stirred solution of tert-butyl 2-methyl-5,8,11,14-tetraoxa-2-azaheptadecan-17-oate (16.5 g, 47.2 mmol) in DCM (100 mL) was added 2,2,2-trifluoroacetic acid (30 mL, 47.2 mmol) at ambient temperature. Then the reaction solution was stirred at ambient temperature for 2 h. The reaction progress was monitored by LCMS and TLC. LCMS showed major was desired product. The solvent was concentrated under reduced pressure and the residue was used directly for next step MS ESI calculated for C₁₃H₂₈NO₆ [M+H]⁺ 294.19, found 294.35.

Step 3: To a stirred solution of 2-methyl-5,8,11,14-tetraoxa-2-azaheptadecan-17-oic acid (13.77 g, 46.9 mmol) in DMF (80 mL) was added N-ethyl-N-isopropylpropan-2-amine (12.13 g, 94 mmol) at −40° C. under nitrogen atmosphere. The solution was stirred at −40° C. for 2 min. HATU (21.42 g, 56.3 mmol) and tert-butyl (((9H-fluoren-9-yl)methoxy)carbonyl)-L-lysinate (17.93 g, 42.2 mmol) were added to the solution and stirred at −40° C. for 3 h. The reaction was quenched with saturated NH₄Cl (100 mL), extracted with EtOAc (2×300 mL). The combined organic layer was washed with brine (3×80 mL), dried over anhydrous Na₂SO₄ and filtered. The filtrate was concentrated under reduced pressure and the residue was purified by RP flash with the following conditions: Column, C18 330 g, mobile phase: ACN in water (0.5% TFA), 5%-5% in 5 min, 5%-95% in 35 min; Detector, UV 254&210 nm. RT: 26 min. The collected fractions were combined and concentrated under reduced pressure to give tert-butyl (S)-23-((((9H-fluoren-9-yl)methoxy)carbonyl)amino)-2-methyl-17-oxo-5,8,11,14-tetraoxa-2,18-diazatetracosan-24-oate. MS ESI calculated for C₃₈H₅₉N₃O₉ [M+H]⁺ 700.42, found 700.40. ¹H-NMR (300 MHz, Methanol-d₄) δ 7.84-7.81 (m, 2H), 7.72-7.68 (m, 2H), 7.44-7.39 (m, 2H), 7.36-7.31 (m, 2H), 4.46-4.32 (m, 2H), 4.27-4.00 (m, 2H), 3.81-3.60 (m, 16H), 3.32-3.28 (m, 2H), 3.22-3.18 (m, 2H), 2.91 (s, 6H), 2.43 (t, J=6.3 Hz, 2H), 1.83-1.78 (m, 1H), 1.71-1.66 (m, 1H), 1.57-1.52 (m, 2H), 1.47 (s, 9H).

Step 4: To a stirred solution of tert-butyl (S)-23-((((9H-fluoren-9-yl)methoxy)carbonyl)amino)-2-methyl-17-oxo-5,8,11,14-tetraoxa-2,18-diazatetracosan-24-oate (13 g, 18.57 mmol) in MeCN (80 mL) were added NaHCO₃ (3.12 g, 37.1 mmol) and iodomethane (21.09 g, 149 mmol) at ambient temperature. Then the reaction solution was stirred at 70° C. The reaction progress was monitored by LCMS and TLC. The reaction mixture was filtered and the filtrate was concentrated under reduced pressure. The residue was used directly in next step. MS ESI calculated for C₃₉H₆₀N₃O₉[M]⁺ 714.43, found 714.41.

Step 5: To a stirred solution of (S)-5-(tert-butoxycarbonyl)-1-(9H-fluoren-9-yl)-N, N, N-trimethyl-3,11-dioxo-2,14,17,20,23-pentaoxa-4,10-diazapentacosan-25-aminium iodide (12 g, 14.25 mmol) in DCM (50 mL) was added 2,2,2-trifluoroacetic acid (30 mL, 14.25 mmol) at ambient temperature. Then the reaction solution was stirred at ambient temperature for 6 h. The reaction progress was monitored by LCMS. The reaction solution was concentrated under reduced pressure and the residue was purified by RP flash with the following conditions: Column, C18 330 g, mobile phase: ACN in water (0.5% TFA), 5%-5% in 5 min, 5%-95% in 25 min, 21% hold 8.2 min.; Detector, UV 254 nm. RT: 35 min. The collected fractions were combined and concentrated under reduced pressure to give (S)-5-carboxy-1-(9H-fluoren-9-yl)-N, N, N-trimethyl-3,11-dioxo-2,14,17,20,23-pentaoxa-4,10-diazapentacosan-25-aminium 2,2,2-trifluoroacetate. MS ESI calculated for C₃₅H₅₂N₃O₉ [M]⁺ 658.37, found 658.30. ¹H NMR (300 MHz, Methanol-d4) δ 7.82-7.79 (m, 2H), 7.70-7.66 (m, 2H), 7.42-7.29 (m, 4H), 4.39-4.35 (m, 2H), 4.26-4.23 (m, 2H), 3.93-3.88 (m, 2H), 3.72-3.52 (m, 16H), 3.20-3.17 (m, 11H), 2.43 (t, J=6.3 Hz, 2H), 1.89-1.87 (m, 1H), 1.79-1.71 (m, 1H), 1.54-1.42 (m, 4H).

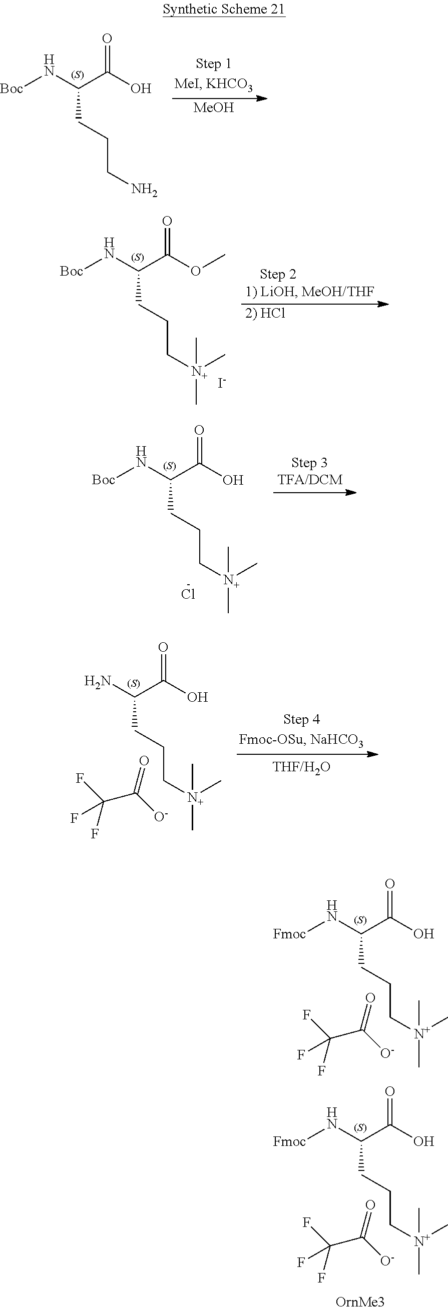

### (S)-4-((((9H-fluoren-9-yl)methoxy)carbonyl)amino)-4-carboxy-N,N,N-trimethylbutan-1-aminium 2,2,2-trifluoroacetate

Step 1: To a mixture of (S)-5-amino-2-((tert-butoxycarbonyl)amino)pentanoic acid (4.6 g, 19.80 mmol) in MeOH (50 mL) were added Mel (33.7 g, 238 mmol) and KHCO₃ (1.983 g, 19.80 mmol) at ambient temperature. The resulting mixture was warmed to 50° C. and stirred for 12 h. The reaction was cooled to room temperature and filtered. The filtrate was concentrated in vacuo and the residue was dissolved in DCM (80 mL) and then filtered again. The filtrate was concentrated in vacuo to afford (S)-4-((tert-butoxycarbonyl)amino)-5-methoxy-N,N,N-trimethyl-5-oxopentan-1-aminium iodide. MS ESI calculated for C₁₄H₂₉N₂O₄ [M-I]⁺ 289.21, found 289.35.

Step 2: To a mixture of (S)-4-((tert-butoxycarbonyl)amino)-5-methoxy-N,N,N-trimethyl-5-oxopentan-1-aminium iodide (7.4 g, 16.00 mmol) in MeOH (48 mL) and THF (24 mL) was added LiOH (48.0 mL, 48.0 mmol, 1 M in water) at ambient temperature. The reaction was stirred at ambient temperature for 2 h then concentrated in vacuo. Then 48 mL 1 N HCl was added. The solvent was concentrated in vacuo to afford (S)-4-((tert-butoxycarbonyl)amino)-4-carboxy-N,N,N-trimethylbutan-1-aminium chloride. MS ESI calculated for C₁₃H₂₇N₂O₄ [M-Cl]⁺ 275.20, found 275.20.

Step 3: To a mixture of (S)-4-((tert-butoxycarbonyl)amino)-4-carboxy-N,N,N-trimethylbutan-1-aminium chloride (7.5 g, 14.48 mmol) in DCM (50 mL) was added TFA (25 mL, 324 mmol) at ambient temperature. The reaction was stirred at ambient temperature for 1 h then concentrated in vacuo to afford (S)-4-amino-4-carboxy-N,N,N-trimethylbutan-1-aminium 2,2,2-trifluoroacetate. MS ESI calculated for C₈H₁₉N₂O₂ [M-CF₃COO]⁺ 175.14, found 175.20.

Step 4: To a mixture of (S)-4-amino-4-carboxy-N,N,N-trimethylbutan-1-aminium 2,2,2-trifluoroacetate (10 g, 13.88 mmol) in THF (30 mL) and water (30 mL) were added NaHCO₃ (9.33 g, 111 mmol) and Fmoc-OSu (4.21 g, 12.49 mmol) at ambient temperature. The reaction was stirred at ambient temperature for 2 h. The resulting solution was acidified with aqueous HCl to pH 3-4 and then filtered. The filtrate was purified by Rp-flash with the following conditions: Column: Flash C¹⁸ (330 g); Mobile Phase A: water (0.05% TFA), Mobile Phase B: ACN; (Gradient: 0% B hold 5 min, up to 33% B within 18 min, 33% B hold 7 min; up to 95% B within 5 min, 95% B hold 5 min); Flow rate: 90 mL/min; Detector: UV 210 nm; RT=40 min. The product-containing fractions were collected and lyophilized to afford (S)-4-((((9H-fluoren-9-yl)methoxy)carbonyl)amino)-4-carboxy-N,N,N-trimethylbutan-1-aminium 2,2,2-trifluoroacetate. MS ESI calculated for C₂₃H₂₉N₂O₄ [M-CF₃COO]⁺ 397.21, found 397.15. ¹H NMR (400 MHz, DMSO-d₆) δ 12.85 (br, 1H), 7.92-7.89 (m, 2H), 7.74-7.63 (m, 3H), 7.44-7.40 (m, 2H), 7.33-7.31 (m, 2H), 4.38-4.22 (m, 3H), 4.02-3.98 (m, 1H), 3.34-3.20 (m, 2H), 3.09 (s, 9H), 1.79-1.51 (m, 4H). ¹⁹F-NMR (376 MHz, DMSO-d₆) −73.64.

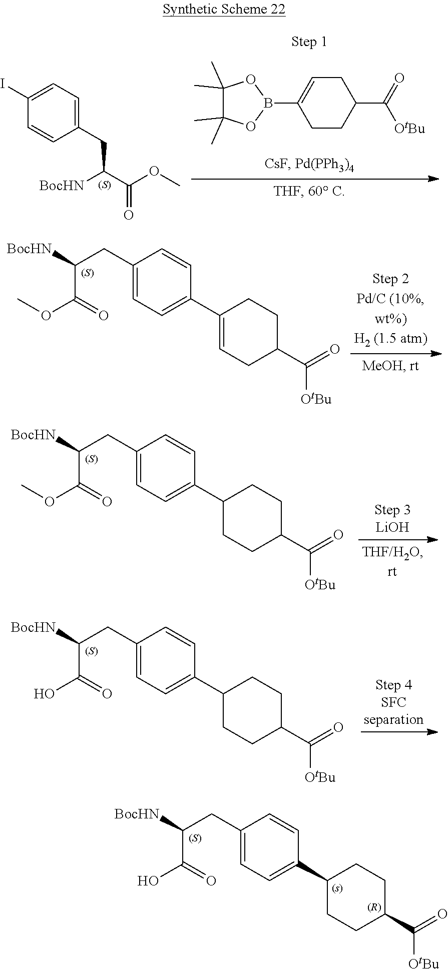

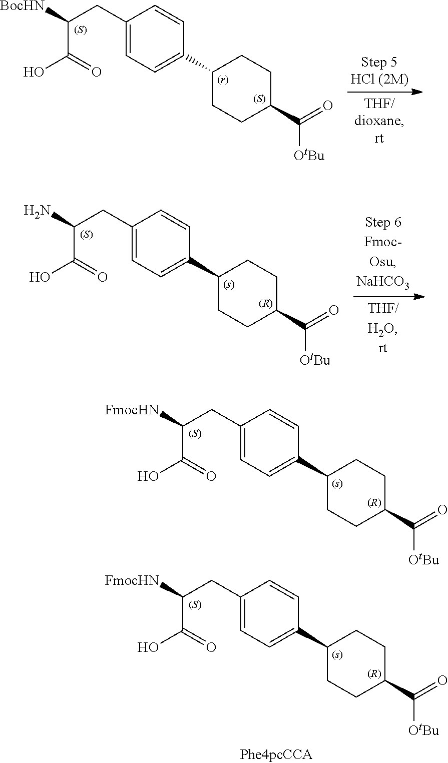

### (S)-2-((((9H-fluoren-9-yl)methoxy)carbonyl)amino)-3-(4-((1s,4R)-4-(tert-butoxycarbonyl)cyclohexyl)phenyl)propanoic acid

Step 1: To a mixture of tert-butyl 4-(4,4,5,5-tetramethyl-1,3,2-dioxaborolan-2-yl) cyclohex-3-ene-1-carboxylate (29.5 g, 96 mmol) in THF (30 mL) were added methyl (S)-2-((tert-butoxycarbonyl) amino)-3-(4-iodophenyl) propanoate (15.5 g, 38.2 mmol), and Pd(Ph₃P)₄ (2.210 g, 1.912 mmol) at room temperature. The reaction was warmed to 60° C. for 4 h. The resulting solution was quenched with water (100 mL) and extracted with ethyl acetate (3×300 mL). The organic layers were combined, washed with brine (2×200 mL), dried over anhydrous sodium sulfate and filtered. The filtrate was concentrated in vacuo to afford crude product. The residue was purified by silica gel chromatography, eluting with a gradient of ethyl acetate: petroleum ether—0:1 to 1:4 to afford tert-butyl 4′-((S)-2-((tert-butoxycarbonyl)amino)-3-methoxy-3-oxopropyl)-2,3,4,5-tetrahydro-[1,1′-biphenyl]-4-carboxylate. MS ESI calculated for C₂₆H₃₈NO₆Na [M+Na]⁺, 482.26 found 482.10.

Step 2: To a mixture of tert-butyl 4′-((S)-2-((tert-butoxycarbonyl)amino)-3-methoxy-3-oxopropyl)-2,3,4,5-tetrahydro-[1,1′-biphenyl]-4-carboxylate (16 g, 34.8 mmol) in methanol (160 mL) was added Pd—C (10% on carbon, wetted with ca. 55% water, 5.3 g, 4.98 mmol) at room temperature. The mixture was degassed with H₂ three times and stirred for 1 h at room temperature under the atmosphere of H₂ (1.5 atm). The resulting mixture was filtered. The filtrate was concentrated in vacuo to afford tert-butyl(S)-4-(4-(2-((tert-butoxycarbonyl) amino)-3-methoxy-3-oxopropyl)phenyl)cyclohexane-1-carboxylate. MS ESI calculated for C₂₆H₄₁NO₆ [M+H]⁺, 462.28, found 462.30.

Step 3: To a stirred solution of tert-butyl(S)-4-(4-(2-((tert-butoxycarbonyl) amino)-3-methoxy-3-oxopropyl)phenyl)cyclohexane-1-carboxylate (15 g, 32.5 mmol) in THF (300 mL) was added LiOH (65.0 mL, 65.0 mmol) at room temperature. The solution was stirred at 20° C. for 1 h. The pH value of the solution was adjusted to 3 with 1 N HCl. The reaction was concentrated under reduced pressure to give (S)-2-((tert-butoxycarbonyl)amino)-3-(4-(4-(tert-butoxycarbonyl)cyclohexyl)phenyl)propanoic acid. MS ESI calculated for C₂₅H₃₈NO₆Na [M+Na]⁺, 470.26, found 470.30.

Step 4: The (S)-2-((tert-butoxycarbonyl)amino)-3-(4-(4-(tert-butoxycarbonyl) cyclohexyl) phenyl) propanoic acid (14 g, 31.3 mmol) was separated with Prep-SFC with following conditions: Column: CHIRAL ART Cellulose-SB, 3×25 cm, 5 μm; Mobile Phase A: CO₂, Mobile Phase B: MeOH (0.1% 2M NH₃-MEOH); Flow rate: 80 mL/min; Gradient: 10% B; 220 nm; RT1: 7.45; RT2: 8.38; Injection Volume: 1.3 ml; Number Of Runs: 131; to afford (S)-2-((tert-butoxycarbonyl) amino)-3-(4-((1s,4R)-4-(tert-butoxycarbonyl) cyclohexyl) phenyl) propanoic acid (fast-eluting peak). MS ESI calculated for C₂₅H₃₈NO₆Na [M+Na]⁺, 470.26 found 470.30. 1H NMR (300 MHz, Chloroform-d) δ 7.11 (d, J=3.0 Hz, 4H), 4.27 (s, 1H), 3.18-3.12 (m, 1H), 2.91 (s, 1H), 2.60 (s, 1H), 2.49 (s, 1H), 2.20 (d, J=10.9 Hz, 2H), 1.78-1.52 (m, 6H), 1.49 (d, J=0.7 Hz, 9H), 1.36 (s, 9H).

As a slow-eluting peak, (S)-2-((tert-butoxycarbonyl)amino)-3-(4-((1r,4S)-4-(tert-butoxycarbonyl)cyclohexyl)phenyl)propanoic acid was isolated. MS ESI calculated for C₂₅H₃₈NO₆Na [M+Na]⁺, 470.26 found 470.30. 1H NMR (300 MHz, Chloroform-d) δ 7.14-7.06 (m, 4H), 4.25 (s, 1H), 3.14 (d, J=12.3 Hz, 1H), 2.88 (s, 1H), 2.49-2.41 (m, 1H), 2.29-2.15 (m, 1H), 2.05 (d, J=12.2 Hz, 2H), 1.90 (d, J=12.1 Hz, 2H), 1.69-1.47 (m, 2H), 1.47 (s, 11H), 1.34 (s, 9H).

Step 5: To a mixture of (S)-2-((tert-butoxycarbonyl) amino)-3-(4-((1s,4R)-4-(tert-butoxycarbonyl) cyclohexyl) phenyl) propanoic acid (8.3 g, 18.54 mmol) in THF (80 mL) was added hydrogen chloride (M/dioxane) (9.27 mL, 18.54 mmol) in portions at room temperature. The reaction was concentrated under reduced pressure to afford (S)-2-amino-3-(4-((1s,4R)-4-(tert-butoxycarbonyl)cyclohexyl) phenyl)propanoic acid. MS ESI calculated for C₂₀H₃₀NO₄ [M+H]⁺, 348.21 found 348.25.

Step 6: To a stirred solution of (S)-2-amino-3-(4-((1s,4R)-4-(tert-butoxycarbonyl) cyclohexyl) phenyl) propanoic acid (6 g, 17.27 mmol) and NaHCO₃ (7.25 g, 86 mmol) in THF (60 mL) and water (60.0 mL) was added n-(9-fluorenylmethoxycarbonyloxy)succinimide (5.24 g, 15.54 mmol) at room temperature. The mixture was stirred at room temperature for 1 h. The pH value of the solution was adjusted to 3 with 1 N HCl. The aqueous phase was extracted with ether acetate (2×200 mL). The combined organic layer was washed with brine (150 mL), dried over anhydrous Na₂SO₄ and filtered. The filtrate was concentrated under reduced pressure, and the residue was purified by RP flash to give (S)-2-((((9H-fluoren-9-yl)methoxy)carbonyl)amino)-3-(4-((1s,4R)-4-(tert-butoxycarbonyl)cyclohexyl)phenyl)propanoic acid. MS ESI calculated for C₃₅H₄₁NO₆ [M+H]⁺, 570.28 found 570.15. ¹H NMR (300 MHz, Methanol-d₄) δ 7.81 (d, J=7.5 Hz, 2H), 7.61 (d, J=7.5 Hz, 2H), 7.43-7.27 (m, 4H), 7.16 (d, J=7.8 Hz, 2H), 7.07 (d, J=7.9 Hz, 2H), 4.45-4.33 (m, 2H), 4.22-4.06 (m, 2H), 3.26-3.14 (m, 1H), 2.96-2.88 (m, 1H), 2.59 (s, 1H), 2.48 (s, 1H), 2.15 (s, 2H), 1.61 (d, J=5.5 Hz, 6H), 1.49 (s, 9H).

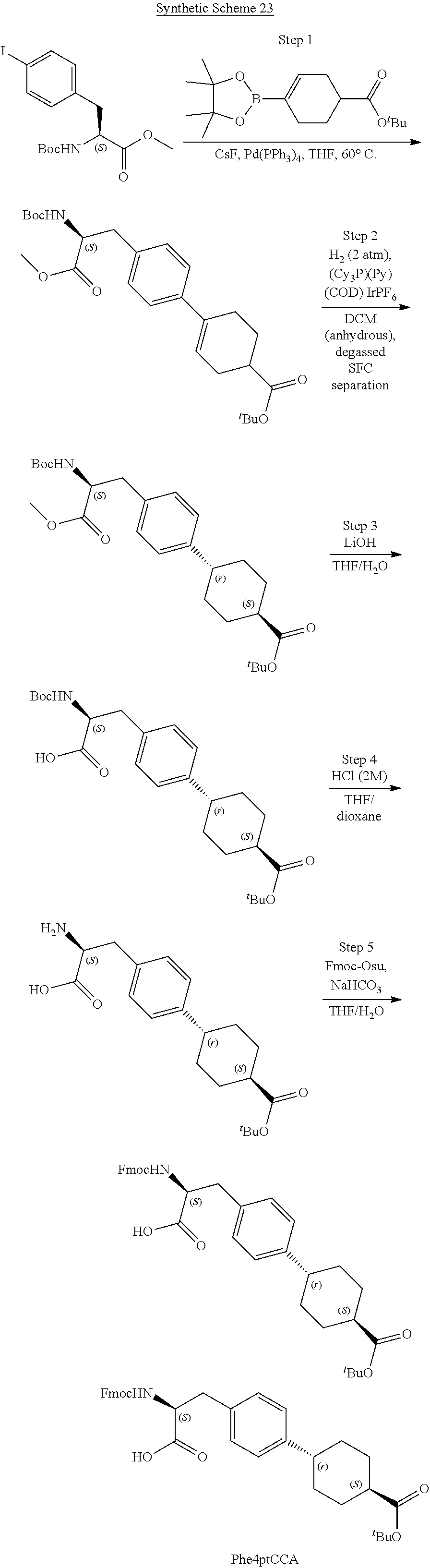

### (S)-2-((((9H-fluoren-9-yl)methoxy)carbonyl)amino)-3-(4-((1r,4S)-4-(tert-butoxycarbonyl)cyclohexyl)phenyl)propanoic acid

Step 1: To a mixture of tert-butyl 4-(4,4,5,5-tetramethyl-1,3,2-dioxaborolan-2-yl)cyclohex-3-ene-1-carboxylate (18.25 g, 59.2 mmol) in THF (200 mL) were added methyl (S)-2-((tert-butoxycarbonyl)amino)-3-(4-iodophenyl)propanoate (20 g, 49.4 mmol), Pd(PPh₃)₄(2.85 g, 2.468 mmol) and CsF (30.0 g, 197 mmol) under argon. The reaction was stirred at 60° C. for 16 h under argon. The reaction was quenched with water (150 mL) and extracted with EA (3×500 mL). The combined organic layers were dried over anhydrous MgSO₄ and filtered. The residue was purified by silica gel chromatography, eluting with a gradient of ethyl acetate: petroleum ether—0:1 to 1:5 to afford tert-butyl 4′-((S)-2-((tert-butoxycarbonyl)amino)-3-methoxy-3-oxopropyl)-2,3,4,5-tetrahydro-[1,1′-biphenyl]-4-carboxylate. MS ESI calculated for C₂₆H₃₇NO₆Na [M+Na]⁺ 482.26, found 482.30.

Step 2: To a mixture of tert-butyl (R)-4′-((S)-2-((tert-butoxycarbonyl)amino)-3-methoxy-3-oxopropyl)-2,3,4,5-tetrahydro-[1,1′-biphenyl]-4-carboxylate (21 g, 45.7 mmol) in dichloromethane (200 mL) was added Crabtree's catalyst (14.71 g, 18.28 mmol) under argon. The suspension was degassed under vacuum and purged with H₂ several times, the reaction solution was stirred for 24 h at room temperature under 2 atm H₂. The reaction was filtered. The filtrate was concentrated in vacuo to afford racemic product. The racemic product was separated with a Prep-SFC Column: CHIRALPAK IG, 5*25 cm, 10 m; Mobile Phase A: CO₂, Mobile Phase B: MeOH (0.1% 2M NH₃-MeOH); Flow rate: 200 mL/min; Gradient: isocratic 25% B; Column Temperature (° C.): 35; Back Pressure (bar): 100; Wave Length: 220 nm; RT1(min): 4.15 to afford tert-butyl (1S,4R)-4-(4-((S)-2-((tert-butoxycarbonyl)amino)-3-methoxy-3-oxopropyl)phenyl)cyclohexane-1-carboxylate as an orange oil. MS ESI calculated for C₂₆H₃₉NO₆Na [M+Na]⁺ 484.28, found 484.35.

Step 3: To a mixture of tert-butyl (1S,4R)-4-(4-((S)-2-((tert-butoxycarbonyl)amino)-3-methoxy-3-oxopropyl)phenyl)cyclohexane-1-carboxylate (10.5 g, 22.75 mmol) in THF (45.5 mL) was added LiOH (1M in water) (45.5 mL, 45.5 mmol) at ambient temperature. The reaction was stirred at ambient temperature for 2 h. The resulting solution was acidified with aqueous HCl (1M) and extracted with EA (4×100 mL). The combined organic layers were dried over anhydrous MgSO₄ and filtered. The filtrate was concentrated in vacuo to afford (S)-2-((tert-butoxycarbonyl)amino)-3-(4-((1R,4S)-4-(tert-butoxycarbonyl)cyclohexyl)phenyl)propanoic acid. MS ESI calculated for C₂₅H₃₇NO₆Na [M+Na]⁺ 470.26, found 470.25.

Step 4: To a mixture of (S)-2-((tert-butoxycarbonyl)amino)-3-(4-((1R,4S)-4-(tert-butoxycarbonyl)cyclohexyl)phenyl)propanoic acid (9.5 g, 21.23 mmol) in THF (100 mL) was added HCl (4M in 1,4dioxane) (100 mL, 400 mmol) at ambient temperature. The reaction was stirred at ambient temperature for 4 h. The resulting solution was concentrated in vacuo to afford (S)-2-amino-3-(4-((1R,4S)-4-(tert-butoxycarbonyl)cyclohexyl)phenyl)propanoic acid. MS ESI calculated for C₂₀H₃₀NO₄ [M+H]⁺ 348.21, found 348.25.

Step 5: To a mixture of (S)-2-amino-3-(4-((1R,4S)-4-(tert-butoxycarbonyl)cyclohexyl)phenyl)propanoic acid (7 g, 20.15 mmol) in THF (30 mL) and water (30 mL) were added sodium bicarbonate (8.46 g, 101 mmol) and n-(9-fluorenylmethoxycarbonyloxy)succinimide (6.12 g, 18.13 mmol). The reaction was stirred at ambient temperature for 2 h. The resulting solution was acidified with aqueous HCl (1 M) and extracted with EA (5×100 mL). The combined organic layers were dried over anhydrous MgSO₄ and filtered. The filtrate was concentrated in vacuo to afford crude product. The residue was purified by RP flash with the following conditions: Column: Flash C 18 (330 g); Mobile Phase A: water (0.05% TFA), Mobile Phase B: ACN; (Gradient: 2% B hold 5 min, up to 75% B within 15 min, 76.5% B hold 6 min; up to 95% B within 5 min, 95% B hold 5 min); Flow rate: 90 mL/min; Detector: UV 210 nm; RT=31 min. The product-containing fractions were collected and concentrated in vacuo to give (S)-2-((((9H-fluoren-9-yl)methoxy)carbonyl)amino)-3-(4-((1R,4S)-4-(tert-butoxycarbonyl)cyclohexyl)phenyl)propanoic acid. MS ESI calculated for C₃₅H₄₀NO₆ [M+H]⁺ 570.29, found 570.25. ¹H NMR (400 MHz, methanol-d₄) δ 7.80-7.78 (m, 2H), 7.60-7.58 (m, 2H), 7.40-7.38 (m, 2H), 7.37-7.28 (m, 2H), 7.16-7.14 (m, 2H), 7.06-7.04 (m, 2H), 4.45-4.41 (m, 1H), 4.32-4.28 (m, 1H), 4.15-4.055 (m, 2H), 3.21-3.17 (m, 1H), 2.91-2.88 (m, 1H), 2.41-2.37 (m, 1H), 2.19-2.17 (m, 1H), 1.96-1.91 (m, 2H), 1.78-1.75 (m, 2H), 1.45-1.42 (m, 10H), 1.40-1.32 (m, 3H).

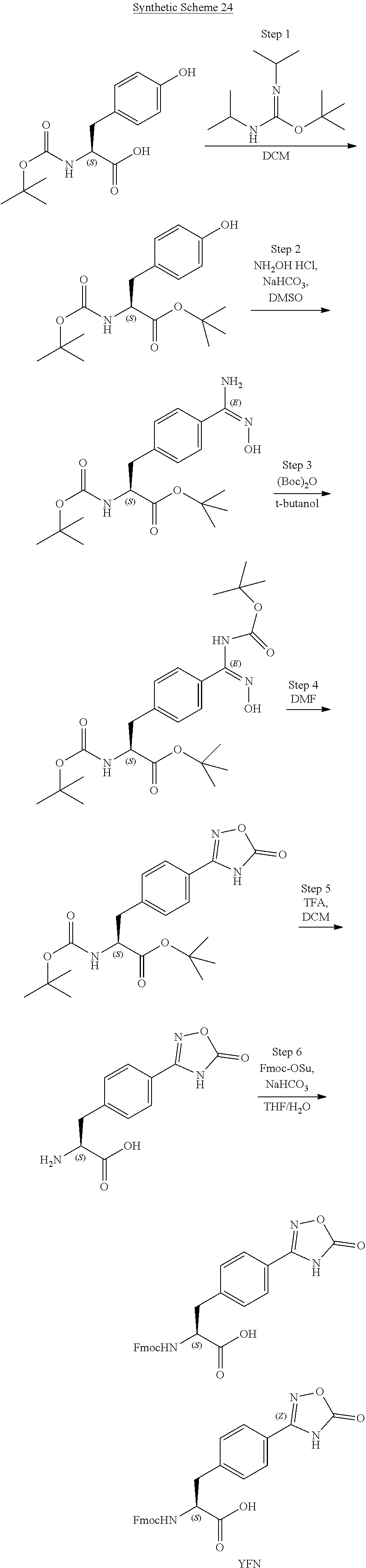

### (S)-2-((((9H-fluoren-9-yl)methoxy)carbonyl)-λ²-azaneyl)-3-(4-(5-oxo-4,5-dihydro-1,2,4-oxadiazol-3-yl)phenyl)propanoic acid

Step 1: To a mixture of (S)-2-((tert-butoxycarbonyl)amino)-3-(4-cyanophenyl)propanoic acid (5 g, 17.22 mmol) in DCM (10 mL) was added tert-butyl N,N-diisopropylcarbamimidate (17.25 g, 86 mmol). The reaction was refluxed at 40° C. for 2 h. The resulting solution was concentrated under vacuum. The residue was purified by silica gel chromatography, eluting with a gradient of ethyl acetate: petroleum ether—0:1 to 1:3 to afford tert-butyl (S)-2-((tert-butoxycarbonyl) amino)-3-(4-cyanophenyl)propanoate. MS ESI calculated for C₁₉H₂₆N₂O₄Na [M+Na]⁺ 369.19, found 369.10.

Step 2: Sodium bicarbonate (10.91 g, 130 mmol) was added to a solution of hydroxylamine hydrochloride (6.62 g, 95 mmol) in DMSO (120 mL) at room temperature and then the temperature was increased to 45-50° C. At this temperature, tert-butyl (S)-2-((tert-butoxycarbonyl) amino)-3-(4-cyanophenyl) propanoate (6 g, 17.32 mmol) was added to the resulting solution and the mixture was heated up to 100° C. followed by stirring at the same temperature for 18 h. After completion, the reaction mass was cooled to 15-20° C., then poured into water. The solution was stirred for 15-20 min at 15-20° C. and filtered. The collected solid was washed with water and dried to give tert-butyl (S,E)-2-((tert-butoxycarbonyl) amino)-3-(4-(N-hydroxycarbamimidoyl) phenyl)propanoate. MS ESI calculated for C₁₉H₃₀N₃O₅ [M+H]⁺ 380.21, found 380.15.

Step 3: tert-butyl (S,E)-2-((tert-butoxycarbonyl)amino)-3-(4-(N-hydroxycarbamimidoyl)phenyl)propanoate (5.5 g, 14.49 mmol) was dissolved in t-butanol (100 mL), then BOC₂O (3.37 mL, 14.49 mmol) was added slowly through an addition funnel at 25-30° C. After completion of addition, the temperature was increased to 50-55° C. and stirring was continued at the same temperature for 12 h. After completion of reaction, the reaction mixture was cooled to 15-20° C. and stirred for 60 min at 15-20° C. to obtain the solid. The product was filtered, washed with tertiary-butanol and dried under vacuum at 55-60° C. to afford the crude product. MS ESI calculated for C₂₄H₃₈N₃O₇ [M+H]⁺ 480.26, found 480.35.

Step 4: tert-butyl (S,E)-3-(4-(N-(tert-butoxycarbonyl)-N-hydroxycarbamimidoyl)phenyl)-2-((tert-butoxycarbonyl)amino)propanoate (5.5 g, 11.47 mmol) was dissolved in DMF (60 mL) and then the reaction was heated up to 110-115° C. The resulting solution was stirred at the same temperature for 12 h. The reaction mixture was cooled to room temperature and diluted with 600 mL EtOAc and washed with aqueous saturated NaCl (4×100 mL), dried over Na₂SO₄, filtered. The filtrate was concentrated under reduced pressure. The residue was purified by silica gel column chromatography, eluting with gradient 0%-70% EtOAc in PE. The fractions containing desired product were combined and concentrated under reduced pressure to afford tert-butyl (S)-2-((tert-butoxycarbonyl)amino)-3-(4-(5-oxo-4,5-dihydro-1,2,4-oxadiazol-3-yl) phenyl) propanoate. MS ESI calculated for C₂₀H₂₈N₃O₆ [M+H]⁺ 406.19, found 406.10. ¹H NMR (400 MHz, DMSO-d₆) δ 12.91 (s, 1H), 7.73-7.72 (m, 2H), 7.48-7.43 (m, 2H), 7.26-7.24 (m, 1H), 4.07-4.05 (m, 1H), 3.03-3.01 (m, 1H), 2.95-2.89 (m, 1H), 1.33 (s, 9H), 1.32 (s, 9H).

Step 5: To a mixture of tert-butyl (S)-2-((tert-butoxycarbonyl)amino)-3-(4-(5-oxo-4,5-dihydro-1,2,4-oxadiazol-3-yl)phenyl)propanoate (4 g, 9.87 mmol) in DCM (40 mL) was added TFA (40 mL, 519 mmol) under argon at room temperature. The reaction was stirred at room temperature for 1 h. The mixture was concentrated under reduced pressure to afford the crude product. MS ESI calculated for C₁₁H₁₂N₃O₄ [M+H]⁺ 250.07, found 249.95.

Step 6: To a mixture of (S)-2-amino-3-(4-(5-oxo-4,5-dihydro-1,2,4-oxadiazol-3-yl)phenyl)propanoic acid (2.1 g, 8.43 mmol) in THF (20 mL) and H₂O (20 mL) were added sodium bicarbonate (3.54 g, 42.1 mmol) and N-(9-fluorenylmethoxycarbonyloxy)succinimide (2.56 g, 7.58 mmol) under argon at room temperature. The reaction was stirred at room temperature for 1 h. The pH value of the solution was adjusted to 4 with 1 N HCl. The aqueous phase was extracted with ethyl acetate (2×200 mL). The combined organic layer was washed with brine (3×50 mL), dried over anhydrous Na₂SO₄ and filtered. The filtrate was concentrated under reduced pressure and the residue was purified by RP flash with the following conditions: Column: AQ 18 gel column (330 g), 20-35 m; Mobile Phase A: 5 mM aq. TFA; Mobile Phase B: MeCN; (Gradient: 0% B hold 5 min, up to 55.3% B within 35 min, 55.3% B hold 3.2 min; up to 95% B within 2 min, 95% B hold 10 min); Flow rate: 60 mL/min; Detector: UV 254 & 210 nm; RT: 35.32 min. The fractions containing desired product were combined and concentrated under reduced pressure to give (S)-2-((((9H-fluoren-9-yl)methoxy)carbonyl)amino)-3-(4-(5-oxo-4,5-dihydro-1,2,4-oxadiazol-3-yl)phenyl)propanoic acid. MS ESI calculated for C₂₆H₂₀N₃O₆ [M−H]⁺ 470.14, found 470.15. ¹H NMR (400 MHz, DMSO-d₆) δ 12.91 (s, 1H), 12.82 (s, 1H), 7.93-7.87 (m, 2H), 7.78-7.72 (m, 3H), 7.68-7.62 (m, 2H), 7.51-7.30 (m, 4H), 7.28-7.26 (m, 2H), 4.27-4.15 (m, 4H), 3.20-3.12 (m, 1H), 2.99-2.92 (m, 1H).

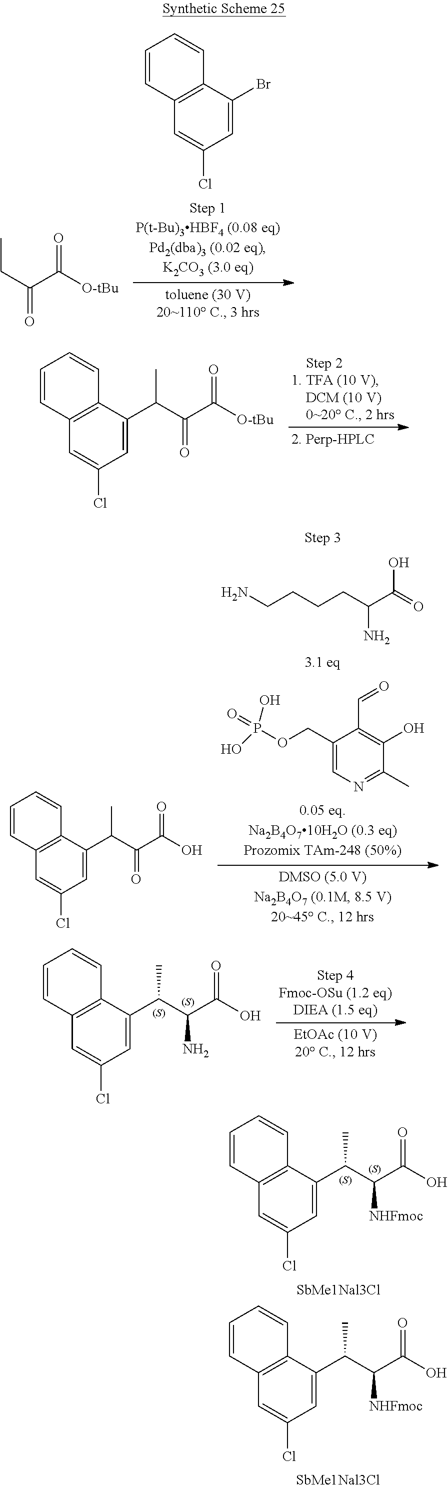

### (2S,3S)-2-((((9H-fluoren-9-yl)methoxy)carbonyl)amino)-3-(3-chloronaphthalen-1-yl)butanoic acid

Step 1: To a mixture of tert-butyl 2-oxobutanoate (80.0 g, 506 mmol, 1.00 eq) and K₂CO₃ (210 g, 1520 mmol, 3.00 eq) in toluene (2.40 L) were added 1-bromo-3-chloronaphthalene, P(t-Bu)₃·HBF₄ (11.7 g, 40.5 mmol, 0.08 eq) and the mixture was purged with N₂ for three times. Pd₂(dba)₃ (9.26 g, 10.1 mmol, 0.02 eq) was added and the reaction mixture was purged with N₂ three times, and stirred at 110° C. for 3 h under N₂. The reaction mixture was cooled to room temperature, poured into aq. NH₄Cl (3.00 L) and extracted with EtOAc (2.00 L×2). The organic layers were combined, washed with brine, dried over Na₂SO₄, filtered and concentrated under reduced pressure. The crude product was purified by silica chromatography (SiO₂, petroleum ether/ethyl acetate=1/0 to 0/1).

Step 2: To a mixture of tert-butyl 3-(3-chloronaphthalen-1-yl)-2-oxobutanoate (100 g, 314 mmol, 1.00 eq) in DCM (1.00 L) at 0° C. was dropwise added TFA (1.00 L), and the mixture was stirred for 2 h at ambient temperature. The reaction mixture was concentrated and the crude product was purified by prep-HPLC (column: Phenomenex luna C18 (250*70 mm, 15 um); mobile phase: [H₂O (0.1% TFA)-ACN]; gradient: 35%-65% B over 20.0 min).

Step 3: To a mixture of lysine (114 g, 779 mmol, 3.10 eq) in DMSO (270 mL) and Na₂B₄O₇ (0.1M, 561 mL) at 20° C. was added Na₂B₄O₇·10H₂O (28.8 g, 75.4 mmol, 0.30 eq) followed by compound 3-(3-chloronaphthalen-1-yl)-2-oxobutanoic acid (66.0 g, 251 mmol, 1.00 eq). Pyridoxal phosphate (2.44 g, 9.85 mmol, 0.05 eq) and transaminase enzyme Prozomix TAm-248 (35.0 g) were added to the reaction mixture at 35° C. The reaction mixture was stirred at 45° C. for 12 h. The reaction mixture was cooled to 20° C., adjusted to pH=5.0 with 5 N HCl (60.0 mL), and stirred at 20° C. for 1 hr. The mixture was filtered and the filter cake washed with H₂O (500 mL×3), and dried under reduced pressure to provide (2S,3S)-2-amino-3-(3-chloronaphthalen-1-yl)butanoic acid, used in the next step without purification.

Step 4: To a mixture of (2S,3S)-2-amino-3-(3-chloronaphthalen-1-yl)butanoic acid (54.0 g, 205 mmol, 1.00 eq) in EtOAc (540 mL) were added DIPEA (39.7 g, 307 mmol, 1.50 eq) and (9H-fluoren-9-yl)methyl (2,5-dioxopyrrolidin-1-yl) carbonate (82.9 g, 246 mmol, 1.20 eq) and the mixture was stirred for 12 h. The precipitate was filtered off and the filtrate washed with 2.0 L of EtOAc. The filtrate was concentrated to obtain the crude product, which was purified by prep-HPLC (column: Phenomenex luna C18 250*150 mm*15 um; mobile phase: [H₂O (0.04% HCl)-ACN]; gradient: 50%-95% B over 25.0 min). ¹H NMR: (400 MHz, DMSO-d₆) δ 12.8 (s, 1H), 8.22 (d, J=8.2 Hz, 1H), 7.84-8.01 (m, 5H), 7.53-7.72 (m, 4H), 7.31-7.47 (m, 3H), 7.18-7.30 (m, 2H), 4.54 (t, J=8.8 Hz, 1H), 4.21-4.32 (m, 1H), 3.95-4.19 (m, 3H), 3.82 (s, 3H), 1.36 (d, J=6.8 Hz, 3H).

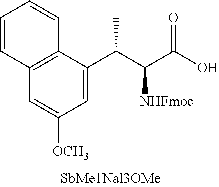

### (2S,3S)-2-((((9H-fluoren-9-yl)methoxy)carbonyl)amino)-3-(3-methoxynaphthalen-1-yl)butanoic acid

The title compound was obtained from appropriate starting materials using a process similar to the synthesis of SbMe1Nal3Cl. ¹H NMR: (400 MHz, DMSO-d₆) δ 12.77 (s, 1H), 8.10 (d, J=8.2 Hz, 1H), 7.84 (t, J=8.8 Hz, 4H), 7.60 (d, J=7.4 Hz, 1H), 7.55 (d, J=7.4 Hz, 1H), 7.34-7.50 (m, 4H), 7.13-7.29 (m, 3H), 7.05 (s, 1H), 4.53 (t, J=8.2 Hz, 1H), 4.18-4.32 (m, 1H), 4.07-4.17 (m, 2H), 3.93-4.01 (m, 1H), 3.82 (s, 3H), 1.33 (d, J=6.8 Hz, 3H).

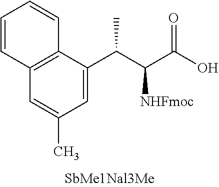

### (2S,3S)-2-((((9H-fluoren-9-yl)methoxy)carbonyl)amino)-3-(3-methylnaphthalen-1-yl)butanoic acid

The title compound was obtained from appropriate starting materials using a process similar to the synthesis of SbMe1Nal3Cl. ¹H NMR: (400 MHz, DMSO-d₆) δ 8.24 (d, J=6.8 Hz, 1H), 7.83-7.90 (m, 2H), 7.77-7.82 (m, 1H), 7.57-7.64 (m, 1H), 7.50-7.56 (m, 2H), 7.34-7.49 (m, 5H), 7.18-7.30 (m, 3H), 4.43 (t, J=7.6 Hz, 1H), 4.22-4.31 (m, 1H), 4.08-4.17 (m, 2H), 3.95-4.04 (m, 1H), 2.40 (s, 3H), 1.32 (d, J=6.8 Hz, 3H).

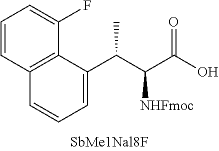

### (2S,3S)-2-((((9H-fluoren-9-yl)methoxy)carbonyl)amino)-3-(8-fluoronaphthalen-1-yl)butanoic acid

The title compound was obtained from appropriate starting materials using a process similar to the synthesis of SbMe1Nal3Cl. ¹H NMR: (400 MHz, DMSO-d₆) δ 7.84 (d, J=7.5 Hz, 3H), 7.78 (d, J=8.1 Hz, 1H), 7.71 (d, J=7.0 Hz, 1H), 7.57 (d, J=7.3 Hz, 1H), 7.43-7.53 (m, 4H), 7.33-7.41 (m, 2H), 7.17-7.33 (m, 3H), 4.49 (s, 1H), 4.19-4.37 (m, 2H), 4.04-4.09 (m, 2H), 1.32 (d, J=6.6 Hz, 3H).

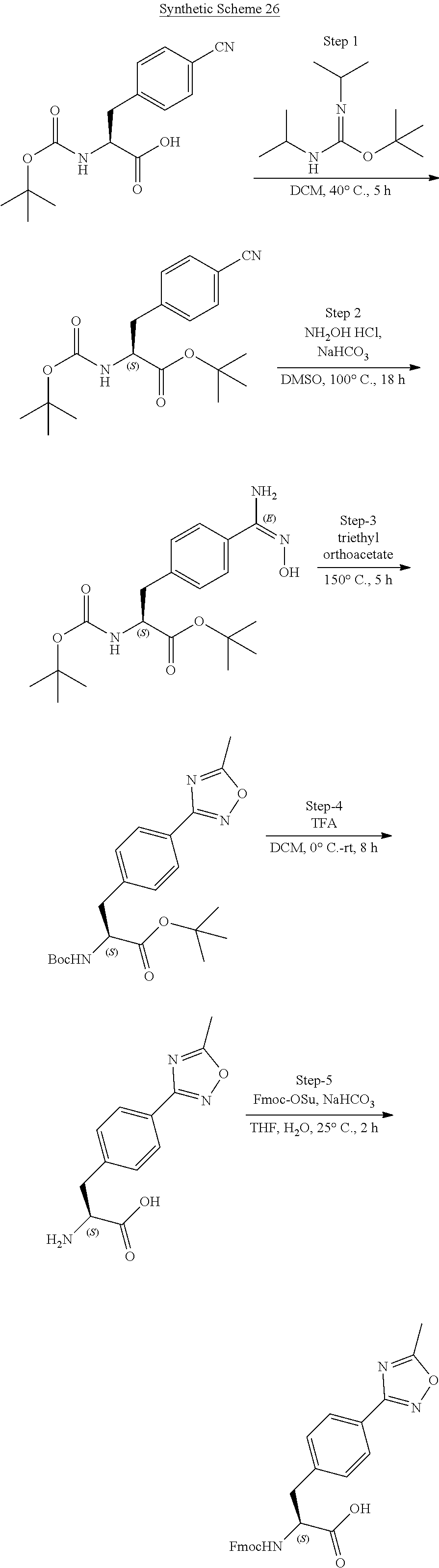

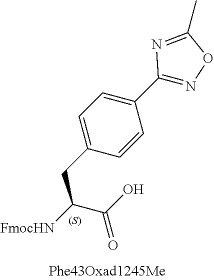

### (S)-2-((((9H-fluoren-9-yl)methoxy)carbonyl)amino)-3-(4-(5-methyl-1,2,4-oxadiazol-3-yl)phenyl)propanoic acid

Step 1: To a stirred solution of (S)-2-((tert-butoxycarbonyl)amino)-3-(4-cyanophenyl)propanoic acid (20.0 g, 68.9 mmol) in DCM (200 mL) was added tert-butyl (E)-N,N′-diisopropylcarbamimidate (69.0 g, 344 mmol) at room temperature. The reaction mixture was stirred at 40° C. for 5 h. The reaction mixture was concentrated under reduced pressure. The crude compound was purified by Biotage-isolera using silica column and the compound eluted with 30% of ethyl acetate in petroleum ether. Pure fractions were combined and concentrated under reduced pressure to afford tert-butyl (S)-2-((tert-butoxycarbonyl)amino)-3-(4-cyanophenyl)propanoate. MS ESI calculated for C₁₉H₂₆N₂O₄ [M−H]⁺ 345.19, found 345.25.

Step 2: To a stirred solution of hydroxylammoniumchloride (21.0 g, 302 mmol) in DMSO (400 mL) was added sodium bicarbonate (34.6 g, 411 mmol) at room temperature. Then tert-butyl (S)-2-((tert-butoxycarbonyl)amino)-3-(4-cyanophenyl)propanoate (19.0 g, 54.8 mmol) was added to the reaction mixture at 45-50° C. The reaction mixture was stirred at 100° C. for 18 h. The reaction mixture was cooled to 15-20° C., quenched with water and stirred at 15-20° C. for 15-20 min. The compound was filtered, washed with water and diethyl ether and dried under vacuum to afford tert-butyl (S,E)-2-((tert-butoxycarbonyl)amino)-3-(4-(N-hydroxycarbamimidoyl)phenyl)propanoate. MS ESI calculated for C₁₉H₂₉N₃O₅ [M+H]⁺ 380.21, found 380.29.

Step 3: A solution of tert-butyl (S,E)-2-((tert-butoxycarbonyl)amino)-3-(4-(N-hydroxycarbamimidoyl)phenyl)propanoate (19.0 g, 50.1 mmol) in triethyl orthoacetate (190 mL) was stirred at 150° C. for 5 h. The reaction mixture was concentrated under reduced pressure. The crude compound was purified by Biotage-isolera using a silica column and the compound eluted with 30% of ethyl acetate in petroleum ether. Pure fractions were combined and concentrated under reduced pressure to afford tert-butyl (S)-2-((tert-butoxycarbonyl)amino)-3-(4-(5-methyl-1,2,4-oxadiazol-3-yl)phenyl)propanoate. MS ESI calculated for C₂₁H₂₉N₃O₅ [M+H]⁺ 402.21, found 402.29.

Step 4: To a stirred solution of tert-butyl (S)-2-((tert-butoxycarbonyl)amino)-3-(4-(5-methyl-1,2,4-oxadiazol-3-yl)phenyl)propanoate (8.00 g, 19.8 mmol) in DCM (80 mL) was added TFA (80.0 mL, 1.04 mol) at 0° C. The reaction mixture was stirred at 24° C. for 8 h. The reaction mixture was concentrated under reduced pressure. The crude compound was triturated with diethyl ether and dried under reduced pressure to afford (S)-2-amino-3-(4-(5-methyl-1,2,4-oxadiazol-3-yl)phenyl)propanoic acid. MS ESI calculated for C₁₂H₁₃N₃O₃ [M+H]⁺ 248.10, found 248.07.

Step 5: To a stirred solution of (S)-2-amino-3-(4-(5-methyl-1,2,4-oxadiazol-3-yl)phenyl)propanoic acid (4.50 g, 18.2 mmol) in THF (50 mL) and water (50 mL) were added sodium bicarbonate (7.64 g, 91.0 mmol) and (9H-fluoren-9-yl)methyl (2,5-dioxopyrrolidin-1-yl) carbonate (5.53 g, 16.4 mmol) at room temperature. The reaction mixture was stirred at 25° C. for 2 h. The reaction mixture was acidified with 1 N HCl (pH ˜4) and extracted with ethyl acetate (2×300 mL). The combined organic layer was washed with brine (200 mL), dried over Na₂SO₄, filtered and concentrated under reduced pressure. The crude compound was purified by reverse phase flash chromatography (conditions: Column: C18 gel column (100 g) Mobile Phase A: Water; Mobile Phase B: Acetonitrile) and the compound eluted with a gradient of 45-100% acetonitrile in water. Pure fractions were combined and concentrated under reduced pressure to afford (S)-2-((((9H-fluoren-9-yl)methoxy)carbonyl)amino)-3-(4-(5-methyl-1,2,4-oxadiazol-3-yl)phenyl)propanoic acid. MS ESI calculated for C₂₇H₂₃N₃O₅ [M+H]⁺ 470.16, found 470.25. ¹H NMR (400 MHz, DMSO-d₆): δ (ppm) 12.85 (br s, 1H), 7.88 (t, J=8.0 Hz, 4H), 7.50-7.76 (m, 3H), 7.35-7.47 (m, 4H), 7.24-7.33 (m, 2H), 4.10-4.30 (m, 4H), 3.16 (dd, J=13.8 Hz, 4.2 Hz, 1H), 2.90-3.00 (m, 1H), 2.66 (s, 3H).

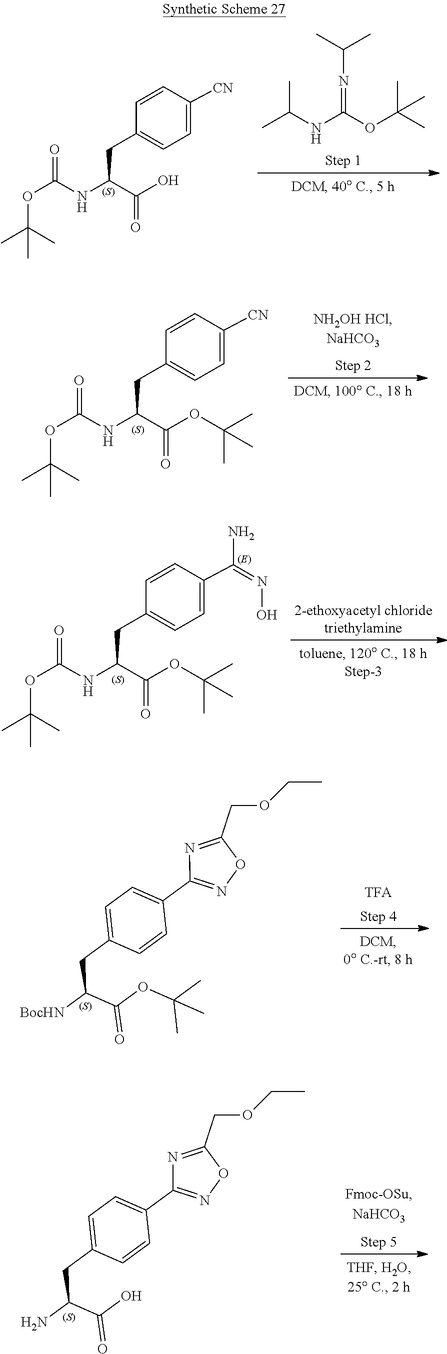

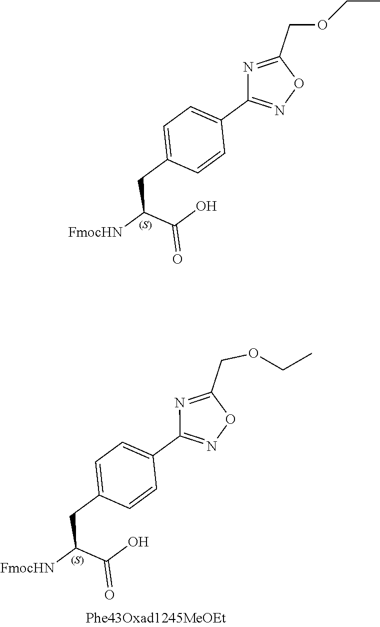

### (S)-2-((((9H-fluoren-9-yl)methoxy)carbonyl)amino)-3-(4-(5-methyl-1,2,4-oxadiazol-3-yl)phenyl)propanoic acid

Step 1: To a stirred solution of (S)-2-((tert-butoxycarbonyl)amino)-3-(4-cyanophenyl)propanoic acid (20.0 g, 68.9 mmol) in DCM (200 mL) was added tert-butyl (E)-N,N′-diisopropylcarbamimidate (69.0 g, 344 mmol) at room temperature. The reaction mixture was stirred at 40° C. for 5 h. The reaction mixture was concentrated under reduced pressure. The crude compound was purified by Biotage-isolera using a silica column and the compound eluted with 30% of ethyl acetate in petroleum ether. Pure fractions were combined and concentrated under reduced pressure to afford tert-butyl (S)-2-((tert-butoxycarbonyl)amino)-3-(4-cyanophenyl)propanoate. MS ESI calculated for C₁₉H₂₆N₂O₄ [M−H]⁺ 345.19, found 345.25.

Step 2: To a stirred solution of hydroxylammoniumchloride (21.0 g, 302 mmol) in DMSO (400 mL) was added sodium bicarbonate (34.6 g, 411 mmol) at room temperature. Then tert-butyl (S)-2-((tert-butoxycarbonyl)amino)-3-(4-cyanophenyl)propanoate (19.0 g, 54.8 mmol) was added to the reaction mixture at 45-50° C. The reaction mixture was stirred at 100° C. for 18 h. The reaction mixture was cooled to 15-20° C., quenched with water and stirred at 15-20° C. for 15-20 min. The compound was filtered, washed with water and diethyl ether and dried under vacuum to afford tert-butyl (S,E)-2-((tert-butoxycarbonyl)amino)-3-(4-(N-hydroxycarbamimidoyl)phenyl)propanoate (as a white solid. MS ESI calculated for C₁₉H₂₉N₃O₅ [M+H]⁺ 380.21, found 380.29.

Step 3: To a solution of tert-butyl (S,E)-2-((tert-butoxycarbonyl)amino)-3-(4-(N-hydroxycarbamimidoyl)phenyl)propanoate (4.0 g, 10.5 mmol) in toluene (100 mL) was added triethylamine (3.20 g, 4.41 mL, 31.6 mmol) followed by dropwise of 2-ethoxyacetyl chloride (1.55 g, 1.39 mL, 12.6 mmol). The mixture was stirred at 120° C. for 18 h. The reaction mixture was concentrated under reduced pressure. The crude compound was purified by Biotage-isolera using asilica column and the compound eluted with 20% of ethyl acetate in hexane. Pure fractions were combined and concentrated under reduced pressure to afford tert-butyl (S)-2-((tert-butoxycarbonyl)amino)-3-(4-(5-(ethoxymethyl)-1,2,4-oxadiazol-3-yl)phenyl)propanoate. LC-MS found for C₂₃H₃₃N₃O₆: m/z 448.3 [M+H]^{+ (}1.40 min).

Step 4: To a stirred solution of tert-butyl (S)-2-((tert-butoxycarbonyl)amino)-3-(4-(5-(ethoxymethyl)-1,2,4-oxadiazol-3-yl)phenyl)propanoate (3.10 g, 6.93 mmol) in DCM (50 mL) was added TFA (7.90 g, 5.34 mL, 69.3 mmol) at 0° C. The reaction mixture was stirred at 24° C. for 24 h. The reaction mixture was concentrated under reduced pressure to afford (S)-2-amino-3-(4-(5-(ethoxymethyl)-1,2,4-oxadiazol-3-yl)phenyl)propanoic acid. LC-MS found for Cl₄H₁₇N₃O₄: m/z 292.0 [M+H]^{+ (}0.34 min).

Step 5: To a stirred solution of (S)-2-amino-3-(4-(5-(ethoxymethyl)-1,2,4-oxadiazol-3-yl)phenyl)propanoic acid (2.50 g, 8.58 mmol) in THF (50 mL) and water (50 mL) were added sodium bicarbonate (3.60 g, 42.9 mmol) and (9H-fluoren-9-yl)methyl (2,5-dioxopyrrolidin-1-yl) carbonate (2.89 g, 8.58 mmol) at room temperature. The reaction mixture was stirred at 25° C. for 2 h. Reaction mixture was acidified with 1N HCl (pH ˜2) and extracted with ethyl acetate (2×200 mL). The combined organic layer was washed with brine (150 mL), dried over Na₂SO₄, filtered, and concentrated under reduced pressure. The crude compound was purified by Teledyne Isco (80 g silica gel, eluting with 0 to 100% ethyl acetate in hexanes). Pure fractions were combined and concentrated under reduced pressure to afford (S)-2-((((9H-fluoren-9-yl)methoxy)carbonyl)amino)-3-(4-(5-(ethoxymethyl)-1,2,4-oxadiazol-3-yl)phenyl)propanoic acid. LC-MS found for C₂₉H₂₇N₃O₆: m/z 514.3 [M+H]^{+ (}1.31 min).

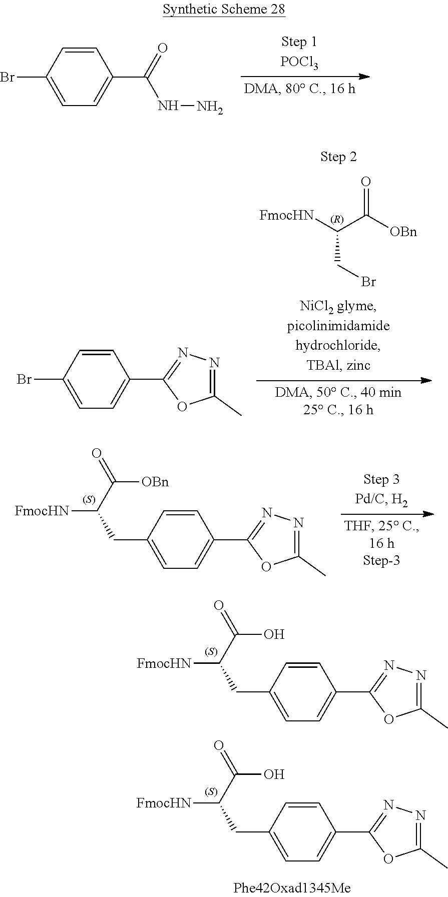

### (S)-2-((((9H-fluoren-9-yl)methoxy)carbonyl)amino)-3-(4-(5-methyl-1,3,4-oxadiazol-2-yl)phenyl)propanoic acid

Step 1: To a stirred solution of 4-bromobenzohydrazide (30.0 g, 140 mmol) in DMA (150 mL) was added POCl₃ (14.3 mL, 153 mmol) dropwise at 80° C. The reaction mixture was stirred at 80° C. for 16 h. The reaction mixture was diluted with ice water (1000 mL), quenched with Na₂CO₃ solution and extracted with DCM (1000 mL). The combined organic layer was washed with brine (500 mL), dried over anhydrous sodium sulphate, filtered and concentrated under reduced pressure. The crude compound was purified by Biotage using a silica gel column and the compound was eluted with 30% ethyl acetate in pet ether. Pure fractions were combined and concentrated under reduced pressure to afford 2-(4-bromophenyl)-5-methyl-1,3,4-oxadiazole. MS ESI calculated for C₉H₇BrN₂O [M+H]⁺ 238.97, found 238.95.

Step 2: A stirred solution of nickel chloride, dimethoxyethane adduct (457 mg, 2.08 mmol) and picolinimidamide hydrochloride (656 mg, 4.16 mmol) in DMA (40 mL) under argon was stirred at 50° C. for 40 min. Then TBAI (3.84 g, 10.4 mmol), (R)-2-((((9H-fluoren-9-yl)methoxy)carbonyl)amino)-3-bromopropanoate (5.00 g, 10.4 mmol), 2-(4-bromophenyl)-5-methyl-1,3,4-oxadiazole (2.49 g, 10.4 mmol) and zinc (1.36 g, 20.8 mmol) were added to the reaction mixture at room temperature. The reaction mixture was stirred at 25° C. for 16 h. The reaction mixture was quenched with ice cold water (150 mL) and extracted with EtOAc (3×70 mL). The combined organic layer was washed with ice cold water (100 mL), brine (100 mL), dried over anhydrous sodium sulphate, filtered and concentrated under reduced pressure. The crude compound was purified by Biotage using a silica gel column and the compound was eluted with 30% ethyl acetate in petroleum ether. Pure fractions were combined and concentrated under reduced pressure to afford benzyl (S)-2-((((9H-fluoren-9-yl)methoxy)carbonyl)amino)-3-(4-(5-methyl-1,3,4-oxadiazol-2-yl)phenyl)propanoate. MS ESI calculated for C₃₄H₂₉N₃O₅ [M+H]⁺ 560.21, found 560.33.

Step 3: To a stirred solution of benzyl (S)-2-((((9H-fluoren-9-yl)methoxy)carbonyl)amino)-3-(4-(5-methyl-1,3,4-oxadiazol-2-yl)phenyl)propanoate (5.00 g, 8.93 mmol) in THF (75 mL) was added Pd/C (2.35 g, 625 mol) at room temperature. The reaction mixture was stirred under hydrogen atmosphere at 25° C. for 16 h. The reaction mixture was filtered through a CELITE bed. The filtrate was concentrated under reduced pressure. The crude compound was stirred in DCM (10 mL) and diethyl ether was added. The precipitated solid was filtered and dried under reduced pressure to afford (S)-2-((((9H-fluoren-9-yl)methoxy)carbonyl)amino)-3-(4-(5-methyl-1,3,4-oxadiazol-2-yl)phenyl)propanoic acid. MS ESI calculated for C₂₇H₂₃N₃O₅ [M+H]⁺ 470.16, found 470.22. ¹H NMR (400 MHz, DMSO-d₆): δ (ppm) 12.82 (br s, 1H), 7.83-7.94 (m, 4H), 7.77 (d, J=8.4 Hz, 1H), 7.62 (d, J=6.4 Hz, 2H), 7.47 (d, J=8.0 Hz, 2H), 7.39 (td, J=7.4 Hz, 2.8 Hz, 2H), 7.23-7.35 (m, 2H), 4.07-4.36 (m, 4H), 3.18 (dd, J=14.0 Hz, 4.4 Hz, 1H), 2.83-3.02 (m, 1H), 2.57 (s, 3H).

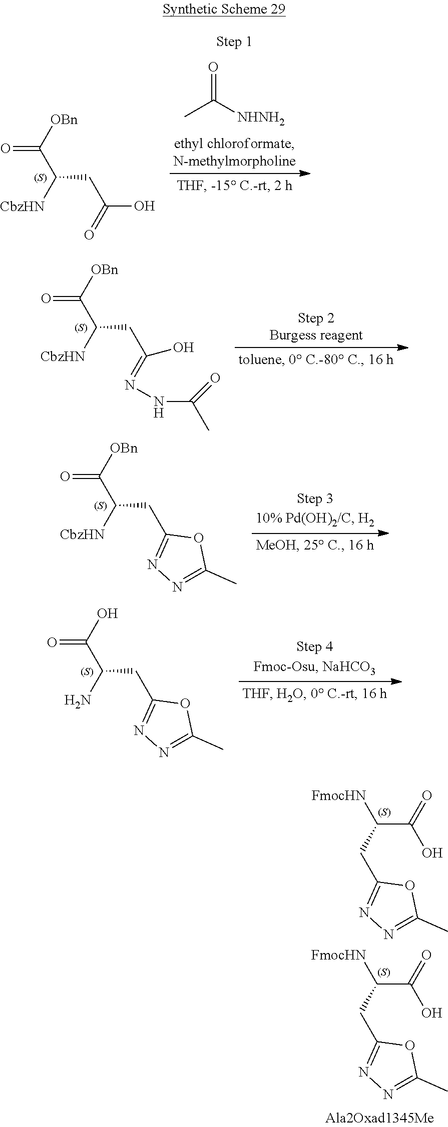

### (S)-2-((((9H-fluoren-9-yl)methoxy)carbonyl)amino)-3-(5-methyl-1,3,4-oxadiazol-2-yl)propanoic acid

Step 1: To a stirred solution of (S)-4-(benzyloxy)-3-(((benzyloxy)carbonyl)amino)-4-oxobutanoic acid (35.0 g, 97.9 mmol) in THF (1 L) were added N-methylmorpholine (10.8 mL, 97.9 mmol) and ethyl chloroformate (9.41 mL, 97.9 mmol) at −15° C. The reaction mixture was stirred at −15° C. for 15 min. Then a solution of acetohydrazide (7.26 g, 97.9 mmol) in THF (200 mL) was added dropwise to the reaction mixture at −15° C. The reaction mixture was stirred at 25° C. for 2 h. Reaction mixture was quenched with ice water (500 mL) and extracted with EtOAc (3×150 mL). The combined organic layer was washed with brine (100 mL), dried over sodium sulfate, filtered and concentrated under reduced pressure to afford (S,Z)—N-acetyl-4-(benzyloxy)-3-(((benzyloxy)carbonyl)amino)-4-oxobutanehydrazonic acid. MS ESI calculated for C₂₁H₂₃N₃O₆ [M+H]⁺ 414.16, found 414.14.

Step 2: To a stirred solution of (S,Z)—N-acetyl-4-(benzyloxy)-3-(((benzyloxy)carbonyl)amino)-4-oxobutanehydrazonic acid (10.00 g, 21.77 mmol) in toluene (50 mL) was added Burgess Reagent (7.781 g, 32.65 mmol) at 0° C. The reaction mixture was stirred at 80° C. for 16 h. The reaction mixture was quenched with cold water (100 mL) and extracted with ethyl acetate (2×100 mL). The combined organic layer was dried over anhydrous sodium sulphate, filtered and concentrated under reduced pressure. The crude compound was purified by Biotage using a 120 g silica cartridge and the compound was eluted with 100% EtOAc. Pure fractions were combined and concentrated under reduced pressure to afford benzyl (S)-2-(((benzyloxy)carbonyl)amino)-3-(5-methyl-1,3,4-oxadiazol-2-yl)propanoate. MS ESI calculated for C₂₁H₂₁N₃O₅ [M+H]⁺ 396.15, found 396.26.

Step 3: To a stirred solution of benzyl (S)-2-(((benzyloxy)carbonyl)amino)-3-(5-methyl-1,3,4-oxadiazol-2-yl)propanoate (11.0 g, 25.0 mmol) in MeOH (250 mL) was added 10% Pearlman's catalyst (5.00 g, 4.70 mmol) at room temperature. The mixture was stirred at room temperature for 10 min. The reaction mixture was purged with N₂ gas at room temperature for 5 min. The reaction mixture was stirred under H₂ balloon at 25° C. for 16 h. The reaction mixture was filtered through a CELITE pad and washed with MeOH (50 mL) and DCM (100 mL). The organic layer was concentrated under reduced pressure. The crude compound was washed with n-pentane (3×50 mL) and dried under reduced pressure to afford (S)-2-amino-3-(5-methyl-1,3,4-oxadiazol-2-yl)propanoic acid. ¹H NMR (400 MHz, DMSO-d₆): δ (ppm) 7.50-8.89 (m, 2H), 3.64 (d, J=6.8 Hz, 1H), 3.56 (dd, J=9.0 Hz, 4.6 Hz, 1H), 3.37-3.42 (m, 1H), 2.45 (s, 3H).

Step 4: To a stirred solution of (S)-2-amino-3-(5-methyl-1,3,4-oxadiazol-2-yl)propanoic acid (4.20 g, 22.1 mmol) in THF (35 mL) and H₂O (35 mL) were added sodium bicarbonate (2.84 g, 33.1 mmol) and (9H-fluoren-9-yl)methyl (2,5-dioxopyrrolidin-1-yl) carbonate (11.4 g, 33.1 mmol) at 0° C. The reaction mixture was stirred at 25° C. for 16 h and then diluted with EtOAc (100 mL) and acidified with 1M HCl (pH ˜2-3). The aqueous layer was extracted with EtOAc (2×100 mL). The combined organic layer was washed with saturated brine (50 mL), dried over anhydrous sodium sulfate, filtered and concentrated under reduced pressure. The crude compound was purified by Biotage using a 80 g silica cartridge and the compound was eluted with 100% EtOAc. Pure fractions were combined, concentrated under reduced pressure and the isolated compound was washed with n-pentane (2×100 mL) and again triturated with ether and pentane (90:10). A few drops of EtOAc (three times) were added to the reaction mixture and a solid was filtered. The solid was washed with ether and dried under reduced pressure to afford (S)-2-((((9H-fluoren-9-yl)methoxy)carbonyl)amino)-3-(5-methyl-1,3,4-oxadiazol-2-yl)propanoic acid. MS ESI calculated for C₂₁H₁₉N₃O₅ [M+H]⁺ 394.13, found 394.22. ¹H NMR (400 MHz, DMSO-d₆): δ (ppm) 13.10 (br s, 1H), 7.89 (d, J=7.6 Hz, 2H), 7.58-7.82 (m, 3H), 7.42 (t, J=7.4 Hz, 2H), 7.32 (td, J=7.4 Hz, 0.9 Hz, 2H), 4.33-4.45 (m, 1H), 4.17-4.32 (m, 3H), 3.27-3.30 (m, 1H), 3.13-3.22 (m, 1H), 2.43 (s, 3H).

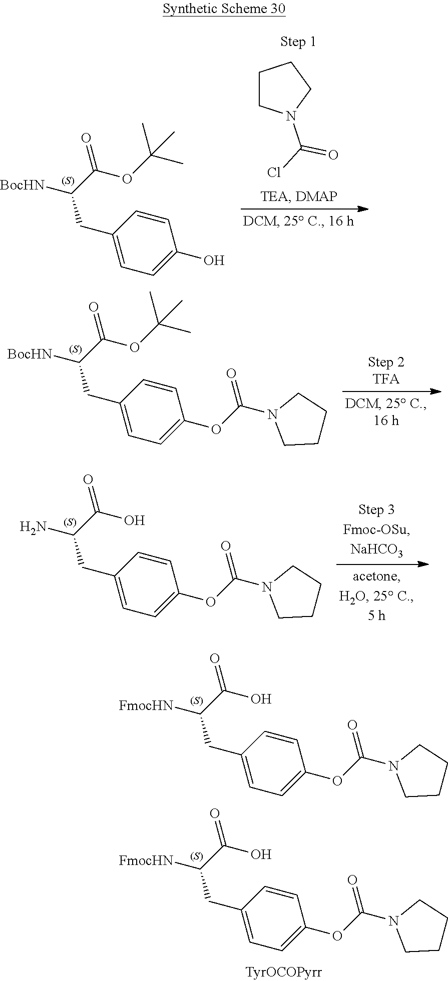

### (S)-2-((((9H-fluoren-9-yl)methoxy)carbonyl)amino)-3-(4-((pyrrolidine-1-carbonyl)oxy)phenyl)propanoic acid

Step 1: To a stirred solution of tert-butyl (tert-butoxycarbonyl)-L-tyrosinate (20.00 g, 59.27 mmol) in DCM (100 mL) were added DMAP (7.241 g, 59.27 mmol), TEA (16.5 mL, 118.5 mmol) and pyrrolidine-1-carbonyl chloride (8.710 g, 65.20 mmol) at room temperature. The reaction mixture was stirred at 25° C. for 16 h. The reaction mixture was concentrated under reduced pressure. The crude compound was diluted with MeOH (100 mL) and stirred at room temperature for 30 min. The precipitated solid was filtered and washed with MeOH (2×20 mL) and dried under vacuum to afford (S)-4-(3-(tert-butoxy)-2-((tert-butoxycarbonyl)amino)-3-oxopropyl)phenyl pyrrolidine-1-carboxylate. MS ESI calculated for C₂₃H₃₄N₂O₆ [M+H]⁺ 435.24, found 435.31.

Step 2: To a stirred solution of (S)-4-(3-(tert-butoxy)-2-((tert-butoxycarbonyl)amino)-3-oxopropyl)phenyl pyrrolidine-1-carboxylate (12.00 g, 27.62 mmol) in DCM (60 mL) was added TFA (63.83 mL, 828.5 mmol) at room temperature. The reaction mixture was stirred at 25° C. for 16 h. The reaction mixture was concentrated under reduced pressure, co-distilled with toluene (50 mL) and dried under vacuum to afford (S)-2-amino-3-(4-((pyrrolidine-1-carbonyl)oxy)phenyl)propanoic acid. MS ESI calculated for C₁₄H₁₈N₂O₄ [M+H]⁺ 279.13, found 279.20.

Step 3: To a stirred solution of (S)-2-amino-3-(4-((pyrrolidine-1-carbonyl)oxy)phenyl)propanoic acid (8.00 g, 28.7 mmol) in acetone (50 mL) and water (50 mL) were added sodium bicarbonate (7.24 g, 86.2 mmol) and (9H-fluoren-9-yl)methyl (2,5-dioxopyrrolidin-1-yl) carbonate (10.7 g, 31.6 mmol) at room temperature. The reaction mixture was stirred at 25° C. for 5 h. The reaction mixture was concentrated under reduced pressure. The aqueous layer was washed with diethyl ether (2×100 mL), acidified with 2N HCl (pH-4) and extracted with 10% MeOH in DCM (2×200 mL). The combined organic layer was dried over anhydrous Na₂SO₄, filtered and concentrated under reduced pressure. The crude compound was diluted with 10% diethyl ether in hexane (100 mL) and stirred at 25° C. for 30 min. The mixture was filtered and dried under reduced pressure to afford (S)-2-((((9H-fluoren-9-yl)methoxy)carbonyl)amino)-3-(4-((pyrrolidine-1-carbonyl)oxy)phenyl)propanoic acid. MS ESI calculated for C₂₉H₂₈N₂O₆ [M+H]⁺ 501.19, found 501.29. ¹H NMR (400 MHz, DMSO-d₆): δ (ppm) 7.88 (d, J=7.6 Hz, 2H), 7.56-7.72 (m, 2H), 7.41 (t, J=7.6 Hz, 2H), 7.27-7.36 (m, 2H), 7.15 (d, J=8.0 Hz, 2H), 6.94 (d, J=8.4 Hz, 3H), 3.71-4.39 (m, 4H), 3.44-3.50 (m, 2H), 3.35-3.37 (m, 3H), 3.05 (dd, J=13.4 Hz, 4.6 Hz, 1H), 2.84-2.97 (m, 1H), 1.79-1.96 (m, 4H).

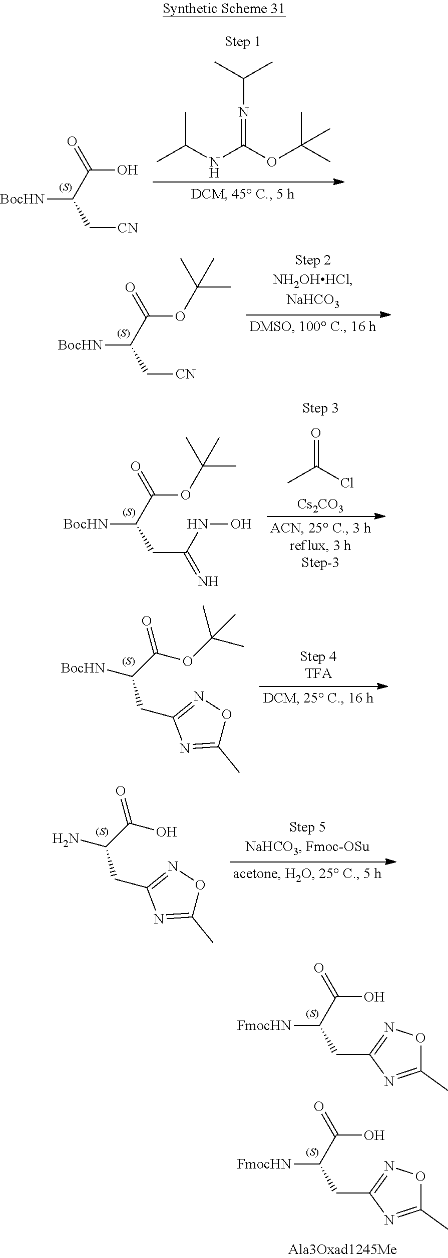

### (S)-2-((((9H-fluoren-9-yl)methoxy)carbonyl)amino)-3-(5-methyl-1,2,4-oxadiazol-3-yl)propanoic acid

Step 1: To a stirred solution of (S)-2-((tert-butoxycarbonyl)amino)-3-cyanopropanoic acid (24.00 g, 112.0 mmol) in DCM (100 mL) was added tert-butyl (E)-N,N′-diisopropylcarbamimidate (80 mL, 336.1 mmol) at room temperature. A curdy white precipitate was formed during the reaction. The reaction mixture was stirred at 45° C. for 5 h. The reaction mixture was filtered and washed with DCM (2×50 mL). The filtrate was concentrated under reduced pressure. The crude compound was purified by Biotage-isolera using a 120 g silica cartridge and the compound eluted with 30% of ethyl acetate in petroleum ether. Pure fractions were combined and concentrated under reduced pressure to afford tert-butyl (S)-2-((tert-butoxycarbonyl)amino)-3-cyanopropanoate. MS ESI calculated for C₁₃H₂₂N₂O₄ [M+H]⁺ 271.16, found 271.20.

Step 2: To a stirred solution of tert-butyl (S)-2-((tert-butoxycarbonyl)amino)-3-cyanopropanoate (20.0 g, 74.0 mmol) in DMSO (150 mL) were added sodium bicarbonate (46.6 g, 555 mmol) and hydroxylammoniumchloride (28.3 g, 407 mmol) at room temperature. The reaction mixture was stirred at 100° C. for 16 h. The reaction mixture was cooled to room temperature, quenched with water (200 mL) and extracted with EtOAc (2×300 mL). The combined organic layer was washed with water (2×100 mL), brine (100 mL), dried over Na₂SO₄, filtered and concentrated under reduced pressure to afford tert-butyl (S)-2-((tert-butoxycarbonyl)amino)-4-(hydroxyamino)-4-iminobutanoate. MS ESI calculated for C₁₃H₂₅N₃O₅ [M+H]⁺ 304.18, found 304.22.

Step 3: To a stirred solution of tert-butyl (S)-2-((tert-butoxycarbonyl)amino)-4-(hydroxyamino)-4-iminobutanoate (9.50 g, 31.3 mmol) in ACN (100 mL) were added cesium carbonate (20.4 g, 62.6 mmol) and acetyl chloride (2.45 mL, 34.4 mmol) at room temperature. The reaction mixture was stirred at 25° C. for 3 h. The reaction was monitored by TLC which showed new spot and also by LCMS which showed 93% of intermediate mass. The reaction mixture was stirred at reflux for 3 h. The reaction mixture was filtered through a CELITE pad and washed with ACN (2×50 mL). The filtrate was concentrated under reduced pressure. The crude compound was purified by Biotage-isolera using a silica column and the compound eluted with 30% of ethyl acetate in petroleum ether. Pure fractions were combined and concentrated under reduced pressure to afford tert-butyl (S)-2-((tert-butoxycarbonyl)amino)-3-(5-methyl-1,2,4-oxadiazol-3-yl)propanoate. MS ESI calculated for C₁₅H₂₅N₃O₅ [M+H]⁺ 328.18, found 328.25.

Step 4: To a stirred solution of tert-butyl (S)-2-((tert-butoxycarbonyl)amino)-3-(5-methyl-1,2,4-oxadiazol-3-yl)propanoate (5.80 g, 17.7 mmol) in DCM (20 mL) was added TFA (20.5 mL, 266 mmol) at room temperature. The reaction mixture was stirred at 25° C. for 16 h. The reaction mixture was concentrated under reduced pressure, co-distilled with toluene (10 mL) and dried under reduced pressure to afford (S)-2-amino-3-(5-methyl-1,2,4-oxadiazol-3-yl)propanoic acid. MS ESI calculated for C₆H₉N₃O₃[M+H]⁺ 172.06, found 171.99.

Step 5: To a stirred solution of (S)-2-amino-3-(5-methyl-1,2,4-oxadiazol-3-yl)propanoic acid (4.20 g, 24.5 mmol) in acetone (20 mL) and H₂O (20 mL) were added sodium bicarbonate (4.12 g, 49.1 mmol) and (9H-fluoren-9-yl)methyl (2,5-dioxopyrrolidin-1-yl) carbonate (9.11 g, 27.0 mmol) at room temperature. The reaction mixture was stirred at 25° C. for 5 h. The reaction mixture was concentrated under reduced pressure, given acid-base treatment and extracted with 10% MeOH in DCM (2×200 mL). The combined organic layer was dried over Na₂SO₄, filtered and concentrated under reduced pressure. The crude compound was purified twice by Biotage-isolera using a 120 g silica cartridge and the compound eluted with 10% MeOH in DCM. Pure fractions were combined and concentrated under reduced pressure. The isolated compound was washed with 10% diethyl ether in hexane and dried under reduced pressure to afford (S)-2-((((9H-fluoren-9-yl)methoxy)carbonyl)amino)-3-(5-methyl-1,2,4-oxadiazol-3-yl)propanoic acid. MS ESI calculated for C₂₁H₁₉N₃O₅ [M+H]⁺ 394.13, found 394.19. ¹H NMR (400 MHz, DMSO-d₆): δ (ppm) 12.95 (br s, 1H), 7.89 (d, J=7.6 Hz, 2H), 7.76 (d, J=8.0 Hz, 1H), 7.68 (d, J=7.6 Hz, 2H), 7.42 (t, J=7.4 Hz, 2H), 7.31 (t, J=7.4 Hz, 2H), 4.33-4.64 (m, 1H), 4.10-4.33 (m, 3H), 2.97-3.21 (m, 2H), 2.55 (s, 3H).

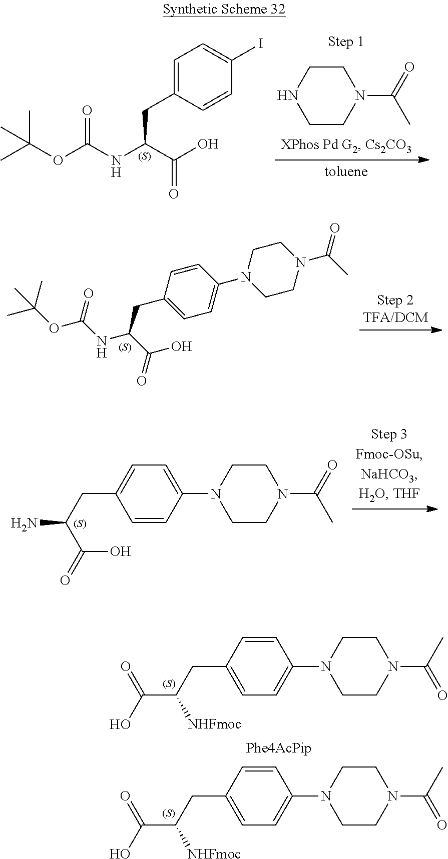

### (S)-2-((((9H-fluoren-9-yl)methoxy)carbonyl)amino)-3-(4-(4-acetylpiperazin-1-yl)phenyl)propanoic acid

Step 1: To a stirred solution of (S)-3-(4-bromophenyl)-2-((tert-butoxycarbonyl)amino)propanoic acid (6 g, 17.43 mmol) in toluene (180 mL) was added XPhos Pd G₂ (2.057 g, 2.61 mmol) at 25° C. under nitrogen. The resulting solution was stirred at 100° C. for 10 min. 1-(Piperazin-1-yl)ethan-1-one (2.234 g, 17.43 mmol) and Cs₂CO₃ (5.04 g, 26.1 mmol) were added and the resulting solution was stirred at 110° C. for 2 h. The reaction was cooled to rt and quenched with H₂O (500 mL) and extracted with EtOAc (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-60% EtOAc in PE to give (S)-3-(4-(4-acetylpiperazin-1-yl)phenyl)-2-((tert-butoxycarbonyl)amino)propanoic acid. MS ESI calculated for C₂₀H₃₀N₃O₅ [M+H]⁺ 392.21, found 392.25. ¹H NMR (400 MHz, Methanol-d₄) δ 7.12 (d, J=8 Hz, 2H), 6.89 (d, J=8 Hz, 2H), 4.16-4.13 (m, 1H), 3.72-3.65 (m, 4H), 3.14-3.04 (m, 4H), 2.93-2.81 (m, 2H), 2.13 (s, 3H), 1.38-1.29 (m, 9H).

Step 2: To a stirred solution of (S)-3-(4-(4-acetylpiperazin-1-yl)phenyl)-2-((tert-butoxycarbonyl)amino)propanoic acid (10 g, 25.5 mmol) in DCM (30 mL) was added TFA (30 mL) at rt. The solution was stirred at 25° C. for 1 h then concentrated under reduced pressure. The crude (S)-3-(4-(4-acetylpiperazin-1-yl)phenyl)-2-aminopropanoic acid was used to the next step directly without any further purification. MS ESI calculated for C₁₅H₂₂N₃O₃[M+H]⁺ 292.16, found 292.20.

Step 3: To a stirred solution of (S)-3-(4-(4-acetylpiperazin-1-yl)phenyl)-2-(carboxyamino)propanoic acid (7 g, 20.87 mmol) in THF (25 mL) and water (25 mL) was added Fmoc-OSu (6.34 g, 18.79 mmol) at 25° C. under nitrogen. Then, NaHCO₃ (8.77 g, 104 mmol) was added. The resulting mixture was stirred at 25° C. for 2 h. The pH was adjusted to 5 with 1 N HCl and the mixture was extracted with EtOAc (2×200 mL). The combined organic layer was washed with brine (3×100 mL), dried over anhydrous Na₂SO₄ and filtered. The filtrate was concentrated under reduced pressure and the residue was purified by reverse phase flash chromatography with the following conditions: Column: C18 silica gel (330 g); Mobile Phase A: water (0.1% TFA), Mobile Phase B: MeCN; (Gradient: 5% B hold 5 min, up to 55% B within 15 min, 55% B hold 5 min; up to 95% B within 20 min, 95% B hold 5 min); Flow rate: 90 mL/min; Detector: UV 210 nm; RT=45 min. The product-containing fractions were collected and concentrated in vacuo to give (S)-2-((((9H-fluoren-9-yl)methoxy)carbonyl)amino)-3-(4-(4-acetylpiperazin-1-yl)phenyl)propanoic acid. MS ESI calculated for C₃₀H₃₂N₃O₅ [M+H]⁺ 514.23, found 514.30. ¹H NMR (400 MHz, Methanol-d₄) δ 7.78-7.76 (m, 2H), 7.61-7.52 (m, 2H), 7.40-7.37 (m, 2H), 7.32-7.22 (m, 4H), 7.08-6.98 (m, 2H), 4.47-4.43 (m, 1H), 4.33-4.31 (m, 1H), 4.14-4.02 (m, 2H), 3.68-3.63 (m, 4H), 3.23-3.08 (m, 5H), 2.91-2.85 (m, 1H), 2.11 (s, 3H).

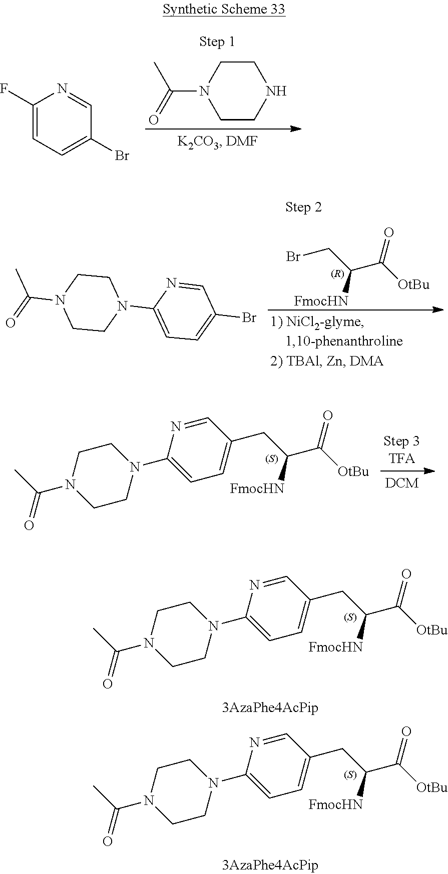

### (S)-2-((((9H-fluoren-9-yl)methoxy)carbonyl)amino)-3-(6-(4-acetylpiperazin-1-yl)pyridin-3-yl)propanoic acid

Step 1: To a mixture of 1-(piperazin-1-yl)ethan-1-one (21.85 g, 170 mmol) in DMF (150 mL) was added 5-bromo-2-fluoropyridine (15 g, 85 mmol) under argon at rt. The reaction was stirred at 100° C. for 2 h, then diluted with 300 mL EtOAc and washed with H₂O (3×80 mL), aqueous saturated NaCl (80 mL), dried over Na₂SO₄ and filtered. The filtrate was concentrated under reduced pressure. The residue was purified by silica gel column chromatography, eluting with a gradient of 0%-100% EtOAc in PE. The fractions containing desired product were combined and concentrated under reduced pressure to afford 1-(4-(5-bromopyridin-2-yl)piperazin-1-yl)ethan-1-one. MS ESI calculated for C₁₁H₁₅BrN₃O [M+H]⁺ 284.03 and 286.03, found 283.90 and 285.90.

Step 2: A mixture of nickel (II) chloride ethylene glycol dimethyl ether complex (0.696 g, 3.17 mmol) and 1,10-phenanthroline (0.571 g, 3.17 mmol) in DMA (2 mL) was heated at 50° C. for 0.5 hours. The mixture of 1-(4-(5-bromopyridin-2-yl)piperazin-1-yl)ethan-1-one (4.5 g, 15.84 mmol), tert-butyl (R)-2-((((9H-fluoren-9-yl)methoxy)carbonyl)amino)-3-bromopropanoate (7.78 g, 17.42 mmol) and tetrabutylammonium iodide (5.85 g, 15.84 mmol) in DMA (2 mL) were added at 25° C. Then zinc powder (2.071 g, 31.7 mmol) was added. The resulting mixture was stirred for 1 h at 50° C. The resulting mixture was poured into water (300 mL) and extracted with EtOAc (3×300 mL). The organic layer was washed with water (100 mL) and brine (2×80 mL), dried over anhydrous sodium sulfate and concentrated under reduced pressure. The residue was purified by column chromatography on silica gel eluting with DCM-MeOH (10:1) to afford tert-butyl (S)-2-((((9H-fluoren-9-yl)methoxy)carbonyl)amino)-3-(6-(4-acetylpiperazin-1-yl)pyridin-3-yl)propanoate. MS ESI calculated for C₃₃H₃₉N₄O₅ [M+H]⁺ 571.28, found 571.40.

Step 3: To a mixture of tert-butyl (S)-2-((((9H-fluoren-9-yl)methoxy)carbonyl)amino)-3-(6-(4-acetylpiperazin-1-yl)pyridin-3-yl)propanoate (7.3 g, 12.79 mmol) in DCM (70 mL) was added TFA (70 mL, 909 mmol) under argon at rt. The reaction was stirred at rt for 1 h then concentrated under reduced pressure. The residue was dissolved in THF (20 mL) and the resultant mixture was purified by reverse phase Combi-Flash with the following conditions: Column C18 silica gel column (330 g), 20-35 m; Mobile Phase A: 5 mM aq. TFA; Mobile Phase B: MeCN; (Gradient: 0% B hold 10 min, up to 42.3% B within 35 min, 42.3% B hold 3.2 min; up to 95% B within 2 min, 95% B hold 10 min); Flow rate: 60 mL/min; Detector: UV 254 & 210 nm; RT: 35.32 min. The product-containing fractions were collected and concentrated under vacuum to afford (S)-2-((((9H-fluoren-9-yl)methoxy)carbonyl)amino)-3-(6-(4-acetylpiperazin-1-yl)pyridin-3-yl)propanoic acid. MS ESI calculated for C₂₉H₃₁N₄O₅ [M+H]⁺ 515.22, found 515.15. ¹H NMR (300 MHz, Methanol-d₄) δ 7.87-7.79 (m, 4H), 7.62-7.55 (m, 2H), 7.42-7.27 (m, 4H), 7.11-7.09 (m, 1H), 4.51-4.46 (m, 1H), 4.32-4.09 (m, 3H), 3.65-3.54 (m, 8H), 3.29-3.20 (m, 1H), 2.94-2.89 (m, 1H), 2.12 (s, 3H).

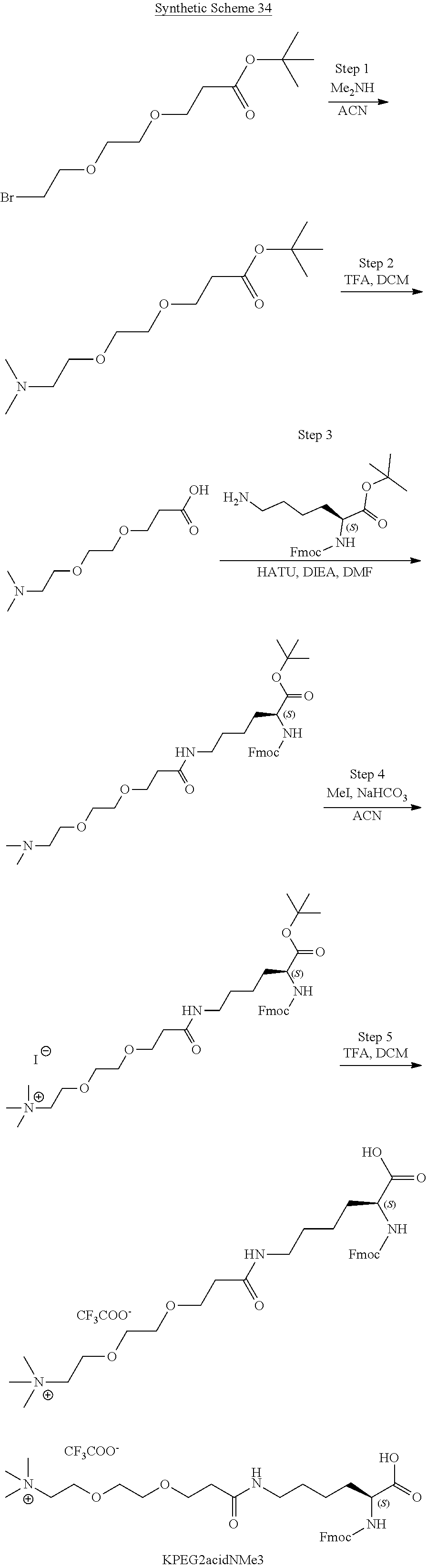

### (S)-5-carboxy-1-(9H-fluoren-9-yl)-N,N,N-trimethyl-3,11-dioxo-2,14,17-trioxa-4,10-diazanonadecan-19-aminium 2,2,2-trifluoroacetate

Step 1: To a solution of tert-butyl 3-(2-(2-bromoethoxy)ethoxy)propanoate (10 g, 33.6 mmol) in ACN (100 mL) was added Me₂NH (5.49 g, 67.3 mmol) at room temperature. The reaction mixture was stirred at 90° C. for 16 h. The resulting solution was diluted with water (200 mL) and extracted with EA (3×300 mL) and the combined organic layer was washed with brine (3×300 mL), dried over anhydrous Na₂SO₄ and filtered. The residue was purified by silica gel column chromatography, eluted with gradient 0-10% MeOH in DCM to afford tert-butyl 3-(2-(2-(dimethylamino)ethoxy)ethoxy)propanoate. MS ESI calculated for C₁₃H₂₈NO₄ [M+H]⁺ 262.20, found 262.15.

Step 2: A solution of tert-butyl 3-(2-(2-(dimethylamino)ethoxy)ethoxy)propanoate (8 g, 30.6 mmol) in DCM (100 mL) and TFA (100 mL) at 0° C. was prepared. The reaction mixture was stirred at room temperature for 2 h. The resulting solution was concentrated under reduced pressure to afford 3-(2-(2-(dimethylamino)ethoxy)ethoxy)propanoic acid. MS ESI calculated for C₉H₂₀NO₄ [M+H]⁺ 206.13, found 206.10.

Step 3: To a stirred solution of 3-(2-(2-(dimethylamino)ethoxy)ethoxy)propanoic acid (6.5 g, 31.7 mmol) in DMF (100 mL) were added HATU (24.08 g, 63.3 mmol), DIEA (6.08 ml, 34.8 mmol) and tert-butyl (((9H-fluoren-9-yl)methoxy)carbonyl)-L-lysinate (13.44 g, 31.7 mmol) at −40° C. After the resulting mixture was stirred at −40° C. for 4 h, it was quenched with water (100 mL) and extracted with EA (3×500 mL). The combined organic layer was washed with brine (3×100 mL), dried over anhydrous Na₂SO₄ and filtered. The filtrate was concentrated under reduced pressure and the residue was purified by silica gel column chromatography, eluted with gradient 0-50% EA in PE to afford tert-butyl (S)-17-((((9H-fluoren-9-yl)methoxy)carbonyl)amino)-2-methyl-11-oxo-5,8-dioxa-2,12-diazaoctadecan-18-oate. MS ESI calculated for C₃₄H₅₀N₃O₇[M+H]⁺ 612.36, found 612.45. ¹H NMR (300 MHz, Chloroform-d) δ 7.79-7.76 (m, 2H), 7.63-7.61 (m, 2H), 7.44-7.41 (m, 2H), 7.35-7.30 (m, 2H), 6.92 (s, 1H), 5.56-5.53 (m, 1H), 4.26-4.21 (m, 2H), 3.87-3.85 (m, 2H), 3.73-3.71 (m, 2H), 3.62-3.53 (m, 5H), 3.30-3.20 (m, 4H), 2.88 (s, 6H), 2.50-2.48 (m, 2H), 1.84-1.82 (m, 1H), 1.70-1.68 (m, 1H), 1.57-1.40 (m, 13H).

Step 4: To a solution of tert-butyl (S)-17-((((9H-fluoren-9-yl)methoxy)carbonyl)amino)-2-methyl-11-oxo-5,8-dioxa-2,12-diazaoctadecan-18-oate (1 g, 1.635 mmol) in ACN (50 mL) were added NaHCO₃ (0.137 g, 1.635 mmol) and Mel (0.102 ml, 1.635 mmol) at room temperature. The reaction mixture was stirred at room temperature for 5 h. The resulting solution was diluted with water (100 mL) and extracted with EtOAc (3×300 mL) and the combined organic layer was washed with brine (3×100 mL), dried over anhydrous Na₂SO₄ and filtered. The residue was concentrated under reduced pressure to afford (S)-5-carboxy-1-(9H-fluoren-9-yl)-N,N,N-trimethyl-3,11-dioxo-2,14,17-trioxa-4,10-diazanonadecan-19-aminium iodide. MS ESI calculated for C₃₅H₅₂N₃O₇ [M-tBu]⁺ 570.32, found 570.30.

Step 5: To a stirred solution of (S)-5-carboxy-1-(9H-fluoren-9-yl)-N,N,N-trimethyl-3,11-dioxo-2,14,17-trioxa-4,10-diazanonadecan-19-aminium iodide (7 g, 10.03 mmol) in DCM (100 mL) was added TFA (100 mL) at room temperature. The reaction mixture was stirred at room temperature for 1 h. The filtrate was concentrated under reduced pressure and the residue was purified by RP Flash as following conditions: (Column: Flash C¹⁸ 330 g; Mobile Phase A: water (0.1% TFA); Mobile Phase B: ACN; Flow rate: 100 mL/min; 2% B in 10 min; 2% to 20% B in 5 min; 20% to 50% B in 20 min; Detector: UV 210 nm. RT: 35 min) to afford (S)-5-carboxy-1-(9H-fluoren-9-yl)-N,N,N-trimethyl-3,11-dioxo-2,14,17-trioxa-4,10-diazanonadecan-19-aminium 2,2,2-trifluoroacetate. MS ESI calculated for C₃₁H₄₄N₃O₇⁺ [M]⁺ 570.32, found 570.30. ¹H NMR (300 MHz, CD₃OD) δ 7.83-7.80 (m, 2H), 7.71-7.67 (m, 2H), 7.43-7.30 (m, 4H), 4.44-4.32 (m, 2H), 4.27-4.12 (m, 2H), 3.92-3.89 (m, 2H), 3.74-3.70 (m, 2H), 3.62-3.53 (m, 6H), 3.26-3.17 (m, 11H), 2.44-2.40 (m, 2H), 1.89-1.70 (m, 2H), 1.59-1.39 (m, 4H).

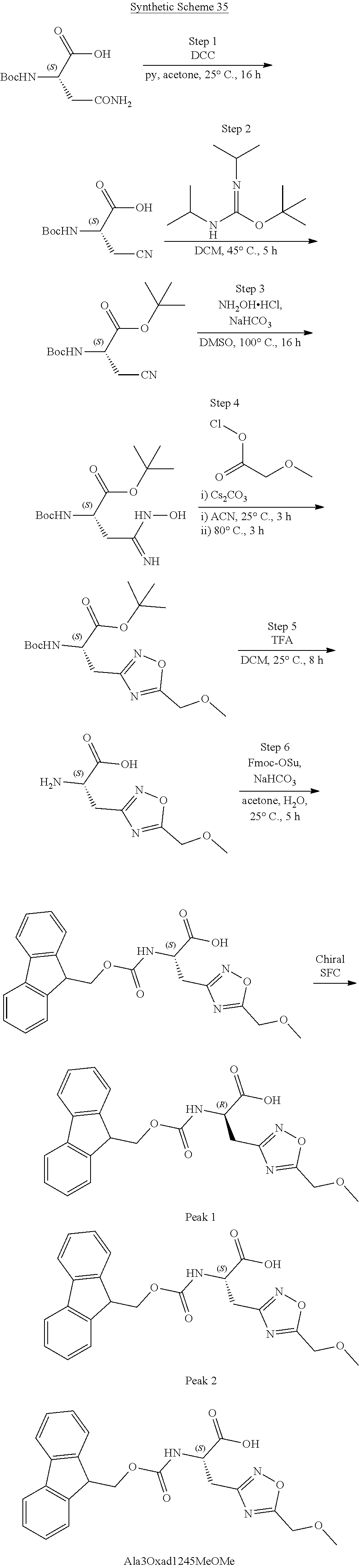

### (S)-2-((((9H-fluoren-9-yl)methoxy)carbonyl)amino)-3-(5-(methoxymethyl)-1,2,4-oxadiazol-3-yl)propanoic acid

Step 1: To a stirred solution of (tert-butoxycarbonyl)-L-asparagine (100.0 g, 430.6 mmol) in pyridine (250 mL) was added dropwise a solution of DCC (88.84 g, 430.6 mmol) in acetone (500 mL) at room temperature. The reaction mixture was stirred at 25° C. for 16 h. The reaction mixture was filtered and washed with acetone (2×100 mL). The filtrate was concentrated under reduced pressure. The residue was diluted with DCM (500 mL) and washed with 2N HCl (300 mL) and water (200 mL). The combined organic layer was dried over over Na₂SO₄, filtered and concentrated under reduced pressure. The crude compound was purified by acid base treatment (using NaHCO₃ and 2N HCl) to afford (S)-2-((tert-butoxycarbonyl)amino)-3-cyanopropanoic acid. MS ESI calculated for C₉H₁₄N₂O₄ [M+H]⁺ 215.10, found 215.11.

Step 2: To a stirred solution of (S)-2-((tert-butoxycarbonyl)amino)-3-cyanopropanoic acid (37.00 g, 172.7 mmol) in DCM (200 mL) was added tert-butyl (E)-N,N-diisopropylcarbamimidate (34.60 g, 172.7 mmol) at room temperature. During the reaction formation of a curdy white precipitate was formed. The reaction mixture was stirred at 45° C. for 5 h. Reaction mixture was filtered and washed with DCM (2×100 mL). The filtrate was concentrated under reduced pressure. The crude compound was purified by Biotage-isolera using a 120 g silica cartridge and the compound eluted with 30% of ethyl acetate in petroleum ether. Pure fractions were combined and concentrated under reduced pressure to afford tert-butyl (S)-2-((tert-butoxycarbonyl)amino)-3-cyanopropanoate. MS ESI calculated for C₁₃H₂₂N₂O₄ [M+H]⁺ 271.16, found 271.16.

Step 3: To a stirred solution of tert-butyl (S)-2-((tert-butoxycarbonyl)amino)-3-cyanopropanoate (45.0 g, 166 mmol) in DMSO (200 mL) were added sodium bicarbonate (105 g, 1.25 mol) and hydroxylammonium chloride (63.6 g, 916 mmol) at room temperature. The reaction mixture was stirred at 100° C. for 16 h. The reaction mixture was cooled to 25° C., then quenched with water (200 mL) and extracted with EtOAc (2×500 mL). The combined organic layer was dried over Na₂SO₄, filtered and concentrated under reduced pressure tert-butyl (S)-2-((tert-butoxycarbonyl)amino)-4-(hydroxyamino)-4-iminobutanoate. MS ESI calculated for C₁₃H₂₅N₃O₅ [M+H]⁺ 304.18, found 304.30.

Step 4: To a stirred solution of tert-butyl (S)-2-((tert-butoxycarbonyl)amino)-4-(hydroxyamino)-4-iminobutanoate (19.00 g, 62.63 mmol) in ACN (150 mL) were added cesium carbonate (40.81 g, 125.3 mmol), 2-methoxyacetyl chloride (8.156 g, 75.16 mmol) at room temperature. The reaction mixture was stirred at 25° C. for 3 h. Then the reaction mixture was stirred at 80° C. for 3 h. The reaction mixture was quenched with water (100 mL) and extracted with EtOAc (2×150 mL). The combined organic layer was dried over Na₂SO₄, filtered and concentrated under reduced pressure. The crude compound was purified by Biotage-isolera using a 120 g silica cartridge and the compound eluted with 40% of ethyl acetate in petroleum ether. Pure fractions were combined and concentrated under reduced pressure to afford tert-butyl (S)-2-((tert-butoxycarbonyl)amino)-3-(5-(methoxymethyl)-1,2,4-oxadiazol-3-yl)propanoate. MS ESI calculated for C₁₆H₂₇N₃O₆ [M+H]⁺ 358.19, found 358.41.

Step 5: To a stirred solution of tert-butyl (S)-2-((tert-butoxycarbonyl)amino)-3-(5-(methoxymethyl)-1,2,4-oxadiazol-3-yl)propanoate (11.0 g, 30.8 mmol) in DCM (60 mL) was added TFA (47.4 mL, 616 mmol) at room temperature. The reaction mixture was stirred at 25° C. for 8 h. The reaction mixture was concentrated under reduced pressure. The residue was co-distilled with toluene (20 mL) and dried under reduced pressure to afford (S)-2-amino-3-(5-(methoxymethyl)-1,2,4-oxadiazol-3-yl)propanoic acid. MS ESI calculated for C₇H₁₁N₃O₄ [M+H]⁺ 202.07, found 202.17.

Step 6: To a stirred solution of (S)-2-amino-3-(5-(methoxymethyl)-1,2,4-oxadiazol-3-yl)propanoic acid (6.20 g, 30.8 mmol) in acetone (30 mL) and H₂O (30 mL) were added sodium bicarbonate (5.18 g, 61.6 mmol) and (9H-fluoren-9-yl)methyl (2,5-dioxopyrrolidin-1-yl) carbonate (11.4 g, 33.9 mmol) at room temperature. The reaction mixture was stirred at 25° C. for 5 h. The reaction mixture was concentrated under reduced pressure. The residue was subjected to acid-base treatment and extracted with 10% MeOH in DCM (2×200 mL). The combined organic layer was dried over Na₂SO₄, filtered and concentrated under reduced pressure to provide crude compound. The crude compound was purified twice by Biotage-isolera using a 120 g silica cartridge and the compound eluted with 10% of MeOH in DCM. Pure fractions were combined and concentrated under reduced pressure. The isolated compound was washed with 10% diethyl ether in hexane and dried under reduced pressure to afford the compound.

The compound was further re-purified by Chiral SFC on Chiralpak-AS-H Column, 3*25 cm, 5μ, isocratic with 80% CO₂ and 20% (0.5% Formic Acid in IPA), flow rate 90 g/min, temperature 30.0° C., Back Pressure 120.0 bar, 75.0 mg per injection, 15.0 min stack time.

Pure fractions were combined, concentrated under reduced pressure and lyophilized separately to afford Peak 1 (R)-2-((((9H-fluoren-9-yl)methoxy)carbonyl)amino)-3-(5-(methoxymethyl)-1,2,4-oxadiazol-3-yl)propanoic acid (MS ESI calculated for C₂₂H₂₁N₃O₆ [M+H]⁺ 424.14, found 424.22. ¹H NMR (400 MHz, DMSO-d₆) VT at 90° C.: δ (ppm) 12.91 (br s, 1H), 7.87 (d, J=7.6 Hz, 2H), 7.50-7.71 (m, 3H), 7.41 (t, J=7.4 Hz, 2H), 7.31 (td, J=7.4 Hz, 1.0 Hz, 2H), 4.69 (s, 2H), 4.32-4.50 (m, 1H), 4.15-4.30 (m, 3H), 3.35 (s, 3H), 3.04-3.17 (m, 2H)) and Peak 2 (S)-2-((((9H-fluoren-9-yl)methoxy)carbonyl)amino)-3-(5-(methoxymethyl)-1,2,4-oxadiazol-3-yl)propanoic acid. MS ESI calculated for C₂₂H₂₁N₃O₆ [M+H]⁺ 424.14, found 424.22. ¹H NMR (400 MHz, DMSO-d₆) VT at 90° C.: δ (ppm) 12.80 (br s, 1H), 7.87 (d, J=7.6 Hz, 2H), 7.66 (d, J=7.2 Hz, 2H), 7.58 (br s, 1H), 7.41 (t, J=7.4 Hz, 2H), 7.25-7.36 (m, 2H), 4.69 (d, J=2.8 Hz, 2H), 4.38-4.50 (m, 1H), 4.14-4.32 (m, 3H), 3.35 (s, 3H), 3.05-3.18 (m, 2H).

### Preparation of Final Compounds:

### A. Generalized Procedure for Synthesizing Linear Peptide Precursors

Peptides in FIG. 1 were synthesized using standard solid-phase synthesis using Fmoc/tBu chemistry as exemplified in Chan, W. C.; White, P. D. “Fmoc Solid-Phase Synthesis: a Practical Approach”, Oxford University Press, Oxford, 2000; Steward, J.; Young, J. “Solid Phase Peptide Synthesis”, Pierce Chemical Company, Rockford, 1984.; Benoiton, N. L. “Chemistry of Peptide Synthesis”, CRC Press, New York, 2006; and Lloyd-Williams, P.; Albericio, F.; Giralt, E. “Chemical Approaches to the Synthesis of Peptides and Proteins”, CRC Press, New York, 1997.

During peptide chain elongation, the α-amino group of each amino acid was protected with a 9H-fluoren-9-ylmethoxycarbonyl group (Fmoc). To avoid any side reactions during the chain elongation steps, any reactive amino acid side chains also carry acid-labile protecting groups, effectively masking the reactive groups until removal upon treatment with strong acid. After completion of each coupling step, the Fmoc group of the N-terminal amino acid was removed with piperidine or 4-methylpiperidine and the resin was thoroughly washed to prepare for the coupling of the subsequent Fmoc-protected amino acid derivative.

The side chain protecting groups used were: tert-butyl (tBu) for 3Pal4CO2H, 3Pal4Ph4CO2H, alT, aMeD, aMeS, Bip4CO2H, daMeD, daMeS, hS, Phe4COOH, Phe4Pyrim5CO2H, Proc4OH, PyrimAla4Ph4CO2H, S, T; tert-butoxy-carbonyl (Boc) for Ac4cN, AlaPiperaz, aMeDab, aMeK, Dab, daMeK, K, PipH, Orn; trityl (Trt) for Q; 2,2,4,6,7-pentamethyldihydrobenzofuran-5-sulfonyl (Pbf) for R; and, β-methylpentyl ester (OMpe) for D.

Fmoc-protected amino acids were typically obtained from vendors such as Sigma-Aldrich, Novabiochem, Chem-Impex, and Combi-Block.

B. Synthetic Procedures used to Prepare Cyclic Peptides

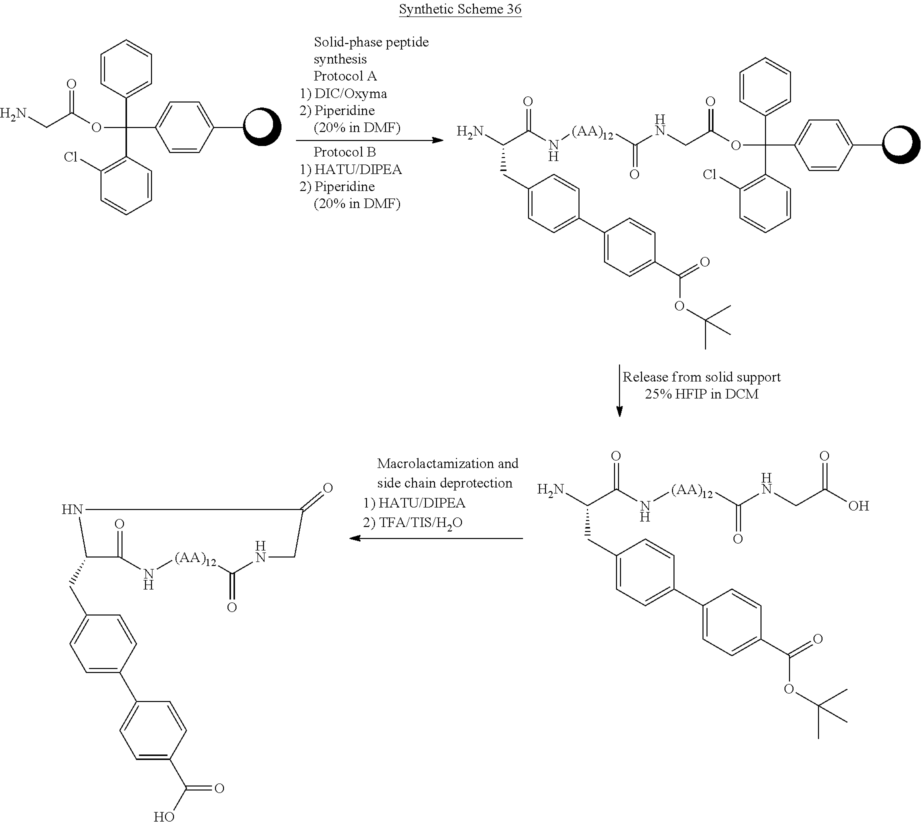

### Solid-Phase Synthesis of Peptides Protocol A

Peptides were synthesized on a Liberty Blue™ synthesizer from CEM Corporation, using standard solid-phase synthesis using Fmoc/tBu chemistry as summarized above in Scheme 36.

N,N′-Diisopropylcarbodiimide (DIC) with ethyl cyano(hydroxyimino)acetate (Oxyma) were used as coupling agents to form the amide bond between the free amino terminus of the resin-bound protected peptide and the carboxylic acid of the Fmoc-protected amino acid.

H-Gly-loaded 2-chlorotrityl resin (200-400 mesh, 0.79 mmol/g loading, 1% cross-linked polystyrene, Novabiochem) was used for synthesis. All the amino acids were dissolved at a 0.2 M concentration in DMF (N,N-dimethylformamide). The amino acids were activated with equimolar amounts of Oxyma solution (0.5 M in DMF), and a 2-fold molar excess of DIC solution (1.0 M in DMF). Alternatively, amino acids were dissolved at a 0.125 M concentration in DMF (N,N-dimethylformamide). The amino acids were activated with equimolar amounts of Oxyma Pure solution (0.125 M in DMF; with 0.05 M DIEA), and a 2-fold molar excess of DIC solution (0.25 M in DMF). Reactions were typically performed at the 25 μmol scale.

Every synthesis cycle included: Fmoc-amino acid deprotection by 20% piperidine in DMF (90° C. microwave assisted heating, 2 min) and coupling (potentially repeated twice for difficult couplings) with Fmoc-protected amino acid/DIC/Oxyma (5, 5, and 10 eq respectively; 90° C. microwave assisted heating, 2 min or 4 min). Cycles of Fmoc deprotection and Fmoc-protected amino acid coupling were repeated with the desired monomers until the full linear peptide was formed.

### Solid-Phase Synthesis of Peptides Protocol B

Alternatively, peptides were synthesized manually on a Biotage® Syro II peptide synthesizer using standard solid-phase synthesis using Fmoc/tBu chemistry as summarized above in Scheme 36. HATU with DIPEA were used as coupling agents to create the amide bond between the free amino terminus of the resin-bound protected peptide and the carboxylic acid of the Fmoc-protected amino acid. H-Gly-loaded 2-chlorotrityl resin (200-400 mesh, 0.79 mmol/g loading, 1% cross-linked polystyrene, Novabiochem) was used for synthesis. All the amino acids were dissolved at a 0.2 M concentration in 1:1 DMF:NMP. Reactions were typically performed at the 12 μmol scale.

Every synthesis cycle included: (1) Coupling (repeated twice) with Fmoc-protected amino acid/HATU/DIPEA (4, 4 and 8 eq, respectively; rt; 15 min). The mixture was filtered, and the peptidyl resin was washed with DMF (2×1 mL); (2) Fmoc deprotection (repeated three times): 20% 4-methyl piperidine in DMF (1 mL; rt; 3 min). The mixture was filtered, and the peptidyl resin was washed with DMF (4×1 mL). Cycles of Fmoc deprotection and Fmoc-protected amino acid coupling were repeated with the desired monomers until the full linear peptide was formed.

### Selective Cleavage of Protected Peptide and Macrolactamization

For the cleavage of the protected linear peptide from the solid support, the peptidyl resin (˜16 mg) was treated with 25% hexafluoroisopropanol (HFIP) in DCM for 20 min at rt, filtered, and the solvent was removed under reduced pressure. The resulting residue was dissolved in DMF (5 mL). HATU (0.44 eq) and DIPEA (2.5 eq) were added. The mixture was stirred for 5 min at rt. Then an additional 0.66 eq of HATU was added. Upon completion of the macrolactamization, monitored by UPLC-MS, the solvent was removed under reduced pressure.

### Final Side Chain Deprotection

A solution of TFA/H₂O/TIS (90/8/2, v/v/v, 1 mL) was added to the crude protected cyclic peptide. The mixture was stirred for 10 min at rt. Cold diethyl ether (15 mL) was added to the solution. The peptide was precipitated by centrifugation (3200 rpm, −10° C.). The precipitate was washed with diethyl ether (2×10 mL) and dried under vacuum overnight to give the crude deprotected cyclic peptide as a solid.

### HPLC Purification

Purification was performed by preparative reversed-phase high performance liquid chromatography (RP-HPLC) on Waters X-Bridge Prep C18 OBD Prep column (130 Å, 5 pm, column size 19*100 mm) using a Waters MS-Directed AutoPurification HPLC/MS system. Mobile phase: (A) 0.16% TFA in HPLC water and (B) 0.16% TFA in HPLC acetonitrile; flow rate: 25 mL/min; UV wavelength λ=215 nm; gradient: 25-50% B over 5 min. Alternatively purification was performed on Waters CSH-C18 Column (19×250 mm, 5 μM) using an Agilent, with 1290 infinity II preparative LC system and LC-MSD XT mass spectrometer. Mobile phase: (A) 0.1% formic acid in HPLC water and (B) 0.1% formic acid in HPLC acetonitrile; flow rate: 25 mL/min; UV wavelength λ=215 nm; gradient: 20% B over 2.5 min, 55% B over 2.5-20 min. UV absorbing fractions containing the target m/z ions were collected and the fractions containing product were confirmed by LC/MS.

Purity of fractions were confirmed by UPLC, which was measured by a reverse phase Waters Acquity UPLC-MS system. Column: Waters XSelect CSH C18 Column (130 Å, 2.5 μm, column size 2.1*50 mm). Mobile phase: (A) 0.05% TFA in HPLC water and (B) 0.05% TFA in HPLC acetonitrile; injection volume: 1 μL; flow rate: 1 mL/min; UV wavelength λ=215 nm; gradient: 5-100% B in 5 min. Lyophilization of combined fractions containing pure peptide resulted in the final cyclized product as a powder.

### Biological Assays:

### Procedure for IL-6 Assay in MRC5 Cells

The inhibition of IL-1β induced IL-6 secretion was evaluated in MRC5 cells. Recombinant human IL-13 (BioLegend 579404) at 2×EC₈₀ concentration was prepared in seeding medium, EMEM (ATCC 30-2003) with 0.025% BSA (Sigma A9576), 1× penicillin/streptomycin (Gibco 15070-063), 1×NEAA (Gibco 11140-050), 1×GlutaMax (Gibco 35050-061), and 1× sodium pyruvate (Gibco 11360-070). 20 μL of IL-1β was added to a 384-well collagen-coated plate (Corning 354664) and pre-incubated at ambient temperature for 1 hour with 200 nL of compound dispensed using an ECHO 555 liquid handler. Human lung fibroblast MRC5 cells (ATCC CCL-171) were added at a density of 3000 cells/20 μL per well. The cells were prepared by passaging three times in growth medium, EMEM (ATCC 30-2003) with 10% fetal bovine serum (Gibco 16140-071), 1× penicillin/streptomycin (Gibco 15070-063), 1×NEAA (Gibco 11140-050), 1× GlutaMax (Gibco 35050-061), and 1×sodium pyruvate (Gibco 11360-070) in collagen-coated T175 flasks (Greiner 661950) and harvested in seeding medium after 5 minutes of 0.25% trypsin-EDTA (Gibco 25200-056) digestion. The 384-well collagen-coated plate, containing a final volume of 40 μL, was incubated at 37° C., 5% CO₂ overnight. 5 μL of the conditioned medium was transferred to a 384-well AlphaLISA plate (PerkinElmer 6005350) for detection of IL-6 using the human AlphaLISA IL-6 kit (PerkinElmer AL223F) as per the manufacturer's protocol. 20 μL of the acceptor bead/biotinylated antibody mix was added to the 384-well AlphaLISA plate and incubated for 1 hour at ambient temperature. The donor bead mix was protected from light and 25 μL was added to the plate and incubated for 30 minutes at ambient temperature. The AlphaLISA plate was read on an EnVision multimode plate reader (Perkin Elmer model 2104) using the AlphaScreen setting (laser exc at 680 nm and emis at 570 nm). Dose response curves and IC₅₀ values were analyzed using a 4-parameter logistic equation in Spotfire software (Tibco, Palo Alto, CA).

The amino acid sequences, biological activities (MRC IC₅₀s), calculated monoisotopic masses, molecular formulas, calculated molecular weights and mass spectral data ([M+2H]/2+ or [M+H]+) of Example Nos. 1-103 (SEQ ID NOS: 1-465) are provided in FIG. 1.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A compound of the Formula (I) 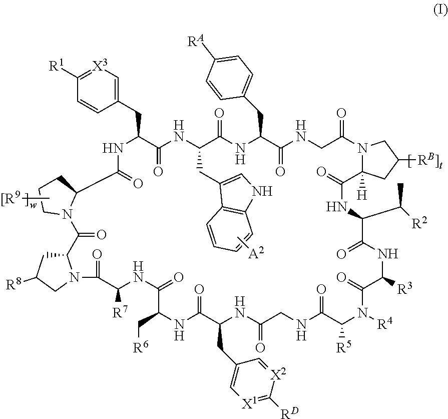
wherein:
R¹ is
(i) R^{1a}—C(O)N(H)—CH₂CH₂—O—, wherein
R^{1a} is
(a) C₁-C₃ alkyl or
(b) (CH₃)₃N—CH₂CH₂-M-, wherein:
M is —CH₂—, —CH₂CH₂—, —CH₂CH₂CH₂—, —O—CH₂CH₂—, or —O—CH₂CH₂—O—CH₂CH₂—;
(ii) (CH₃)₃N—CH₂CH₂—O—;
(iii) C¹, wherein C¹ is:
(a) phenyl or a 5- to 6-membered monocyclic heteroaryl, wherein said 5- to 6-membered monocyclic heteroaryl contains 1 to 3 heteroatoms independently selected from the group consisting of N, O, and S;
(b) a 5- to 6-membered heterocycloalkyl, wherein said 5- to 6-membered heterocycloalkyl is saturated and contains 1 or 2 heteroatoms selected from the group consisting of N, O, and S;
wherein C¹ is unsubstituted or substituted by 1 to 3 R^{C1} substituents independently selected from the group consisting of halo, C₁-C₃ alkyl, C₁-C₃ fluoroalkyl, carboxy, C₁-C₃ alkoxy, C₂-C₃ acyl, and C₁-C₃ alkoxymethyl;
(iii) (CH₃)₃N—CH₂CH₂—O—; or
(iv) a group 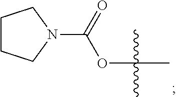 [SMILES: *OC(=O)N1CCCC1]
R^{A} is:
(i) —CO₂H;
(ii) —CO₂NH₂;
(iii) —S(O)₂NH₂; or
(iv) a 5-membered heteroaryl containing 2 or 3 heteroatoms independently selected from the group consisting of N, O, and S;
wherein the 5-membered heteroaryl is unsubstituted or substituted by 1 to 2 substituents independently selected from the group consisting of halo, C₁-C₃ alkyl, C₁-C₃ alkoxy, oxo, and C₁-C₃ alkoxymethyl;
R^{B} is amino, hydroxy, or fluoro;
R² is
(i) naphthyl; or
(ii) a 9- to 10-membered bicyclic heteroaryl, wherein said 9- to 10-membered bicyclic heteroaryl contains 1 to 2 heteroatoms independently selected from the group consisting of N, O, and S;
wherein R² is unsubstituted or substituted by 1 to 2 R^{2a} substituents independently selected from the group consisting of halo, C₁-C₃ alkyl and C₁-C₃ alkoxy;
R³ is —(CH₂)ₘCO₂H, —(CH₂)ₘCO₂NH₂, —(CH₂)ₘCO₂N(CH₃)₂, —(CH₂)ₘCH₂N(H)C(O)NH₂, or —(CH₂)ₘ—C³;
wherein C³ is a 5-membered heteroaryl containing 2 to 4 heteroatoms selected from the group consisting of N, O, and S;
wherein C³ is unsubstituted or substituted by 1 to 2 substituent selected from the group consisting of halo and C₁-C₃ alkyl;
R⁴ is C₁-C₃ alkyl;
R⁵ is H, C₁-C₃ alkyl, or —CH₂CO₂H;
or alternatively R⁴ and R⁵ together with the atoms to which they are attached form a pyrrolidinyl, piperidinyl or azepinyl ring;
X¹, X², and X³ are independently C(H) or N;
R^{D} is:
(i) ring CD, wherein ring CD is:
(a) phenyl; or
(b) cyclohexyl;
wherein ring CD is substituted by one A¹; wherein A¹ is —CO₂H, —CONH₂, or —N(H)SO₂CH₃;
(ii) hydroxy;
(iii) —CO₂NH₂; or
(iv) —OCH₂CH₂C(O)N(H)C(O)CH₃;
A² is H, halo or C₁-C₃ alkyl;
R⁶ is —H, —NH₂, —C(O)N(CH₃)₂; —(CH₂)ₙNH₂; —(CH₂)ₙN(H)CH₃; —(CH₂)ₙN(CH₃)₃; —OH; —(CH₂)ₙOH; —OCH₃; —(CH₂)ₙOCH₃; —(CH₂)ₙCH₃; —(CH₂)ₙN(H)C(O)NH₂; —(CH₂)ₙCH₂C(O)OH; —(CH₂)ₙN(H)C(O)CH₂CH₂O(CH₂CH₂—O)q-CH₂CH₂N(CH₃)₃; or —C⁶;
C⁶ is
(i) a 5- to 6-membered monocyclic aryl or heteroaryl, wherein said 5- to 6-membered monocyclic heteroaryl contains 1 to 3 heteroatoms independently selected from the group consisting of N, O, and S;
(ii) a 9- to 10-membered bicyclic aryl or heteroaryl, wherein said 9- to 10-membered bicyclic heteroaryl contains 1 to 4 heteroatoms independently selected from the group consisting of N, O, and S; and
(iii) a 3- to 8-membered mono- or bicyclic cycloalkyl;
wherein C⁶ is unsubstituted or substituted by 1 to 3 R^{C6} substituents independently selected from the group consisting of halo, amino, hydroxy, carboxy, C₁-C₃ alkyl, C₁-C₃ fluoroalkyl, C₁-C₃ alkoxy, and C₁-C₃ alkoxymethyl;
R⁷ is H, C₁-C₄ alkyl, —(CH₂)ᵤOH, —(CH₂)ᵤC(O)N(CH₃)₂, —(CH₂)ᵤN(CH₃)₃, —(CH₂)ᵤOCH₃, —(CH₂)ᵤC(O)NC(O)NH₂, —(CH₂)ᵤN(H)C(O)CH₂CH₂O(CH₂CH₂—O)ᵥ—CH₂CH₂N(CH₃)₃, or —CH₂—C⁷;
C⁷ is:
(i) a 5-membered heteroaryl containing 2 to 4 heteroatoms selected from the group consisting of N, O, and S; or
(ii) a 5- to 6-membered heterocycloalkyl, wherein said 5- to 6-membered heterocycloalkyl is saturated and contains 1 O atom;
wherein C⁷ is unsubstituted or substituted by 1 to 2 substituents selected from the group consisting of halo, C₁-C₃ alkyl, C₁-C₃ alkoxy, and C₁-C₃ alkoxymethyl;
R⁸ is —H, —F, —OH, —NH₂, —N(CH₃)₃, (CH₃)₃N—(CH₂)ᵣ—C(O)—, or (CH₃)₃N—CH₂CH₂—O—(CH₂CH₂—O)ₛ—CH₂CH₂—C(O)—
R⁹ is —C⁹ or —CH₂—C⁹;
C⁹ is phenyl or a 5- to 6-membered monocyclic heteroaryl, wherein said 5- to 6-membered monocyclic heteroaryl contains 1 to 2 heteroatoms independently selected from the group consisting of N, O, and S;
wherein C⁹ is unsubstituted or substituted by 1 to 3 R^{C9} substituents independently selected from the group consisting of halo, amino, hydroxy, C₁-C₃ alkyl, C₁-C₃ fluoroalkyl, and C₁-C₃ alkoxy;
wherein C⁹ is optionally substituted by 1 ring R^{C9C}, the ring R^{C9C} is:
(a) phenyl; or
(b) a 5- to 6-membered monocyclic heteroaryl containing 1 to 2 heteroatoms independently selected from the group consisting of N, O, and S;
subscript m is 1 or 2;
subscript n is 1, 2, or 3; and
subscript q is 1, 2, or 3;
subscript r is 3, 4, 5, or 6;
subscript s is 1 or 2;
subscript t is 0 or 1;
subscript u is 0, 1, 2, 3, or 4;
subscript v is 1, 2, or 3; and
subscript w is 0 or 1; or
a pharmaceutically acceptable salt thereof.

2. The compound of claim 1 or a pharmaceutically acceptable salt thereof, wherein:
C¹ is phenyl;
R² is indolyl or naphthyl;
C⁶ is phenyl, indolyl, pyridyl, pyridazinyl, or bicyclo[1.1.1]pentanyl; and
C⁹ is phenyl or pyrimidinyl.

3. The compound of claim 1 or a pharmaceutically acceptable salt thereof, wherein R¹ is CH₃—C(O)N(H)—CH₂CH₂—O— or 4-fluorophenyl.

4. The compound of claim 1 or a pharmaceutically acceptable salt thereof, wherein R² is unsubstituted or substituted naphthyl or indolyl.

5. The compound of claim 1 or a pharmaceutically acceptable salt thereof, wherein R³ is —(CH₂)ₘCO₂H.

6. The compound of claim 5 or a pharmaceutically acceptable salt thereof, wherein subscript m is 1.

7. The compound of claim 1 or a pharmaceutically acceptable salt thereof, wherein R⁴ and R⁵ are methyl.

8. The compound of claim 1 or a pharmaceutically acceptable salt thereof, wherein:
X¹ and X² are C(H) and
A¹ is carboxy.

9. The compound of claim 1 or a pharmaceutically acceptable salt thereof, wherein R⁶ is:
(i) a substituted or unsubstituted 5- to 6-membered monocyclic aryl or heteroaryl, wherein said 5- to 6-membered monocyclic heteroaryl contains 1 to 2 N atoms; or
(ii) a substituted or unsubstituted 9- to 10-membered bicyclic heteroaryl, wherein said bicyclic heteroaryl contains 1 to 2 N atoms.

10. The compound of claim 9 or a pharmaceutically acceptable salt thereof, wherein R⁶ is indolyl.

11. The compound of claim 1 or a pharmaceutically acceptable salt thereof, wherein R⁷ is H.

12. The compound of claim 1 or a pharmaceutically acceptable salt thereof, wherein R⁸ is H or —NH₂.

13. The compound of claim 1 or a pharmaceutically acceptable salt thereof, wherein R⁹ is substituted on position 4 of the illustrated pyrrolidinyl ring.

14. The compound of claim 13 or a pharmaceutically acceptable salt thereof, wherein C⁹ is substituted or unsubstituted phenyl or pyrimidinyl.

15. The compound of claim 1 or a pharmaceutically acceptable salt thereof, wherein R⁹ is —CH₂-(4-fluorophenyl) or pyrimidin-5-yl.

16. The compound of claim 1 or a pharmaceutically acceptable salt thereof, wherein:
R¹ is CH₃—C(O)N(H)—CH₂CH₂—O— or 4-fluorophenyl;
R² is unsubstituted or substituted naphthyl or indolyl;
R³ is —(CH₂)ₘCO₂H;
R⁴ and R⁵ are methyl;
X¹ and X² are C(H);
A¹ is carboxy;
A² is H;
R⁶ is:
(i) a substituted or unsubstituted 5- to 6-membered monocyclic aryl or heteroaryl, wherein said 5- to 6-membered monocyclic heteroaryl contains 1 to 2 N atoms; or
(ii) a substituted or unsubstituted 9- to 10-membered bicyclic heteroaryl, wherein said 9- to 10-membered bicyclic heteroaryl contains 1 to 2 N atoms;
R⁷ is H;
R⁸ is H or —NH₂; and
R⁹ is substituted on position 4 of the illustrated pyrrolidinyl ring.

17. The compound of claim 16 or a pharmaceutically acceptable salt thereof, wherein:
R⁶ is indolyl;
R⁹ is —CH₂-(4-fluorophenyl) or pyrimidin-5-yl, substituted on the 4-position of the illustrated pyrrolidinyl ring; and
subscript m is 1.

18. The compound of claim 1 or the pharmaceutically acceptable salt thereof, wherein the compound of Formula (I) has Formula (IA) 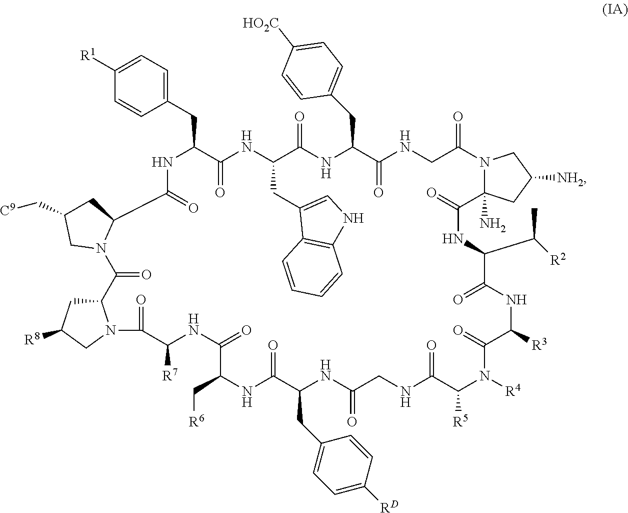
wherein:
R¹ is:
(i) R^{1a}—C(O)N(H)—CH₂CH₂—O—, wherein R^{1a} is:
(a) methyl; or
(b) (CH₃)₃N—CH₂CH₂—O—CH₂CH₂—;
(ii) (CH₃)₃N—CH₂CH₂—O—; or
(iii) 4-carboxyphenyl;
R² is a group 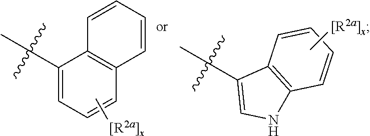
R^{2a} is halo or methyl;
subscript x is 0 or 1;
R³ is —CH₂CO₂H or —CH₂-tetrazolyl;
R⁴ and R⁵ are methyl;
or alternatively R⁴ and R⁵ together with the atoms to which they are attached form a piperidinyl ring;
R^{D} is 4-carboxyphenyl or hydroxy;
R⁶ is a group selected from: 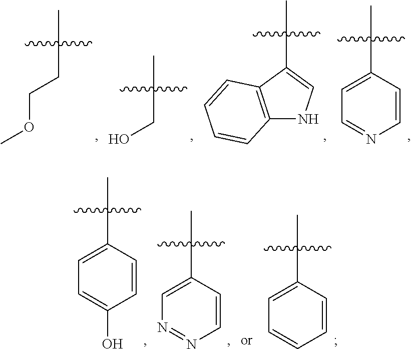
R⁷ is H or methyl;
R⁸ is —H, —NH₂, or —OH;
C⁹ is a group selected from

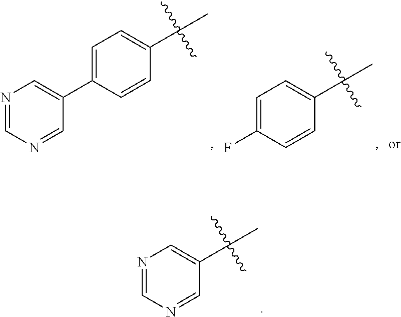 [SMILES: *c1ccc(-c2cncnc2)cc1 ; *c1ccc(F)cc1 ; *c1cncnc1]

19. The compound of claim 1 selected from the group consisting of SEQ ID NOS: 1-465, or a pharmaceutically acceptable salt thereof.

20. The compound of claim 1 selected from the group consisting of (SEQ ID NOs: 1, 2, 78, 80, 82, 92, 94, 95, 98, 99, 100, 101, 102, 215, 216, 217, and 218, respectively): 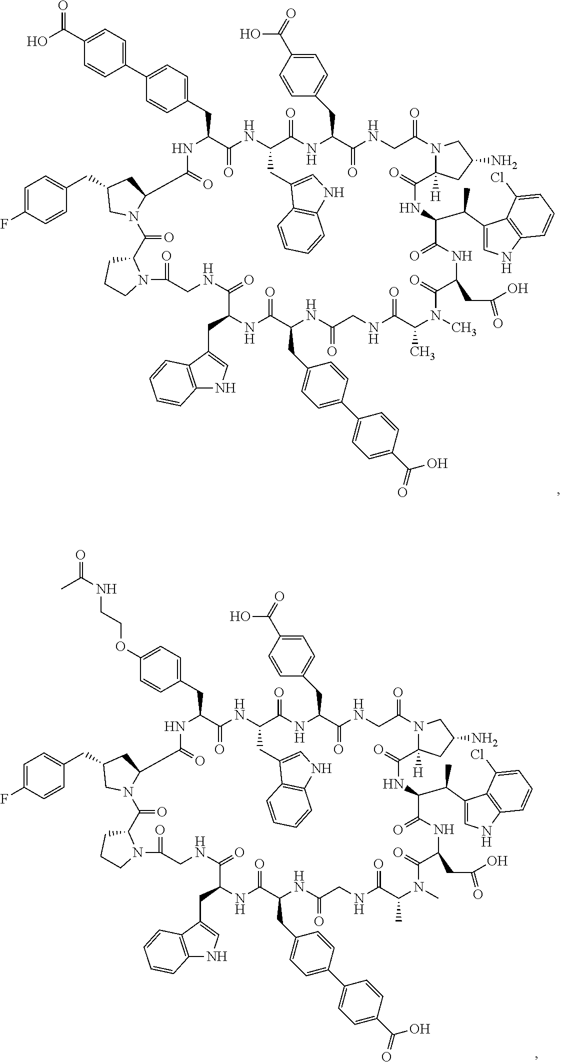 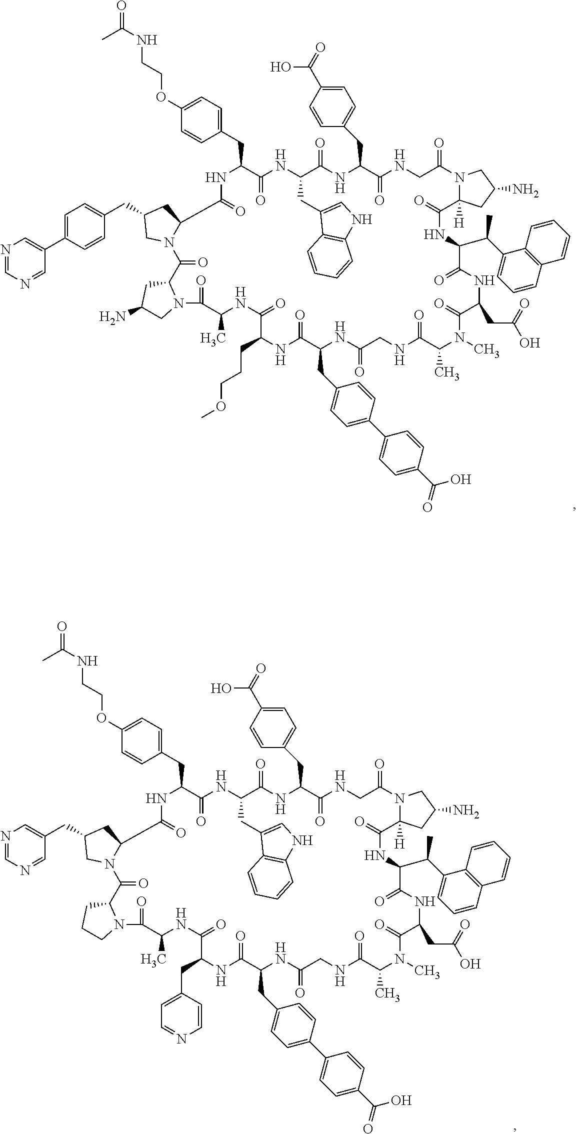 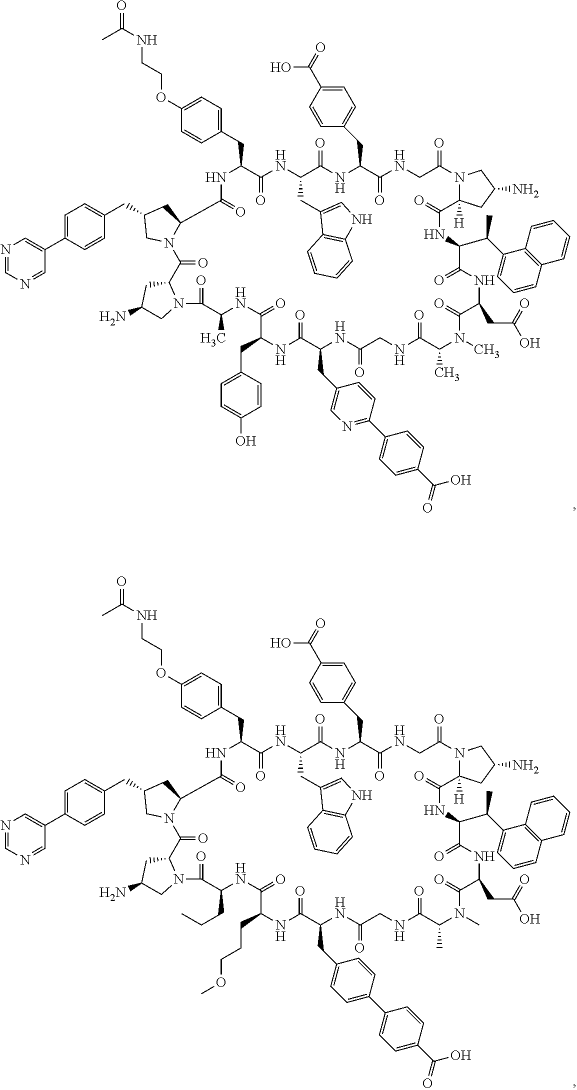 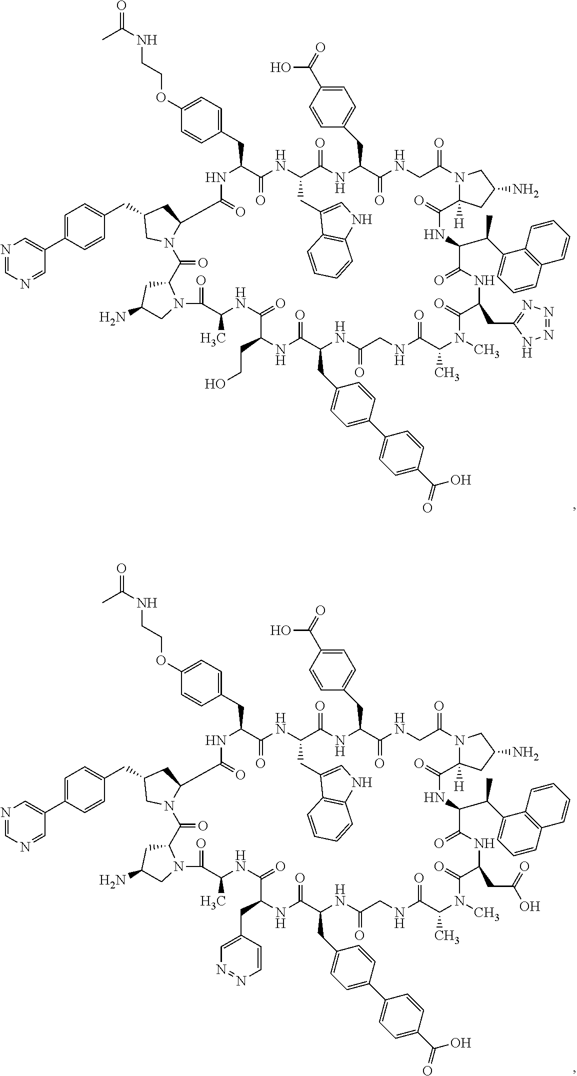 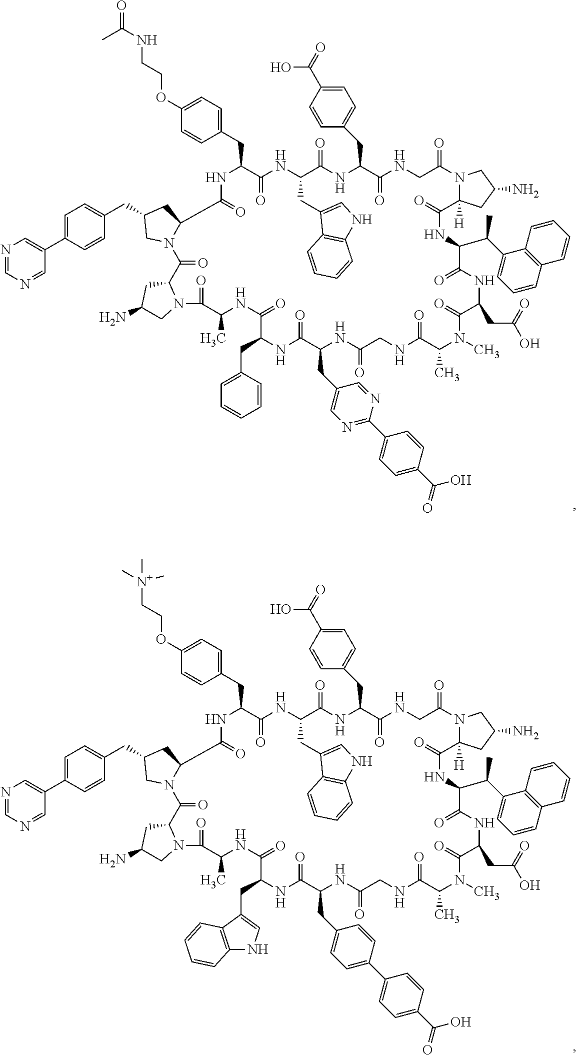 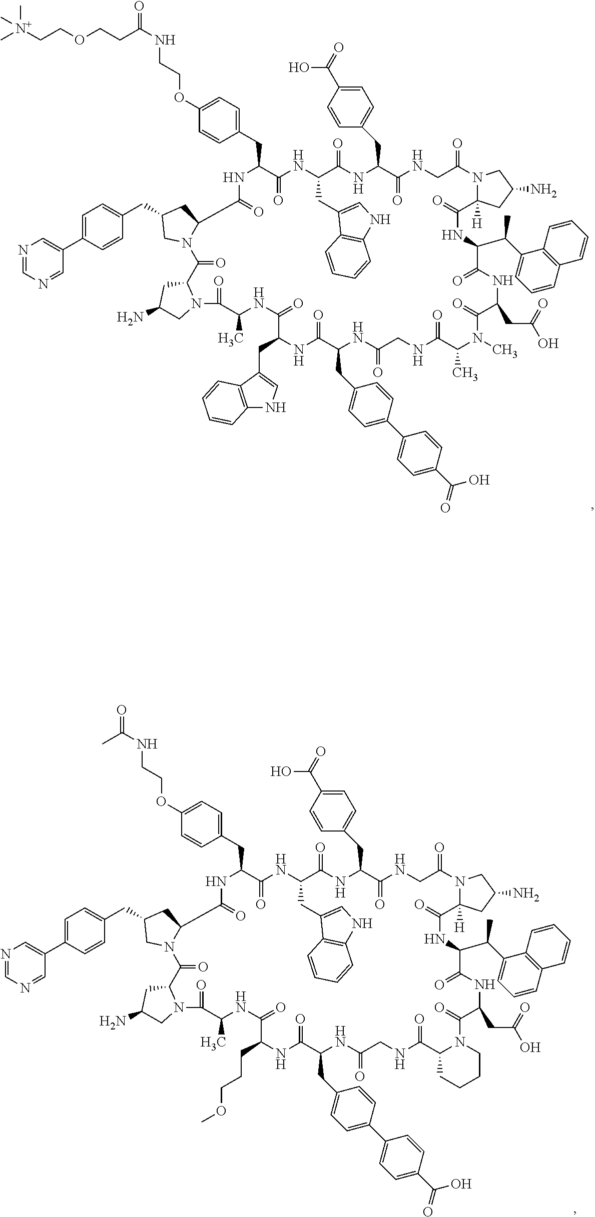 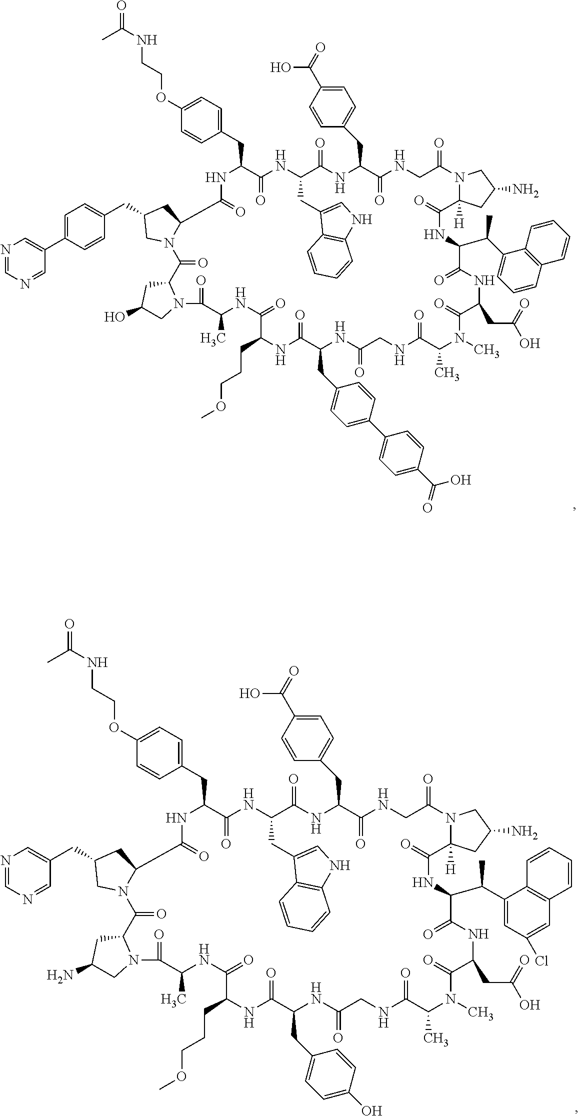 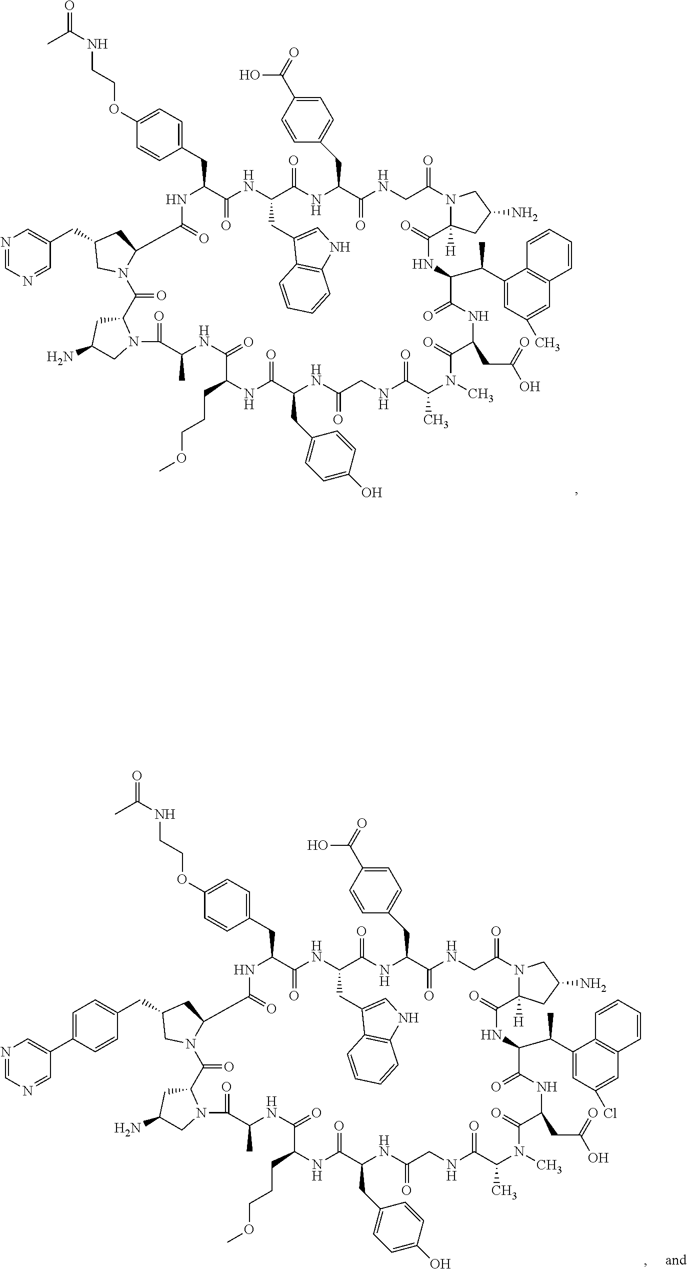 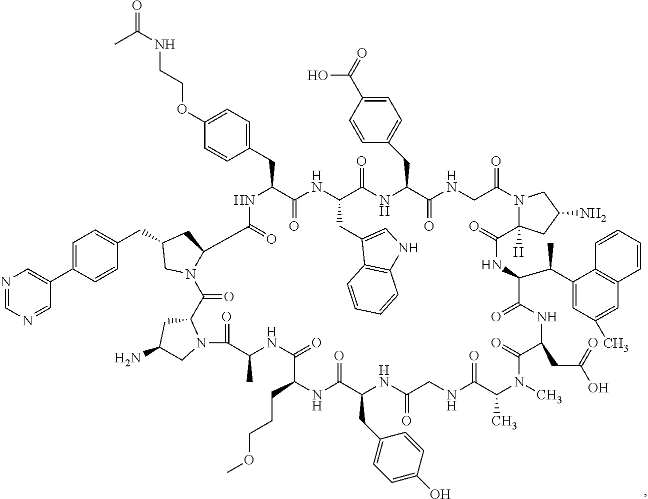 [SMILES: COCCC[C@@H]1NC(=O)[C@H](Cc2ccc(O)cc2)NC(=O)CNC(=O)[C@@H](C)N(C)C(=O)[C@H](CC(=O)O)NC(=O)[C@H]([C@@H](C)c2cc(C)cc3ccccc23)NC(=O)[C@@H]2C[C@@H](N)CN2C(=O)CNC(=O)[C@H](Cc2ccc(C(=O)O)cc2)NC(=O)[C@H](Cc2c[nH]c3ccccc23)NC(=O)[C@H](Cc2ccc(OCCNC(C)=O)cc2)NC(=O)[C@@H]2C[C@@H](Cc3ccc(-c4cncnc4)cc3)CN2C(=O)[C@H]2C[C@H](N)CN2C(=O)[C@H](C)NC1=O]
or a pharmaceutically acceptable salt thereof.

21. A pharmaceutical composition comprising the compound of claim 1 or a pharmaceutically acceptable salt thereof, and a pharmaceutically acceptable carrier.

22. A method of treating atherosclerosis, comprising administering a therapeutically effective amount of the compound of claim 1 or a pharmaceutically acceptable salt thereof to a subject in need thereof.

23. A method of treating vascular inflammation, comprising administering a therapeutically effective amount of the compound of claim 1 or a pharmaceutically acceptable salt thereof to a subject in need thereof.

24. A method of treating an inflammatory disorder, comprising administering a therapeutically effective amount of the compound of claim 1 or a pharmaceutically acceptable salt thereof to a subject in need thereof.
